(19) United States
(12) Patent Application Publication
(10) Pub. No.: US 2022/0267805 A1
(43) Pub. Date: Aug. 25, 2022

(54) CELLS, TISSUES, ORGANS, AND/OR ANIMALS HAVING ONE OR MORE MODIFIED GENES FOR ENHANCED XENOGRAFT SURVIVAL AND/OR TOLERANCE

(71) Applicants: eGenesis, Inc., Cambridge, MA (US); Hangzhou Qihan Biotechnology Co., Ltd., Hangzhou Zhejiang (CN)

(72) Inventors: Luhan YANG, Cambridge, MA (US); Yangbin GAO, Hangzhou (CN); Marc GUELL, Cambridge, MA (US); Yinan KAN, Cambridge, MA (US); Wenning QIN, Cambridge, MA (US)

(21) Appl. No.: 17/611,838

(22) PCT Filed: May 15, 2020
(86) PCT No.: PCT/CN2020/090440

§ 371 Date: Nov. 16, 2021

(30) Foreign Application Priority Data
May 16, 2019 (CN) PCT/CN2019/087310
May 16, 2019 (CN) PCT/CN2019/087314
Oct. 18, 2019 (CN) PCT/CN2019/112038
Oct. 18, 2019 (CN) PCT/CN2019/112039

(51) Int. Cl. C12N 15/877; C12N 15/90; A01K 67/027; C07K 14/74; C07K 14/705

(52) U.S. Cl. CPC C12N 15/8778; C12N 15/90; A01K 67/0275; C07K 14/70539; C07K 14/70596; C12N 2310/20; A01K 2217/075; A01K 2217/15; A01K 2227/108; A01K 2267/025; A01K 2217/052

(57) ABSTRACT

Provided are cells, tissues, organs, and/or animals having one or more modified genes for enhanced xenograft survival and/or tolerance. And methods of producing and using the cells, tissues, organs, and/or animals.

### CROSS REFERENCE TO RELATED APPLICATIONS

This application incorporates the disclosures of PCT/CN19/87310, filed May 16, 2019, PCT/CN19/87314 filed May 16, 2019, PCT/CN19/112038 filed Oct. 18, 2019, and PCT/CN19/112039, filed Oct. 18, 2019, in their entirety for all purposes.

### CROSS REFERENCE TO SEQUENCE LISTING

The contents of the text file submitted electronically herewith are incorporated herein by reference in their entirety: A computer readable format copy of the Sequence Listing (filename: EGEN_037_00 WO_SeqList_ST25.txt, date recorded: May 14, 2020, file size 189 kilobytes).

### BACKGROUND

The shortage of human organs and tissues for transplantation has grown over the last several decades and represents one of the most significant unmet medical needs. Xenotransplantation has the potential to provide an almost unlimited supply of transplant organs for patients with chronic organ failure. Similarities in organ size and physiology, coupled with genetic engineering to eliminate molecular incompatibilities, makes the pig the donor of choice for renal xenograft. Preclinical studies have demonstrated that porcine renal xenografts have supported life for weeks to months in non-human primate recipients (Higginbotham 2015, Iwase 2015b). However, as a result of the evolutionary distance between pigs and humans, porcine organs trigger rejection by the human immune system in a number of forms, including (i) hyperacute rejection, (ii) acute humoral rejection consisting of disordered thromboregulation and type II endothelial cell (EC) activation with leukocyte recruitment, (iii) thrombotic microangiopathy consisting of intravascular thrombosis with platelet consumption and EC activation, fibrin deposition, and thrombosis due to lack of thromboregulation, and (iv) chronic vasculopathy. These adverse events are due, at least in part, to molecular incompatibilities between the donor and the recipient, particularly with regard to genes involved in complement, coagulation, inflammatory, and immune response systems. The clinical use of xeno-organs (e.g., porcine) has been hindered by these immunological incompatibilities, which have thus far prevented the use of porcine cells, tissue, and vascularized porcine organs in clinical xenotransplantation.

Over the last two decades, several genetic modifications that diminish inter-species incompatibility between porcine and humans have been identified. However, these previously identified genetic modifications have not achieved long-term xenograft survival. Moreover, technical limitations with large-scale genome engineering have hindered the integration of these modifications in a single animal.

### SUMMARY

There is a need for developing porcine cells, tissues, organs, and/or porcine animals having a novel combination of gene modifications for use in xenotransplantation and for developing associated methods.

Accordingly, the present disclosure provides cells, tissues, organs, and animals comprising genetic modifications that result in enhanced immunological compatibility, as well as vectors and methods for use in generating these cells, tissues, organs, and animals, and the use of these cells, tissues, organs, and animals in xenotransplantation. In certain embodiments, the genetic modifications giving rise to enhanced immunological compatibility include one or more complement response genes (interchangeably referred to herein as complement toxicity genes), coagulation response genes (interchangeably referred to herein as coagulation genes), inflammatory response genes (interchangeably referred to herein as apoptosis/inflammation genes), immune response genes (interchangeably referred to herein as cellular toxicity genes), and/or immunomodulator genes.

In some aspects, the present disclosure provides isolated cells, tissues, organs, and animals comprising a plurality of transgenes of at least two types selected from the group consisting of inflammatory response transgenes, immune response transgenes, immunomodulator transgenes, or combinations thereof. In some aspects, the present disclosure provides for an isolated cell, tissue, organ, or animal comprising a plurality of transgenes, wherein the plurality of transgenes comprises at least one inflammatory response transgene, at least one immune response transgene, and at least one immunomodulator transgene. In some embodiments, the plurality of transgenes comprises at least three transgenes selected from the group consisting of inflammatory response transgenes, immune response transgenes, immunomodulator transgenes, or combinations thereof. In some embodiments, the inflammatory response transgenes are selected from the group consisting of tumor necrosis factor α-induced protein 3 (A20), heme oxygenase (HO-1 or HMOX1), Cluster of Differentiation 47 (CD47), and combinations thereof. In some embodiments, the immune response transgenes are selected from the group consisting of human leukocyte antigen-E (HLA-E), beta-2 microglobulin (B2M), and combinations thereof. In some embodiments, the immunomodulator transgene is selected from the group consisting of programmed death ligand 1 (PD-L1), Fas ligand (FasL), and combinations thereof. In some embodiments, the plurality of transgenes further comprises at least one coagulation response transgene. In some embodiments, the coagulation response transgenes are selected from the group consisting of Cluster of Differentiation 39 (CD39), thrombomodulin (THBD, TBM, or TM), tissue factor pathway inhibitor (TFPI), and combinations thereof. In some embodiments, the plurality of transgenes further comprises at least one complement response transgene. In some embodiments, the complement response transgene is selected from the group consisting of human membrane cofactor protein (hCD46 or simply CD46); human complement decay accelerating factor (hCD55 or simply CD55), human MAC-inhibitor factor (hCD59 or simply CD59), and combinations thereof.

In one aspect, the present disclosure provides isolated cells, tissues, organs, and animals comprising one or more transgenes, each independently selected from the group consisting of complement response transgenes (e.g., CD46, CD55, CD59); coagulation response transgenes (e.g., CD39, THBD or TBM, TFPI); inflammatory response transgenes (e.g., A20, HO-1, CD47); immune response transgenes (e.g., HLA-E, B2M); and/or immunomodulator transgenes (e.g., PD-L1, FasL). In certain embodiments, the cells, tissues, organs, or animals may further comprise one or more additional transgenes from other gene categories.

In certain embodiments, the isolated cells, tissues, organs, and animals provided herein comprise one or more complement response transgenes selected from the group consisting of hCD46, hCD55, and hCD59. In some of these embodiments, expression of one or more of the complement response transgenes is driven by a ubiquitous promoter.

In certain embodiments, the isolated cells, tissues, organs, and animals provided herein comprise one or more coagulation response transgenes selected from the group consisting of CD39, THBD, and TFPI. In some of these embodiments, expression of one or more of the coagulation response transgenes is driven by a tissue-specific promoter. In certain of these embodiments, the tissue-specific promoter is an endothelial-specific promoter, and in certain of these embodiments, the endothelial-specific promoter is a low expression endothelial-specific promoter.

In certain embodiments, the isolated cells, tissues, organs, and animals provided herein comprise one or more inflammatory response transgenes selected from the group consisting of A20, HO-1, and CD47. In some of these embodiments, expression of one or more of the inflammatory response transgenes is driven by a ubiquitous promoter, a tissue-specific promoter such as an endothelial-specific promoter, or any combination thereof.

In certain embodiments, the isolated cells, tissues, organs, and animals provided herein comprise one or more immune response transgenes selected from the group consisting of HLA-E and B2M. In some of these embodiments, expression of one or more of the immune response transgenes is driven by a ubiquitous promoter.

In certain embodiments, the isolated cells, tissues, organs, and animals provided herein comprise one or more immunomodulator transgenes, including but not limited to PD-L1, FasL, or both.

Expression of at least six of these transgenes at clinically effective levels in the cell, tissue, organ, or animals results in enhanced immunology compatibility. Accordingly, in certain embodiments, the isolated cells, tissues, organs, and animals provided herein comprise six or more transgenes, e.g., 6, 7, 8, 9, 10, 11, or 12 transgenes, selected from the group consisting of complement response, coagulation response, inflammatory response, immune genes, and immunomodulator transgenes. In certain of these embodiments, the cells, tissues, organs, or animals may comprise at least one transgene from each category. In other embodiments, certain categories of transgenes may be excluded. In certain embodiments, the complement response, coagulation response, inflammatory response, immune response, and/or immunomodulator transgenes may all be expressed at detectable and/or clinically effective levels simultaneously. In other embodiments, only specific subsets of transgenes may be expressed at clinically effective levels at certain timepoints or in response to certain signals. In these embodiments, expression of one or more of the transgenes may drop below detectable and/or clinically effective levels at certain timepoints.

In certain embodiments, the isolated cells, tissues, organs, and animals provided herein comprise the transgenes CD46, CD55, HLA-E, CD47, CD39, THBD, and TFPI.

In certain embodiments, the isolated cells, tissues, organs, and animals provided herein comprise the transgenes CD46, CD55, CD59, HLA-E, B2M, CD47, CD39, THBD, and TFPI.

In certain embodiments, the isolated cells, tissues, organs, and animals provided herein comprise the transgenes CD46, CD55, CD59, HLA-E, B2M, CD47, CD39, THBD, TFPI, A20, PD-L1, and HO-1.

Proteins or genes referred to herein may be those according to the following table. Sequences are incorporated by reference.

| | | | Example Human or |
|---|---|---|---|
| Protein/ | | | Pig UniProtKB |
| Gene | Name | Also Known As | reference |
| GGTA | N- | GGTA1 | P50127 |
| | acetyllactosaminide | | (GGTA1_PIG) |
| | alpha-1,3- | | |
| | galactosyltransferase | | |
| β4GALNT2 | β1,4 N- | B4GALNT2, | |
| | acetylgalactosaminyl | B4GAL | |
| | transferase | | |
| CMAH | Cytidine | | O19074 |
| | monophosphate-N- | | (CMAH_PIG) |
| | acetylneuraminic | | |
| | acid hydroxylase | | |
| CD46 | CD46 complement | | P15529 |
| | regulatory protein | | (MCP_HUMAN) |
| CD55 | CD55 Molecule | Decay- | P08174 |
| | (Cromer Blood | Accelerating | (DAF_HUMAN) |
| | Group) | Factor, DAF | |
| CD59 | CD59 Molecule | | P13987 |
| | (CD59 Blood | | (CD59_HUMAN) |
| | Group) | | |
| THBD | Thrombomodulin | CD141 Antigen | P07204 |
| | | | (TRBM_HUMAN) |
| TFPI | Tissue factor | Lipoprotein- | P10646 |
| | pathway inhibitor | Associated | (TFPI1_HUMAN) |
| | | Coagulation | |
| | | Inhibitor | |
| CD39 | CD39 antigen | Ectonucleoside | P55772 |
| | | triphosphate | (ENTP1_MOUSE) |
| | | diphospho- | |
| | | hydrolase 1, | |
| | | ENTPD1 | |
| HLA-E | Major | MHC Class I | P13747 |
| | Histocompatibility | Antigen E, | (HLAE_HUMAN) |
| | Complex Class I, E | MHC Class Ib | |
| | | antigen | |
| B2M | Beta-2- | Beta Chain Of | P61769 |
| | Microglobulin | MHC Class I | (B2MG_HUMAN) |
| | | Molecules | |
| CD47 | Cluster of | integrin | Q08722 |
| | Differentiation 47, | associated | (CD47_HUMAN) |
| | | protein | |
| A20 | A20 | TNF Alpha | P21580 |
| | | Induced Protein | (TNAP3_HUMAN) |
| | | 3, TNFAIP3 | |
| PD-L1 | Programmed cell | CD274, B7 | Q9NZQ7 |
| | death 1 ligand 1 | Homolog 1, | (PD1L1_HUMAN) |
| | | B7H1 | |
| FasL | Fas Ligand | FASL, CD95, | P48023 |
| | | Tumor Necrosis | (TNFL6_HUMAN) |
| | | Factor | |

In certain embodiments, the isolated cells, tissues, organs, and animals disclosed herein further comprise one or more modifications to a complement response gene, coagulation response genes, inflammatory response genes, immune response genes, and/or immunomodulator genes. For example, in certain embodiments in which the cell, tissue, organ, or animal is porcine, the cell, tissue, organ, or animal may comprise an alteration of the von Willebrand factor (vWF) gene, including in some instances alterations that result in humanization of the gene.

In certain embodiments, the cells, tissues, organs, and animals disclosed herein further comprise one or more modifications to other categories of genes. These modifications may include, for example, deletion or excision of all or part of the gene (i.e., knockout), or any other inactivation, disruption, or alteration. For example, in certain embodiments, the cells, tissues, organs, and animals may comprise a knockout, inactivation, or disruption of asialoglycoprotein receptor 1 (ASGR1). In certain embodiments, the cells, tissues, organs, and animals may be genetically modified to exhibit a reduced carbohydrate antigen response. For example, the cells, tissues, organs, or animals may comprise a knockout, inactivation, or disruption of one or more carbohydrate antigen-producing genes (e.g., glycoprotein α-galactosyltransferase 1 (GGTA), β1,4 N-acetylgalactosaminyltransferase 2 (B4GalNT2), cytidine monophosphate-N-acetylneuraminic acid hydroxylase (CMAH)).

In certain embodiments, the isolated cells, tissues, organs, and animals provided herein comprise the transgenes CD46, CD55, HLA-E, CD47, CD39, THBD, and TFPI, and further comprise a knockout, inactivation, or disruption of GGTA, B4GalNT2, and CMAH. In certain embodiments, the isolated cells, tissues, organs, and animals further comprise the transgenes CD59 and B2M, and in certain of those embodiments the isolated cells, tissues, organs, and animals further comprise the transgenes A20, PD-L1, and HO-1. In certain embodiments, these cells, tissues, organs, and animals exhibit enhanced immunological compatibility comprising reduced carbohydrate antigen response and enhanced coagulation, complement, inflammatory, and/or immune response.

In some embodiments, the isolated cells, tissues, organs, and animals provided herein are porcine, i.e., a porcine cell, porcine tissue, porcine organ, or a pig or progeny thereof. In certain of these embodiments, the cells, tissues, organs, or animals are free of porcine endogenous retroviruses (“PERV-free”). In certain of these embodiments, the “PERV-free” cells, tissues, organs, or animals do not produce xenotropic PERV virions. In certain of these embodiments, the “PERV-free” cells, tissues, organs, or animals do not produce PERV virions. In certain of these embodiments, the “PERV-free” cells, tissues, organs, or animals do not produce infectious PERV virions. In certain of these embodiments, the PERV-free cells, tissues, organs, and animals comprise the transgenes CD46, CD55, HLA-E, CD47, CD39, THBD, and TFPI, and optionally further comprise a knockout, inactivation, or disruption of GGTA, B4GalNT2, and and/or CMAH. In other embodiments, the PERV-free cells, tissues, organs, and animals comprise the transgenes CD46, CD55, CD59, HLA-E, B2M, CD47, CD39, THBD, and TFPI, and optionally further comprise a knockout, inactivation, or disruption of GGTA, B4GalNT2, and/or CMAH. In still other embodiments, the PERV-free cells, tissues, organs, and animals comprise the transgenes CD46, CD55, CD59, HLA-E, B2M, CD47, CD39, THBD, TFPI, A20, PD-L1, and HO-1, and optionally further comprise a knockout, inactivation, or disruption of GGTA, B4GalNT2, or CMAH.

In certain embodiments of the isolated cells and tissues provided herein, the cells or tissues are kidney or liver cells or tissues. In certain embodiments of the isolated organs provided herein, the organ is a kidney or a liver.

In another aspect, the present disclosure provides vectors comprising a plurality of transgenes of at least two types selected from the group consisting of inflammatory response transgenes, immune response transgenes, immunomodulator transgenes, or combinations thereof. In some embodiments, the plurality of transgenes comprises three types selected from the group consisting of inflammatory response transgenes, immune response transgenes, immunomodulator transgenes, or combinations thereof. In some aspects, the present disclosure provides for a vector comprising a plurality of transgenes, wherein the plurality of transgenes comprises at least one inflammatory response transgene, at least one immune response transgene, and at least one immunomodulator transgene. In some embodiments, the inflammatory response transgene is selected from the group consisting of A20, HO-1, CD47, and combinations thereof. In some embodiments, the immune response transgene is selected from the group consisting of HLA-E, B2M, and combinations thereof. In some embodiments, the immunomodulator transgene is selected from the group consisting of PD-L1, FasL, and combinations thereof. In some embodiments, the plurality of transgenes further comprises at least one coagulation response transgene. In some embodiments, the coagulation response transgene is selected from the group consisting of CD39, THBD, TFPI, and combinations thereof. In some embodiments, the plurality of transgenes further comprises at least one complement response transgene. In some embodiments, the complement response transgene is selected from the group consisting of CD46, CD55, CD59, and combinations thereof.

In other aspects, the present disclosure provides vectors for use in genetically modifying cells, tissues, organs, or animals to produce the cells, tissues, organs, or animals provided herein, including, for example, vectors for inserting (i.e., knocking in) one or more complement response, coagulation response, inflammatory response, immune response, and/or immunomodulator transgenes. In certain of these embodiments, the vectors comprise at least 6, 7, 8, 9, 10, 11, or 12 of the transgenes. In some of these embodiments, at least six of the transgenes are expressed from a single locus. Also provided herein are other components for use in genetically modifying cells, tissues, organs, or animals to produce the cells, tissues, organs, or animals provided herein, including, for example, CRISPR-based editing components such as guide RNAs (gRNAs) or endonucleases.

In certain embodiments, the vectors provided herein comprise the transgenes CD46, CD55, HLA-E, CD47, CD39, THBD, and TFPI. In certain of these embodiments, the vectors further comprise the transgenes CD59 and B2M. In certain of these embodiments, the vectors further comprise the transgenes A20, PD-L1, and HO-1, and in certain of these embodiments the vectors comprise the components set forth in FIG. 17-20, 31, or 48-50. In certain embodiments, the vectors comprise a sequence set forth in any of SEQ ID NOs:212-214.

Also provided herein in certain embodiments are methods of generating the isolated cells, tissues, organs, and animals provided herein. In certain of these embodiments, the methods comprise introducing one or more of the vectors provided herein. Accordingly, in certain embodiments, the cells, tissues, organs, and animals provided herein comprise one or more of the vectors disclosed herein.

In some embodiments, the methods disclosed and described herein comprise single copy polycistronic transgene integration through transposition, mono/bi-allelic site-specific integration through recombinase-mediated cassette exchange (RMCE), genomic replacement, endogenous gene humanization, or any combination thereof.

In certain embodiments of the methods provided herein wherein the cells, tissues, organs, and animals being generated are porcine, the methods further comprise knocking out or otherwise disrupting or inactivating one or more PERV genes, for example PERV pol, and in certain of these embodiments the resultant porcine cells, tissues, organs, or animals are PERV-free.

In another aspect, the present disclosure provides a transgenic pig liver having reduced liver damage and/or stable coagulation when exposed to non-pig blood, wherein reduced liver damage is assessed by determining the levels of bile production, one or more metabolic enzymes, and/or one or more serum electrolytes, and wherein stable coagulation is assessed by determining the levels of Prothrombin Time (PT) and International Normalized Ratio (PT-NIR), fibrinogen levels (FIB), and/or lower activated partial thromboplastin time (APTT). In some embodiments, the metabolic enzymes are selected from the group consisting of alanine aminotransferase (ALT), aspartate aminotransferase (AST), and albumin (ALB). In some embodiments, the serum electrolytes are potassium (K) and/or sodium (Na).

In some embodiments, the transgenic pig livers disclosed and described herein comprise native metabolic enzymes selected from the group consisting of alanine aminotransferase (ALT), aspartate aminotransferase (AST), and albumin (ALB).

### BRIEF DESCRIPTION OF THE DRAWINGS

FIGS. 1A-1C are charts displaying genotyping results of a complement factor 3 knockout (“C3-KO”) pig. FIG. 1A shows the sizes of the deletions introduced.

FIG. 1B illustrates the position of the indels. FIG. 1C lists sequences of the indels generated (SEQ ID NOs: 253-289).

FIG. 2 is a block diagram of a scheme depicting a Major Histocompatibility Complex class I (“MHC class I”) replacement strategy where the locus containing the SLA-1, SLA-2, and SLA-3 genes was flanked with IoxP sites.

FIGS. 3A and 3B are charts displaying genotyping results of a Major Histocompatibility Complex (MHC) class II knockout (“MHCII-KO”) pig genotype, specifically the MHCII gene DQA. FIG. 3A shows the positions and sized and indels having two insertions of 1 bp in positions 126 and 127 of the amplicon. FIG. 3B illustrates the position of one of the insertions.

FIGS. 4A and 4B are charts displaying genotyping results of another MHC class II-KO pig genotype, specifically the MHCII gene DRA. FIG. 4A shows the positions and sized and indels having two insertions of 1 bp in positions 106 and 107 of the amplicon. FIG. 4B illustrates the position of one of the insertions (SEQ ID NOs: 290-327).

FIG. 5 includes six charts showing the results of a fluorescence assisted cell sorting (FACS) analysis of an MHCII-KO pig (“H3-9P01”) and a wild-type (“WT”) pig.

FIG. 6 is a series of images depicting one or more phenotypes associated with the MHCII-KO phenotype.

FIG. 7 is a series of block diagrams illustrating a scheme for altering the PD-L1 gene.

FIG. 8 is a chart illustrating expression of PD-L1 as measured by qPCR using two amplicons.

FIG. 9 is a sequence listing showing alignment of porcine (SEQ ID NO: 329) and human (SEQ ID NO: 328) vWF protein. The A1 domain is highlighted in the box, whereas the potential glycosylation sites in the flanking region are labeled by dashes. The human specific residues that are deleted in pvWF is labeled with a horizontal line. The A1 and flanking region that were humanized is labeled with the half parenthesis.

FIG. 10 depicts a design of a homology-directed repair (“HDR”) vector targeting pvWF and two sgRNAs (SEQ ID NOs: 5 and 6).

FIG. 11 shows the screening results for HDR via SphI and BspEI digestion.

FIGS. 12A and 12B show sequencing results of a biallelic HDR clone obtained from FIG. 11 where vWF was targeted (SEQ ID NOs: 330-333). The chromatography of both sequencing results is illustrated with one line of overlapping sequences. The humanized A1 and flanking region is labeled with half parenthesis.

FIG. 13 is a graph depicting a species-specific platelet aggregation response induced by shear stress and monitored by light transmission for platelets isolated from WT (porcine A1-domain) or HDR targeted (human A1-domain) pigs.

FIG. 14 is a schematic of the porcine MHC class I locus. All classical MHCI genes are color coded. Unique flanking regions immediately next to the UTRs of the MHCI genes are labeled as green parenthesis. Four highly active sgRNAs (SEQ ID NOs: 1-4) selected from these regions are also shown.

FIG. 15 depicts fragmental deletion of the MHCI classical cluster induced using the sgRNAs in FIG. 14. FIG. 15A shows PCR amplicon across the unique regions of MHCI 5′, 3′ and 5′-3′ deletion junctions in the population of sgRNA transfected cells.

FIG. 15B shows that the 5′-3′ junction PCR was TOPO cloned and the sequencing results were aligned (SEQ ID NOs: 335-343) to the expected MHCI 5′-3′ junctions generated by MHC5′_sg1 and MHC3′_sg2.

FIG. 16 shows enrichment of MHCI negative cells using a porcine specific SLA-1 antibody.

FIG. 17 shows a transgene expression vector for expressing multiple transgenes (e.g. humanized transgenes) according to an embodiment disclosed and described herein. Payload 5 (Pig2.1): 12 transgenes, ubiquitous expression.

FIG. 18 shows a transgene expression vector for expressing multiple transgenes (e.g. humanized transgenes) according to an embodiment disclosed and described herein. Payload 9 (Pig2.2): 12 transgenes, endothelial-specificity.

FIG. 19 shows a transgene expression vector for expressing multiple transgenes (e.g. humanized transgenes) according to an embodiment disclosed and described herein. Payload 10 (Pig2.3): 12 transgenes, endothelial/islet-specificity.

FIG. 20 shows a transgene expression vector for expressing multiple transgenes (e.g. humanized transgenes) according to an embodiment disclosed and described herein. Payload 10-Exo (Pig2.4): 12 transgenes, endothelial-/islet-specificity, with pancreatic exocrine ablation.

FIG. 21 is a schematic showing pedigrees of genetically engineered source donor pigs described herein.

FIG. 22 demonstrates that genetically engineered pig fibroblasts having enhanced compatibility with human tissues show a significantly reduced binding affinity to human antibodies.

FIG. 23 demonstrates tissue-specific mRNA expression from genetically engineered pig primary fibroblasts or endothelial cells described herein. FIG. 23A is a schematic of a transgenic construct assembled using molecular cloning techniques. The CD46, CD55, and CD59 cassette was placed under control of the ubiquitous EF1α promoter, the HLA-E, B2M, and CD47 cassette was placed under control of the ubiquitous CAG promoter, the A20, PD-L1, HO-1 cassette was placed under control of the islet specific NeuroD promoter, and the THBD, TFPI, and CD39 cassette was placed under control of endothelial specific ICAM2 promoter. The transgenic construct was electroporated into porcine primary fibroblasts (FIG. 23B) or an immortalized porcine aortic endothelial cell line (PEC-A) (FIG. 23C) and mRNA expression determined by qRT-PCR.

FIG. 24 depicts transgene protein expression in Pig 2.0 (“3KO+12TG”) spleen and fibroblast cells.

FIG. 25 demonstrates that the genetically engineered pig fibroblasts having enhanced compatibility with human cells exhibited a significantly lower level of complement-mediated cell death.

FIG. 26 demonstrates that pig fibroblasts genetically engineered to express human HLA-E exhibit a reduced susceptibility to NK-mediated lysis.

FIG. 27 demonstrates that endothelial cells derived from GGTAKO+CD55KI pigs exhibit decreased formation of thrombin-antithrombin III (TAT) complexes.

FIG. 28 demonstrates that livers isolated from 4-7 pigs and perfused with human blood have increased bile production as compared to wild type (WT) livers.

FIG. 29 demonstrates that livers isolated from 4-7 pigs and perfused with human blood have improved liver function as assessed by makers of liver damage and serum electrolyte levels as compared to WT livers.

FIG. 30 demonstrates that livers isolated from 4-7 pigs and perfused with human blood have improved coagulation as compared to WT livers.

FIG. 31 shows a transgene expression vector according to an embodiment disclosed and described herein. Payload 13 (Pig2.5): 10 transgenes, bicistronic.

FIGS. 32A-B demonstrate that host monkeys transplanted with kidneys isolated from Payload 9 (FIG. 32A) and Payload 10 (FIG. 32B) donor pigs exhibit stable serum creatinine levels.

FIGS. 33A-B show hematocrit levels in host monkeys transplanted with kidneys isolated from Payload 9 (FIG. 33A) and Payload 10 (FIG. 33B) donor pigs.

FIGS. 34A-B show platelet counts in host monkeys transplanted with kidneys isolated from Payload 9 (FIG. 34A) and Payload 10 (FIG. 34B) donor pigs.

FIGS. 35A-B show fluctuations in white blood cell (WBC) counts in host monkeys transplanted with kidneys isolated from Payload 9 (FIG. 35A) and Payload 10 (FIG. 35B) donor pigs

FIG. 36 shows RNAseq expression data showing complement and cellular toxicity genes are expressed in samples collected from Payload 9 and Payload 10 pigs.

FIG. 37 shows FACS data showing complement and cellular toxicity proteins expressed in samples collected from Payload 5, Payload 9, and Payload 10 pigs.

FIGS. 38A-I show clinical labs following pig-to-baboon orthotopic liver xenotransplants (OLTx).

FIGS. 39A-F are representative images of H+E staining liver samples from OLTx.

FIGS. 40A-E demonstrate clinical labs following ex vivo xenoperfusion of genetically modified pig livers with human whole blood.

FIGS. 41A-H are representative images of H+E staining of xenoperfused pig livers.

FIG. 42 demonstrates that pulmonary vascular resistance (PVR) rise was significantly attenuated and delayed in ‘untreated’ Pig 2.0 (“3KO+12TG”) lungs perfused with human blood, relative to GalTKO.hCD55 lungs.

FIGS. 43A-D demonstrate binding of a panel of human serum to human T cells (FIG. 43A) and B cells (FIG. 43C), showing that high PRA sera are more likely than low PRA to stain human cells and binding of a panel of human sera to porcine T cells (FIG. 43B) and B cells (FIG. 43D). Sera from both low PRA patients and high PRA patients show high levels of binding to porcine targets.

FIG. 44 shows a panel of high PRA human sera show significantly lower levels of binding to genetically modified porcine aortic endothelial cells (Pig 2.0 (“3KO+12TG”) pAEC) compared to wild-type cells (WT pAEC). The Pig 2.0 cells lack aGal, Neu5Gc, and Sda.

FIGS. 45A-C demonstrate staining of Pig 2.0 (“3KO+12TG”) pAEC with serum taken from kidney (FIG. 45A), heart (FIG. 45B), and liver (FIG. 45C) xenotransplant recipient animals at various time points. Serum samples taken post-transplantation show a reduced level of binding, particularly the post liver xenotransplants.

FIGS. 46A-C demonstrate binding of human serum to wild-type (WT) and Pig 2.0 (“3KO+12TG”) pAEC (FIG. 46A), binding of human serum (FIG. 46B) or cynomolgus serum (FIG. 46C) to pAEC before and after IdeS treatment. IdeS effectively reduces human and cynomolgus IgG binding, while having no impact on the binding of intact IgM.

FIG. 47 shows a transgene expression vector according to an embodiment disclosed and described herein (SEQ ID NOs: 344 and 345). Payload 12F: 12 transgenes.

FIG. 48 shows a transgene expression vector according to an embodiment disclosed and described herein. Payload 12G: 12 transgenes.

FIG. 49 shows a transgene expression vector according to an embodiment disclosed and described herein. Payload 13A: 10 transgenes.

FIG. 50 shows RNAseq results demonstrating expression of complement & cellular toxicity genes.

FIG. 51A shows a scheme for CRISPR gene knockout and PiggyBac integration. CRISPR/Cas9 targeting 2 copies of GGTA1 gene, 2 copies of CMAH gene and 4 copies of B4GALNT2 gene were used to generate the 3KO, and CRISPR/Cas9 targeting the copies of PERV in Pig 2.0 (“3KO+9TG”) were used to generate PERV-KO cells. PiggyBac-mediated random integration was used to insert the 9 transgenes into the pig genome. The transgenes were expressed in 3 cassettes, with each cassette expressing 3 genes linked by Porcine 2A (P2A) peptide.

FIG. 51B shows results of sequencing of GGTA1 (SEQ ID NOs: 346-348), CMAH (SEQ ID NOs: 349-351), and B4GALNT2 (SEQ ID NOs: 352-356) knockout. The whole genome sequencing analysis revealed that in pig 2.0 (3KO+9TG) and pig 3.0 (3KO+9TG), i) the GGTA1 gene has −10 bp deletion in one allele and transgene vector insertion in another gene, ii) the CMAH gene has −391 bp deletion in one allele and 2 bp (AA) insertion in another allele and iii) the B4GALNT2 has −13, −14, −13, −14 in each of the 4 alleles of B4GALNT2 genes. All the modification occurs at the gRNA target sites, indicating the modification are mediated by on target activity of the CRISPR/Cas9 used.

FIG. 51C shows results of sequencing analysis of PERV knockout. The raw reads for Pig 2.0 (3KO+9TG) (˜2,000×) and 3.0 (˜20,000×) are shown below a schematic PERV gene structure. Reads are grouped by their sequence composition and shown proportionally to their coverage. The vertical line in red, blue, green and orange in the coverage track represent single nucleotide change from reference allele to T, C, A, G respectively.

FIG. 51D shows PCR analysis of the 9TG integration. Transgene integration of Pig 2.0 (3KO+9TG) and Pig 3.0 (3KO+9TG) have been validated at the genomic DNA (gDNA) level by PCR. The PCR gel image shows the presence of 9 human transgenes in gDNA from Pig 2.0 and Pig 3.0 fetus fibroblasts, whereas WT Pig fetus fibroblast and NTC (without the addition of gDNA) groups serve as negative control.

FIG. 51E shows normal karyotype for Pig 2.0 (3KO+9TG) and 3.0 (3KO+9TG) cells. Pig 2.0 (A) and Pig 3.0 (B) fibroblasts were karyotyped using Giemsa-staining-based G-banding technique. Metaphase spreads were analyzed using SmartType software. Both Pig 2.0 and Pig 3.0 show normal [36+XY] karyotypes.

FIG. 52A shows a heatmap of expression of the 9 transgenes. Transgene expression was measured by RNA-Seq in HUVEC endothelium, PUVEC endothelium, Pig 2.0 (3KO+9TG) PUVEC endothelium, Pig 2.0 ear fibroblast and Pig3.0 fetal fibroblast. Each row represents one transgene and each column represents one sample. The expression level is colored coded in blue-yellow-red to represent low-medium-high. The tissue type and payload information for each sample is labeled on top of the heatmap as color bars.

FIG. 52B shows analysis of 3KO and 9TG expression by FACS. Genetic modifications (KO and TG) of Pig 2.0 (3KO+9TG) and Pig 3.0 (3KO+9TG) have been validated at the protein level by FACS. Pig 2.0 and Pig 3.0 PUVECs show comparable TG expression level to human endogenous (HUVEC) in general, except for hCD39 (higher than human endogenous) and hTHBD (lower than human endogenous).

FIG. 52C shows immunofluorescence analysis of 3KO and 9TG expression. Genetic modifications (KO and TG) of Pig 2.0 (3KO+9TG) and Pig 3.0 (3KO+9TG) have been validated at the protein level in kidney cryosections by immunofluorescence (IF).

FIG. 53A shows binding of human antibodies to Pig 2.0 (3KO+9TG) and 3.0 (3KO+9TG) cells. Pig 2.0 and Pig 3.0 PUVECs substantially attenuate the antibody binding to human IgG and IgM compared to their WT counterpart. Antibody binding of pooled human serum to PUVECs and HUVECs (positive control) was measured by FACS, respectively. Error bars indicate mean±s.d. (n=3).

FIG. 53B shows complement toxicity to WT pig, Pig 2.0 (3KO+9TG), Pig 3.0 (3KO+9TG) and HUVEC cells. Pig 2.0 and Pig 3.0 PUVECs reveal comparable antibody-dependent complement toxicity compared to HUVEC, which is significantly lower compared to WT PUVEC. Error bars indicate mean±s.d. (n=4).

FIG. 53C shows NK-mediated cytotoxicity to WT pig, Pig 2.0 (3KO+9TG), Pig 3.0 (3KO+9TG) and HUVEC cells. Pig 2.0 and Pig 3.0 PUVECs reveal significantly lower NK-mediated cytotoxicity compared to their WT counterpart. Error bars indicate mean±s.d. (n=3).

FIG. 53D shows phagocytosis of Pig 2.0 (3KO+9TG) and 3.0 (3KO+9TG) splenocytes by human macrophages. Pig 2.0 and Pig 3.0 splenocytes show reduced phagocytosis by human macrophage cell line. CFSE-labeled Pig 2.0 and Pig 3.0 splenocytes (target cells, T) were incubated with CD11b-labeled human macrophage cell line (effector cells, E) for 4 hours at 37° C., respectively. 2 different E:T ratios, 1:1 and 1:5, were performed. Phagocytosis of CFSE-labeled targets were measured by FACS, where the region of non-phagocytosing macrophages is shown in the upper left quadrants (Q1), and region of phagocytosing macrophages is shown in the upper right quadrants (Q2). Phagocytic activity was calculated as Q2/(Q1+Q2)×100%.

FIG. 53E shows level of thrombin-antithrombin (TAT) formation by WT pig, Pig 2.0 (3KO+9TG), Pig 3.0 (3KO+9TG) and HUVEC cells. Pig 2.0 and Pig 3.0 PUVECs mediate very low level of thrombin-antithrombin (TAT) formation, which is comparable to HUVEC and significantly lower than WT PUVEC, upon incubation with whole human blood for indicated time. Error bars indicate mean±s.d. (n=4).

FIG. 53F shows ADPase activity of the CD39 transgene. Pig 2.0 (3KO+9TG) and Pig 3.0 (3KO+9TG) PUVECs show significantly higher CD39 ADPase biochemical activity compared to WT PUVEC and HUVEC. (A) Human transgene CD39 mRNA are expressed higher than endogenous CD39 in Pig 2.0 and Pig 3.0. (B) FACS revealed that Pig 2.0 and Pig 3.0 have higher human CD39 protein expression than WT PUVEC and HUVEC. (C) Pig 2.0 and Pig 3.0 PUVECs have significantly higher ADPase biochemical activity of CD39 as measured by phosphate concentration when incubated with ADP. The higher CD39 ADPase biochemical activity is consistent with its higher CD39 protein expression level in Pig 2.0 and Pig 3.0. Error bars indicate the standard deviation (n=6).

FIG. 53G shows TFPI function in 3.0 cells. Activated Pig 2.0 (3KO+9TG) and Pig 3.0 (3KO+9TG) PUVECs express human TFPI on cell surface and show significantly higher binding ability to human Xa compared to WT PUVEC and HUVECs in vitro. (A) RNA-Seq revealed that Pig 2.0 PUVECs express more human TFPI than its endogenous level in HUVECs, and the porcine TFPI level in WT PUVECs (n=2). (B) Activated Pig 2.0 PUVECs show significantly higher Xa binding ability compared to WT PUVECs and HUVECs in vitro. Left panel: The standard curve measures linear regression between the concentration of human recombinant TFPI (rTFPI) protein and the unbound Xa level. Right panel: The tTFPI level projected from the unbound Xa level using the standard curve on the left measures the TFPI Xa Binding ability in Pig 2.0 EC, WT PUVECs and HUVECs with and without PMA activation. PMA (1 μM): PUVECs and HUVECs were activated by PMA for 6 hours, which leads to the translocation of hTFPI from cytosol to the cell membrane. Error bars indicate the standard deviation (n=4).

FIGS. 54A, 54B, 54C, 54D, and 54E show normal phenotypes of Pig 1.0 and 2.0 pigs (3KO+9TG). Pig 1.0 and Pig 2.0 show similar pathophysiology, compared with WT pigs in terms of complete blood count (A), liver (B), heart (C) and kidney function (D), and coagulation function (E). The sample numbers for Pig 1.0, Pig 2.0 and WT pigs are 18, 16 and 21, respectively. “no sig” denotes no statistical significance among the Pig 1.0, Pig 2.0 and WT groups by student's t-test.

FIG. 55 shows Mendelian inheritance of PERV-KO. The genetic modification of PERV-KO can be inherited following Mendelian genetics during natural mating production. The x-axis represents the total number of shifted bases calculated as the sum of insertions subtracting the sum of deletions. The y-axis represents the percent of reads. The red and green color indicate frameshift or not respectively. One Pig 1.0 pig mated with wild type Bama pig and generated 11 piglets. The liver, kidney, and heart tissue of one offspring piglet were analyzed by high-throughput DNA sequencing together with parental fibroblasts to assess the inheritance of the PERV-KO modifications. Pig 1.0 has 100% of PERV copies to be knockout, while the WT pig has ˜80% PERV copies at the same size as the WT length (Insertions-Deletions=0). Of note, some PERV copies in the WT sample might be non-functional or carry KO. In comparison, the liver, kidney and heart of the offspring pig has only ˜50% PERV copies to carry knockout. The pattern is similar among tissues, indicating that the PERV-KO modification is stably inherited following Mendelian genetics among different tissues.

FIGS. 56A, 56B, and 56C show Mendelian inheritance of the 9TG construct and the 3KO through breeding. The genetic modifications (3KO and 9TG) of this iteration of Pig 2.0 can be transmitted to the next generation following Mendelian genetics through natural mating production, as validated at genomic DNA (A), mRNA (B) and protein Level (C). We mated 9 WT pigs with Pig 2.0, and 11 3KO pigs with Pig 2.0, respectively, and detected the presence of 3KO and 9TG in the F1 progeny. (A) For the 9TG, approximately half of the progeny of Pig 2.0× WT pigs and Pig 2.0×3KO pigs carry the transgenes in the genome. For GGTA1, CMAH and B4GALNT2, the progeny of Pig 2.0× WT pigs are all heterozygous knockout, and the progeny of Pig 2.0×3KO pigs are all homozygous knockout. Of note, B4GALNT2 was analyzed as having four alleles because of the inclusion of its highly homologous pseudogene. (B) Approximately half (5/11) of the progeny of Pig 2.0×3KO pigs carry the mRNA corresponding to the 9TG in their mRNA transcripts. (C) FACS analysis validated the inheritance of 3KO and 9TG for Pig 2.0×3KO and Pig 2.0× WT pigs as reduced or absence of cell surface glycans or presence of human proteins.

### DETAILED DESCRIPTION

### I. Definitions

The terms “pig”, “swine” and “porcine” are used herein interchangeably to refer to anything related to the various breeds of domestic pig, species Sus scrofa.

The term “biologically active” when used to refer to a fragment or derivative of a protein or polypeptide means that the fragment or derivative retains at least one measurable and/or detectable biological activity of the reference full-length protein or polypeptide. For example, a biologically active fragment or derivative of a CRISPR/Cas9 protein may be capable of binding a gRNA, sometimes also referred to herein as a single guide RNA (sgRNA), binding a target DNA sequence when complexed with a guide RNA, and/or cleaving one or more DNA strands.

The terms “treatment,” “treating,” “alleviation” and the like, when used in the context of a disease, injury or disorder, are used herein to generally mean obtaining a desired pharmacologic and/or physiologic effect, and may also be used to refer to improving, alleviating, and/or decreasing the severity of one or more symptoms of a condition being treated. The effect may be prophylactic in terms of completely or partially delaying the onset or recurrence of a disease, condition, or symptoms thereof, and/or may be therapeutic in terms of a partial or complete cure for a disease or condition and/or adverse effect attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms).

The term “simultaneously” is used herein to refer to an event that occurs at the same time as another event, such as within seconds, milliseconds, microseconds, or less when compared to the occurrence of another event.

The term “knockout” (“KO”) or “knocking out” is used herein to refer to a deletion, deactivation, or ablation of a gene or deficient gene in a pig or other animal or any cells in the pig or other animal. KO, as used herein, can also refer to a method of performing, or having performed, a deletion, deactivation or ablation of a gene or portion thereof.

The term “knockin” (“KI”) or “knocking in” is used herein to refer to an addition, replacement, or mutation of nucleotide(s) of a gene in a pig or other animal or any cells in the pig or other animal. KI, as used herein, can also refer to a method of performing, or having performed, an addition, replacement, or mutation of nucleotide(s) of a gene or portion thereof.

### II. Cells, Tissues, Organs, and Animals

Porcine xenografts are broadly compatible with human organ size and physiology and are ethically acceptable to the US general population. However, xenotransplanted porcine tissue elicits a complex series of events leading to graft rejection including: hyperacute rejection due to the presence of preformed antibodies to pig antigens, complement activation and hypercoagulability, and heightened innate and adaptive immune responses due to molecular incompatibilities. The present disclosure uses genetic engineering approaches to address current shortcomings of xenotransplantation.

In particular, a number of immunological and functional challenges exist involving innate and adaptive immune function. Complement- and coagulation-mediated dysfunction arises due to molecular incompatibility between the donor porcine tissue and human physiology and leads to acute xenograft failure. Pre-formed antibodies to α-1, 3-galactosyl-galactose (aGal) epitopes initiate hyperacute graft rejection through activation of complement. Genetic inactivation of the glycoprotein α-galactosyltransferase 1 gene (GGTA1) can reduce this rapid graft destruction. Protection is further improved through over-expression of genes for human complement regulatory proteins (hCRPs) CD46 (membrane cofactor protein), CD55 (complement decay accelerating factor), and CD59 (MAC-inhibitory protein).

Most non-Gal xenoantibodies recognize the sialic acid N-glycolylneuraminic acid (Neu5Gc) which is synthesized by the cytidine monophosphate-N-acetylneuraminic acid hydrolase (CMAH) gene. This gene is inactive in humans and, as such, porcine Neu5Gc is immunogenic in humans. Therefore, porcine CMAH likely must be inactivated for clinical success in xenotransplantation. While expression of complement regulators and knockout of GGTA1 (GTKO) reduces hyperacute rejection, these genetic modifications do not impact acute vascular rejection (AVR).

Coagulation dysfunction, including thrombotic microangiopathy and systemic consumptive coagulopathy, has persisted even with GTKO and overexpression of hCRP due primarily to molecular incompatibilities in the coagulation system between pig and non-human primates (NHP).

Despite attempts by others to generate transgenic pigs for safe xenotransplantation, these transgenic pigs carried only a limited number of transgenes due to construct capacity constraints and transcription interference between transgenes. These methods proved insufficient to overcome xenograft incompatibility. For example, US Patent Publ. No. 2018/0249688 utilized multi-cistronic expression vectors with different combinations of transgenes. Importantly, these multi-cistronic vectors comprised only 4 transgenes and were used to produce pigs having 6 genetic modifications, including KO of alpha Gal (GTKO). In the present disclosure, a combination of KO, KI, and genomic replacement strategies are utilized. For the first time, PERV-free pigs have been produced expressing more than 6 transgenes from a single locus.

The examples described and disclosed herein demonstrate that porcine complement factors can be KO′d and that viable pigs can be produced having one or more modified MHC Class I genes, inactivation of MHC Class II genes, KI of PD-L1 to reduce adaptive immunity-based rejections, modified porcine vWF to modulate platelet aggregation, and deletions of porcine MHC Class I genes. These examples provided a platform to achieve a greater number of genetic modifications within the same pig. From this work, porcine cells were genetically modified with more than six transgenes to generate immunologically compatible cells, tissues, organs, pigs, and progeny. Using CRISPR-Cas9, multiple genes were functionally knocked out, including GGTA1, CMAH, and B4GALNT2, to eliminate the glycans that are recognized by human preformed anti-pig antibodies. In addition, either nine or twelve human transgenes were integrated into a single multi-transgene cassette in the pig genome. Specifically, pigs have been produced utilizing CRISPR-mediated non-homologous end joining (NHEJ) to disrupt the 3 major xenogenic carbohydrate antigen-producing genes (“3KO”; GGTA1, B4GALNT2 and CMAH) coupled with PiggyBAC-mediated random integration of the 9 transgenes CD46, CD55, CD59, CD39, CD47, HLA-E, B2M, THBD, and TFPI or the 12 transgenes (CD46, CD55, CD59, HLA-E, B2M, CD47, CD39, THBD, TFPI, A20, PD-L1, and HO-1) into the porcine genome. A further advancement is to use source donor pigs harboring the 3KO and 9T or 12TG modifications on a PERV-free background. From there, source donor pigs will also be genetically engineered to carry additional genetic modifications, including humanization of the vWF gene and deletion or disruption of the asialoglycoprotein receptor 1 (ASGR1) and endogenous B2M genes, among others.

The present disclosure provides cells, tissues, organs, and animals having multiple modified genes, and methods of generating the same. In some embodiments, the cells, tissue, organs, are obtained from an animal, or is an animal. In some embodiments, the animal is a mammal. In some embodiments, the mammal is a non-human mammal, for example, equine, primate, porcine, bovine, ovine, caprine, canine, or feline. In some embodiments, the mammal is a porcine.

Modification of genes in accordance with the present disclosure serves to improve molecular compatibility between the donor and the recipient and to reduce adverse events, including hyperacute rejection, acute humoral rejection, thrombotic microangiopathy, and chronic vasculopathy. For example, hyperacute rejection occurs in a very short time span, typically within minutes to hours after transplantation and results from pre-formed antibodies that activate complement and graft endothelial cells, in turn causing pro-coagulation changes that lead to hemostasis and eventually destruction of the grafted organ. In certain embodiments, the cells, tissues, organs, and animals generate a reduced hyperacute rejection.

In some embodiments, the present disclosure provides for one or more cells, tissues, organs, or animals having multiple modified genes. In some embodiments, the cell, tissue, organ, or animal has been genetically modified such that multiple genes have been added, deleted, inactivated, disrupted, a portion thereof has been excised, or the gene sequence has been altered. In some embodiments, the cell, tissue, organ, or animal has 5, 6, 7, 8, 9, 10, 11, 12, 13, 14, 15, 16, 17, 18, 19, or 20 genes that have been modified. In some embodiments, the 5, 6, 7, 8, 9, 10, 11, 12, 13, 14, 15, 16, 17, 18, 19, or 20 genes that have been modified are expressed from a single locus. In some embodiments, the 5, 10, or 12 genes that have been modified are expressed from a single locus. In some embodiments, the 12 genes that have been modified are expressed from a single locus. In some embodiments, the cell, tissue, organ, or animal has more than 20, more than 15, more than 10, more than 5, more than 3, or 2 genes that have been modified. In some embodiments, the cell, tissue, organ, or animal has more than 10, more than 5, more than 3, more than 2, or more than 1 gene that has been modified. In some embodiments, the cell, tissue, organ, or animal has one copy of the modified gene and in other embodiments, the cell, tissue, organ, or animal has more than one copy of the one or more modified genes, such as more than 2, more than 3, more than 4, more than 5, more than 6, more than 7, more than 8, more than 9, more than 10, more than 15, more than 20, more than 25, more than 30, more than 35, more than 40, more than 50, more than 60, more than 70, more than 80, more than 90, or more than 100 copies of the modified gene. In some embodiments, the cell has between 100 copies and about 1 copy, 90 copies and about 1 copy, 80 copies and about 1 copy, about 70 copies and about 1 copy, 60 copies and about 1 copy, between about 50 copies and about 1 copy, between about 40 copies and about 1 copy, between about 30 copies and about 1 copy, between about 20 copies and about 5 copies, between about 15 copies and about 10 copies, or between about 5 copies and about 1 copy of one or more modified genes.

In some embodiments, the present disclosure provides for one or more cells, tissues, organs, or animals having multiple copies of one or more of the modified genes. For example, the cells, tissues, organs, or animals may have 2, 3, 4, 5, 6, 7, 8, 9, about 10, about 15, about 20, about 25, about 30, or more of one or more of the modified genes.

In some embodiments, the one or more cells is a primary cell. In some embodiments, the one or more cells is a somatic cell. In some embodiments, the one or more cells is a post-natal cell. In some embodiments, the one or more cells is an adult cell (e.g., an adult ear fibroblast). In some embodiments, the one or more cells is a fetal/embryonic cell (e.g., an embryonic blastomere). In some embodiments, the one or more cells is a germ line cell. In some embodiments, the one or more cells is an oocyte. In some embodiments, the one or more cells is a stem cell. In some embodiments, the one or more cells is a cell from a primary cell line. In some embodiments, the one or more cells is selected from the group consisting of: an epithelial cell, a liver cell, a granulosa cell, a fat cell. In particular embodiments, the one or more cells is a fibroblast. In some embodiments, the fibroblast is a female fetal fibroblast. In some embodiments, the one or more cells is in vitro. In some embodiments, the one or more cells is in vivo. In some embodiments, the one or more cells is a single cell. In some embodiments, the one or more cells is a member of a cell colony.

In some embodiments, the one or more cells is a porcine cell. Non-limiting examples of the breeds a porcine cell originates from or is derived from includes any of the following pig breeds: American Landrace, American Yorkshire, Aksai Black Pied, Angeln saddleback, Appalachian English, Arapawa Island, Auckland Island, Australian Yorkshire, Babi Kampung, Ba Xuyen, Bantu, Basque, Bazna, Beijing Black, Belarus Black Pied, Belgian Landrace, Bengali Brown Shannaj, Bentheim Black Pied, Berkshire, Bisaro, Bangur, Black Slavonian, Black Canarian, Breitovo, British Landrace, British Lop, British Saddleback, Bulgarian White, Cambrough, Cantonese, Celtic, Chato Murciano, Chester White, Chiangmai Blackpig, Choctaw Hog, Creole, Czech Improved White, Danish Landrace, Danish Protest, Dermantsi Pied, Li Yan, Duroc, Dutch Landrace, East Landrace, East Balkan, Essex, Estonian Bacon, Fengjing, Finnish Landrace, Forest Mountain, French Landrace, Gascon, German Landrace, Gloucestershire Old Spots, Gottingen minipig, Grice, Guinea Hog, Hampshire, Hante, Hereford, Hezuo, Hogan Hog, Huntington Black Hog, Iberian, Italian Landrace, Japanese Landrace, Jeju Black, Jinhua, Kakhetian, Kele, Kemerovo, Korean Native, Krskopolje, Kunekune, Lamcombe, Large Black, Large Black-White, Large White, Latvian White, Leicoma, Lithuanian Native, Lithuanian White, Lincolnshire Curly-Coated, Livny, Malhado de Alcobaca, Mangalitsa, Meishan, Middle White, Minzhu, Minokawa Buta, Mong Cai, Mora Romagnola, Moura, Mukota, Mulefoot, Murom, Myrhorod, Nero dei Nebrodi, Neijiang, New Zealand, Ningxiang, North Caucasian, North Siberian, Norwegian Landrace, Norwegian Yorkshire, Ossabaw Island, Oxford Sandy and Black, Pakchong 5, Philippine Native, Pietrain, Poland China, Red Wattle, Saddleback, Semirechensk, Siberian Black Pied, Small Black, Small White, Spots, Surabaya Babi, Swabian-Hall, Swedish Landrace, Swallow Belied Mangalitza, Taihu pig, Tamworth, Thuoc Nhieu, Tibetan, Tokyo-X, Tsivilsk, Turopolje, Ukrainian Spotted Steppe, Ukrainian White Steppe, Urzhum, Vietnamese Potbelly, Welsh, Wessex Saddleback, West French White, Windsnyer, Wuzhishanm, Yanan, Yorkshire and Yorkshire Blue and White. In some embodiments, the porcine cells are Yorkshire and Yucatan porcine cells.

In some embodiments, the cells, tissues, organs or animals of the present disclosure have been genetically modified such that one or more genes has been modified by addition, deletion, inactivation, disruption, excision of a portion thereof, or a portion of the gene sequence has been altered.

In some embodiments, the cells, tissues, organs or animals of the disclosure comprise one or more mutations that inactivate one or more genes. In some embodiments, the cells, tissues, organs or animals comprise one or more mutations or epigenetic changes that result in decreased or eliminated expression of one or more genes having the one or more mutations. In some embodiments, the one or more genes is inactivated by genetically modifying the nucleic acid(s) present in the cells, tissues, organs or animals. In some embodiments, the inactivation of one or more genes is confirmed by means of an assay. In some embodiments, the assay is an infectivity assay, reverse transcriptase PCR assay, RNA-seq, real-time PCR, or junction PCR mapping assay.

### Specific Genotypes

To warrant cells, tissues, organs and animals safe and effective for human clinical use, the cells, tissues, organs, and animals (e.g., donor pigs) of the present disclosure are genetically engineered to have enhanced complement (i.e., complement toxicity), coagulation, inflammatory (i.e., apoptosis/inflammation), immune (i.e., cellular toxicity), and/or immunomodulation systems that render them compatible in humans. Novel combinations of knockout (KO), knockin (KI) (alternately referred to herein as transgene (TG)), and/or genomic replacement strategies provide the enhanced complement, coagulation, inflammatory, immune, and/or immunomodulation systems.

Cells, tissues, organs and animals lacking expression of major xenogenic carbohydrate antigens, for example by genetic KO, reduce or eliminate humoral rejection during xenotransplantation. Three of the major xenogenic carbohydrate antigens include those produced by the glycosyltransferases/glycosylhydrolases GGTA1, CMAH, and B4GALNT2. A purpose for the functional loss of these genes is to reduce and/or eliminate the binding of preformed anti-pig antibodies to the endothelium of the porcine grafts.

Insertion of key complement, coagulation, inflammatory, immune, and/or immunomodulation factors into one or more genomic loci, for example safe harbor genomic loci such as AAVS1, will aid in regulating the human complement system, and natural killer (NK), macrophage, and T cell function. Nonlimiting examples include, overexpression by KI of hCD46, hCD55, and hCD59 to inhibit the human complement cascade; humanization of vWF to prevent unregulated platelet sequestration and thrombotic microangiopathy, for example, by humanizing the A1 domain and/or flanking regions of the porcine vWF sequence; KI of B2M-HLA-E SCT to provide protection against human NK cell cytotoxicity and humanization of porcine cells; and KI of CD47, CD39, THBD, TFPI, A20 to function as immunosuppressants, immunomodulators, and/or anticoagulants.

In some embodiments, the cells, tissues, organs or animals of the present disclosure have been genetically modified such that one or more genes has been modified by addition, deletion, inactivation, disruption, excision of a portion thereof, or a portion of the gene sequence has been altered. In some embodiments, the present disclosure provides an isolated cell, tissue, organ, or animal having multiple modified genes. In some embodiments, the modified genes include one or more of alpha 1,3, galactosyltransferase (GGTA), Beta-1,4-N-Acetyl-Galactosaminyltransferase 2 (B4GalNT2), cytidine monophosphate-N-acetylneuraminic acid hydroxylase (CMAH), THBD, TFPI, CD39, HO-1, CD46, CD55, CD59, major histocompatibility complex, class I, E single chain trimer (HLA-E SCT), A20, PD-L1, CD47, swine leukocyte antigen 1 (SLA-1), SLA-2, SLA-3, vWF, B2M, DQA, DRA, and CD47.

In some embodiments, the modified genes are GGTA, B4GalNT2, CMAH, or any combination thereof. In some embodiments, the GGTA, B4GalNT2, and/or CMAH are genetically KO. In some embodiments, the modified genes are THBD, TFPI, CD39, HO-1, or any combination thereof. In some embodiments, the THBD, TFPI, CD39, and/or HO-1 are genetically KI. In some embodiments, the modified genes are CD46, CD55, CD59, B2M-HLA-E SCT, A20, PD-L1, CD47, or any combination thereof. In some embodiments, the CD46, CD55, CD59, B2M-HLA-E SCT, A20, PD-L1, and/or CD47 are genetically KI. In some embodiments, the modified genes are SLA-1, SLA-2, SLA-3, B2M, or any combination thereof. In some embodiments, the modified genes are DQA and/or DRA. In some embodiments, the modified genes are PD-L1, exogenous vWF, HLA-E, HLA-G, B2M, CIITA-DN, and or any combination thereof. In some embodiments, the modified genes are TBM, PD-L1, HLA-E, CD47, or any combination thereof. In some embodiments, the TBM, PD-L1, HLA-E, and/or CD47 are genetically KI. In some embodiments, the modified genes are MHC-I genes SLA-1, SLA-2, and SLA-3, MHC-II genes DQA and DRA, endogenous vWF, CD9, asialoglycoprotein receptor, at least one complement inhibitor gene (e.g., C3, CD46, CD55, and CD59), and any combination thereof. In some embodiments the CD46, CD55 and/or CD59 are genetically KI.

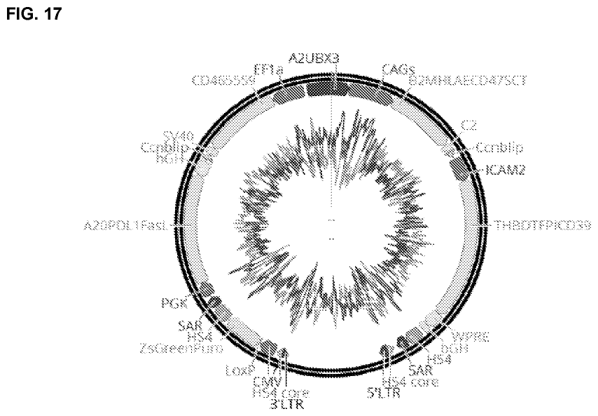

In one embodiment, the cells, tissues, organs or animals of the present disclosure have been genetically modified with a transgene expression vector comprising B2M, HLA-E SCT, CD47, THBD, TFPI, CD39, A20, PD-L1, FasL, CD46, CD55, CD59, or any combination thereof. In one embodiment, the cells, tissues, organs or animals of the present disclosure have been genetically modified with a transgene expression vector comprising each of B2M, HLA-E SCT, CD47, THBD, TFPI, CD39, A20, PD-L1, FasL, CD46, CD55, and CD59. One embodiment of a transgene expression vector is depicted in FIG. 17. In one embodiment, the cells, tissues, organs or animals of the present disclosure have been further genetically modified to have reduced or no expression of GGTA, B4GalNT2, CMAH, or any combination thereof, for example by genetic KO.

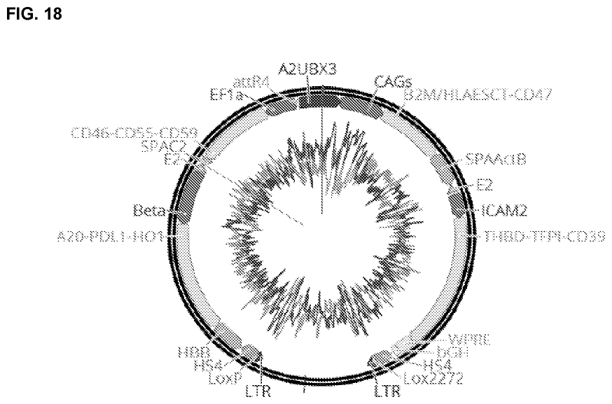

In one embodiment, the cells, tissues, organs or animals of the present disclosure have been genetically modified with a transgene expression vector comprising B2M, HLA-E SCT, CD47, THBD, TFPI, CD39, A20, PD-L1, HO-1, CD46, CD55, CD59, or any combination thereof. In one embodiment, the cells, tissues, organs or animals of the present disclosure have been genetically modified with a transgene expression vector comprising each of B2M, HLA-E SCT, CD47, THBD, TFPI, CD39, A20, PD-L1, HO-1, CD46, CD55, and CD59. One embodiment of a transgene expression vector is depicted in FIG. 18. In one embodiment, the cells, tissues, organs or animals of the present disclosure have been further genetically modified to have reduced or no expression of GGTA, B4GalNT2, CMAH, or any combination thereof, for example by genetic KO.

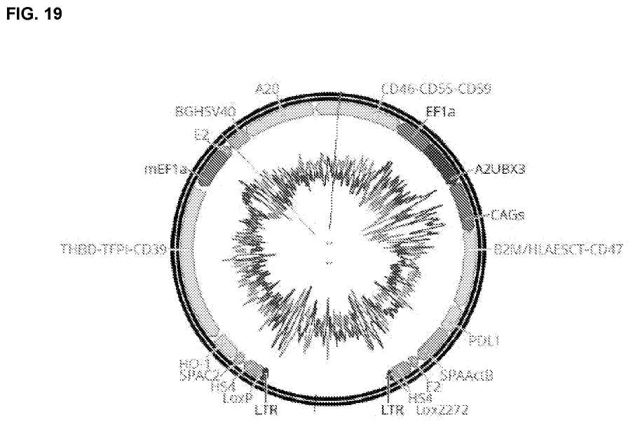

In one embodiment, the cells, tissues, organs or animals of the present disclosure have been genetically modified with a transgene expression vector comprising B2M, HLA-E SCT, CD47, PD-L1, HO-1, THBD, TFPI, CD39, A20, CD46, CD55, CD59, or any combination thereof. In one embodiment, the cells, tissues, organs or animals of the present disclosure have been genetically modified with a transgene expression vector comprising each of B2M, HLA-E SCT, CD47, PD-L1, HO-1, THBD, TFPI, CD39, A20, CD46, CD55, and CD59. One embodiment of a transgene expression vector is depicted in FIG. 19. In one embodiment, the cells, tissues, organs or animals of the present disclosure have been further genetically modified to have reduced or no expression of GGTA, B4GalNT2, CMAH, or any combination thereof, for example by genetic KO.

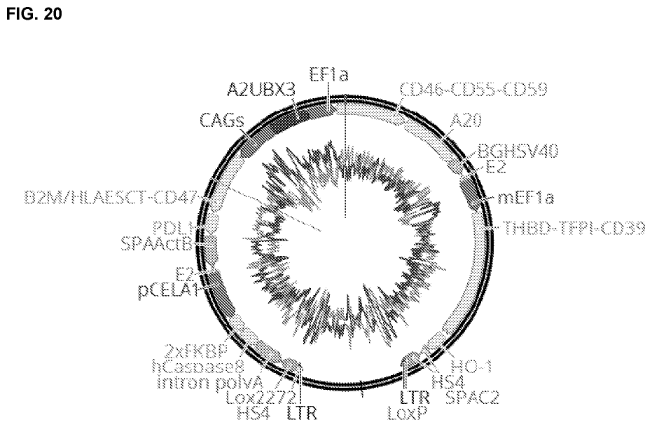

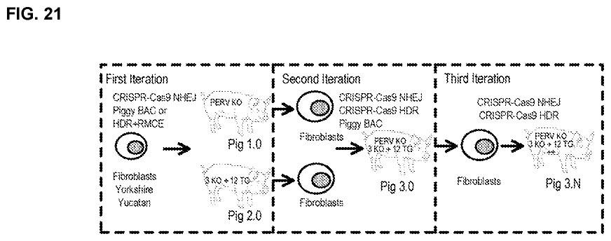

In one embodiment, the cells, tissues, organs or animals of the present disclosure have been genetically modified with a transgene expression vector comprising CD46, CD55, CD59, A20, THBD, TFPI, CD39, HO-1, 2×FKBP (fusion of s FK506 binding protein), hCaspase8, PD-L1, B2M, HLA-E SCT, CD47, or any combination thereof. In one embodiment, the cells, tissues, organs or animals of the present disclosure have been genetically modified with a transgene expression vector comprising each of CD46, CD55, CD59, A20, THBD, TFPI, CD39, HO-1, 2×FKBP, hCaspase8, PD-L1, B2M, HLA-E SCT, and CD47. One embodiment of a transgene expression vector is depicted in FIG. 20. In one embodiment, the cells, tissues, organs or animals of the present disclosure have been further genetically modified to have reduced or no expression of GGTA, B4GalNT2, CMAH, or any combination thereof, for example by genetic KO.

The cells, tissues, organs or animals of the present disclosure can be genetically modified by any method. Non-limiting examples of suitable methods for the knockout (KO), knockin (KI), and/or genomic replacement strategies disclosed and described herein include CRISPR-mediated genetic modification using Cas9, Cas12a (Cpf1), or other CRISPR endonucleases, Argonaute endonucleases, transcription activator-like (TAL) effector and nucleases (TALEN), zinc finger nucleases (ZFN), expression vectors, transposon systems (e.g., PiggyBac transposase), or any combination thereof.

The cells, tissues, organs or animals of the present disclosure can be further modified to be PERV-free. The cells, tissues, organs or animals of the present disclosure can be further modified to have PERV copies functionally deleted from their genome. The cells, tissues, organs or animals of the present disclosure can be further modified to have PERV copies functionally inactivated in their genome. PERVs represent a risk factor if porcine cells, tissues, or organs were to be transplanted into human recipients. PERVs are released from normal pig cells and are infectious. PERV-A and PERV-B are polytropic viruses infecting cells of several species, among them humans (e.g. they are xenotropic); whereas PERV-C is an ecotropic virus infecting only pig cells. Non-limiting methods for functionally deleted PERV copies are disclosed and described in Niu 2017 and WIPO Publ. No. WO2018/195402, both of which are incorporated by reference herein in their entireties. In some embodiments, the pigs are genetically engineered to be PERV-A, PERV-B, or PERV-C (or any combination thereof) free.

In some embodiments, additional genes of cells, tissues, organs or animals of the present disclosure are modified by addition, deletion, inactivation, disruption, excision of a portion thereof, or a portion of the gene sequence has been altered. In some embodiments, the modified genes include deleting one or more of the following genes: MHC-I genes SLA-1, SLA-2, and SLA-3, MHC-II genes DQA and DRA, endogenous vWF, CD9, asialoglycoprotein receptor, and C3, and expressing one or more of the following transgenes: PD-L1, exogenous vWF, HLA-E, HLA-G, B2M, and CIITA-DN. In some embodiments, the modified genes include deleting one or more of the following genes: alpha galactosyltransferase 1, β1,4 N-acetylgalactosaminyltransferase, and cytidine monophosphate-N-acetylneuraminic acid hydroxylase, and expressing one or more of the following transgenes: CD46, CD55, CD59, CD47, HO-1, A20, TNFR1-Ig, CD39, THBD, TFPI, EPCR, PD-1, CTLA-Ig, CD73, SOD3, CXCL12, FasL, CXCR3, CD39L1, GLP-1R, M3R, 1L35, IL12A and EB13. In some embodiments, the modified genes are CD46, CD55, CD59, CD47, HO-1, A20, TNFR1-Ig, CD39, THBD, TFPI, EPCR, PD-1, CTLA-Ig, CD73, SOD3, CXCL12, FasL, CXCR3, CD39L1, GLP-1R, M3R, 1L35, IL12A and EB13.

In some embodiments, the cells, tissues, organs or animals of the present disclosure have been genetically modified such that one or more genes has been modified by addition, deletion, inactivation, disruption, excision of a portion thereof, a portion of the gene sequence has been altered, or introducing a transgene or a portion thereof. In some embodiments, the present disclosure provides an isolated cell, tissue, organ, or animal has one or more modified genes. In some embodiments, the modified genes are MHC Class I genes. In some embodiments, the modified MHC Class I genes include one or more of the following SLA-1, SLA-2, SLA-3, and B2M. In some embodiments, the modified genes are SLA-1, SLA-2, and/or SLA-3. In some embodiments, the modified gene is B2M. In some embodiments, the modified MHC Class I genes include one or more of the following SLA-1, SLA-2, SLA-3, and B2M. In some embodiments, the modified B2M, SLA-1, SLA-2, and/or SLA-3 genes, and/or a portion thereof, are replaced with a human HLA-E gene, a human HLA-G gene, a human B2M gene, and/or a human (dominant-negative mutant class II transactivator) CIITA-DN gene, and/or a portion thereof. In some embodiments, the modified genes are conditionally and/or inducibly modified. In some embodiments, a conditional promoter and/or an inducible promoter is used to conditionally and/or inducibly modify the one or more modified genes. In some embodiments, the isolated cell, tissue, organ, or animal comprises conditionally altering B2M, SLA-1, SLA-2, or SLA-3 genes, or any combination thereof, and replacing the conditionally altered genes with at least a portion of a human HLA-E gene, a human HLA-G gene, a human B2M gene, and/or a human CIITA-DN gene.

In some embodiments, the cells, tissues, organs or animals of the present disclosure have been genetically modified such that one or more genes has been modified by addition, deletion, inactivation, disruption, excision of a portion thereof, a portion of the gene sequence has been altered, or introducing a transgene or a portion thereof. In some embodiments, the present disclosure provides an isolated cell, tissue, organ, or animals has one or more modified genes. In some embodiments, the modified genes are MHC Class II genes. In some embodiments, the modified MHC Class II genes are DRQ, DRA, or any combination thereof. In some embodiments, DRQ and/or DRA is modified by addition, deletion, inactivation, disruption, excision of a portion thereof, a portion of the gene sequence has been altered. In some embodiments, the modified genes are conditionally and/or inducibly modified. In some embodiments, a conditional promoter and/or an inducible promoter is used to conditionally and/or inducibly modify the one or more modified genes. In some embodiments, the isolated cell, tissue, organ, or animal comprises conditionally altering DRQ and/or DRA genes, or any combination thereof.

In some embodiments, the cells, tissues, organs or animals of the present disclosure have been genetically modified such that one or more genes has been modified by addition, deletion, inactivation, disruption, excision of a portion thereof, a portion of the gene sequence has been altered, or introducing a transgene or a portion thereof. In some embodiments, the present disclosure provides an isolated cell, tissue, organ, or animal having a modified vWF gene. In some embodiments, the modified genes are vWF genes and vWF-related genes. In some embodiments, the modified vWF gene, and/or a portion thereof, is replaced with a human vWF gene and/or a portion thereof. In some embodiments, the modified vWF gene, modified vWF-related genes, and/or a portion(s) thereof, is replaced with a human vWF gene, one or more human vWF-related genes, and/or a portion thereof. In some embodiments, the modified vWF gene and/or vWF-related genes are conditionally and/or inducibly modified. In some embodiments, a conditional promoter and/or an inducible promoter is used to conditionally and/or inducibly modify the one or more modified genes. In some embodiments, the isolated cell, tissue, organ, or animal comprises conditionally altering vWF, vWF-related genes, a portion(s) thereof, or any combination thereof, and replacing the conditionally altered genes with the human vWF gene, at least a portion of the human vWF gene, one or more other human vWF-related genes, at least a portion of one or more human vWF-related genes, or any combination thereof. In some embodiments, the vWF gene is modified using gRNAs designed to initiate the HDR replacement in the endogenous porcine genome and cut near the region to be replaced by the human sequences. Non-limiting examples of suitable gRNAs are any one or more of SEQ ID NOs: 5-157.

In some embodiments, the cells, tissues, organs or animals of the present disclosure have been genetically modified such that one or more genes has been modified by addition, deletion, inactivation, disruption, excision of a portion thereof, a portion of the gene sequence has been altered, or introducing a transgene or a portion thereof. In some embodiments, the cells, tissues, organs or animals of the present disclosure have been genetically modified by introduction of one or more exogenous genes, or portions thereof, into the cells, tissues, organs, or animals, such as a transgene. In some embodiments, the present disclosure provides an isolated cell, tissue, organ, or animal having one or more modified genes. In some embodiments, the modified genes are programmed death genes. In some embodiments, the modified gene is PD-L1. In some embodiments, the cells, tissues, organs, or animals are modified to express an exogenous PD-L1 gene, or portion thereof, such as a transgene. In some embodiments, the modified genes are conditionally and/or inducibly modified. In some embodiments, a conditional promoter and/or an inducible promoter is used to conditionally and/or inducibly modify the one or more modified genes. In some embodiments, the isolated cell, tissue, organ, or animal comprises conditionally altering PD-L1. In some embodiments, the PD-L1 comprises the sequence described in SEQ ID NO: 211 or any variant or portion thereof.

In some embodiments, the cells, tissues, organs or animals of the present disclosure have been genetically modified such that one or more genes has been modified by addition, deletion, inactivation, disruption, excision of a portion thereof, a portion of the gene sequence has been altered, or introducing a transgene or a portion thereof. In some embodiments, the present disclosure provides an isolated cell, tissue, organ, or animal has one or more modified genes. In some embodiments, the modified genes are complement genes. In some embodiments, the modified gene is C3. In some embodiments, C3 is modified by addition, deletion, inactivation, disruption, excision of a portion thereof, a portion of the gene sequence has been altered. In some embodiments, the modified C3 gene and/or complement-related genes are conditionally and/or inducibly modified. In some embodiments, a conditional promoter and/or an inducible promoter is used to conditionally and/or inducibly modify the one or more modified genes. In some embodiments, the isolated cell, tissue, organ, or animal comprises conditionally altering C3, complement-related genes, a portion(s) thereof, or any combination thereof. In some embodiments, the C3 gene is modified using gRNAs. Non-limiting examples of suitable gRNAs include any one or more of SEQ ID NOs: 158-210.

In some embodiments, the modified gene is a knockout of C3. In some embodiments, the modified gene is a knock-in of PD-L1. In some embodiments, the modified gene is a humanized vWF of the porcine vWF. In some embodiments, the modified gene is a conditional knock-in of MHC-I genes SLA-1, SLA-2, and SLA-3.

In some embodiments, no or substantially no immune response is elicited by the host against the genetically modified cell, tissue or organ.

In some embodiments, the disclosure provides for nucleic acids obtained from any of the cells disclosed herein. In some embodiments, the nucleic acid(s) in the cell are genetically modified such that one or more genes in the cell are altered or the genome of the cell is otherwise modified. In some embodiments, the genes, or portions thereof, that are genetically modified using any of the genetic modifications systems known in the art and/or disclosed herein. In some embodiments, the genetic modification system is a TALEN, a zinc finger nuclease, and/or a CRISPR-based system. In some embodiments, the genetic modification system is a CRISPR-Cas9 system. In some embodiments, the genetic modification system is a Class II, Type-II CRISPR system. In some embodiments, the genetic modification system is a Class II, Type-V CRISPR system. In some embodiments, the cell is genetically modified such that one or more genes or portions thereof in the cell are inactivated, and the cell is further genetically modified such that the cell has reduced expression of one or more genes, or portions thereof, that would induce an immune response if the cell (or a tissue or organ cloned/derived from the cell) were transplanted to a human. In some embodiments, the cell is genetically modified to have increased expression of one or more human genes, or portions thereof. In some embodiments, the cell is genetically modified to have increased expression of one or more humanized genes, or portions thereof. In some embodiments, the cell is genetically modified such that one or more genes, or portions thereof, in the cell are inactivated, and the cell is further genetically modified such that the cell has increased expression of one or more genes that would suppress an immune response if the cell (or a tissue or organ cloned/derived from the cell) were transplanted to a human. In some embodiments, the cell is genetically modified such that one or more genes, or portions thereof, in the cell are inactivated, and the cell is further genetically modified such that the cell has reduced expression of one or more genes that would induce an immune response if the cell (or a tissue or organ cloned/derived from the cell) were transplanted to a human, and the cell is further genetically modified such that the cell has increased expression of one or more genes that would suppress an immune response if the cell (or a tissue or organ cloned/derived from the cell) were transplanted to a human.

In some embodiments, the disclosure provides for an embryo that was cloned from the genetically modified cell. In some embodiments, the genetically modified nucleic acid(s) are extracted from the genetically modified cell and cloned into a different cell. For example, in somatic cell nuclear transfer, the genetically modified nucleic acid from the genetically modified cell is introduced into an enucleated oocyte. In some embodiments, oocytes can be enucleated by partial zona dissection near the polar body and then pressing out cytoplasm at the dissection area. In some embodiments, an injection pipette with a sharp beveled tip is used to inject the genetically modified cell into an enucleated oocyte arrested at meiosis 2. Oocytes arrested at meiosis-2 are frequently termed “eggs.” In some embodiments, an embryo is generated by fusing and activating the oocyte. Such an embryo may be referred to herein as a “genetically modified embryo.” In some embodiments, the genetically modified embryo is transferred to the oviducts of a recipient female pig. In some embodiments, the genetically modified embryo is transferred to the oviducts of a recipient female pig 20 to 24 hours after activation. See, e.g., Cibelli 1998 and U.S. Pat. No. 6,548,741. In some embodiments, recipient females are checked for pregnancy approximately 20-21 days after transfer of the genetically modified embryo.

In some embodiments, the genetically modified embryo is grown into a post-natal genetically modified animal. In some embodiments, the post-natal genetically modified animal is a neo-natal genetically modified animal. In some embodiments, the genetically modified pig is a juvenile genetically modified animal. In some embodiments, the genetically modified animal is an adult genetically modified animal (e.g., older than 5-6 months). In some embodiments, the genetically modified animal is a female genetically modified animal. In some embodiments, the animal is a male genetically modified animal. In some embodiments, the genetically modified animal is bred with a non-genetically modified animal. In some embodiments, the genetically modified animal is bred with another genetically modified animal. In some embodiments, the genetically modified pig is bred with another genetically modified animal that has reduced or no active virus. In some embodiments, the genetically modified animal is bred with a second genetically modified animal that has been genetically modified such that the cells, tissues or organs from the second genetically modified animal are less likely to induce an immune response if transplanted to a human.

In some embodiments the genetically modified animal is an animal having one or more modified genes and maintains a same or similar level of expression or inactivation of the modified gene(s) for at least a month, at least 6 months, at least 1 year, at least 5 years, at least 10 years post-gestation. In some embodiments, the genetically modified animal remains genetically modified having one or more modified genes as a genetically modified pig even after delivery from a non-viral-inactivated surrogate or after being in a facility/space with other non-viral-inactivated animals.

In some embodiments, the disclosure provides for cells, tissues, or organs obtained from any of the post-natal genetically modified pigs described herein. In some embodiments, the cell, tissue, or organ is selected from the group consisting of liver, kidney, lung, heart, pancreas, muscle, blood, and bone. In particular embodiments, the organ is liver, kidney, lung or heart. In some embodiments, the cell from the post-natal genetically modified pig is selected from the group consisting of: pancreatic islets, lung epithelial cells, cardiac muscle cells, skeletal muscle cells, smooth muscle cells, hepatocytes, non-parenchymal liver cells, gall bladder epithelial cells, gall bladder endothelial cells, bile duct epithelial cells, bile duct endothelial cells, hepatic vessel epithelial cells, hepatic vessel endothelial cells, sinusoid cells, choroid plexus cells, fibroblasts, Sertoli cells, neuronal cells, stem cells, and adrenal chromaffin cells. In some embodiments, the genetically modified organs, tissues or cells have been separated from their natural environment (i.e., separated from the pig in which they are being grown). In some embodiments, separation from the natural environment means a gross physical separation from the natural environment, e.g., removal from the genetically modified donor animal, and alteration of the genetically modified organs', tissues' or cells' relationship with the neighboring cells with which they are in direct contact (e.g., by dissociation).

### III. Methods of Generating Cells, Tissues, Organs, or Animals

The disclosure provides for methods of generating any of the cells, tissues, organs, or animals having one or more modified genes disclosed herein. In some embodiments, the disclosure provides a method of inactivating, deleting, or otherwise disrupting one or more genes, or portions thereof, in any of the cells disclosed herein, comprising administering to the cell a gene editing agent specific to a gene, wherein the agent disrupts transcription and/or translation of the gene. In some embodiments, the agent targets the start codon of the gene and inhibits transcription of the gene. In some embodiments, the agent targets an exon in the gene and the agent induces a frameshift mutation in the gene. In some embodiments, the agent introduces an inactivating mutation into the gene. In some embodiments, the agent represses transcription of the gene.

In some embodiments, the disclosure provides a method of altering one or more genes, or a portion thereof, in vivo, comprising administering to the cell a gene editing agent specific to a gene, wherein the agent alters a sequence of the gene, such as by humanizing the gene or otherwise changing a native (e.g., wild-type) sequence of the gene.

In some embodiments, the disclosure provides a method of expressing one or more genes, or a portion thereof, such as a transgene (e.g., non-native gene) comprising administering to the cell a gene editing agent specific to the transgene gene, wherein the agent introduces a sequence of the transgene. In some embodiments, the agent is a nucleic acid sequence, such as a plasmid, a vector, or the like. In some embodiments, the nucleic acid sequence includes one or more nucleic acid sequences, such as a promoter, a transgene, and/or additional genes. In some embodiments, the nucleic acid sequence, or a portion thereof, is derived from one or more species and/or one or more sources. In some embodiments, the species is a species that will receive the genetically modified cell, tissue, or organ. In some embodiments, the species is a human. In other embodiments, the species is non-human, such as a mammal, an animal, a bacteria, and/or a virus.

In some embodiments, any of the agents disclosed herein is a polynucleotide. In some embodiments, the polynucleotide encodes one or more of the nucleases and/or nickases and/or RNA or DNA molecules described herein. In some embodiments, the polynucleotide agent is introduced to one or more cells. In some embodiments, the polynucleotide is introduced to the one or more cells in a manner such that the polynucleotide is transiently expressed by the one or more cells. In some embodiments, the polynucleotide is introduced to the one or more cells in a manner such that the polynucleotide is stably expressed by the one or more cells. In some embodiments, the polynucleotide is introduced in a manner such that it is stably incorporated in the cell genome. In some embodiments, the polynucleotide is introduced along with one or more transposable elements. In some embodiments, the transposable element is a polynucleotide sequence encoding a transposase. In some embodiments, the transposable element is a polynucleotide sequence encoding a PiggyBac transposase. In some embodiments, the transposable element is inducible. In some embodiments, the transposable element is doxycycline-inducible. In some embodiments, the polynucleotide further comprises a selectable marker. In some embodiments, the selectable marker is a puromycin-resistant marker. In some embodiments, the selectable marker is a fluorescent protein (e.g., GFP).

In some embodiments, the agent is a nuclease or a nickase that is used to target DNA in the cell. In some embodiments, the agent specifically targets and suppresses expression of a gene. In some embodiments, the agent comprises a transcription repressor domain. In some embodiments, the transcription repressor domain is a Krüppel associated box (KRAB).

In some embodiments, the agent is any programmable nuclease. In some embodiments, the agent is a natural homing meganuclease. In some embodiments, the agent is a TALEN-based agent, a ZFN-based agent, or a CRISPR-based agent, or any biologically active fragment, fusion, derivative or combination thereof. CRISPR-based agents include, for example, Class II Type II and Type V systems, including e.g. the various species variants of Cas9 and Cpf1. In some embodiments, the agent is a deaminase or a nucleic acid encoding a deaminase. In some embodiments, a cell is genetically engineered to stably and/or transiently express a TALEN-based agent, a ZFN-based agent, and/or a CRISPR-based agent.

### IV. Methods of Treatment

In some embodiments, any of the genetically modified cells, tissues or organs disclosed herein may be used to treat a subject of a different species as the genetically modified cells. In some embodiments, the disclosure provides for methods of transplanting any of the genetically modified cells, tissues or organs described herein into a subject in need thereof. In some embodiments, the subject is a human. In some embodiments, the subject is a non-human primate.

In some embodiments, a genetically modified organ for use in any of the methods disclosed herein may be selected from the heart, lung, liver, eye, pituitary, thyroid, parathyroid, esophagus, thymus, adrenal glands, appendix, bladder, gallbladder, small intestine, large intestine, small intestine, kidney, pancreas, spleen, stomach, skin, and/or prostate, of the genetically modified pig. In some embodiments, a genetically modified tissue for use in any of the methods disclosed herein may be selected from cartilage (e.g., esophageal cartilage, cartilage of the knee, cartilage of the ear, cartilage of the nose), muscle such as, but not limited to, smooth and cardiac (e.g., heart valves), tendons, ligaments, bone (e.g., bone marrow), cornea, middle ear and veins of the genetically modified pig. In some embodiments, a genetically modified cell for use in any of the methods disclosed herein includes blood cells, skin follicles, hair follicles, and/or stem cells. Any portion of an organ or tissue (e.g., a portion of the eye such as the cornea) may also be administered the compositions of the present disclosure.

In some embodiments, a heart, lung, liver, kidney, pancreas, or spleen is isolated from a pig that has been genetically modified to comprise (a) deletions or disruptions of GGTA1, CMAH, and B4GALNT2; (b) addition of CD46, CD55, CD59, CD39, CD47, A20, PD-L1, HLA-E, B2M, THBD, TFPI, and HO transgenes (e.g. human or humanized copies thereof) expressed from a single multi-transgene cassette in the pig genome; and (c) functional deletion of all PERV copies. In some embodiments, a heart, lung, liver, kidney, pancreas, or spleen is isolated from a pig that has been genetically modified to comprise (a) functional disruption of GGTA1, CMAH, and B4GALNT2; (b) addition of CD46, CD55, CD59, CD39, CD47, A20, PD-L1, HLA-E, B2M, THBD, TFPI, and HO transgenes (e.g. humanized copies thereof) expressed from a single multi-transgene cassette in the pig genome; and (c) functional inactivation of all PERV copies. In certain embodiments, the pig has been further genetically modified to have humanized vWF, deletion of ASGR1, and/or deletion of B2M genes.

In some embodiments, the xenotransplanted organ (e.g., heart, lung, liver, kidney, pancreas, spleen) exhibits sustained function once xenografted into a human or nonhuman primate for more than about 300 days, more than about 1 year, more than about 1.5 years, more than about 2 years, more than about 2.5 years, more than about 3 years, more than about 3.5 years, more than about 4 years, more than about 4.5 years, more than about 5 years, more than about 5.5 years, more than about 6 years, more than about 6.5 years, more than about 7 years, more than about 7.5 years, more than about 8 years, more than about 8.5 years, more than about 9 years, more than about 9.5 years, or more than about 10 years.

In some embodiments, the disclosure provides for treating a subject having a disease, disorder or injury that results in a damaged, deficient or absent organ, tissue or cell function. In some embodiments, the subject has suffered from an injury or trauma (e.g., an automobile accident) resulting in the damage of one or more cells, tissues or organs of the subject. In some embodiments, the subject has suffered a fire or acid burn. In some embodiments, the subject has a disease or disorder that results in a damaged, deficient or absent organ, tissue or cell function. In some embodiments, the subject is suffering from an autoimmune disease. In some embodiments, the disease is selected from the group consisting of: heart disease (e.g., atherosclerosis), dilated cardiomyopathy, severe coronary artery disease, scarred heart tissue, birth defects of the heart, diabetes Type I or Type II, hepatitis, cystic fibrosis, cirrhosis, kidney failure, lupus, scleroderma, IgA nephropathy, polycystic kidney disease, myocardial infarction, emphysema, chronic bronchitis, bronchiolitis obliterans, pulmonary hypertension, congenital diaphragmatic hernia, congenital surfactant protein B deficiency, and congenital cystic emphysematous lung disease, primary biliary cholangitis, sclerosing cholangitis, biliary atresia, alcoholism, Wilson's disease, hemochromatosis, and/or alpha-1 antitrypsin deficiency.

In some embodiments, any of the genetically modified cells, tissues and/or organs of the disclosure are separated from the genetically modified donor and administered into a non-donor subject host. “Administering” or “administration”, as used in this context, includes, but is not limited to, introducing, applying, injecting, implanting, grafting, suturing, and transplanting. According to the disclosure, the genetically modified cells, tissues and/or organs may be administered by a method or route which results in localization of the organs, tissues, cells or compositions of the disclosure at a desired site. The organs, tissues, cells or compositions of the disclosure can be administered to a subject by any appropriate route which results in delivery of the cells to a desired location in the subject where at least a portion of the cells remain viable. In some embodiments, at least 10%, 20%, 30%, 40%, 50%, 60%, 70%, 80%, 90%, or 100% of the cells (whether administered separately or as part of a tissue or organ) remain viable after administration to the subject. Methods of administering organs, tissues, cells or compositions of the disclosure are well-known in the art. In some embodiments, the cells, tissues and/or organs are transplanted into the host. In some embodiments, the cells, tissues and/or organs are injected into the host. In some embodiments, the cells, tissues and/or organs are grafted onto a surface of the host (e.g., bone or skin).

In some embodiments, a heart, lung, liver, kidney, pancreas, or spleen which has been genetically modified to harbor deletions or disruptions of GGTA1, CMAH, and B4GALNT2; expression of CD46, CD55, CD39, CD47, HLA-E, THBD, and TFPI, and optionally one or more of CD59, B2M, A20, PD-L1, and HO-1 from a single multi-transgene cassette in the pig genome; along deletion of all PERV copies is transplanted into the host. In some embodiments, a heart, lung, liver, kidney, pancreas, or spleen which has been genetically modified to harbor deletions of GGTA1, CMAH, and B4GALNT2; expression of CD46, CD55, CD39, CD47, HLA-E, THBD and TFPI, and optionally one or more of CD59, B2M, A20, PD-L1, and HO-1 from a single multi-transgene cassette in the pig genome; and functional inactivation of all PERV copies is transplanted into the host. In some embodiments, the transplanted heart, lung, liver, kidney, pancreas, spleen, or a portion thereof survive and are functional for a period of time of about 1 day, about 1 week, about 2 weeks, about 3 weeks, about 1 month, about 2 months, about 3 months, about 4 months, about 5 months, about 6 months, about 9 months, about 1 year, about 2 years, about 3 years, about 4 years, about 5 years, about 6 years, about 7 years, about 8 years, about 9 years, about 10 years, or more.

In some embodiments, it will be necessary to protect the genetically modified cell(s), tissue(s) or organ(s) from the immune system of the host to whom the genetically modified cell(s), tissue(s) or organ(s) are being administered. For example, in some embodiments, the genetically modified cell(s), tissue(s) or organ(s) is administered with a matrix or coating (e.g., gelatin) to protect the genetically modified cell(s), tissue(s) or organ(s) from an immune response from the host. In some embodiments, the matrix or coating is a biodegradable matrix or coating. In some embodiments, the matrix or coating is natural. In other embodiments, the matrix or coating is synthetic.

In some embodiments, the genetically modified cell(s), tissue(s) or organ(s) is administered with an immunosuppressive compound. In some embodiments, the immunosuppressive compound is a small molecule, a peptide, an antibody, and/or a nucleic acid (e.g., an antisense or siRNA molecule). In some embodiments, the immunosuppressive compound is a small molecule. In some embodiments, the small molecule is a steroid, an mTOR inhibitor, a calcineurin inhibitor, an antiproliferative agent or an IMDH inhibitor. In some embodiments, the small molecule is selected from the group consisting of corticosteroids (e.g., prednisone, budesonide, prednisolone), calcineurin inhibitors (e.g., cyclosporine, tacrolimus), mTOR inhibitors (e.g., sirolimus, everolimus), IMDH inhibitors (azathioprine, leflunomide, mycophenolate), antibiotics (e.g., dactinomycin, anthracyclines, mitomycin C, bleomycin, mithramycin) and methotrexate, or salts or derivatives thereof. In some embodiments, the immunosuppressive compound is a polypeptide selected from the group consisting of: CTLA4, anti-b7 antibody, abatacept, adalimumab, anakinra, certolizumab, etanercept, golimumab, infliximab, ixekizumab, natalizumab, rituximab, seckinumab, tocilizumab, ustekinumab, vedolizumab, basiliximab, daclizumab, and murmonab.

In some embodiments, the genetically modified cell(s), tissue(s) or organ(s) to be administered to the subject have been further genetically modified such that they are less likely to induce an immune response in the subject. In some embodiments, the genetically modified cell(s), tissue(s) or organ(s) have been further genetically modified such that they do not express functional immunostimulatory molecules.

The following examples are provided to illustrate the disclosure and are merely for illustrative purpose only and should not be construed to limit the scope of the disclosure.

### EXAMPLES

The disclosure now being generally described, it will be more readily understood by reference to the following examples, which are included merely for purposes of illustration of certain aspects and embodiments of the present disclosure, and are not intended to limit the disclosure. For example, the particular constructs and experimental design disclosed herein represent exemplary tools and methods for validating proper function. As such, it will be readily apparent that any of the disclosed specific constructs and experimental plan can be substituted within the scope of the present disclosure.

### Example 1: Knockout of Porcine Complement Component 3 (C3) to Inhibit Complement Systems

A highly conserved region of C3 was selected and two sgRNAs that target a C3 domain were designed. The sequences of the two gRNAs sequences are TCTCCAGACGCAGGACGTTG (SEQ ID NO: 158) and GGAGGCCCACGAAGGGCAAG (SEQ ID NO: 159). The C3 sgRNA was transiently transfected together with GGTA sgRNA (GAGAAAATAATGAATGTCAA (SEQ ID NO: 210)) plasmid and cas9 plasmid into porcine fetal fibroblast cells using the neon transfection machine and reagents. Cells lacking C3 (“C3-KO”) were selected using a GGTA antibody counter selection method to co-enrich the C3-KO cells which were then single cell sorted and genotyped to determine the efficiency of knocking down the C3 target using deep sequencing.

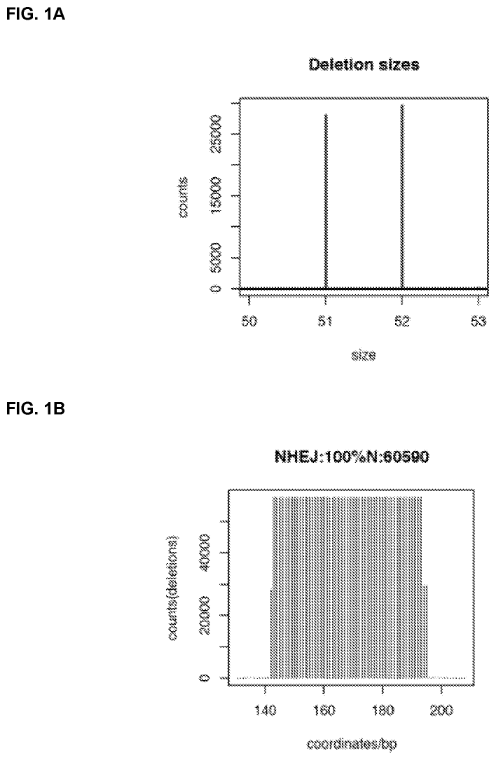

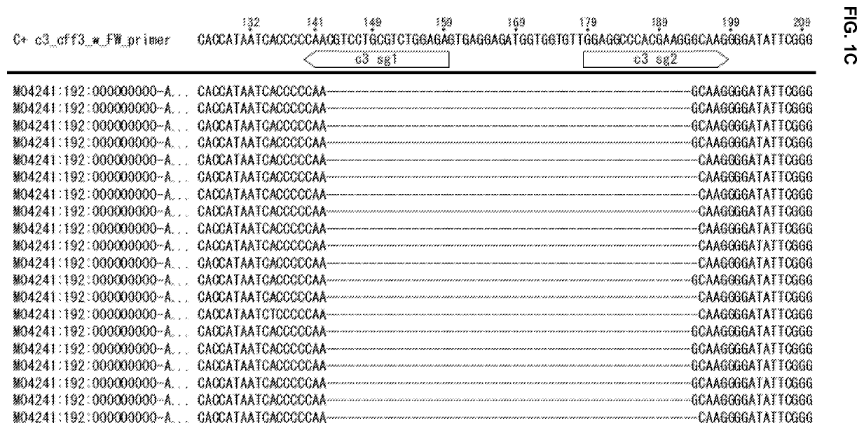

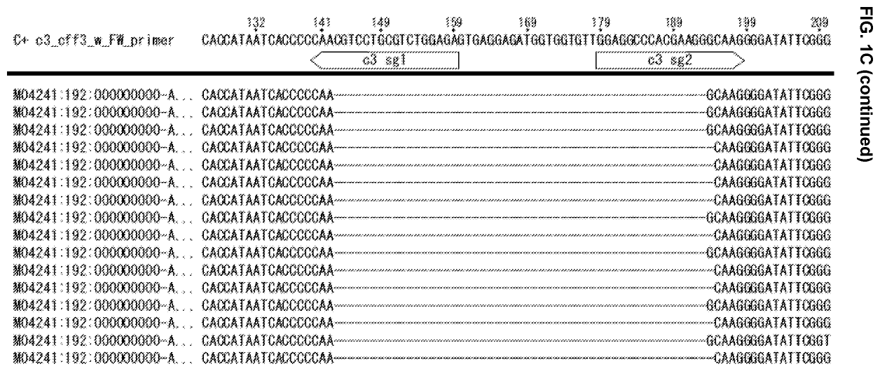

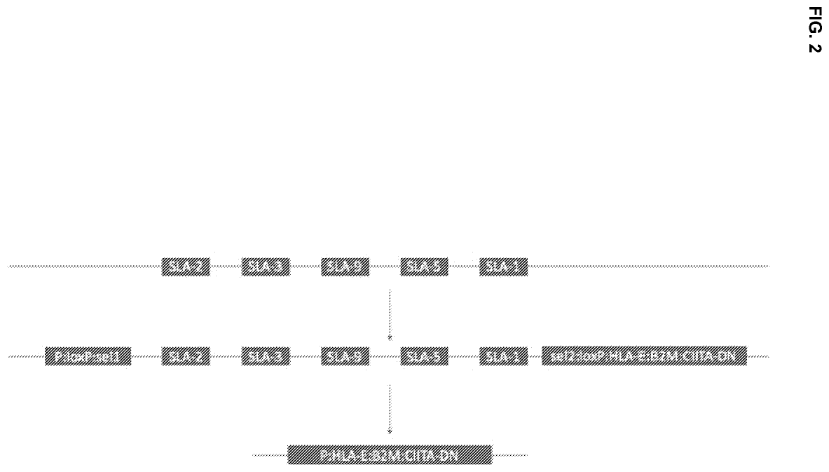

Among the 156 clones screened, 108 clones were bi-allelic C3-KOs. The efficiency of knock-down for the bi-allelic C3-KO cells was 69%. The resultant C3-KO cell line has been used to generate pigs using the somatic cell nuclear transfer method. The C3-KO pig was alive for 63 days and died of liver and lung infection. As shown in FIGS. 1A-C, the C3-KO pig was a 100% NHEJ knockout. FIG. 1A shows the sizes of deletions introduced into C3, FIG. 1B shows the position of the indels, and FIG. 1C shows the sequence of the indels generated in the C3-KO pig.

It is expected that the C3-KO pig described above would not have produced any functional C3 protein. Due to the lack of functional C3 protein, the C3-KO pig's complement system would not be activated thereby decreasing the C3-KO pig's innate immune system. In addition, it is expected that the C3-KO pig might be more prone to bacterial and/or viral infections compared to a wild-type pig. Moreover, xenotransplantation of a C₃-KO pig's cell, tissue, and/or organ into a human is not expected to activate the human complement system. This should therefore minimize the human innate immune response to the C3-KO pig xenograft.

### Example 2: Pigs Having One or More Modified MHC Class I Genes

A pig's MHC major class I alleles were conditionally replaced with human MHC minor class I alleles (“MHC-I pigs”). To do so, a region of the pig's genome containing the SLA-1, SLA-2, and SLA-3 genes was replaced with a modified version of the human minor allele HLA-E.

FIG. 2 depicts a scheme of the MHC class I replacement strategy: the locus containing SLA-1, SLA-2, and SLA-3 genes was flanked with IoxP sites. After treatment with Cre, SLA-1, SLA-2, and SLA-3 were excised and replaced by human HLA-E, such as various combinations of HLA-E, HLA-G, B2M, and CIITA-DN genes. The MHC-I pigs were viable and severely immunocompromised. Therefore, rather than replace the SLA-1, SLA-2, and SLA-3 genes with human genes universally, a conditional knockout was used and the SLA-1, SLA-2, and SLA-3 genes were replaced by the human HLA-E and other human genes prior to harvesting a cell, tissue, and/or an organ.

The MHC I region of the pig was sequenced using long reads technology. Probes to capture the SLA-1, SLA-2, and SLA-3 genes were designed and used to capture the MHC-I genetic region. PacBio sequencing and 10× sequencing were used to accurately determine the MHC-I genetic region. The configuration of SLA-1, SLA-2, and SLA-3 is illustrated in FIG. 9. Two cassettes having IoxP sites to flank the MHC-I region were designed. Cassette 1 contains a promoter, a IoxP site, and a selection agent (i.e., puromycin). Cassette 2 contains a second marker (GFP), a IoxP site, and a promoter-less cassette of genes including HLA-E, B2M and CIITA-DN.

Cassettes 1 and 2 were synthesized from individual components using a Golden Gate Assembly strategy (New England BioLabs) and were flanked with a 800 bp homology sequence corresponding to the insertion sites. Two consecutive rounds of CRISPR-cas9 were used to insert both sites¹⁷. Puromycin selection and GFP FACS sorting were used to isolate clones and junction PCR was used to validate insertions.

Cells were transfected with Cre recombinase and expression of Cre recombinase was induced. Single cell sorting was performed and sorted cells were screened using junction PCR to isolate cells having biallelic replacement of SLA-1, SLA-2, and SLA-3 with human MHC-1.

For in vivo Cre excision, an alternative cassette 1 has been designed and includes a Cre recombinase under control of a tissue specific promoter or an inducible promoter. By using a tissue specific promoter or an inducible promoter, the SLA-1, SLA-2, and SLA-3 genes will be excised in cell, tissue and/or organ of interest or excision can be induced in the animal prior to harvesting the cell, tissue, and/or organ. Pigs having SLA-1, SLA-2, and SLA-3 replaced with the human MHC-I can be generated by somatic cell nuclear transfer (SCNT) and piglets encoding conditional and/or tissue specific conditionally replaced genes can be generated.

### Example 3: MHC Class II Inactivation

Pigs lacking expression of the MHC-II alpha chain (“MHC-II KO pigs”) were generated by excising DQA genes and inactivating DRA genes using established gRNA technology in porcine cells which were then transferred into host pigs via SCNT. Briefly, following gRNA transfer into the porcine cells, the genome was sequenced and variation at the MHC-II loci were identified. Cas9 was delivered to these cells, which were then sorted to isolate single cells. These single cells were sequenced to genotype the targeted DQA and DRA genes. In single cells having DQA and DRA inactivation, embryos were generated following SCNT and were subsequently implanted into a pig to generate the MHC-II KO pig. Four weeks after birth, the MHC-II KO pig remained healthy.

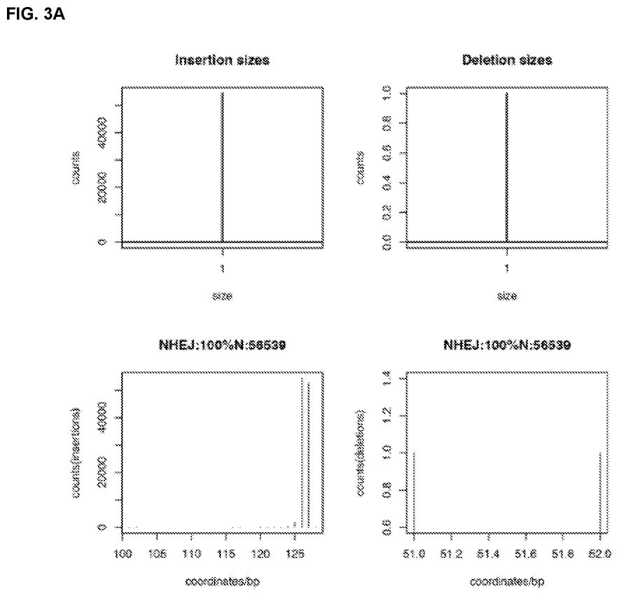

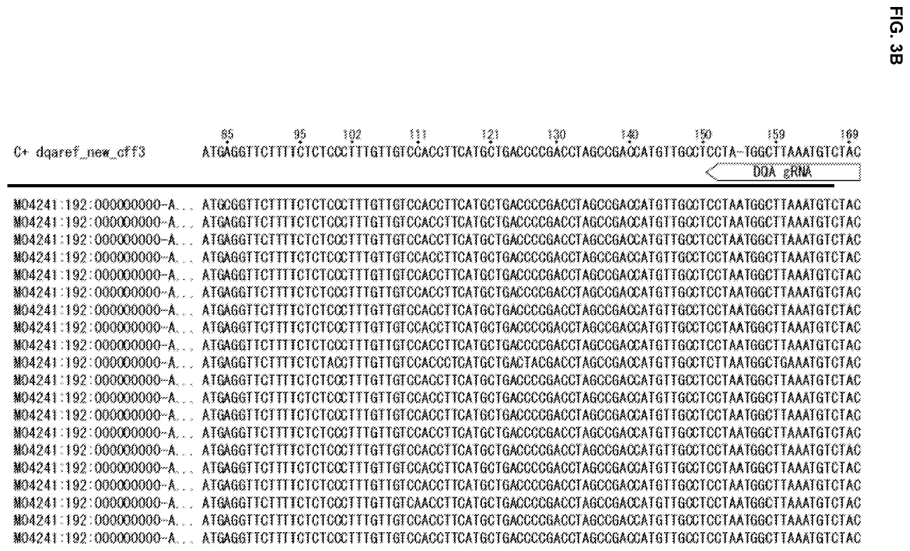

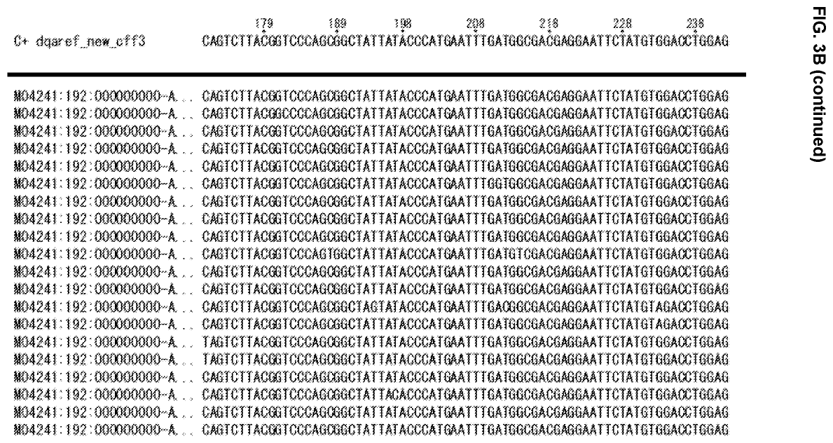

FIGS. 3A and 3B illustrate the genotype of the MHC-II KO based on the DQA gene. The MHC-II KO pig was genotyped by exonic targeting-based amplification and sequencing of the DQA gene as well as sequencing of the DRA gene. As shown in the left panel, the sizes and positions of the indels are located in the DRA gene. Inactivation of the DRA gene was caused by the two single nucleotide insertions at each of positions 126 and 127 in the DRA amplicon as illustrated in the right panel.

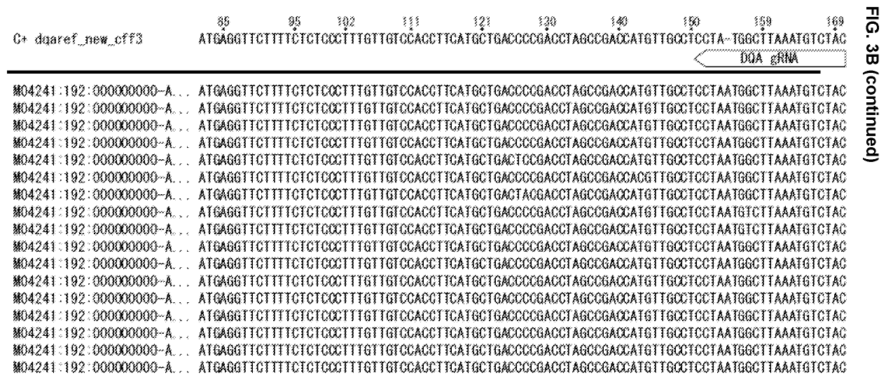

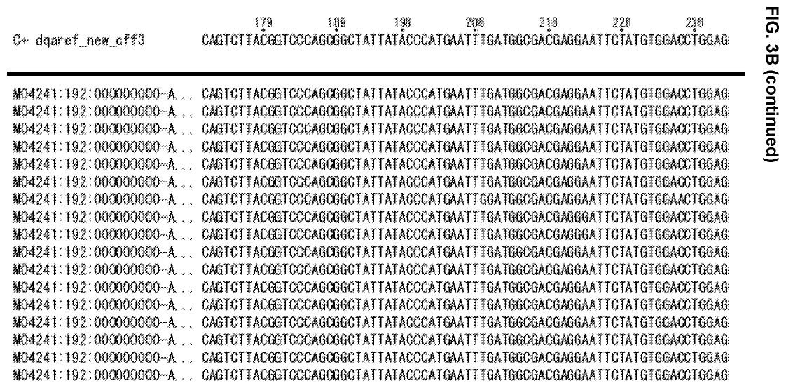

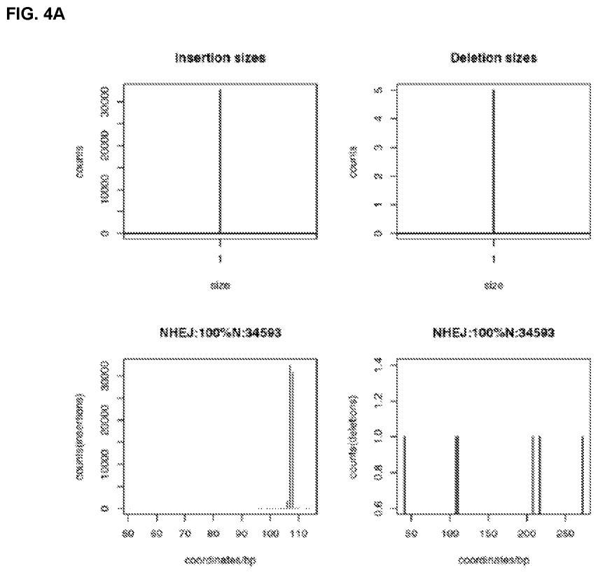

FIGS. 4A and 4B illustrate another genotype of an MHC-II KO pig. The DRA genotype was determined using exonic targeting-based amplification and sequencing of the DRA gene. The exonic targeting area from DRA has been amplified and sequenced. As shown in the left panel, the sizes and positions of the indels are located in the DRA gene. Inactivation of the DRA gene was caused by the two single nucleotide insertions at each of positions 106 and 107 in the DRA amplicon as illustrated in the right panel.

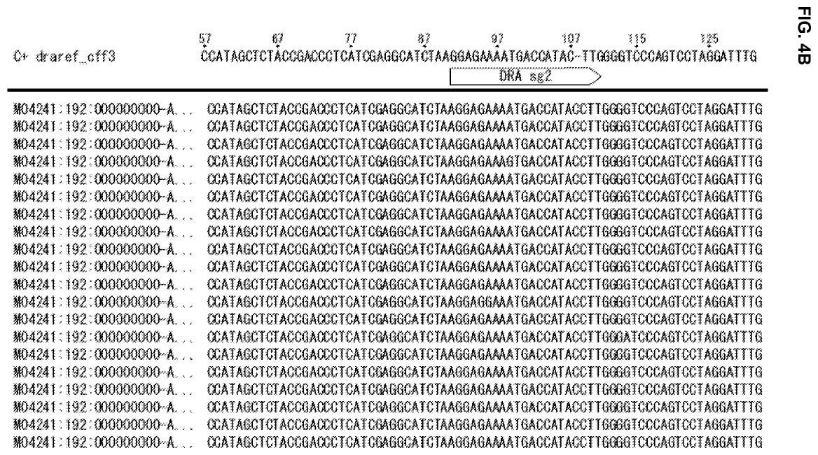

Similar to a human lacking MHC-II expression, the MHC-II KO pig has a decreased population of CD4⁺ T cells however, the CD8⁺ T cell population remains intact (FIG. 5). In addition, the MHC-II KO pig is immunosuppressed, has increased autoimmunity, and lymphoid defects, amongst other issues. These phenotypes are known to be associated with the MHC-II KO phenotype and have been observed in mice lacking MHC-II expression. These similarities confirm that the MHC-II KO pig is a valid MHC-II KO rather than an active gene modification (FIG. 6).

### Example 4: PD-L1 Knockin to Reduce Adaptive Immunity Based Rejection

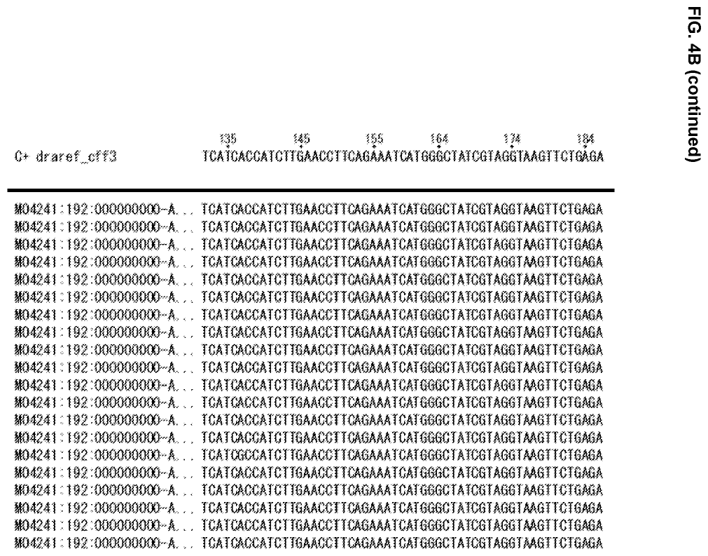

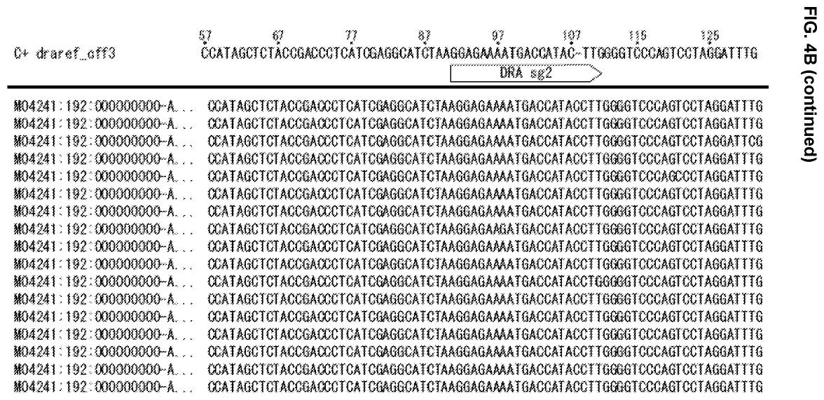

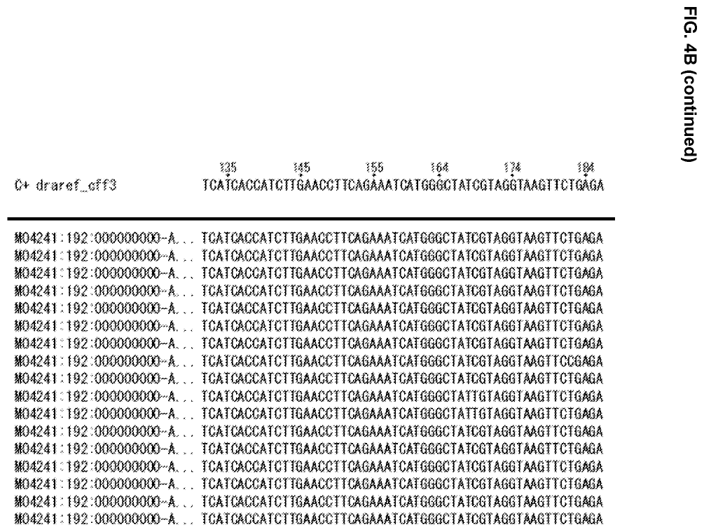

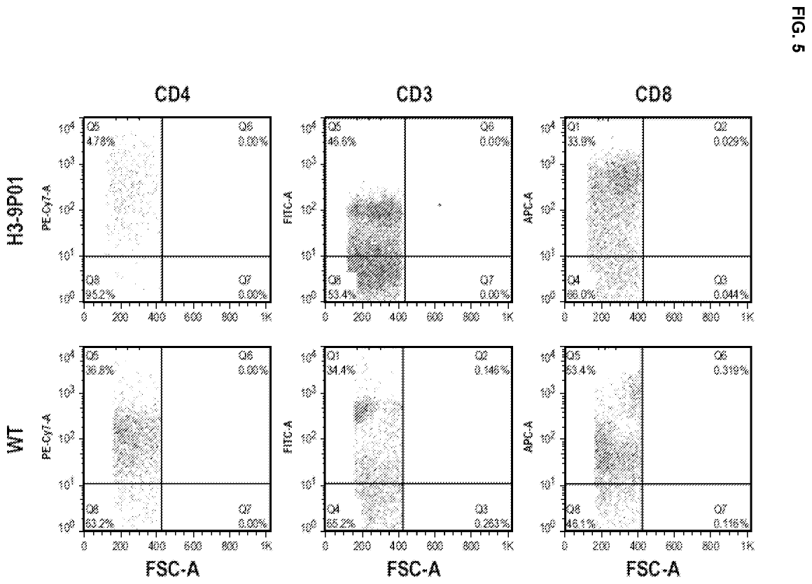

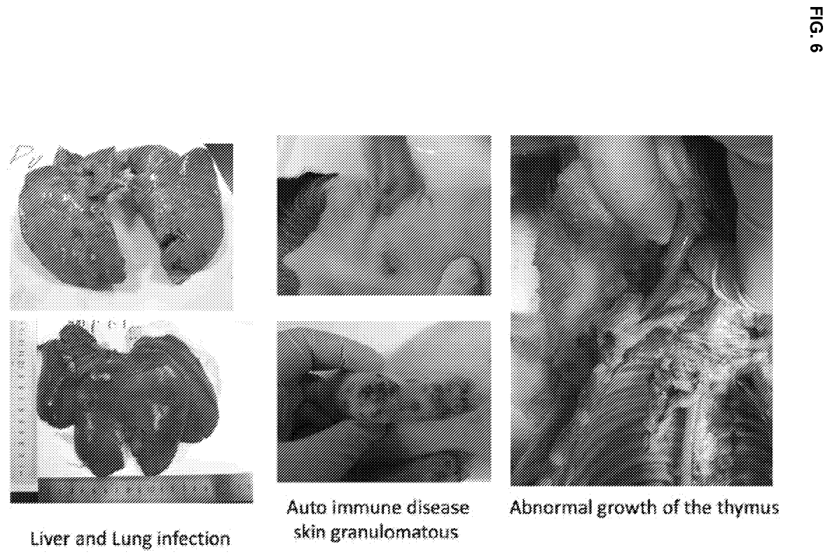

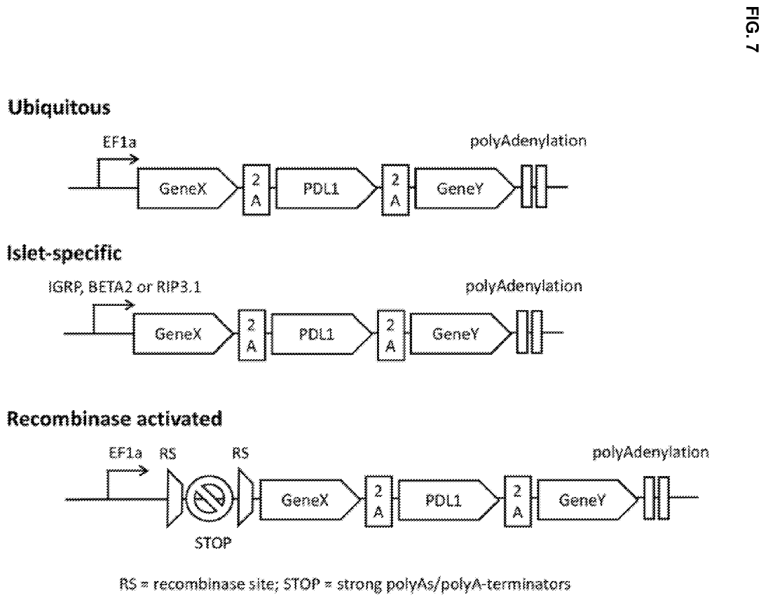

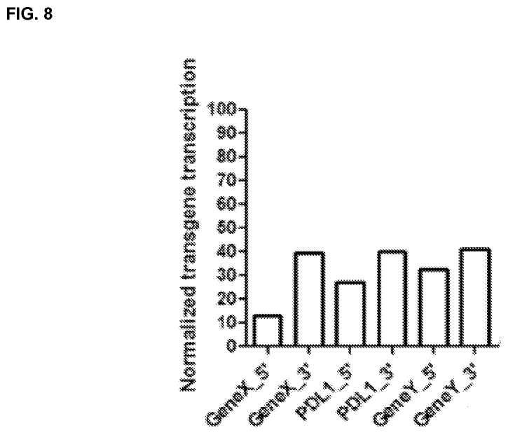

A human PD-L1 gene (e.g., PD-L1 transgene) was delivered to a pig genome. See scheme with structure in FIG. 7. Expression of the human PD-L1 transgene was confirmed by qPCR using two different PD-L1 amplicons (FIG. 8).

Porcine tissues expressing PD-L1 may have reduced rejection by a host, such as a human, following xenotransplantation.

### Example 5: Genetic Modification of Porcine Von Willebrand Factor to Modulate Platelet Aggregation

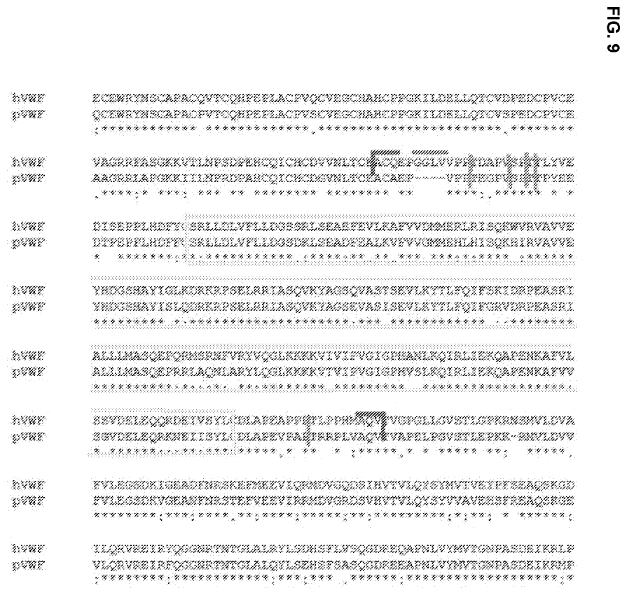

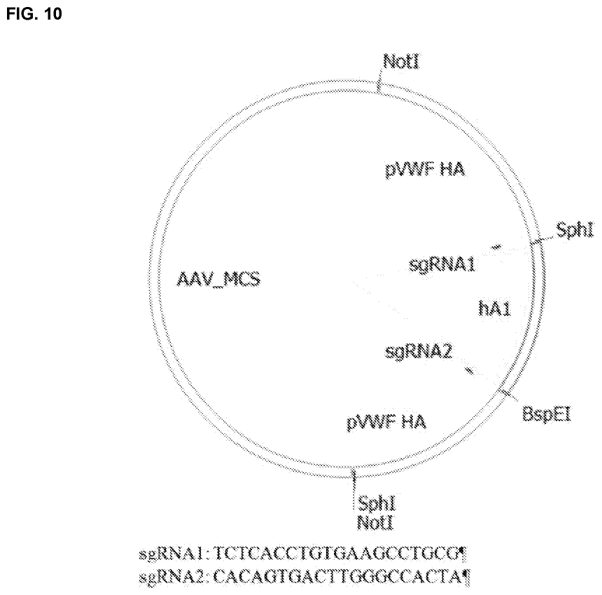

An HDR vector that contains the homology arms from pvWF, the A1 domain, and the certain residues in the flanking regions from hvWF was designed and constructed. (FIG. 10). Two sgRNAs were also designed to initiate the HDR replacement in the endogenous porcine genome and cut near the region to be replaced by the human sequences: TCTCACCTGTGAAGCCTGCG (SEQ ID NO: 5) and CACAGTGACTTGGGCCACTA (SEQ ID NO: 6).

The HDR vector is composed of ˜1 kb homology arms from porcine vWF and the human A1 and flanking domains as well as inactivating mutations in the sgRNA cutting sites to prevent sgRNA from cutting the donor and modified porcine genome. The HDR vector also contains SphI and BspEI sites that can distinguish the HDR vector from the endogenous porcine genome near the sgRNA cutting sites.

Porcine primary fibroblast cells were transfected using the Neon Transfection System (Invitrogen) with 8 μg of Cas9, 1 μg of sgRNA1, and 1 μg of sgRNA2, as well as 10 μg of the HDR vector. Two days after transfection, cells were single cell sub-cloned using FACS. The single cells were cultured for additional 12 days until the episomal form of the HDR vectors are lost during cell division. The A1 and flanking regions of the hvWF were amplified using flanking primers. The PCR product was subjected to SphI and BspEI sequential digestions to screen the clones having HDR replacement which would add novel SphI and BspEI sites to the PCR products having fragments sized at 700 bp, 323 bp and 258 bp following sequential digestion (FIG. 11). The complete bi-allelic HDR eliminates a wild-type product of 1281 bp as well as any partial digestion products larger than 700 bp.

A cell having a bi-allelic HDR was isolated from about 150 single-cell colonies (FIG. 11). As confirmed by sequencing, both alleles of the porcine A1 domain and flanking regions were replaced with the human counterpart (FIGS. 12A and 12B). The A1 domain is highlighted, whereas the potential glycosylation sites in the flanking region are labeled with dashes. The human specific residues that are deleted in pvWF are labeled with a bar and the humanized A1 domain and flanking regions are labeled with half parenthesis. This isolated cell has been expanded into a cell line and may be used to generate a genetically modified pig by SCNT.

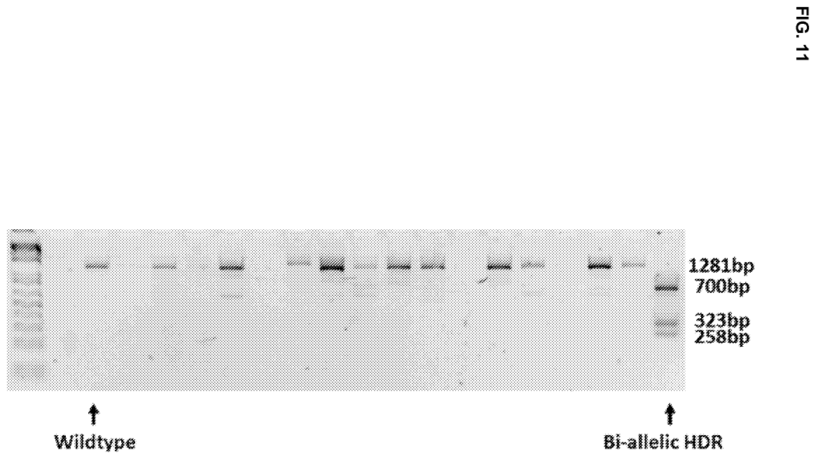

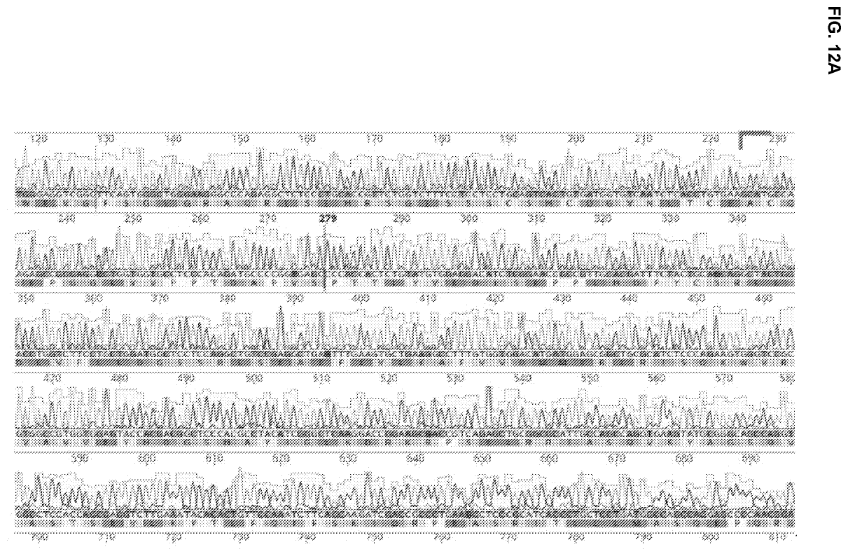

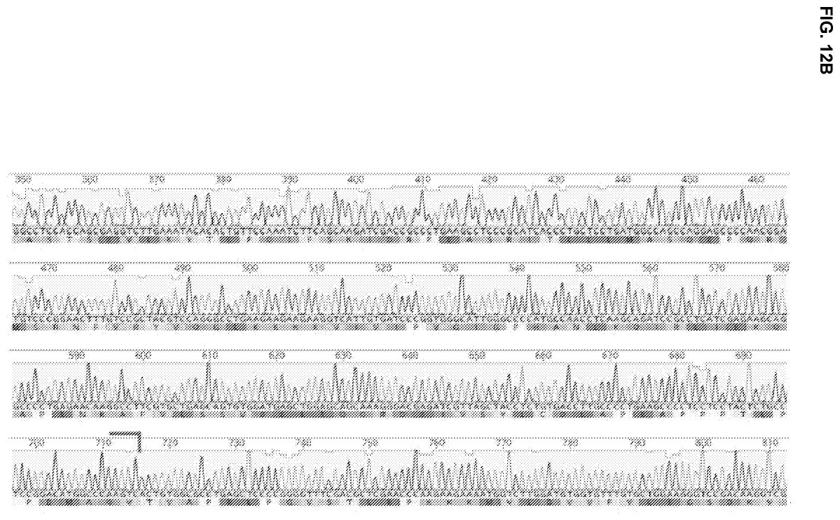

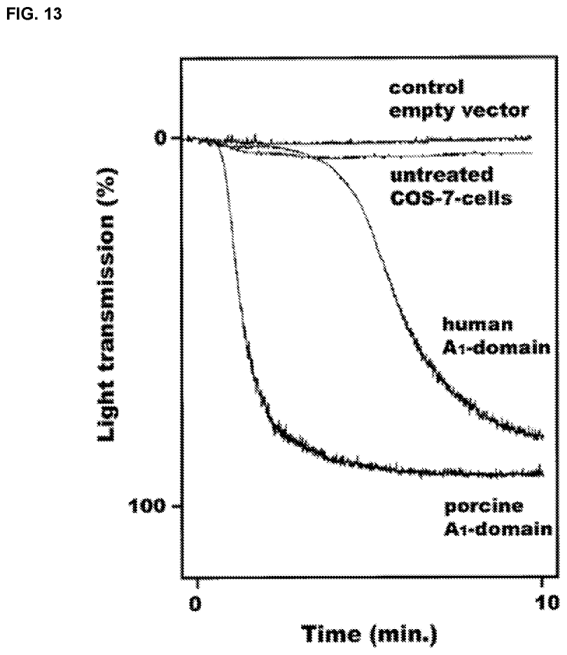

Cells expressing the A1-humanized pvWF had a significantly reduced aggregation response against human platelets during a platelet activation assay (FIG. 13). Briefly, the cells were incubated with human platelets and aggregation was induced by shear stress. The cells expressing the A1-humanized pvWF showed a milder and inducible aggregation curve whereas the cells expressing wildtype pvWF had a stronger aggregation response towards human platelets. Accordingly, porcine organs having A1 hvWF will likely induce a milder coagulation response in human blood compared to porcine organs expressing pvWF and might ameliorate the vascular incompatibility observed in pig-to-human xenotransplantation.

Together these data show that replacing the A1 domain and certain residues in one or more flanking domains of endogenous porcine pvWF with the corresponding residues from the human counterpart (hvWF) may modulate the platelet aggregation response that occurs during xenotransplantation (FIG. 9).

### Example 6: Genomic Deletion of Porcine Classical MHCI Antigens to Prevent CD8+ T Cell Activation

MHC class I molecules play a vital role in the rejection of allotransplantation through their peptide presentation to CD8+ T cells. Here, it was tested whether deletion of the entire ˜200 kb classical MHC class I locus in porcine primary fibroblast cells prevented CD8+ T cell mediated toxicity in xenotransplantation.

Classical MHC class I genes encode highly polymorphic proteins that are widely expressed in cell surface. They present foreign peptides to CD8+T lymphocytes leading to the lysis of target cells. Also, mismatched MHCI molecules also serve as antigens in transplantation. Different strategies of removing the classical MHCI molecular in donor porcine organs for xenotransplantation have been explored. In one attempt, the Tector group knocked out the conserved Exon4 of the SLA-1, SLA-2 and SLA-3 molecular using Cas9 and 3 sgRNAs (Reyes 2014). However, this exon is also share by other classical and non-classical MHCI molecular and it may generate unpredicted off-target effects. Also, the remaining Exon1-3 may still be presented as cell surface antigens. In another attempt, the heterodimerization partner B2M was knocked out using TALENs (Wang 2016). This method may also affect the non-classical MHCI molecules and the remaining MHCI may still be presented de-structured proteins on the cell surface. In the context of xenotransplantation, human HLA-E/B2M molecules are usually complemented in the MHCI deficient cells to prevent NK cell mediated toxicity. The human B2M might dimerize with porcine SLAs and restore their antigenicity in the B2M knockout pigs.

For this example, to specifically and completely remove the classical MHCI antigens, the MHC classical class I cluster with unique flanking sequences in the porcine genome were first identified (FIG. 14). This ˜200 kb cluster contains all the 8 classical MHCI genes without any other protein coding genes. Then, sgRNAs (SEQ ID NOs 1-4) in the unique flanking regions were identified to induce a fragmental deletion of this entire gene cluster. Because frequency of ˜200 kb fragmental deletion is relatively low, enrichment strategies were also designed to isolate bi-allelic deletion clones.

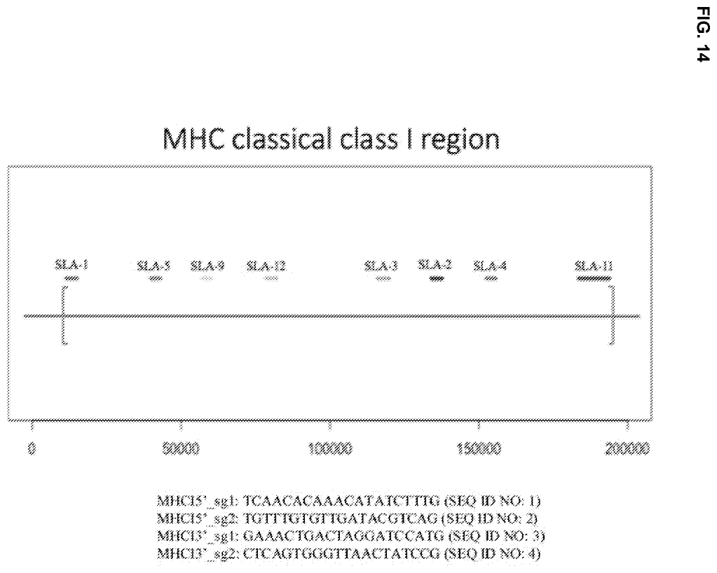

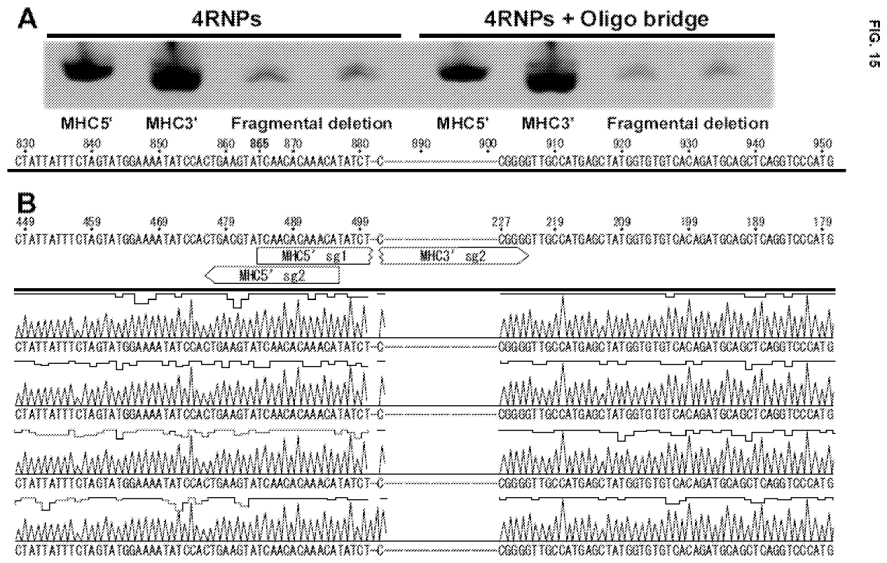

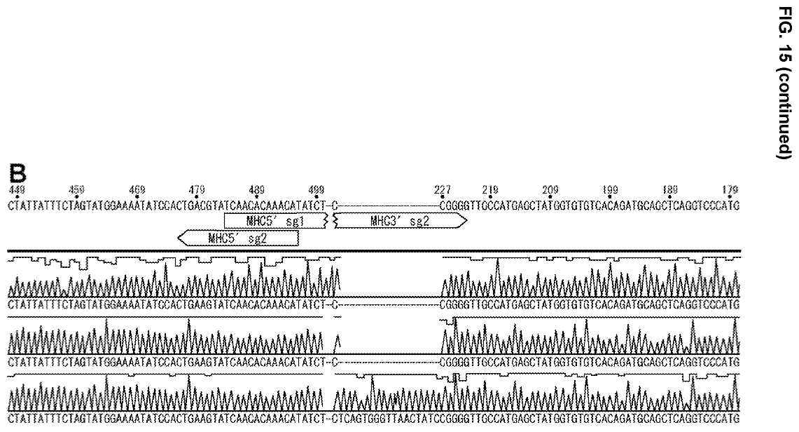

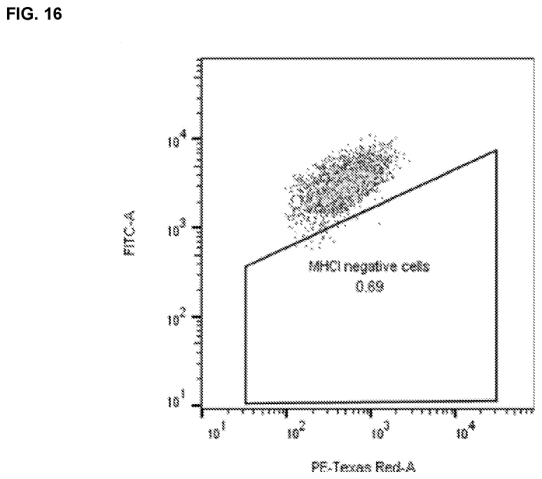

Porcine primary fibroblast cells were transfected with 1.25 μg of TrueCut Cas9 protein and 7.5 nmole of crRNA/tracrRNA duplex (Invitrogen) using the Neon transfection system (Invitrogen). Three days after transfection, genomic DNA was harvested from the transfected cells and subject to PCR using designated primer pairs shown in FIG. 15A. Fragmental deletion was detected using primers flanking the expected deletion junction. This PCR product was subcloned using Toposiomerase based cloning (“TOPO cloning”) and the individual TOPO clones were Sanger sequenced to confirm the sequence of the deletion junctions. The sequences were aligned to the expected junction shown in FIG. 15B. At the same time, an aliquot of the cells were stained with a pig-specific SLA-1 antibody. The portion of MHCI negative cells were shown in FIG. 16.

After single-cell subcloning, the cells containing bi-allelic deletion can be used to produce classical MHCI knockout pigs via somatic cells nuclear transfer. It is contemplated that the pigs are completely deficient in all classical MHCI molecules and proficient for the non-classical MHCI molecules, which might be involved in fertility and other physiological functions. The remaining B2M molecules are unlikely to be antigenic because they are non-polymorphic and highly conserved to the human counterpart. Also, the exogenous expression of human HLA-E/B2M cannot rescue the deficiency of classical MHCI molecules. The resultant pig should have the cleanest classical MHCI knockout background compared to previous reports.

### Example 7: Generation of Immunologically Compatible Porcine Cells, Tissue, Organs, Pigs, and Progeny

Despite many attempts by others to generate transgenic pigs for safe xenotransplantation, to date the most advanced transgenic pigs for xenotransplantation carried a limited number of transgenes due to the compacity of the constructs and transcription interference between transgenes. Here, a combination of KO, KI, and genomic replacement was utilized to generate several iterations of donor pigs. FIG. 21 outlines the progression of donor pig generations through sequential gene editing. As described below, in the case of Pig 2.0 (3KO+12TG) these gene edits included three knockouts and 12 transgene knockins designed to address immunologic, coagulation, and species incompatibilities.

CRISPR-Cas9 mediated NHEJ was used to functionally knock out the three major carbohydrate-producing glycosyltransferase/glycosylhydrolase genes GGTA1, CMAH, and B4GALNT2. Preformed antibodies that bind wild-type pig tissue are the major initial immunologic barrier to xenotransplantation, and these three genes have been identified as being largely responsible for producing the xenogenic antigens targeted by these antibodies (Byrne 2014, Lai 2002, Lutz 2013, Martens 2017, Tseng 2006). Thus, it was predicted that the functional loss of these genes would largely eliminate the binding of preformed anti-pig antibodies to the endothelium of the porcine graft. This was confirmed by flow cytometry results showing decreased binding of host antibodies to target Pig 2.0 (3KO+12TG) fibroblasts (FIG. 22). To demonstrate diminished antibody binding, genetically engineered pig fibroblasts were incubated with pooled human serum, and bound human IgM and IgG were detected with conjugated secondary anti-human antibodies and analyzed by flow cytometry. In contrast to wild-type pig fibroblasts (red contour plot), elimination of the three genes resulted in a significant reduction in antibody binding (green and brown contour plots, ˜98% decrease).

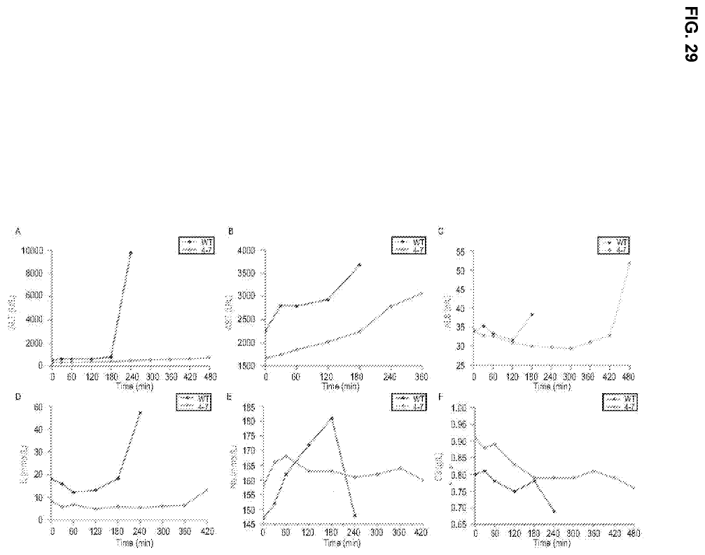

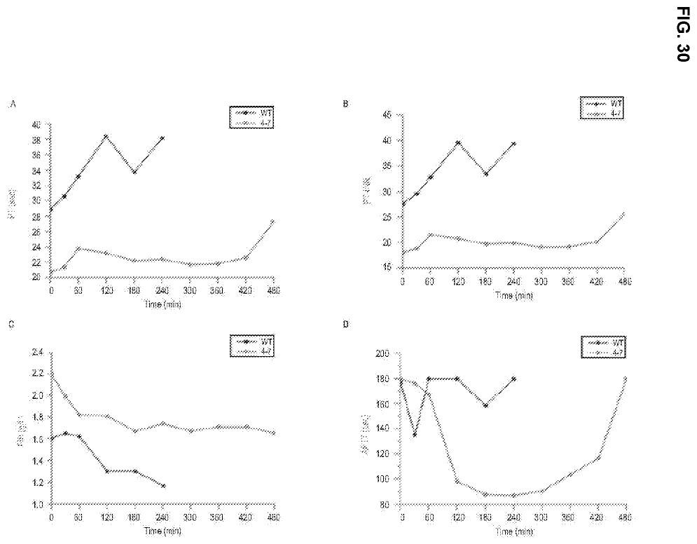

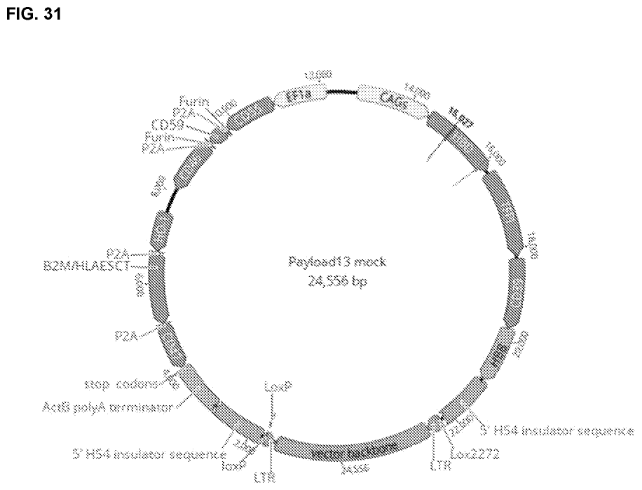

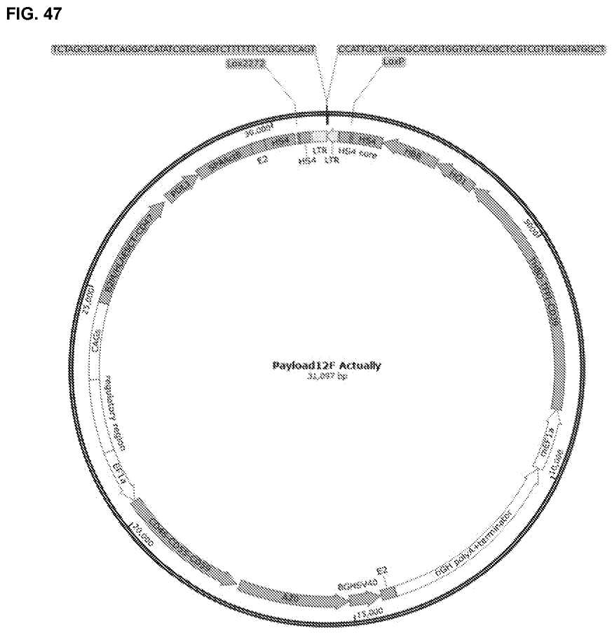

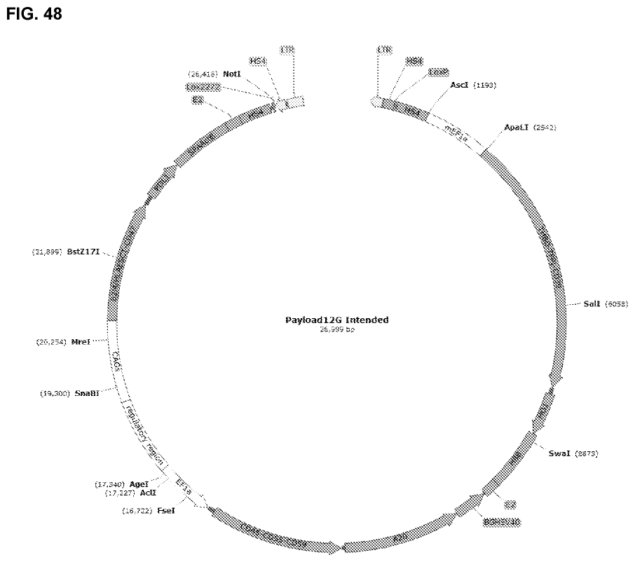

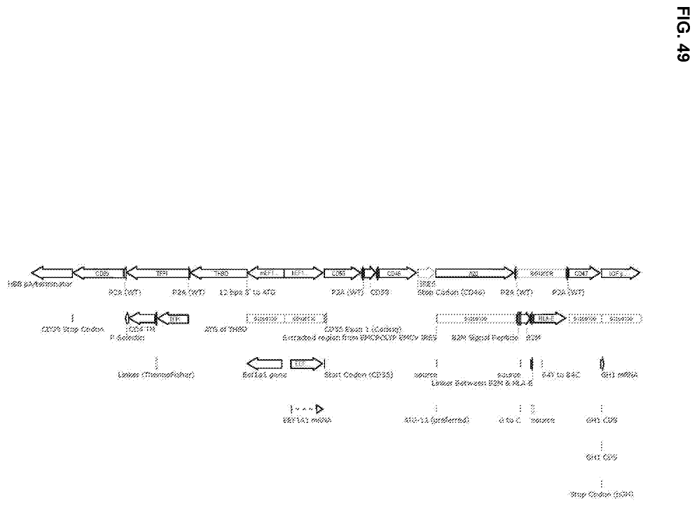

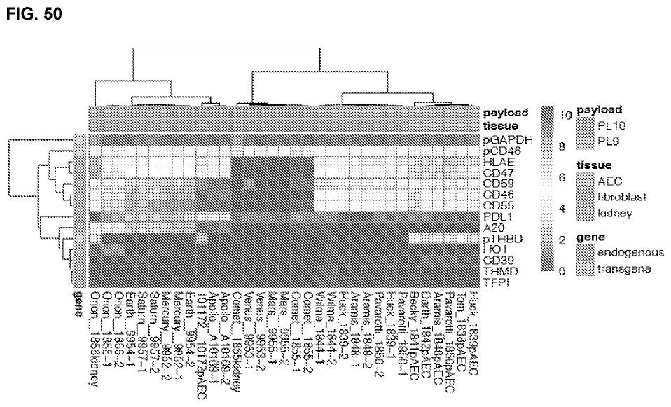

Twelve human transgenes (CD46, CD55, CD59, CD39, CD47, A20, PD-L1, HLA-E, B2M, THBD, TFPI, HO-1) were integrated into a single multi-transgene cassette in the pig genome via PiggyBAC transposon-mediated random integration to generate a first iteration of Pig 2.0 (3KO+12TG) (see FIGS. 17-20, 31, 47-49; SEQ ID NOs:212-214). The transgenes were arranged into 4 different cistrons with desired ubiquitous or tissue-specific promoters. The transgenes within each cistron were separated with ribosomal skipping 2A peptides to ensure expression in a similar molar ratio. Furthermore, a combination of cis-elements such as ubiquitous chromatin opening elements (UCOEs) were introduced to prevent transgene silencing and insulators with strong polyadenylation sites and terminators to minimize the interaction among transgenes and between transgenes and the flanking chromosome.

Transgene expression levels and tissue-specific promoter-driven expression was determined using qPCR (FIG. 23), and integration site and copy number were determined using junction capture based on inverted PCR. As a proof-of-principle, all transgenes in adjacent cistrons demonstrated desired tissue-specificity in fibroblast and endothelial cell lines without detectable transcription interference. In addition, all transgenes showed highly consistent expression levels across clones with various locations of genomic integration, which indicates that the transgene expression is independent of chromosomal context. As expected, the six genes inserted under control of a ubiquitous promoter, including the complement regulatory genes (CD46, CD55, and CD59; EF1α promoter) and B2M, HLA-E, and CD47 (CAG promoter) were expressed in both fibroblast and endothelial cells. In contrast, the six genes (A20, PD-L1, H01, THBD, TFPI, and CD39) expressed under the regulation of tissue-specific promoters (NeuroD or ICAM2), demonstrated lower levels of expression in fibroblasts relative to expression in endothelial cells. Consistent with the qPCR data, cell surface expression of proteins was observed to be expressed by the inserted human transgenes in pig spleen cells as well as in pig fibroblasts (FIG. 24). Briefly, Pig 2.0 (3KO+12TG) spleen cells or fibroblasts were isolated and incubated with antibodies recognizing specific human proteins as indicated and the stained cells analyzed using flow cytometry. In each panel of FIG. 24, the peak on the left represents cells stained with an isotype control and the peak on the right represents cells stained with the specific antibody.

For preclinical experimentation, the transgene knockins are randomly integrated into the genome using PiggyBac transposase, and clones with single copy integration into intergenic regions with no predictable consequences are used for pig production. For clinical development, homozygous female/male pigs will be generated with biallelic site-specific transgene integration into a safe harbor (e.g., the AAVS1 genomic locus) prior to scaled up breeding and production of source donor pigs.

Additional in vitro assessments of innate and adaptive immune cell function and complement and coagulation cascades will include antibody reactivity profiling, mixed lymphocyte reaction, complement-dependent cytotoxicity, NK cell cytotoxicity, macrophage phagocytosis, and effects on coagulation factors and platelet aggregation.

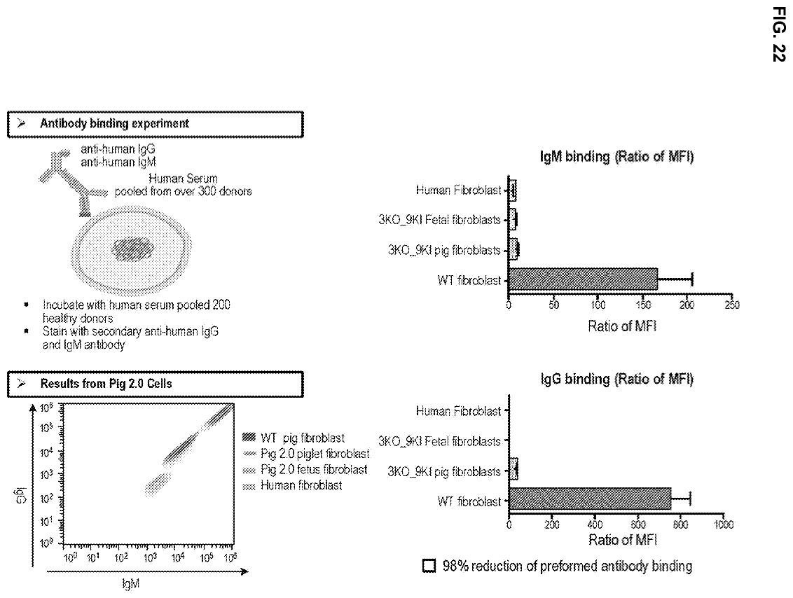

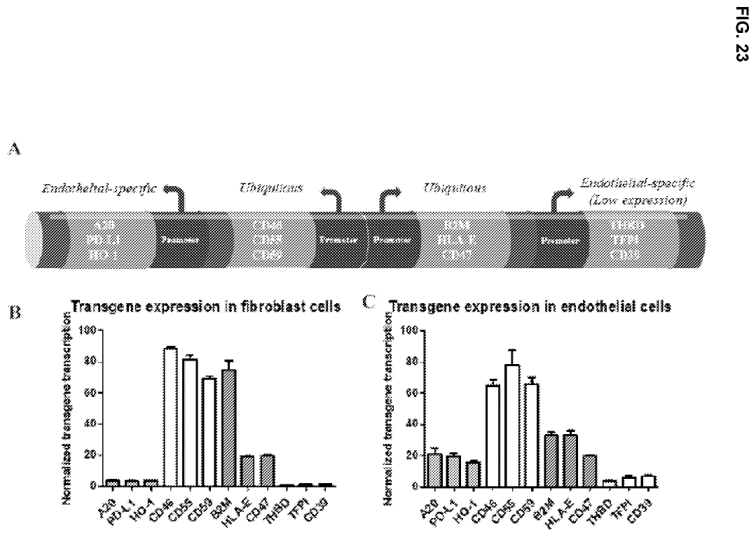

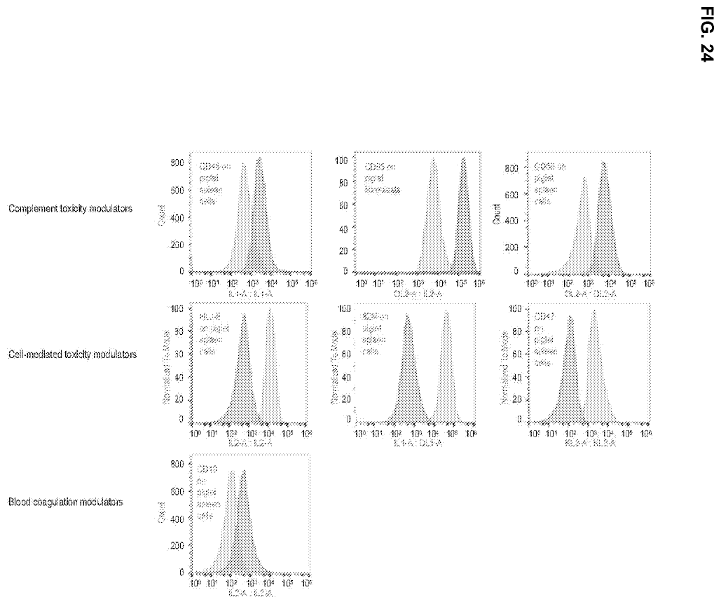

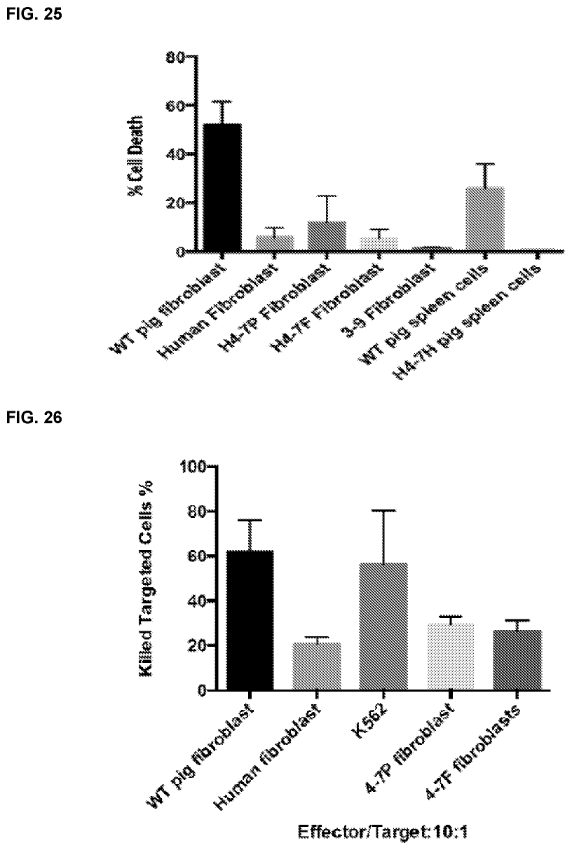

To maintain pig graft function and protect the donor organ from complement-mediated toxicity, human complement regulatory proteins were over-expressed. Briefly, genetically engineered pig fibroblasts and pig splenocytes were incubated with 25% human complement for one hour. Cells were stained with propidium iodide and analyzed by flow cytometry to quantify cell death. Wild-type fibroblasts and splenocytes demonstrated the highest percentage of cell death after culture with human complement. 4-7P and 4-7H cells are derived from Pig 2.0 (3KO+12TG) piglets; 4-7F cells (3KO+12 TG) are derived from a Pig 2.0 (3KO+12TG) fetus. 3-9 is triple carbohydrate antigen-producing enzyme KO, HLA-DQA KO, HLA-DRA KO, and human complement regulatory factor C3 KO. As shown in FIG. 25, pig fibroblasts and splenocytes genetically engineered to express human CD46, CD55, and CD59 exhibited significantly lower levels of complement-mediated cell death compared to control human fibroblasts.

Ligation of MHC I on target cells with Killer Inhibitory Receptors (KIR) on natural killer (NK) cells inhibits NK cell-mediated killing of target cells. Pig MHC I is incapable of transmitting signals through the human NK KIR and thus pig cells are susceptible to targeted cell killing by NK cells. To overcome NK-mediated cell death, human HLA-E, which ligates human NK KIR receptors, was overexpressed in pig cells. Seventy percent of WT pig fibroblast and K562 cells (human MHC-deficient cell line) were targeted for killing by NK cells. As shown in FIG. 26, human HLA-E+ engineered pig fibroblast cells demonstrated significantly lower NK-mediated cell killing. In contrast, HLA-E+ pig fibroblasts demonstrated significantly lower killing by NK cells, suggesting that expression of HLA-E protected these cells from lysis.

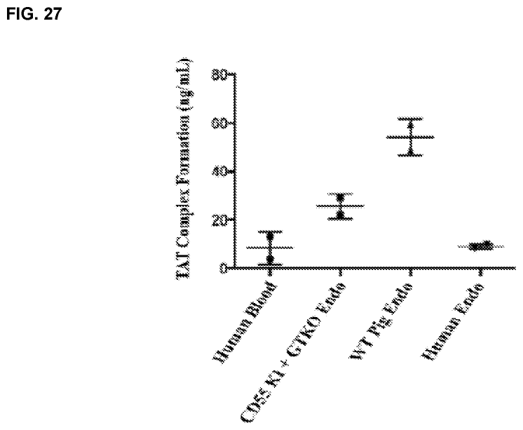

The over-expression of human CD55 in pig cells reduces complement-mediated toxicity which may diminish coagulation and improve xenograft survival. The activation of coagulation ultimately leads to the formation of thrombin which is inactivated by binding antithrombin in a stable thrombin-antithrombin (TAT) complex. Briefly, wild-type, CD55 KI+GGTA1-deficient cells, and human endothelial cells were cultured with human blood. As shown in FIG. 27, human blood alone or human blood incubated with human endothelial cells for 60 min generated approximately 10 ng/mL TAT protein. In addition, co-culture of human blood with wild-type pig endothelial cells activated coagulation and increased TAT complex formation to 58 ng/mL. In contrast, co-culture with CD55 KI+GGTA-deficient pig endothelial cells resulted in a significant decrease in TAT complex formation. These data suggest that human CD55 expression is able to modulate coagulation activation.

RNAseq was performed on samples isolated from pigs genetically modified with Payload 9 or Payload 10. Results demonstrated increased expression of several of the payload immune modifications transgenes, namely the complement transgenes, along with cellular toxicity genes (B2M, HLA-E, CD47) (FIG. 36).

### Example 8: Antibodies in Xenotransplantation and the Potential of Enzymatic Cleavage to Prevent Functional Binding

Antibody-mediated rejection has historically been the primary hinderance to the development of xenotransplantation as a viable treatment for end stage organ failure. However, recent genetic advancements have allowed for development of multiple-gene knockout pigs, which lack established xenoantigen targets. Knockout of aGal, Neu5Gc, and SDa have been linked to improved graft survival. However, further work is needed to fully understand the impact of residual antibody binding to other xenoantigen targets and if the removal of these antigens protects tissues from highly sensitized human serum. Here, it was investigated whether xenoantigen knockout decreases high PRA serum binding and whether functional antibody binding is decreased by enzymatic degradation.

Human and porcine PBMCs were collected from peripheral blood using Ficoll separation. Porcine aortic endothelial cells (pAECs) were processed from WT pigs and the genetically modified Pig 2.0 (3KO+12TG) of Example 7. Anonymous high and low PRA serum samples were generously provided by the Massachusetts General Hospital HLA laboratory. Serum was collected from heart, liver, and kidney xenotransplant recipients. Serum antibody was enzymatically cleaved by IdeS (Genovis Inc.).

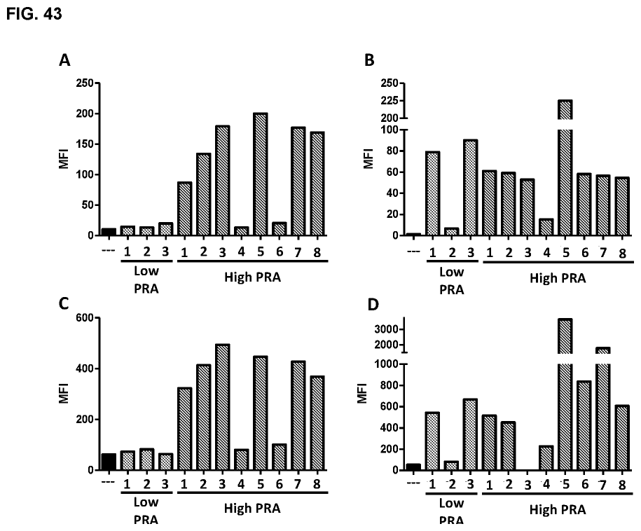

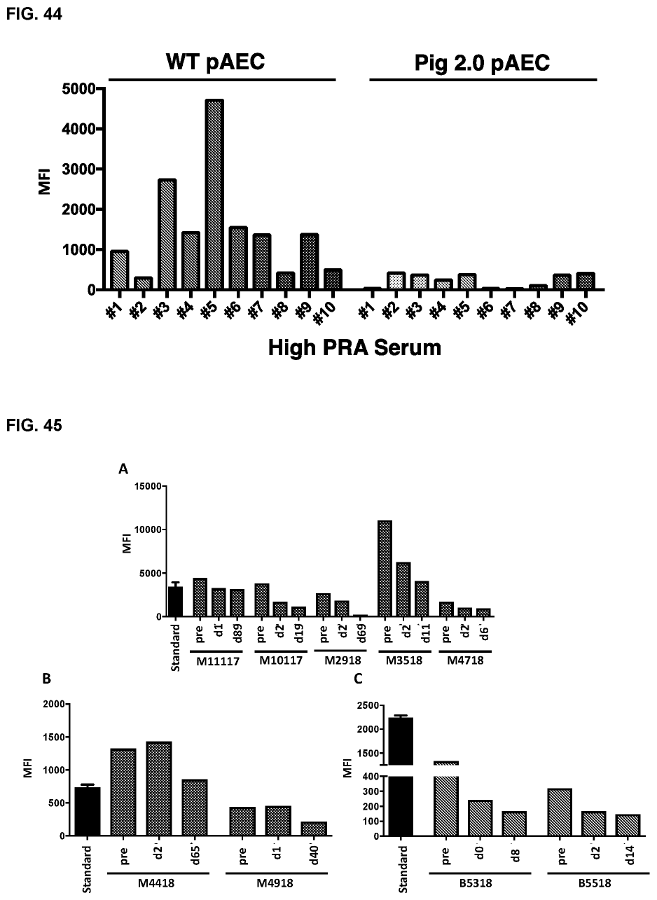

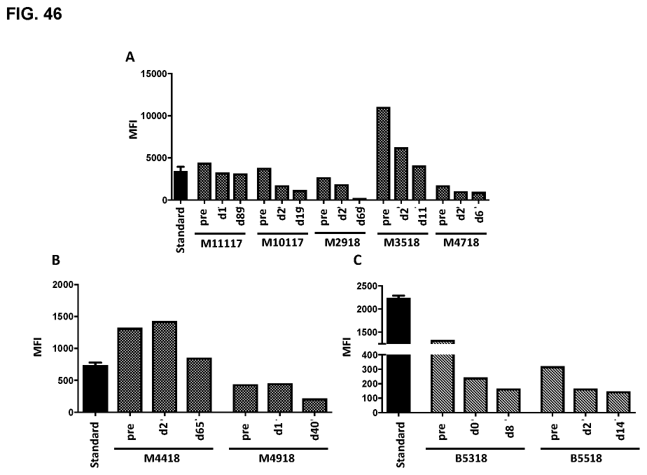

Low PRA human sera show minimal binding to human PBMC target cells, while high PRA human sera bind to the same human PBMC at a high level (FIG. 43A). In contrast, both high and low PRA sera strongly bind porcine PBMC (FIG. 43B). High PRA sera also show significant binding to porcine aortic endothelial cells (pAEC). Genetic modifications dramatically (>95%) reduce the binding of all human sera (FIG. 44). Importantly, in vivo xenotransplant experiments, using heart, liver, and kidney xenotransplants from Pig 2.0 (3KO+12TG), show sequestration of porcine specific antibodies through a reduction of antibody binding from recipient serum taken post-transplant (FIG. 45). These data suggest that a low level of residual xenoantibodies are present. FIGS. 46A-46C show that the IgG-specific protease, IdeS, effectively reduces the binding of functional IgG from human and cynomolgus serum to background levels.

Genetic modifications to remove known xenoantigen targets reduce the binding of human and primate serum to porcine cells, although low level xenoantibody binding remains. High and low PRA sera are similar, suggesting that the binding is likely not related to HLA-SLA cross-reactivity. IdeS treatment of sera from highly sensitized patient demonstrated a negative cross match to Pig 2.0 (3KO+12TG) cells. Other approaches to protect the xenograft targets from antibodies with unknown targets is to use additional genetic modifications to prevent downstream sequelae, such as complement activation and thrombogenesis. This data shows, for the first time, that enzymatic antibody cleavage may successfully reduce the functional binding of the residual IgG, suggesting this treatment may also be an approach to reduce the impact of pre-formed xenoantibody binding.

### Example 9: Generation of PERV-Free and Immunologically Compatible Porcine Cells, Tissue, Organs, Pigs, and Progeny

Porcine organs are considered a favorable resource for xenotransplantation since they are similar to human organs in size and function, and pigs can be bred in large numbers. However, the clinical use of porcine organs has been hindered by the potential risk of porcine endogenous retrovirus (PERV) transmission, and by immunological incompatibilities. PERVs are gamma retroviruses found in the genome of all pig strains. Pig genomes contain from a few to several dozen copies of PERV elements (Lee 2011). Unlike other zoonotic pathogens, PERVs are an integral part of the pig genome. As such, they cannot be eliminated by bio-secure breeding (Schuurman 2009). Although no study has shown PERV transmission to humans in the clinical setting to date, it has been demonstrated that PERVs can infect and propagate in human cells through “copy-and-paste” mechanisms. In cell culture, it has been shown that viral particles can be released and can infect human cells and randomly integrate into the human genome, preferentially in intragenic regions and in areas of active chromatin remodeling (Armstrong 1971, Moalic 2006, Niu 2017, Patience 1997). It has also been demonstrated that both PERV-A and PERV-B can infect human cells. Although PERV-C is ecotropic, the recombinant viral type (A/C) demonstrates the greatest infectivity. In addition, once PERVs adapt to the new host genome environment through elongation of the LTR sequence, infectivity potential may increase. PERVs can also pass horizontally from infected human cells to other human cells that have had no contact with porcine cells. In vivo in immunocompromised mice, it has been demonstrated that PERV can pass from pig cells to mouse cells (Clémenceau 2002). PERV integration could potentially lead to immunodeficiency and tumorigenesis, as reported with other retroviruses. Recent breakthroughs in genetic engineering have demonstrated genome-wide inactivation of PERV in an immortalized pig cell line (Yang 2015; PCT Publ. No. WO17/062723) and production of PERV-free pigs (Niu 2017; PCT Publ. No. WO18/195402).

Leveraging CRISPR-Cas9 technology, the complete elimination of all 62 copies of the PERV elements from the PK15 pig kidney epithelial cell genome (Yang 2015) and all 25 copies from porcine fetal fibroblasts and subsequent generation of live pigs with all PERV elements inactivated (Niu 2017) has been achieved. This success demonstrated that it is now possible to derive PERV-free pigs, which may provide a safe donor pool for xenotransplantation.

To determine whether PERVs remain active and propagate in human cells, PERV copy number was monitored both in a population and in clones of PERV-infected HEK293T-GFP cells (iHEK293T-GFP) for greater than 4 months. PERV copy number was observed to increase over time, as determined by ddPCR (Pinheiro 2012).

Studies to determine whether disruption of all copies of PERV pol in the pig genome could eliminate in vitro transmission of PERVs from pig to human cells have been conducted (Niu 2017). Reverse transcriptase activity could not be detected in the cell culture supernatant of highly engineered PERV fetal fibroblast clones, suggesting that modified cells produce minimal, if any, PERV particles. PK15 clones with >97% PERV pol targeting exhibited up to 1000-fold reduction of PERV infection, similar to background levels. These results were confirmed with PCR amplification of serial dilutions of human embryonic kidney 293 (HEK293) cells that had a history of contact with PK15 clones. Isolated total RNA from a variety of tissues of the pigs has confirmed ˜100% PERV inactivation at the mRNA level.

To date, multiple clones with 100% PERV KO have been produced from the Yorkshire breed and pig cloning is in progress. The PERV-inactivated pig production is robust and 63 PERV inactivated piglets have been produced, among which 47 are female and 16 are male. To date the oldest healthy animal has survived for two years. 43 PERV KO pigs are currently aging for breeding. Consistent with the normal karyotype of the cells used to clone the pigs, abnormal chromosomal structural changes have not been detected in the PERV inactivated pigs.

Long term studies to monitor the impact of PERV-inactivation and gene editing on large animals are being conducted. This technology is being applied to additional pig strains, including both Yorkshire and Yucatan pig strains in the US. Source donor pigs will be genetically engineered on a background line with all PERV elements disabled.

Version Iteration of PERV-Free and Immunologically Compatible Pigs. Studies have been undertaken to engineer donor pigs that do not harbor any active PERVs in the genome as well pigs that have enhanced immunological, inflammatory, and coagulation systems compatible with human tissues. With respect to the former, pigs wherein the function of all the PERVs in the pig genome have been eradicated using CRISPR-Cas9 engineering to disrupt the catalytic domain of the reverse transcriptase gene (pol) in the PERV elements (using the methods as described in Niu 2017 and WIPO Publication No. WO2018/195402) and using a combination of knockout (KO), knockin (KI), and genomic replacement to provide human tissue compatible organs. With respect to the latter, pigs wherein three of the major xenogenic carbohydrate antigen-producing genes/enzymes that trigger humoral rejection, GGTA1, CMAH, and β1,4 N-acetylgalactosaminyl transferase 2 (B4GALNT2) have been genetically inactivated were generated as described herein. It was contemplated that the functional loss of these genes would largely eliminate the binding of preformed anti-pig antibodies to the endothelium of the porcine graft. In addition, key immunological modulatory factors were inserted at a single locus within the PERV-free pig genome to regulate e.g. the human complement system (hCD46, hCD55, and hCD59), the coagulation system (e.g. hCD39, hTHBD, and hTFPI), the inflammation response (e.g. hA-20, hCD47, and hHO-1), and NK (e.g. PD-L1) and T cell responses (e.g. hHLA-E, hB2M). Single copy polycistronic transgene integration through transposition was used to knock in these humanized genes.

It was contemplated that pigs that are both PERV-free and bear an immunocompatibility payload could be generated, which pigs would have a variety of desirable properties. Toward this goal, donor pigs were created through several iterations of genetic modifications. FIG. 21 outlines the progression of donor pig generations through sequential gene editing. In the first iteration, Pig 1.0, porcine fibroblasts have been genetically engineered, using CRISPR-Cas9 mediated non-homologous end joining (NHEJ), to have all PERV copies functionally deleted from or inactivated within the genome. Pig 2.0 was generated through CRISPR-mediated NHEJ to delete the 3 major xenogenic carbohydrate antigen-producing genes (3KO; GGTA1, B4GALNT2 and CMAH) coupled with PiggyBAC-mediated random integration of up to 12 selected transgenes or knock-ins selected from CD46, CD55, CD59, HLA-E, B2M, CD47, CD39, THBD, TFPI, A20, PD-L1, and HO-1 that modify various components of the xenogenic immune response into the porcine genome. For the Pig 3.0 iteration, source donor pigs are then generated to carry the 3KO and up to12 specified transgenes, on the PERV-free background. It is contemplated that the next generations of source donor pigs (Pig 3.1, 3.2, etc.) will be genetically engineered to carry additional modifications, such as humanization of the vWF gene and deletion of the asialoglycoprotein receptor 1 (ASGR1) and endogenous B2M genes.

Once PERV-free 3KO+TG pigs (Pig 3.0, FIG. 21) have been genetically engineered, these pigs will be crossbred to generate progeny and/or a drift, drove, litter, and/or sounder of swine.

Cell Engineering and SCNT to Produce Pig. 3.0 Incorporating an Immunocompatibility Payload, Xenogenic Antigen Disruption, and PERV Disruption

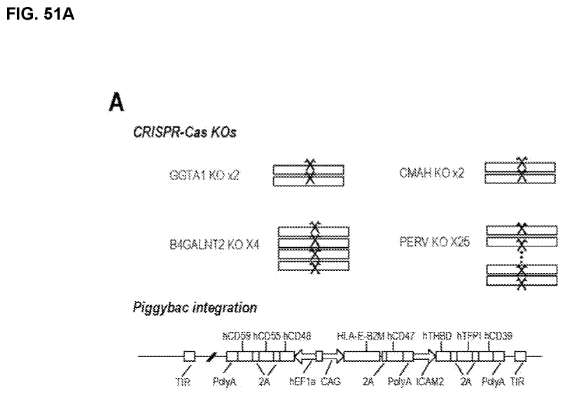

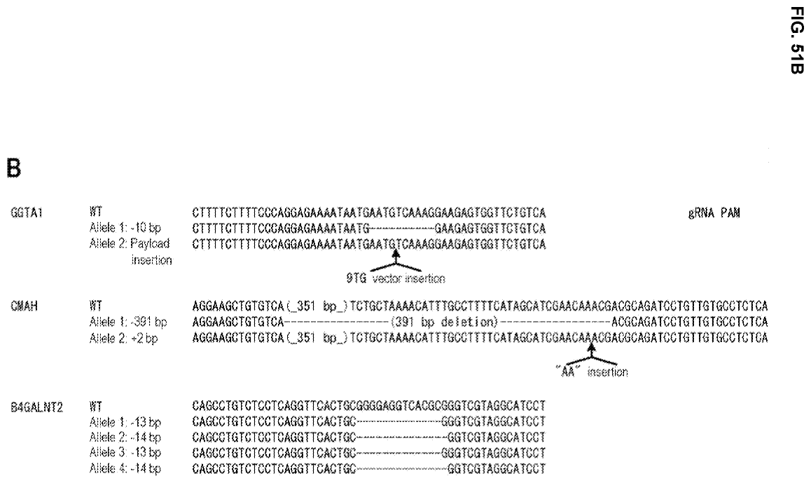

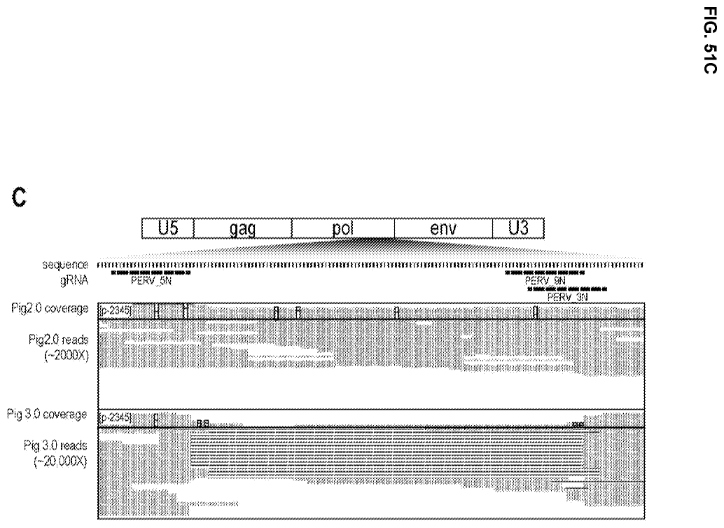

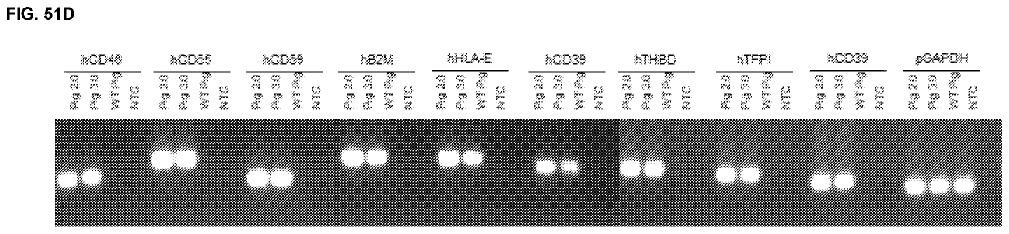

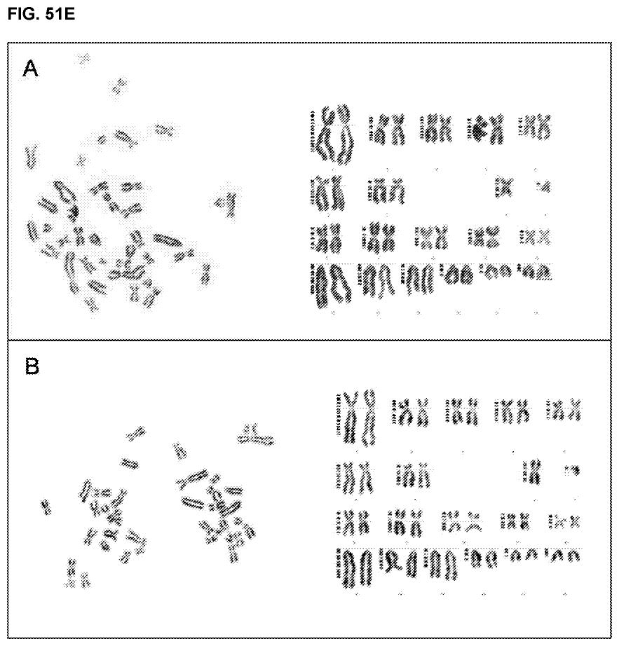

For production of PERV-free Pig 3.0, Pig 2.0 (3KO+9TG) with xenocompatibility modifications were generated first. Pig 2.0 (3KO+9TG) included the transgenes hCD46, hCD55, hCD59, hB2M, hHLA-E, hCD47, hTHBD, hTFPI and hCD39. To generate donor cells for the somatic-cell nuclear transfer (SCNT) to produce Pig 2.0, wild-type porcine ear fibroblasts were first electroporated with both: a) CRISPR-Cas9 reagents targeting the GGTA, CMAH, and B4GALNT2 genes; and b) payload plasmids bearing (i) a PiggyBac transposase cassette (ii) a transgenic construct consisting of the nine human transgenes (hCD46, hCD55, hCD59, hB2M, hHLA-E, hCD47, hTHBD, hTFPI and hCD39) organized into 3 expressible cistrons (see FIG. 51). Single-cell clones of the fibroblasts were generated and screened by a) fragment analysis/whole genome sequencing to identify clones with the desired genomic modifications (see FIG. 51C) and b) conventional PCR (see FIG. 51D). A clone bearing the desired modifications was then used as a donor to produce pig 2.0 by SCNT.

With isolated cells in hand from Pig 2.0 (3KO+9TG), PERV engineering using a CRISPR-Cas9 system was used to generate cells with xenocompatible modifications that are also PERV-free. Pig 2.0 fibroblasts were electroporated with CRISPR-Cas9 reagents targeting the reverse transcriptase (Pol) gene common to all genomic copies of the PERV elements. Single-cell clones of the electroporated cells were generated, and these clones were screened by deep-sequencing to identify clones in which the catalytic core of the Pol gene was disrupted (see FIG. 51C). Clones with the desired disruption in Pol were then subjected to karyotyping (see FIG. 51E); those with a normal karyotype were then used in SCNT to produce the Pig 3.0 (3KO+9TG) embryo and pig.

Characterization of Pig 3.0 Genomic, Biochemical, and Phenotypic Features

A) Evaluation of Transgene and Knockout Integrity

Having produced Pig 3.0 (3KO+9TG), we next sought to examine closely the on-target and off-target effects of genetic modifications therein. To this end, we performed 10× whole genome sequencing (WGS) on WT fibroblasts as well as the Pig 2.0 and Pig 3.0 fibroblasts generated above. Consistent with the deep-sequencing done for screening, the WGS confirmed the mutations introduced into genomic copies of PERV pol and GGTA/B4GALNT2/CMAH genes were all frameshift insertions or deletions that are expected to translate into functional knockouts of the modified gene copies (see FIGS. 51A and 51C). In addition, we confirmed the presence of all nine transgenes in the porcine genome and, surprisingly, the transgenic construct was found to have integrated into one of the GGTA1 alleles at the CRISPR-Cas9 targeted site.

With respect to potentially confounding off-target effects of CRISPR editing, we found no artifacts expected to interfere with the function of our desired edits or with expected deleterious effects on pig health. We did not observe any difference in structural variants between WT and Pig 2.0 (3KO+9TG), or between Pig 2.0 (3KO+9TG) and Pig 3.0 (3KO+9TG), indicating gross genomic stability for these pigs. With respect to smaller genomic changes such as small indels, we examined all 1,211 predicted off-target sites for the guide RNAs used and found two small insertions in the B4GALNT2 gRNA off-target sites in Pig 2.0 compared to WT; however, neither affect protein coding sequences. Additionally, when we compared Pig 3.0 cells to Pig 2.0 cells, we observed no additional genomic alterations expected to be of consequence; we found only two deletions and one insertion within two PERV gRNA off-target sites, both of which occur outside protein coding regions and which may actually represent somatic mutations (see Kim 2014). Given the lack of functional implications and together with largely normal pathophysiology data of our pigs, we conclude that the selected Pig 3.0 maintained genomic stability.

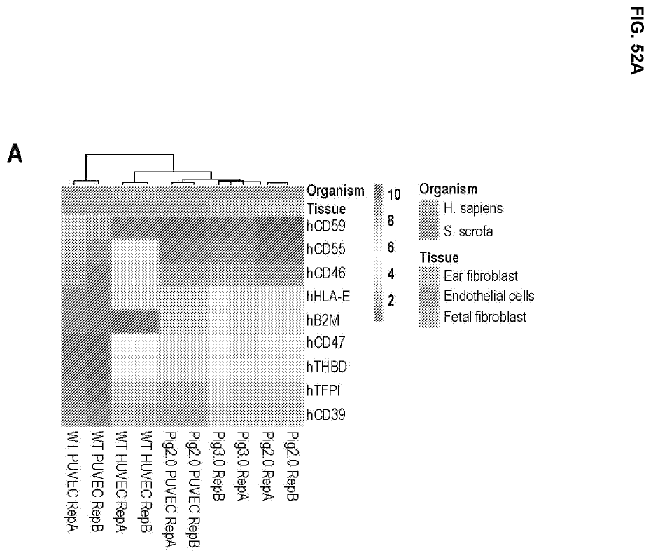

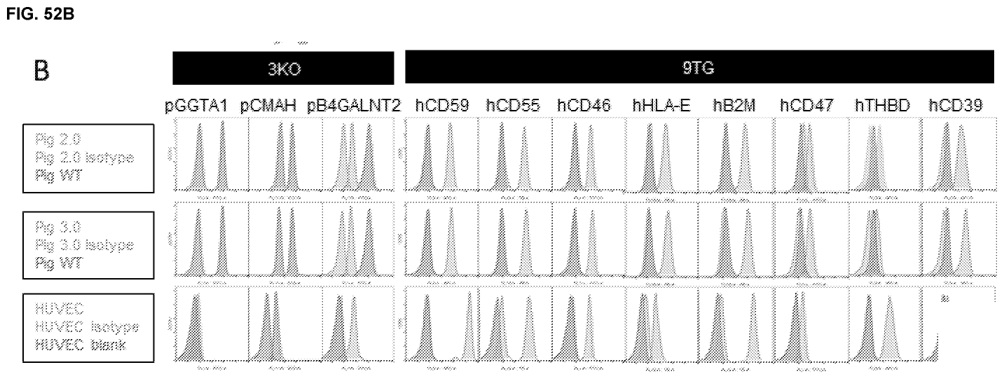

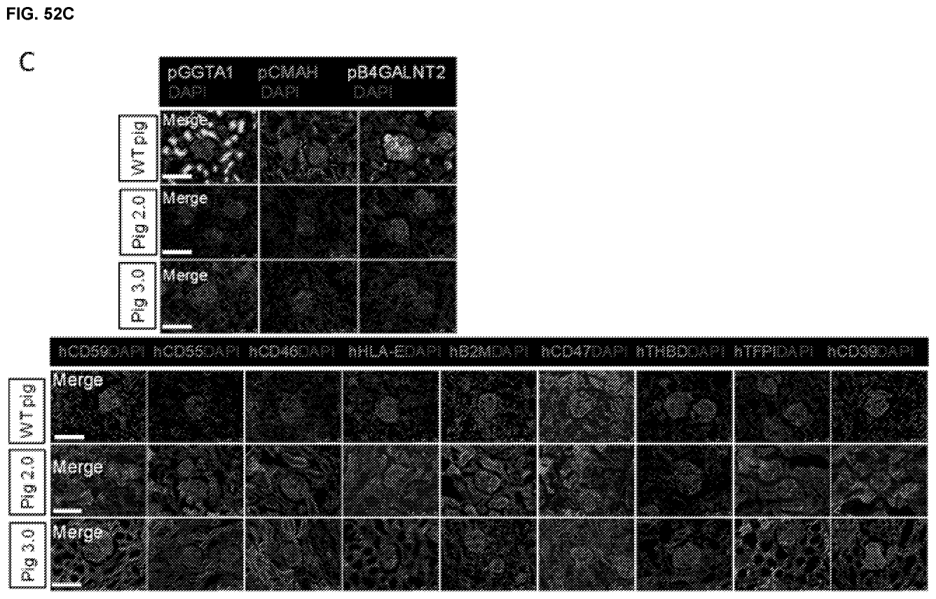

Having confirmed the genomic modifications at DNA level, we went further to examine if Pig 3.0 (3KO+9TG) had the proper triple knockout and 9TG expression using RNA expression and immunoassay methods. We first performed RNA-seq and found that both Pig 2.0 and Pig 3.0 expressed all transgenes at levels comparable to that from human umbilical vein endothelial cells (HUVECs) (FIG. 52A). In addition, we observed comparable transgene expression profile and level in both pig umbilical vein endothelial cells (PUVECs) and fibroblasts, suggesting that the transgenes are ubiquitously expressed among these cell types. We next characterized protein expression in the engineered pigs. We observed diminished glycan markers of α-Gal, Neu5GC, and SDa on cell surface, which suggests functional elimination of the 3 genes responsible for synthesizing these glycan epitopes (GGTA, CMAH, and B4GALNT2, respectively) in both Pig 2.0 (3KO+9TG) and Pig 3.0 cells (FIG. 52B). By FACS analysis of PUVECs, we observed that both Pig 2.0 and Pig 3.0 express all transgenes at the protein level. Indeed, eight out of the nine transgenes are robustly expressed at a level comparable to that of HUVECs. Intriguingly, THBD expression is detectable but at a much lower level. Consistent with FACS analysis, IHC studies showed that Pig 3.0 kidney lacks the three glycan antigens (FIG. 52C). Also consistent with FACS staining, we detected expression of 8 transgenes in Pig 3.0 kidney, with the exception of THBD (FIG. 52C). Taken together, we conclude from the RNA expression and immunoassay data that our triple knockout and 9TG genetic modifications translate into successful RNA and protein expression at the cellular and tissue level in engineered pigs.

B) Evaluation of Xenocompatibility Features of Pig 3.0 Cells

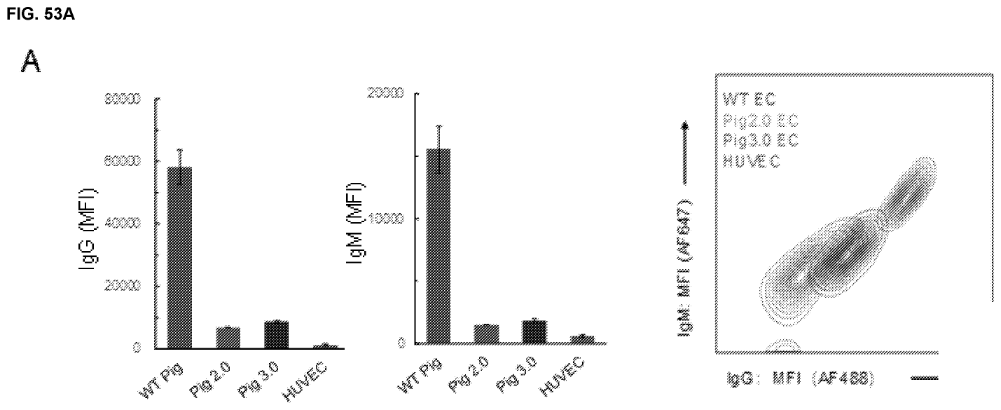

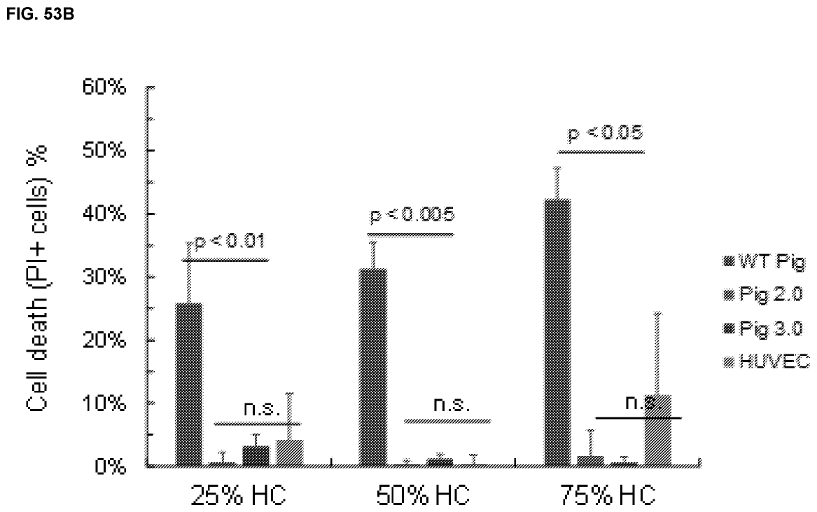

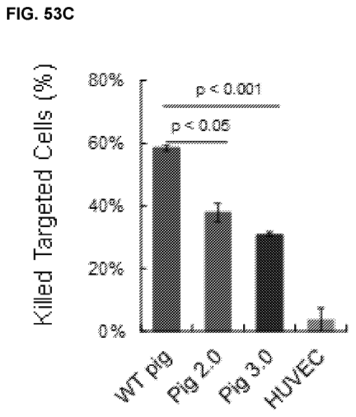

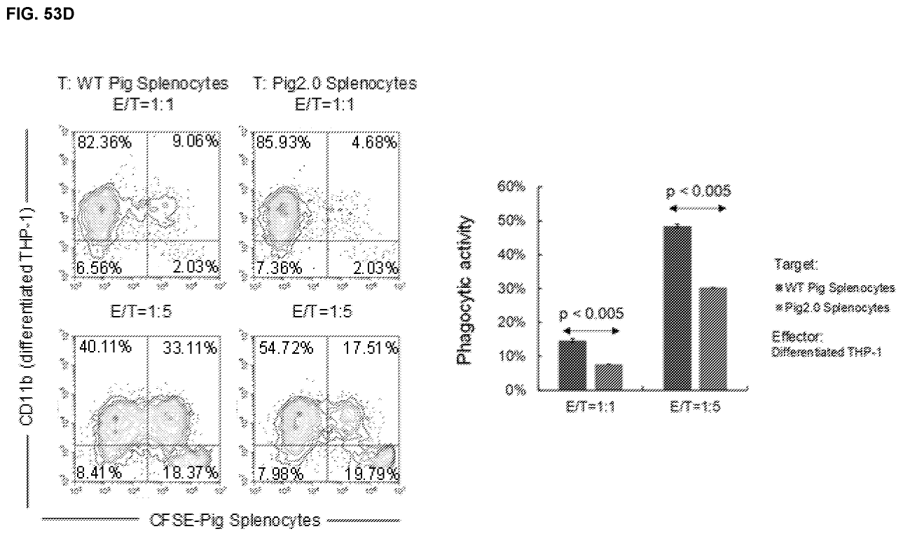

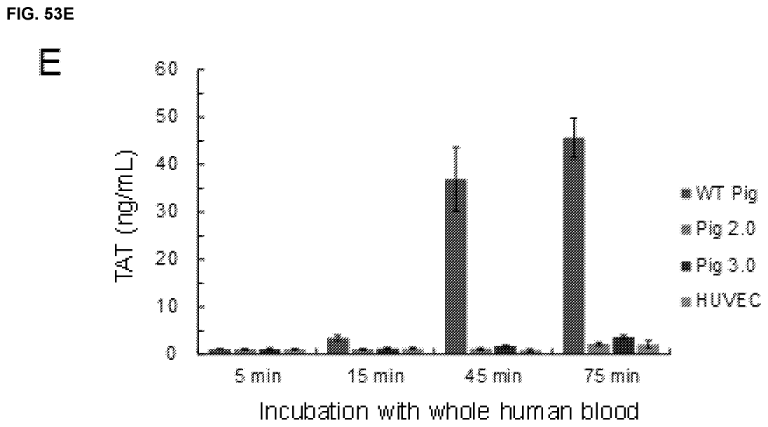

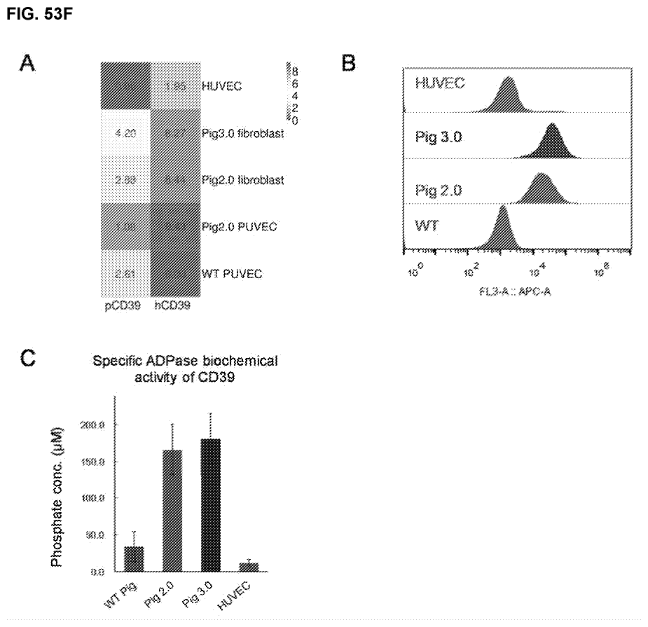

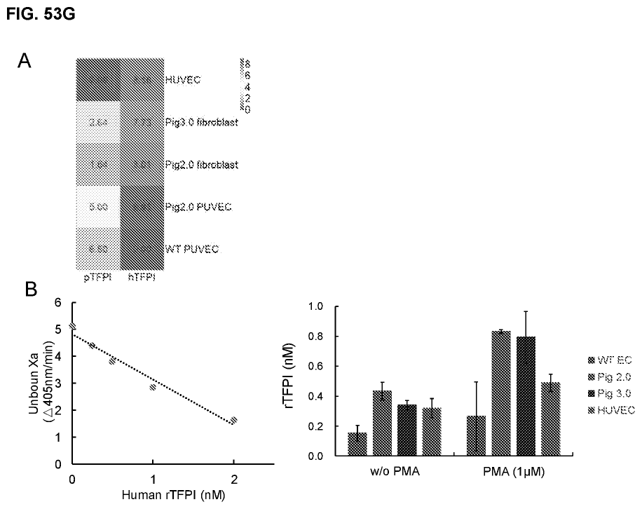

Next, we examined if the genome modified pigs acquired xenocompatibility functions. We first tested if the genetic modifications allow the modified pig cells to evade preformed human antibody binding. Pig 2.0 and Pig 3.0 PUVECs exhibited over 90% reduction in antibody binding to human IgG and IgM, compared to WT PUVECs, confirming that the antibody barrier to xenotransplantation can be greatly mitigated by 3KO (FIG. 53A). In addition, when incubated with human complement from pooled human sera, Pig 3.0 PUVECs with the triple knockout which expressing human complement modulators CD46, CD55, and CD59 demonstrated minimal in vitro human complement toxicity, similar to their human HUVEC counterpart (FIG. 53B). Taken together, these results suggest that, when transplanted, Pig 3.0-derived xenografts are expected to be less susceptible to humoral injury and hyperacute rejection, as a result of significantly reduced antibody binding and complement activation.

Further, we examined if Pig 3.0 was more resistant to injury mediated by human innate cellular immunity. When subjected to ex vivo assays, Pig 3.0 expressing HLA-E/B2M demonstrated significantly stronger resistance to NK-mediated cell killing compared with that of WT PUVECs (FIG. 53C). Taken together, these results suggested that Pig 3.0 cells, when transplanted, are expected to be more resistant to attack by human innate immunity.

Finally, we examined if Pig 3.0 (3KO+9TG) could attenuate the dysregulated activation of platelets and coagulation cascades often observed in xenotransplantation. When vascularized WT porcine organs are transplanted into humans, preformed antibodies, complement, and innate immune cells can induce endothelial cell activation and trigger coagulation and inflammation. The incompatibility between coagulation regulatory factors from pig endothelial cells and human blood leads to abnormal platelet activation and thrombin formation, exacerbating the damage. In addition, molecular incompatibilities of coagulation regulators (e.g., tissue factor pathway inhibitor, TFPI) between pig and human render the extrinsic coagulation regulation ineffective.

To address these xenogeneic coagulation issues, we overexpressed both: a) human CD39 (an ADP hydrolase that counteracts the thrombotic effect of ADP in the coagulation cascade) and b) human TFPI (a factor that translocates to the cell surface following endothelial cell activation) in Pig 3.0 as part of our multi-transgene construct for Pig 3.0. We then performed a variety of in vitro and ex vivo assays to validate the ability of these transgenes to function correctly and modulate clotting pathways when ported to porcine cells. In vitro ADPase biochemical assays showed significantly higher CD39 activity in Pig 3.0 PUVECs when compared with WT PUVECs and HUVECs, consistent with its higher mRNA and protein expression from the transgene (FIG. 53F). Similarly, activated Pig 3.0 PUVECs showed ability to effectively bind and neutralize human Xa, which can mitigate coagulation and reduce the formation of thrombin-antithrombin (TAT) complex (FIG. 53G). Finally, in ex vivo coagulation assays with human whole blood co-cultured with Pig 3.0 PUVECs, minimal TAT (thrombin antithrombin) was formed, and the level of TAT formation was similar to that of HUVECs (FIG. 53E), suggesting that Pig 3.0 gained enhanced coagulation compatibility with human factors.

Collectively, the results of these xenocompatibility experiments indicated that Pig 3.0 (3KO+9TG) gained enhanced compatibility with the human immune system, as evidenced by attenuated human antibody binding, complement toxicity, NK-cell toxicity, phagocytosis, and restored coagulation regulation.

C) Physiological Phenotypes of Pig 3.0 Progenitor/Proof of Concept Pigs

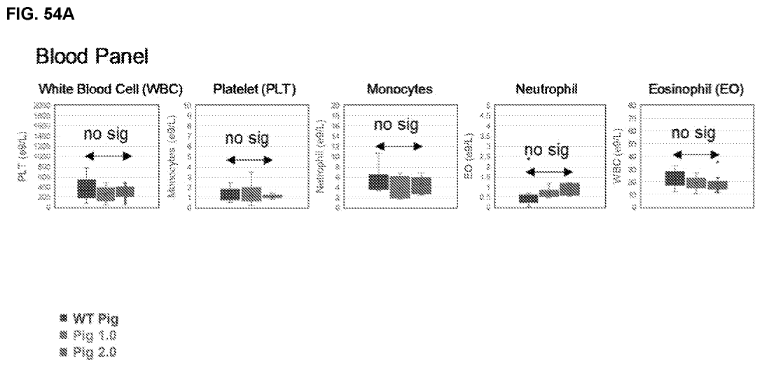

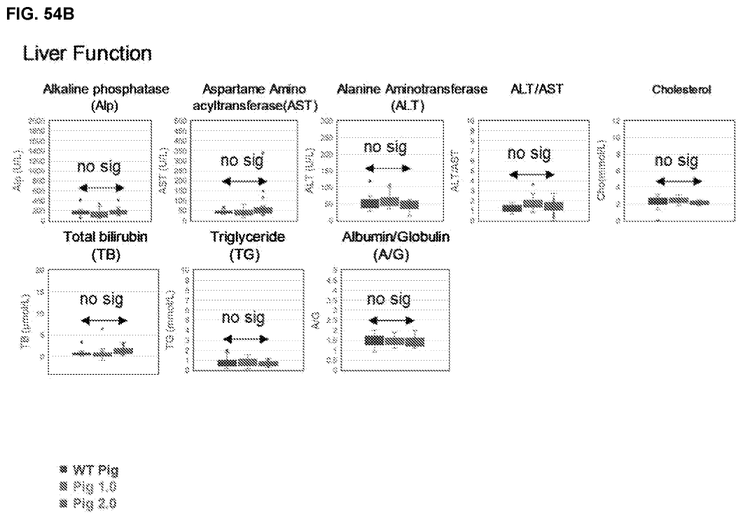

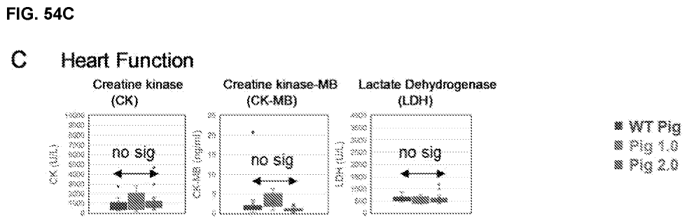

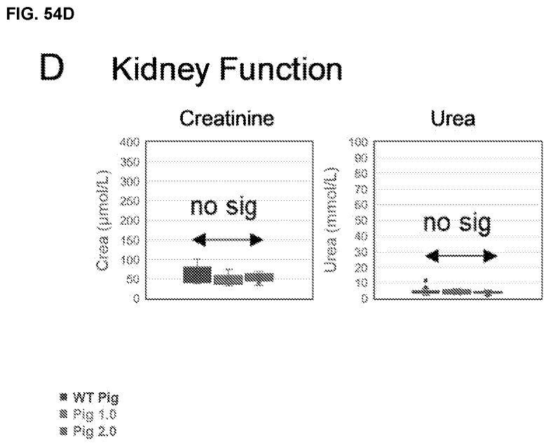

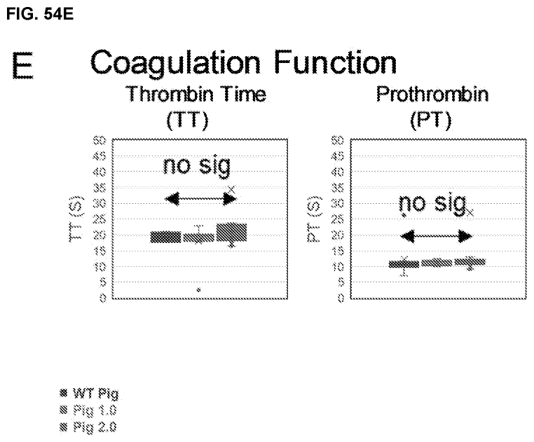

To assess the overall fitness of the engineered pigs, we examined the physiology, fertility, and transmission of the genetic modifications of the engineered pigs to the offspring. We observed that both Pig 1.0 and 2.0 (3KO+9TG), although extensively engineered on PERV elements, immunological and coagulation pathways, show normal blood cell counts, including total white blood cell and platelet, monocyte, neutrophil, and eosinophil counts (FIG. 54A). We also observed normal vital organ functions (liver, kidney, and heart) for engineered pigs (FIGS. 54B, 54C, and 54D). In addition, engineered pigs had similar prothrombin and thrombin time compared with WT pigs (FIG. 54E).

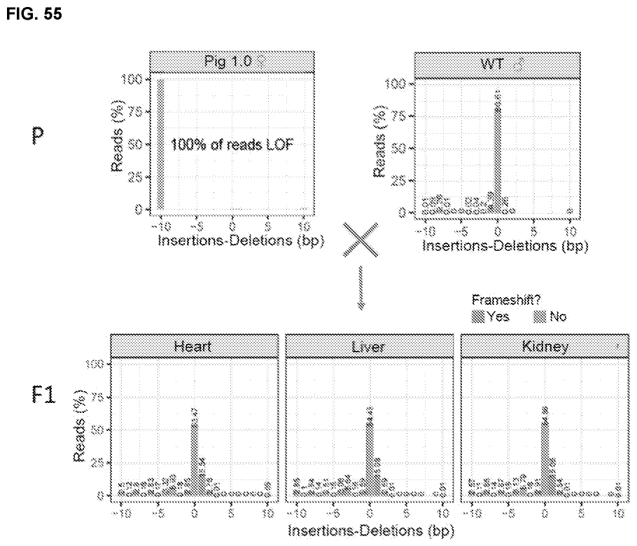

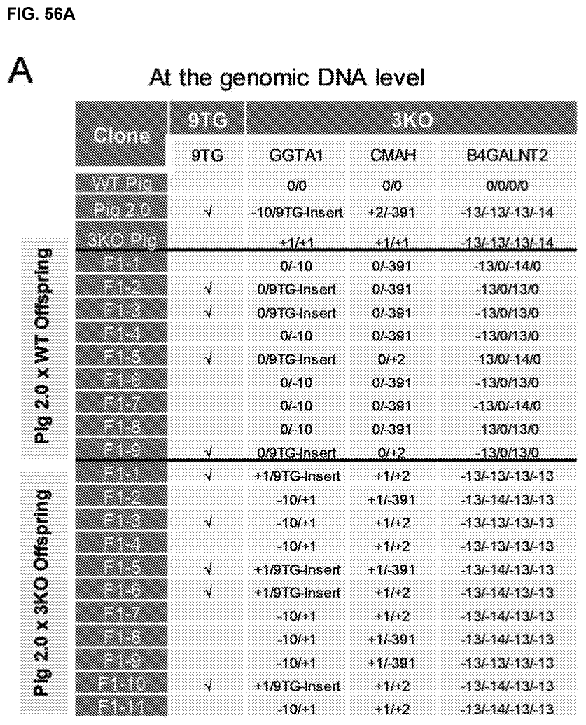

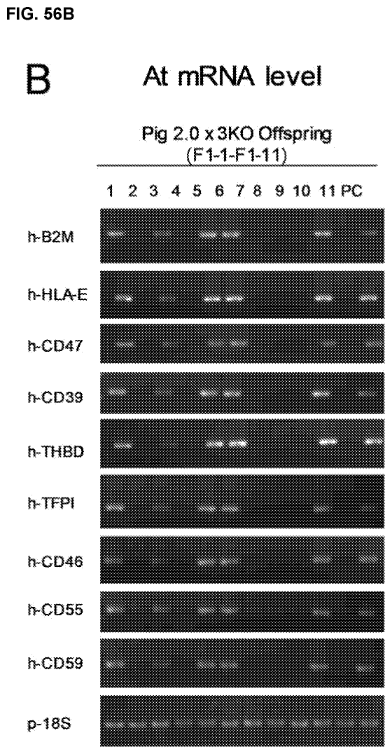

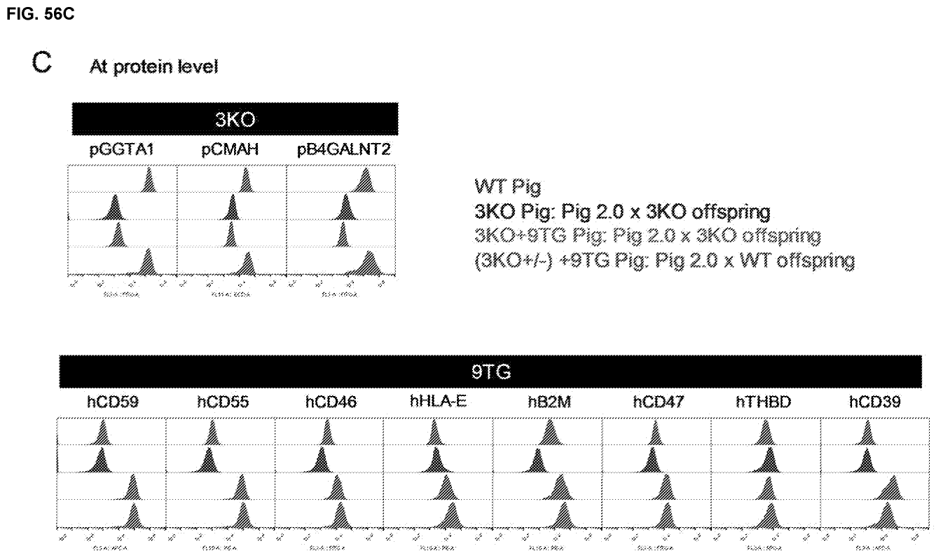

In addition, we found Pig 1.0 and 2.0 were fertile and produced a normal average litter size of seven. The offspring from breeding Pig 1.0 with WT pigs carried ˜50% PERV inactivated alleles in their liver, kidney, and heart tissues, indicating that PERV-KO alleles are stably inherited following Mendelian genetics (FIG. 55). Similarly, all the offspring of Pig 2.0 and WT pigs were heterozygous (FIG. 56A) for 3KO and approximately half carried 9TG, with expression validated at both the mRNA (FIG. 56B) and protein level (FIG. 56C). This suggests that the genetic modifications have not been swept by normal breeding. Therefore, we conclude that the engineered pigs exhibit normal physiology, fertility, and germline transmission of the edited alleles.

D) Conclusion

Genetically engineered pigs hold great promise in addressing the unmet medical need of organ shortage. In this report, we engineered Pig 3.0 (3KO+9TG) with 42 genomic loci modified to eradicate PERV activity and enhance human immune compatibility. Extensive analysis of Pig 3.0 showed that the engineered pig cells exhibit reduced human antibody binding, complement toxicity, NK cell toxicity, and coagulation dysregulation. We also examined and validated the normal pathophysiology, fertility, and genetic inheritability of our engineered pigs. The successful production of Pig 3.0 enhances the ability to provide safe and effective organs for clinical transplantation.

Successful generation of Pig 3.0 (3KO+9TG) demonstrates the power of synthetic biology to extensively engineer the genome and confer novel functions in large animals. In Pig 3.0, we deleted 25 copies of PERV elements, 8 alleles of xenogeneic genes, and concurrently expressed 9 human transgenes to physiologically relevant levels. It extends the record of genome modifications to 42 in large animal models. With the ability to execute complex genetic engineering in this scale, we are in a position to engineer additional edits and ultimately choose the pig with the combination best suited for xenotransplantation. In addition, with the tools, we envision pig 3.0 can be further engineered to achieve additional novel functions, such as immune tolerance, organ longevity, and viral immunity.

E) Methods

CRISPR-Cas9 gRNA Design

We used the R library DECIPHER to design specific gRNAs (PERV-3N: 5′-TCTGGCGGGAGCCACCAAAC-3′, PERV-5N: 5′-GGCTTCGTCAAAGATGGTCG-3′, PERV-9N: 5′-TTCTAAGCAGTCCTGTTTGG-3′) to target specifically all pol catalytic sequences in the Pig 2.0 genome. In addition, we used specific gRNAs (GGTA1: 5′-GCTGCTTGTCTCAACTGTAA-3′, CMAH: 5′-GAAGCTGCCAATCTCAAGGA-3′, B4GALTN2: 5′-GATGCCCGAAGGCGTCACAT-3′) to target GGTA1, CMAH and B4GALNT2 respectively.

Cell Culture

Porcine fetal fibroblast cells and fibroblast cells FFF3 were maintained in Dulbecco's modified Eagle's medium (DMEM, Invitrogen) high glucose with sodium pyruvate supplemented with 15% fetal bovine serum (Invitrogen), 1% penicillin/streptomycin (Pen/Strep, Invitrogen) and 1% HEPES (Thermo Fisher Scientific). All cells were maintained in a humidified tri-gas incubator at 38° C. and 5% CO2, 90% N2, and 5% 02.

Porcine umbilical vein endothelial cells (PUVEC) were freshly isolated from umbilical vein and cultured in PriGrow II Medium (abm) supplemented with 10% fetal bovine serum (Gibco), 1% penicillin/streptomycin (Pen/Strep, Invitrogen) and 1% HEPES (Thermo Fisher Scientific). Human umbilical vein endothelial cells (HUVEC, ATCC, PCS-100-010) were cultured in vascular cell basal medium (ATCC) supplemented with Endothelial Cell Growth Kit-BBE (ECG kit, ATCC). Human NK-92 cell line was cultured in Minimum Essential Medium Alpha (α-MEM, Gibco) supplemented with 12.5% fetal bovine serum (Gibco), 12.5% fetal equine serum (FES, Solarbio) and 1% penicillin/streptomycin (Pen/Strep, Invitrogen). The human macrophage cell line THP-1 was cultured in RPMI 1640 (BI) supplemented with 10% fetal bovine serum (Gibco) and 1% penicillin/streptomycin (Pen/Strep, Invitrogen). Differentiation of THP-1 cells was achieved in 62.5 nM Phorbol-12-myristate-13-acetate (PMA, Sigma) for 3 days and confirmed by attachment of these cells to tissue-culture plastic.

PiggyBac-Cas9/2gRNAs Construction and Cell Line Establishment

Similar to the procedure previously described (Yang 2015), we synthesized a DNA fragment encoding U6-gRNA1-U6-gRNA2 (Genewiz) and incorporated it into a previously constructed PiggyBac-cas9 plasmid. To establish the FFF3 cell lines with PiggyBac-Cas9/2gRNAs integration, we transfected 5×105 FFF3 cells with 14.3 μg PiggyBac-Cas9/2gRNAs plasmid and 5.7 μg Super PiggyBac Transposase plasmid (System Biosciences) using the Neon transfection system, according to the instructions provided by the vendors (Thermo Fisher Scientific). To select the cells carrying the integrated construct, 2 μg/mL puromycin was applied to the transfected cells. Based on the negative control, in which we applied puromycin to wild type FFF3 cells, we determined that puromycin selection was completed in 4 days. The FFF3-PiggyBac cell line was maintained with 2 μg/mL puromycin thereafter and a 2 μg/ml doxycycline was applied to induce Cas9 expression of the doxycycline-inducible FFF3-PiggyBac cell line for one week.

To avoid the constitutive Cas9 expression in the FFF3 cell line, we conducted PiggyBac-Cas9/2gRNAs excision from the FFF3 genome by transfecting 5×105 cells with 3 μg PiggyBac Excision-Only Transposase vector using Lipofectamine 2000 reagent. The PiggyBac-Cas9/2gRNAs-excised FFF3 cells were then single-cell sorted into 96-well plates for clone growth and genotyping.

Genotyping of Single-Cell and Single Cell Clones

First, puromycin selection followed by PiggyBac excision was conducted on the FFF3-PiggyBac-Cas9/2gRNA cell line. Then the cells were sorted into single cells into both 96-well PCR plates for direct genotyping and 96-well cell culture plates for colony growth. To genotype single FF cells without clonal expansion, we directly amplified the PERV loci from sorted single cells. We also conducted genotyping for the clones grown from the sorted single cells. The procedure of genotyping was according to the method of Yang, et al., (6). Briefly, we sorted single cells into 96-well PCR plates with each well carrying a 5 μl lysis mixture, which contained 0.5 μl 10×KAPA express extract buffer (KAPA Biosystems), 0.1 μl of 1 U/μl KAPA Express Extract Enzyme and 4.4 μl water. We incubated the lysis reaction at 75° C. for 15 min and inactivated the reaction at 95° C. for 5 min. All reactions were then added to 20 μl PCR reactions containing 1× KAPA 2G fast (KAPA Biosystems), 0.2 μM PERV Illumina primers (Methods Table 2). Reactions were incubated at 95° C. for 3 min followed by 30 (for single cell) or 25 (for single cell clones) cycles of 95° C., 20 s; 59° C., 20 s and 72° C., 10 s. To add the Illumina sequence adaptors, 3 μl of reaction products were then added to 20 μl of PCR mix containing 1×KAPA 2G fast (KAPA Biosystems) and 0.3 μM primers carrying Illumina sequence adaptors. Reactions were incubated at 95° C. for 3 min, followed by 20 (for single cell) or 10 (for single cell clones) cycles of 95° C., 20 s; 59° C., 20 s and 72° C., 10 s. PCR products were examined on EX 2% gels (Invitrogen), followed by the recovery of ˜360 bp target products from the gel. These products were then mixed at roughly the same amount, purified (QIAquick Gel Extraction Kit), and sequenced with MiSeq Personal Sequencer (Illumina). We then analyzed deep sequencing data and determined the PERV editing efficiency using CRISPR-GA (5).

Primers Used in the PERV Pol Genotyping

Illumina_PERV_pol forward:
5′-ACACTCTTTCCCTACACGACGCTCTTCCGATCTCGACTGCCCCAAG
GGTTCAA-3′
Illumina_PERV_pol reverse:
5′-GTGACTGGAGTTCAGACGTGTGCTCTTCCGATCTTCTCTCCTGCAA
ATCTGGGCC-3′

Somatic cell microinjection to produce SCNT embryos and embryo transfer for pig cloning

The somatic cell microinjection procedure was according to Wei, et al. All animal experiments were performed with the approval of the Animal Care Committee of Yunnan Agricultural University, China. All chemicals were purchased from Sigma Chemical Co. (St. Louis, Mo., USA), unless otherwise indicated. Porcine ovaries were collected from Hongteng Abattoir (Chenggong Ruide Food Co., Ltd, Kunming, Yunnan Province, China). The ovaries were transported to the laboratory at 25° C. to 30° C. in 0.9% (w/v) NaCl solution supplemented with 75 mg/mL potassium penicillin G and 50 mg/mL streptomycin sulfate. The cumulus cell-oocyte complexes (COCs) were isolated from the follicles of 3-6 mm in diameter, and then cultured in 200 μL TCM-199 medium supplemented with 0.1 mg/mL pyruvic acid, 0.1 mg/mL L-cysteine hydrochloride monohydrate, 10 ng/mL epidermal growth factor, 10% (v/v) porcine follicular fluid, 75 mg/mL potassium penicillin G, 50 mg/mL streptomycin sulfate, and 10 IU/mL eCG and hCG (Teikoku Zouki Co., Tokyo, Japan) at 38.5° C. in a humidified atmosphere with 5% CO2 (APC-30D, ASTEC, Japan). After 38 to 42 hours in-vitro maturation, the expanded cumulus cells of the COCs were removed by repeat pipetting of the COCs in 0.1% (w/v) hyaluronidase.

SCNT was conducted as previously described. Briefly, oocytes extruding the first polar body with intact membrane were cultured in NCSU23 medium supplemented with 0.1 mg/mL demecolcine, 0.05 M sucrose, and 4 mg/mL bovine serum albumin (BSA) for 0.5 to 1 hour for nucleus protrusion. The protruded nucleus was then removed along with the polar body by using a bevelled pipette (approximately 20 μm in diameter) in Tyrode's lactate medium supplemented with 10 μM hydroxyethyl piperazineethanesulfonic acid (HEPES), 0.3% (w/v) polyvinylpyrrolidone, and 10% FBS in the presence of 0.1 mg/mL demecolcine and 5 mg/mL cytochalasin B. WT or PERV-free fibroblasts were used as nuclear donors. A single donor cell was injected into the perivitelline space of the enucleated oocyte.

Donor cells were fused with the recipient cytoplasts with a single direct current pulse of 200 V/mm for 20 μs by using an embryonic cell fusion system (ET3, Fujihira Industry Co. Ltd., Tokyo, Japan) in a fusion medium which contains 0.25 M D-sorbic alcohol, 0.05 mM Mg(C2H3O2)2, 20 mg/mL BSA and 0.5 mM HEPES (free acid). The reconstructed embryos were cultured in PZM-3 solution (van′t Veer 1997) for 2 hours to allow nucleus reprogramming and then activated with a single pulse of 150 V/mm for 100 μs in an activation medium containing 0.25 M D-sorbic alcohol, 0.01 mM Ca(C2H3O2)2, 0.05 mM Mg(C2H3O2)2 and 0.1 mg/mL BSA. The activated embryos were then cultured in PZM-3 supplemented with 5 mg/mL cytochalasin B for 2 hours at 38.5° C. in humidified atmosphere with 5% CO2, 5% 02, and 90% N2 (APM-30D for further activation, ASTEC, Japan). Reconstructed embryos were then transferred to new PZM-3 medium and cultured in humidified air with 5% CO2, 5% 02, and 90% N2 at 38.5° C. for 2 and 7 days to detect the embryo cleavage and blastocyst development ratios, respectively.

Crossbred (Large White/Landrace Duroc) sows with one birth history were used as the surrogate mothers of the constructed embryos. They were examined for estrus at 9:00 am and 6:00 pm daily. The SCNT embryos cultured for 6 hours after activation were surgically transferred to the oviducts of the surrogates. Pregnancy was examined 23 days after embryo transfer using an ultrasound scanner (HS-101 V, Honda Electronics Co. Ltd., Yamazuka, Japan).

Characterization of Protein Expression by Immunofluorescence

Neonatal (3-6 days old) porcine kidney cryosections of WT, Pig 2.0 and Pig 3.0 were subject to immunofluorescence to characterize the genetic modification (3KO and 9TG) at tissue level. Cryosections were fixed with ice-cold acetone, blocked and then stained using either one-step direct or two-step indirect immunofluorescence techniques. The primary and secondary antibodies used were summarized in Supplementary Table 2. Nuclear staining was performed using ProLong Gold DAPI (Thermo Fisher, P36931). Sections were imaged using a Leica Fluorescence Microscope, and analyzed using ImageJ software. All pictures were taken under the same conditions to allow correct comparison of fluorescence intensities among WT, Pig 2.0 and Pig 3. 0 cryosections.

Human antibody binding to porcine endothelial cells

Antibody binding of human IgG and IgM antibodies to the porcine and human endothelial cells were assessed by flow cytometry as previously described (Xenotransplantation, Methods and Protocols, Editors: Costa, Cristina, Máñez, Rafael, ISBN 978-1-61779-845-0). In brief, Pig 2.0, Pig 3.0, WT PUVEC and HUVEC were collected, washed twice and resuspended in staining buffer (PBS containing 1% BSA). Normal human male AB serum (Innovative Research, IPLA-SERAB-H26227) were heat-inactivated at 56° C. for 30 min and diluted 1:4 in staining buffer. Pig 2.0, Pig 3.0, WT PUVEC and HUVEC (1×105 cells per test) were incubated with diluted human serum for 30 min at 37° C., respectively. Cells were then washed with cold staining buffer and incubated with goat anti-human IgG Alexa Fluor 488 (Invitrogen, A11013, 1:200 dilution) and goat anti-human IgM Alexa Fluor 647 (Invitrogen, A21249, 1:200 dilution) for 30 min at 4° C. After washing with cold staining buffer, cells were resuspended in staining buffer containing 7-AAD (BD, 559925, 1:100 dilution) in order to include a dead/live gating. Fluorescence was acquired on CytoFLEX S flow cytometer and data were analyzed using FlowJo analysis software. For each sample, 5,000 events were collected in the live cell gate and plotted as specific median fluorescence intensity (MFI) which is generated by “test MFI (IgG or IgM)—control (secondary antibody only) MFI”.

Human Complement Cytotoxicity Assay

Pig 2.0, Pig 3.0, WT PUVEC and HUVEC were harvested, washed twice with PBS, and resuspended in serum-free culture medium. Cells (1×10⁵ cells per test) were incubated with a uniform pool of human serum complement (Quidel, A113) at different concentrations (0%, 25%, 50% and 75%) for 45 min at 37° C. and 5% CO2. Afterwards, cells were stained with propidium iodide (Invitrogen, P3566, 1:500 dilution) for 5 min and analyzed by using a CytoFLEX S flow cytometer. 5,000 events were collected for each sample, and the percentage of PI positive cells was used as the percentage of cell death mediated by human complement.

NK Cytotoxicity Assay

PUVEC and HUVEC were used as target cells and labeled with anti-pig CD31-FITC antibody (Bio-Rad) and anti-human CD31-FITC antibody (BD), respectively. Meanwhile, human NK 92 cells were used as effector cells and labeled with anti-human CD56-APC antibody (eBioscience). The effector (E) and target cells (T) were cocultured for 4 hours at 37° C. and 5% CO2, at an E/T ratio of 3. Cells were stained with propidium iodide for 5 min and then subject to FACS analysis. The percentage of PI positive cells in CD31+ gate was used to calculate the percentage of killed target cells.

Phagocytosis Assay

Differentiation of human macrophage cell line THP-1 was achieved by 62.5 μM of phorbol myristate acetate (PMA) for 3 days and confirmed by attachment of these cells to tissue-culture plastic. Porcine splenocytes (target cells) were stained with the fluorescent dyes 5/6-CFSE (Molecular Probes) according to the manufacturer's protocol. CFSE-labeled target cells were incubated with human differentiated THP-1 cells (effector cells) at E/T ratios of 1:1 and 1:5, respectively, for 4 hours at 37° C. Macrophages were counterstained with anti-human CD11b antibody and phagocytosis of CFSE-labeled targets were measured by FACS. Phagocytic activity was calculated as previously described (Ide 2007).

CD39 Biochemical ADPase Assay

Pig 2.0, Pig 3.0 and WT PUVEC and HUVEC were seeded at 2×104 per well in a 96-well plate, 1 day before the assay. Cells were incubated with 500 μM ADP (Chrono-Log Corp, #384) for 30 min at 37° C. and 5% CO2. Malachite green (Sigma, MAK307) was added to stop the reaction, and absorbance was measured at 630 nm to determine levels of phosphate generation against the standard curve of KH2PO4.

TFPI Activity and Human Factor Xa Binding Assay

Before the assay, cells were treated with 1 μM PMA for 6 hours to induce the hTFPI expression on the cell surface of Pig 2.0 and Pig 3.0 PUVEC. TFPI activity and human factor Xa binding assay was then performed as previously described (Xenotransplantation, Methods and Protocols, Editors: Costa, Cristina, Máñez, Rafael, ISBN 978-1-61779-845-0). All assays were performed in quadruplicate.

TAT Formation Assay

Pig 2.0, Pig 3.0 and WT PUVEC and HUVEC were seeded at 3×105 per well in 6-well plates. After 1 day, cells were incubated with 1 mL of fresh whole human blood (containing 0.5 U/mL heparin) at 37° C. with gentle shaking. At different indicated time points, blood was aspirated, from which plasma was isolated. TAT content in plasma was measured by using a Thrombin-Antithrombin Complex Human ELISA Kit (Abcam, ab108907).

Variant Calling from Whole Genome Sequencing Data

Paired reads are mapped to the Sus Scrofa 11.1 genome (ftp://ftp.ensembl.org/pub/release-91/fasta/sus_scrofa/dna/) by BWA (v0.7.17-r1188). Variants (SNPs and INDELs) are called using GATK (v4.0.7.0) following the GATK best practice recommendation with the standard filter plus requiring a minimum depth of 10.

In Silico Prediction of on/Off-Target Sites

Genome-wide on-target and off-target sites are predicted using CRISPRSeek (v1.22.1) in R (v3.5.0) allowing up to 6-mismatches. The input genome is either Sus Scrofa 11.1 (ftp://ftp.ensembl.org/pub/release-91/fasta/sus_scrofa/dna/).

Off-Target Calling from Whole Genome Sequencing Data

Filtered variants from GATK fall within 20 bp flanking the PAM sites of predicted off-targets by CRISPRSeek (v1.22.1) are called as potential off-target modifications. When a parental line WGS data is available, variants with allele frequency deviate from the parental line significantly more or less than 0.5 are filtered out using an in-house developed statistical test. The assumption for this test is the chance for both alleles to be simultaneously modified is highly unlikely because off-target mutation is a rare event.

Functional Impact Analysis of Mutations

Regardless a variant is an off-target or germline mutation, it is annotated for sequence change at transcript level and amino acid change at protein level to assess its potential functional impact using VEP (variant effect predictor, v93.3). High impact mutations are specially selected if they can result in frameshift, start gain/lost, stop gain/lost, splice donor/acceptor shift or splice region changes. Whenever available, the mutation will be annotated to indicate whether it's impacting principle or alternative transcripts using the APPRIS database.

Transcription Analysis from RNA-Seq

RNA-Seq reads are aligned to the Sus Scrofa 11.1 genome using STAR (v2.6.1a) under the splicing-aware mode. The expression level is quantified as TPM (transcripts per million) using Salmon (v0.11.3) with both pig transcriptome and transgenes as input transcripts.

PERV Knock-Out Efficiency Analysis by Amplicon-Seq

Paired reads are merged into fragments if their overlap is over 100 bases after trimming 3′-end low-quality bases below Q20. Merged fragments are further scanned to hard mask low-quality bases below Q20 and aligned to the PERV amplicon target sequence using STAR (v2.6.1a) under the splicing-aware mode. The output BAM file is then analyzed by an in-house R script (v3.5.0) to digest the alignment pattern to assess the distribution of INDELs within the PERV amplicon target sequence (with respective to the catalytic center) and derive the knock-out efficiency.

PERV Knock-Out Efficiency Analysis by Capture-Seq

Paired reads are first aligned to the PERV target sequence using STAR (v2.6.1a) under the splicing-aware mode, followed by alignment position dependent deduplication by Picard (v2.18.14). Deduped paired reads are then merged into fragments by an in-house script. Merged fragments are then re-aligned to the PERV capture target sequence using STAR (v2.6.1a) under the splicing-aware mode. The output BAM file is then analyzed by an in-house R script (v3.5.0) to digest the alignment pattern to assess the distribution of INDELs within the capture target sequence and derive the knock-out efficiency.

PERV Haplotype Analysis by Capture-Seq

Paired reads are first aligned to the PERV target sequence using STAR (v2.6.1a) under the splicing-aware mode. Somatic variants are called using Mutect2 (v4.1.2.0) and filtered for variants with minor allele frequency over a given threshold (MAF>0.01). Filtered variants from multiple samples are merged to derive the collection of variant sites for typing haplotypes. Next, properly aligned paired reads were merged into fragments by an in-house scrip. Merged fragments are then re-aligned to the PERV target sequence using STAR (v2.6.1a) under the splicing-aware mode. For each fragment covering the region of interest, we extract the alleles for the collection of variant sites to define the haplotype of the fragment. Finally, the distribution of haplotypes is derived by counting all the fragments covering the region of interest.

Identification of Payload Integration Sites Using Whole Genome Sequencing Data

Paired reads are aligned to a reference library composed of the Sus Scrofa 11.1 genome, PERV haplotypes and the payload plasmid sequence using STAR (v2.6.1a) under the splicing-aware mode. Structure variants (SVs) are called from the BAM file using Lumpy (v0.2.13) to detect DNA fusion point. Next, we screen for SVs that bridge pig genome and the payload sequence with mismatch reads at the integration site.

Statistical Analysis

All the statistical analyses are performed by R (v3.5.0) and Excel (v2016). A p-value <0.05 is significant unless otherwise specified. When multiple tests are involved simultaneously, a p-value correction is performed following the Benjamini-Hochberg procedure to control the overall false discovery rate (FDR). An FDR <0.05 is typically used unless otherwise specified.

### Example 10: Perfusion of Immunologically Compatible Pig Liver with Human Blood

Liver perfusion experiments were performed with immunologically compatible pig livers isolated from Pig 2.0 (4-7; 3KO+12TG) as a proxy experiment to xenotransplantation for analyzing organ function. Wild type livers and 4-7 livers (approximately 80 kg) were isolated from 12-month-old pigs. Livers were perfused with human whole blood and human fresh frozen plasma (FFP). A brief liver perfusion protocol is outlined in Table 1.

TABLE 1

| | Protocol A | | Protocol B | |
|---|---|---|---|---|
| Human Whole Blood | 3 | units | 800 | ml |
| Human FFP | 5 | units | 5 | units |
| Clinimix (4.25/5) | 50 mL initial, then | | 50 mL initial, then | |
| | 10 mL/hr | | 10 mL/hr | |
| Heparin | 10,000 | units bolus | 10,000 | units bolus |
| Insulin | 100 | units | 100 | units |
| As Needed Additives: | | | | |
| 8.4% NaHCO3 | goal pH <7.2 | | goal pH <7.2 | |
| 10% CaCl₂ | goal iCa <1.05 | | goal iCa <1.05 | |
| Heparin | goal ACT >400 | | goal ACT >400 | |
| Insulin | goal glucose <300 | | goal glucose <300 | |
| Temp: | 38° C. | | 38° C. | |
| HA pressure: | 75 | mmHg | 75 | mmHg |
| PV pressure: | 7 | mmHg | 7 | mmHg |

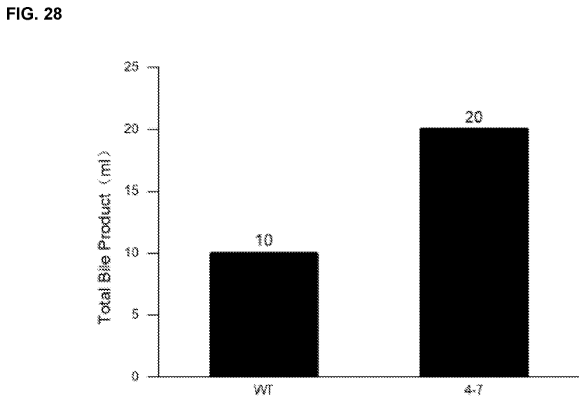

Bile was collected from livers at various time points and analyzed. As shown in FIG. 28, total bile production increased approximately 2-fold in 4-7 liver as compared to WT liver. In addition, 4-7 liver showed stable serum levels of metabolic enzymes that are markers of liver damage including alanine aminotransferase (ALT), aspartate aminotransferase (AST), and albumin (ALB) (FIGS. 29A-C). Furthermore, 4-7 liver showed stable serum electrolyte levels, including potassium (K) and sodium (Na) (FIGS. 29D-E). 4-7 and WT livers were also tested for complement (C3) expression persisted at a higher, more stable level in 4-7 liver compared to WT liver (FIG. 29F). When analyzed for coagulation, 4-7 livers showed stable Prothrombin Time (PT) and International Normalized Ratio (PT-NIR), fibrinogen levels (FIB), and lower activated partial thromboplastin time (APTT) (FIGS. 30A-D). Taken together, these data demonstrate 4-7 livers have improved liver function.

### Example 11: Pig to Non-Human Primate (NHP) Renal Transplantation

Prior to 2014, the longest pig to non-human primate (NHP) renal xenograft was 90 days, with graft survival >30 days being highly unusual. Recent advances in induction and maintenance immunosuppressive therapy regimens coupled with the increased availability of donor pigs with genetic alterations that target host innate and adaptive immune responses has resulted in graft survival extension to >125 days (Higginbotham 2015, Iwase 2015b). Further genetic engineering to compensate for molecular incompatibilities in immune, coagulation, complement, and inflammatory response pathways is beginning to advance the field of xenotransplantation. Despite genetic modification to produce GTKO and overexpression of one hCRP, coagulation dysfunction including thrombotic microangiopathy and systemic consumptive coagulopathy persisted, due primarily to molecular incompatibilities between pig and NHP.

Preclinical renal transplant studies. For preclinical renal transplant studies, safety and efficacy studies will be in NHP. For safety and efficacy examination, kidneys from 8- to 10-week-old Pig 2.0 donors will be transplanted to NHP (cynomolgus monkey) recipients that will undergo bilateral nephrectomy at the time of transplant. Xenograft function will be monitored by serum creatinine values, complete blood counts, and urine analysis for protein as well as serial biopsies and examinations for weight and general well-being. Immunosuppression will consist of clinically relevant reagents in a combination and intensity that would be acceptable in allotransplantation. These will include induction treatment with steroids, anti-NHP thymocyte globulin, anti-CD20, and maintenance immunosuppression with steroids, anti-CD40, MMF and Rapamycin. Prophylactic anti-viral, anti-bacterial, and anti-coagulation therapy will be administered and supplemental Epogen will be given as needed based on hematocrit levels.

It is contemplated that a six-month well-functioning xenograft survival indicated by a normal creatinine with absence of or low-level proteinuria and a biopsy free of acute antibody- or cell-mediated injury will provide sufficient evidence of efficacy.

By analogy with allotransplantation, it is expected that the period of greatest risk for preformed antibody-mediated injury will be in the first weeks post-transplant, and that acute cell-mediated rejection is most likely to occur in the first three months post-transplant with the risk waning thereafter (Cowan 2014).

Allograft Rejection. Per the draft guidance ‘Source Animal, Product, Preclinical, and Clinical Issues Concerning the Use of Xenotransplantation Products in Humans’ revised December 2016 (FDA 2016), the possibility exists that rejection of the xenotransplantation product might pre-dispose the recipient to rejection of subsequent xenotransplantation products or allotransplants (Section IX.C.1.g).

In vitro antibody reactivity and mixed lymphocyte reaction (MLR) assays will be used to demonstrate lack of reactivity following xenotransplantation in preclinical models. To test for possible cross-reactivity between the response to a xenograft and subsequent allograft, flow cytometry cross-matching will be performed using serum from male NHP receiving kidney transplants from normal pig and Pig 2.0 donors as described above. The reactivity of serum to lymphocytes from a panel of NHP donors as well as to lymphocytes from the porcine donors will be tested. Reponses to the porcine cells will confirm that a xeno-sensitizing event has occurred by elevations in anti-porcine antibody levels. Samples from the NHP pretransplant (naïve) will be compared with post-rejection samples to assess for changes in antibody binding to the NHP lymphocyte panel. In parallel, direct and indirect T cell responses by pre- and posttransplant (post-rejection) NHP recipient T cells to a panel of allogeneic stimulators will be evaluated to determine if the cell-mediated allogeneic response is augmented post-rejection of a xenograft (Baertschiger 2004, Cooper 2004, Ye 1995).

It is anticipated that at least a low level of cross-reactivity between xenogeneic and allogeneic responses will be observed. However, these results should be considered in the context of the proposed trials. For the kidney trial, transplantations are planned with highly sensitized patients that have been unable to receive a transplant due to an inability to identify a suitable match. A modest additional sensitization would be unlikely to alter the chances of an opportunity for receiving a subsequent allograft. Moreover, T cell sensitization has not been identified as a significant barrier to re-transplantation and hence may not be possible to monitor clinically (Baertschiger 2004, Cooper 2015). Therefore, it seems unlikely that xenogeneic cell-mediated sensitization will impede allograft survival.

Biodistribution. The migration of donor cells to distal tissues/organs in the recipient remains a possible consequence of xenotransplantation. Chimera studies demonstrate that this may actually increase the success of engraftment reducing the probability of rejection (Starzl 1993, Vagefi 2015). However, there may be unknown consequences of pig donor cell migration and therefore strategies have been developed to determine if migration of cells has occurred. Biodistribution will be studied as part of the pig-NHP xenotransplant research studies according to principles outlined in FDA guidance documents including Source Animal, Product, Preclinical, and Clinical Issues Concerning the Use of Xenotransplantation Products in Humans, December 2016 (Section IX.C.5; FDA December 2016), Gene Therapy Clinical Trials—Observing Subjects for Delayed Adverse Events, November 2016 (Section IV.B.2; FDA November 2016), and Preclinical Assessment of Investigational Cellular and Gene Therapy Products, November 2013 (Section V.C.5.; FDA 2013).

Tumorigenicity. All animals included in the SCNT and assisted reproduction facilities will be routinely monitored for evidence of tumorigenesis. All animals found moribund or dead will have a full necropsy and gross and microscopic pathology examinations by a veterinary pathologist. Records of all genetically engineered animal health and pathology will be maintained and compiled to determine the risk for tumorigenicity potential due to specific or unintended genetic modification.

### Example 12: Pig to Human Renal Transplantation

Renal xenotransplantation has been studied for several decades and porcine xenografts have been evaluated in early clinical trials (Starzl 1964). The challenge is to enable xenograft procedures that provide clinical benefit equivalent to allograft survival.

Clinical Study Design. The proposed clinical study population will include transplant patients age 18-65 with end-stage renal disease who are unlikely to find a suitable kidney donor in a timely manner due to the presence of high levels of panel reactive anti-HLA antibodies (PRA). High PRA creates substantial challenges in matching a suitable deceased or live donor, causing extended waiting times for a transplant and excess morbidity from additional years on hemodialysis. Despite being given allocation priority on waitlists, >90% PRA patients still experience markedly prolonged wait times compared to lesser sensitized patients. Subjects that have >90% PRA sensitization to HLA antigens and who manifest a negative flow cross-match to porcine donor lymphocytes (or endothelial cells) will be targeted.

Patients will receive porcine donor kidneys of 120±10 gm providing an expected glomerular filtration rate (GFR) of 40-50 mL/min/1.73 m². Single porcine kidneys from 9- to 12-month-old donors will be transplanted to the right or left iliac fossa, in a manner identical to that used for allogeneic renal transplantation. The primary endpoint will be freedom from hemodialysis for one year post transplant. Patients will be assessed by serial blood testing for creatinine levels, urinary protein and calculation of GFR using the MDRD equation: GFR (mL/min/1.73 m²)=175×(Scr)^{-1.154}×(Age)^{-0.203}×(0.742 if female)×(1.212 if African American). Protocol-designated graft biopsies will be performed every three months and for-cause based on >20% rise in creatinine from baseline, defined as the mean of the best three consecutive creatinine measures in the first month post-transplant, or proteinuria greater than 300 mg/day. Safety measures will include monitoring of coagulation parameters, clinical chemistry, hematology, and adventitious infections.

Organs for Porcine-Human Renal Transplant. Data suggest that porcine kidneys manifest similar functional potency by kidney weight as human kidneys, thus allowing transplant of kidneys by graft and recipient weight comparable to that used clinically with allografts. In humans, transplantation of allogeneic renal grafts is performed over a broad range of kidney weight to recipient weight. On average, adult male kidneys weigh 125-170 grams and adult female kidneys weigh 115-155 grams (Boron 2003). In considering the upper range for dosing for kidney weight to recipient weight ratio, there is no evidence that an excess of renal function is harmful in any way. Rather, the upper boundary of transplantable renal mass is limited by technical issues. For example, a single adult kidney may be transplanted successfully into a 10 kg infant equating to a 12-17 gm of kidney/kg, which is approximately 3-4 times the renal mass ratio for an average adult (3-4 gm of kidney/kg; Donati-Bourne 2014). This upper graft weight to recipient weight range is relevant to the proposed preclinical studies detailed below. In experimental preclinical studies, 50-75 gm kidneys will be transplanted from 8- to 10-week-old porcine donors into 5-12 kg NHP recipients (˜10 gm of kidney/kg).

Glomerular filtration rate (GFR; mL/min/1.73m2) is a standard measure of renal function or kidney potency that is used to stage the progress of chronic kidney disease (CKD) and renal failure qualifying for dialysis and/or transplantation. In determining the lower range of dosing for kidney weight to recipient weight, the goal is to achieve a GFR of 45-60 mL/min/1.73 m² (CKD stage 3A; Levey 2011). This target range for GFR is based on data suggesting that renal function in CKD 3A is comparable to that achieved by single kidney allotransplantation in humans and is stable, whereas lower GFR in the CKD stage 3B (GFR 30-45 mL/min/1.73 m²) is associated with an increase in end-stage renal disease and all-cause and cardiovascular mortality (Sharma 2010). The targeted GFR range of 45-60 mL/min/1.73 m² is comparable to that achieved by single kidney allotransplantation in humans (50-65 mL/min/1.73 m²; Gourishankar 2003, Marcén 2010).

This will require xenotransplantation of a kidney mass comparable to that routinely used in allotransplantation (115-170 gm) given the comparability of human and porcine kidneys in GFR per renal mass. It should be considered that some renal function may be lost in the donation process and post-transplant due to treatment of the recipient with nephrotoxic immunosuppression in the form of calcineurin inhibitors.

Pharmacology and Toxicology Information. Efficacy and safety will be evaluated using pharmacology studies with both rodent and NHP models. A variety of integrated safety endpoints will be used, as well as an assessment of clinical pathology and pathophysiology in genetically engineered donor porcine tissues. A tiered approach will be taken involving in vitro cellular and tissue function, and assessments of clinical pathology and histopathology in donor pigs and NHP xenografts. Endpoints will include graft function and rejection, and recipient safety related to functions of innate and adaptive immunity, inflammation, as well as complement and coagulation cascades.

Somatic Cell Nuclear Transfer and Assisted Reproduction of Genetically Engineered Donor Pigs. Genetically engineered donor pigs will be monitored routinely for safety considerations with full clinical pathology including clinical chemistry and hematology as well as gross and microscopic histopathology. Reproductive capability, embryo-fetal development, organ and tissue development, and potential tumorigenesis will be monitored and recorded for all donor pigs in the breeding colony.

Animals are identified by unique ear tags printed with permanent ink (placed at Place of Origin). The flow of pigs includes a quarantine area, which is an open-air, group-housed barn with a bedding of wood shavings. The feed trough is wooden and kept clean from debris and waste. Fresh, free-choice water is available at all times via nipple drinkers. The barn relies on outdoor wind movement to circulate the air and temperature is maintained above 10° C. Biosecurity requires at least 24 hours of no other swine contact, specific barn attire, and boot dipping in disinfectant before and after barn contact. The quarantine period includes 35-40 days of quarantine, vaccination with Parvo Shield L5E, FluSure XP/ER Bac Plus, Ingelvac FLEX combo (Circovirus and Mycovirus), and Dectomax, and includes 2 blood draws demonstrating no increase in disease antibodies (PRRSV, PRRSX3). After clearance from quarantine, pigs are moved into a buffer area at the facility. This area is a closed-barn, group-housed, sawdust-bedded pen in groups of up to 12. Bedding is replaced weekly. Temperature is controlled by thermostat-controlled fans and propane heater to a range of 15-24° C. Pigs are fed in a stainless-steel trough and fresh, free-choice water is available at all times via nipple drinkers. Pigs are observed at least once a day and as health status dictates.

Pigs with observed health issues are housed in single pens for individualized care and attention and treated as directed by the Attending Veterinarian and Director of Embryology. Biosecurity requires at least 24 hours of no other swine-herd contact. Coveralls limited to use in the barn area and boots are disinfected either with Virkon-S or Synergize before and after barn contact. Generation of source donor pigs for use in clinical studies will follow all relevant guidance and regulations.

Validation of Genetic Engineering. The endogenous gene KOs and human transgene expression will be validated at genomic, mRNA, and protein levels. For gene KOs, either Sanger sequencing or deep sequencing will be performed to confirm the genetic mutations at the intended target site. Second, RNA-seq and/or RT-PCR will be performed to ensure that the mRNAs contain the intended mutations and are subject to non-sense mediated decay. RT assays will be performed to demonstrate the elimination of RT activity in PERV KO cells. Moreover, immunohistochemistry (IHC) staining and/or flow cytometry will be performed to ensure that gene products are absent in the cell or at the cell surface.

Off-target mutations may still exist despite advances in the field of precision gene editing and must be understood in order to generate safe and efficacious donor organs for clinical xenotransplantation. In order to ascertain the potential off-target effects of CRISPR-Cas9 gene editing, the following multi-tiered assessment approach has been employed:
1. Karyotype of the modified cell clones to determine chromosomal structural integrity;
2. CIRCLE-Seq: A sensitive, in-vitro screening strategy that comprehensively detects genome-wide CRISPR-Cas9 off-target mutations of any given gRNA. The potential off-target sites will be censored in any derived cell line from the specific gRNA using subsequent targeted amplicon sequencing;
3. Whole Genome Sequencing (WGS): to examine single point mutations as well as small structure variations of the genetically engineered cell lines or pigs. Table 2 lists the resolution and sensitivity of the detection methods employed.

TABLE 2

| | Resolution | | Sensitivity | Platform | Notes |
|---|---|---|---|---|---|
| Whole | 1 | bp | 95% (SNV) | Broad | CRO & |
| Genome | | | 80% (indels) | Institute | internal |
| Sequencing | | | | Clinical | analysis |
| | | | | Services | |
| Whole | 100 bp-100 kbp | | depends on | CN.MOPS | CRO & |
| Genome | | | regional | methodology | internal |
| Sequencing | | | exome | | analysis |
| | | | density | | |
| Circle-Seq | 1-20 | bp | all sites | Beacon Bio | CRO/ |
| | | | with >1% off- | | internal |
| | | | target | | develop- |
| | | | activity | | ment |
| Karyotype | 5 | Mbp | 10% | Cell Line | CRO |
| | | | mosaicism | Genetics, | |
| | | | | Inc. | |

For transgene expression, intactness and expression of human transgenes in genomic, mRNA, and protein levels will be validated using sequencing, RT-PCR/RNA-seq, and IHC/flow cytometry technologies. Moreover, the location of random transgene integration will be determined by inverted PCR-based junction capture and the results will be validated by junction PCR.

Clones will be chosen with a single-copy transgene integrated into intergenic regions at least 10,000 bp from any known genes and ncRNAs, and at least 50,000 bp from any oncogenes and tumor suppressors. For site-specific integration and endogenous gene humanization, biallelic site-specific integration/replacement will be validated by junction PCR and droplet digital PCR (ddPCR).

### Example 13: Non-Human Primate (NHP) Renal Transplantation

Preclinical transplant studies. For preclinical transplant studies, safety and efficacy studies were performed in NHP. Hearts, kidneys, and livers from 8-10 week-old Pig 2.0 donors were used for transplanted solid organ studies and liver and lungs were used for perfused organ studies. In a span of 5 months, 15 organ transplants and 11 organ perfusions were performed. Specifically, 7 kidney transplants, 4 heart transplants, 4 liver transplants were performed while 4 livers and 7 lung perfusions were performed, as summarized in Table 3.

TABLE 3

| | | Transplanted | | | | |
|---|---|---|---|---|---|---|
| | Donor | solid | | | Perfused | |
| Payload | ID | organ | | | organ | |
| 2.9 | 1839 | X | X | X | | X |
| | 1841 | X | X | | X | |
| | 1844 | X | X | | X | X |
| | 1848 | | X | X | | X |
| | 1850 | X | X | | X | X |
| 2.10 | 1856 | | | X | | X |
| | A10169 | | X | | X | X |
| | A9956 | | X | X | | |
| | A9954 | | | | X | |

Immunosuppression regimen for kidney transplantations consisted of clinically relevant reagents in a combination and intensity that was acceptable in allotransplantation. Clinical monitoring included: abdominal ultrasound at days 2, 5, 7, 9, 12, and 14 and clinical labs (CBC, Chem 17, coags, serum) at days 2, 5, 7, 9, 12, and 14 and weekly.

Survival of transplanted kidneys from Pig2.0 donors and control pigs (GTKO.hCD55) were analyzed. A summary of the results is provided in Table 4.

TABLE 4

| | | Kidney Graft Survival |
|---|---|---|
| Pig ID | Donor strain | (days) |
| 33-7 | GTKO.hCD55 | 15 (aCD40) |
| 32-2 | GTKO.hCD55 | 11 (aCD40) |
| 53-5 | GTKO.hCD55 | 76 (aCD40L) |
| 53-1 | GTKO.hCD55 | 93 (aCD40L) |
| 1839 | 9 | In life >190 (aCD40L) |
| 1841 | 9 | 20 (aCD40L) |
| 1844 | 9 | 72 (aCD40L) |
| 1848 | 9 | 15 (aCD40L) |
| 1850 | 9 | 6 (aCD40L) |
| A10169 | 10M | 2 (aCD40L) |
| 9956 | 10M | In life >30 (aCD40L) |

The two longest surviving recipients of GTKO.hCD55 pig kidneys survived until days 76 and 93 when they were euthanized due to renal failure and weight loss, respectively. Of these two, one was found to have thrombotic microangiopathy (TMA), chronic antibody-mediated rejection (AMR) and borderline T-cell Mediated Rejection (TCMR); while the other had C4d deposition, but otherwise no histologic evidence of frank rejection. The remaining seven recipients received kidneys from Pig 2.0. In these pigs, transduced human proteins that regulate immune responses or complement activation were expressed at high levels. The NHP recipients of these genetically modified pig kidneys survived >190, 72, 20, 15 and 6 days with immunosuppression regimen for kidney transplantations.

One recipient is currently doing well with normal kidney function (Creatinine 0.6 mg/dl) at day 190 with immunosuppression regimen for kidney transplantation. Multiple biopsies showed no evidence of rejection or TMA.

Together these data demonstrate long-term survival of a kidney xenograft having triple xenoantigen KO with multiple transduction of human genes encoding regulatory proteins in the innate responses and complement pathways, that is free from rejection or TMA has been achieved with minimal maintenance immunosuppression.

Compromised health of monkeys contributed to early termination of several of the xenograft monkeys. Complications included blood transfusions, injection site abscess and infection, wound healing. Several cases presented bleeding in bladder and/or ureter, possibly due to over-anti-coagulation. A summary of the Pig2.0 grafts is provided in Table 5.

TABLE 5

| | Donor | |
|---|---|---|
| Pig ID | strain | Current Status |
| 1839 | 9 | In-life: Ongoing, BUN/Cr stable; 1/wk anti-CD154, |
| | | DHPG & MMF daily |
| 1841 | 9 | Terminated: rejection possible but not confirmed |
| 1844 | 9 | Terminated: renal function fluctuating |
| 1848 | 9 | Terminated: Ureter rejection and ischemic injury |
| | | possible but not confirmed |
| 1850 | 9 | Terminated: Clot in bladder, and later in ureter. Rising |
| | | creatinine. |
| A10169 | 10M | Terminated: Little perfusion to kidney, rising creatinine |
| 9956 | 10M | In-life: Ongoing, BUN/Cr stable; 1/wk anti-CD154, |
| | | DHPG & MMF daily |

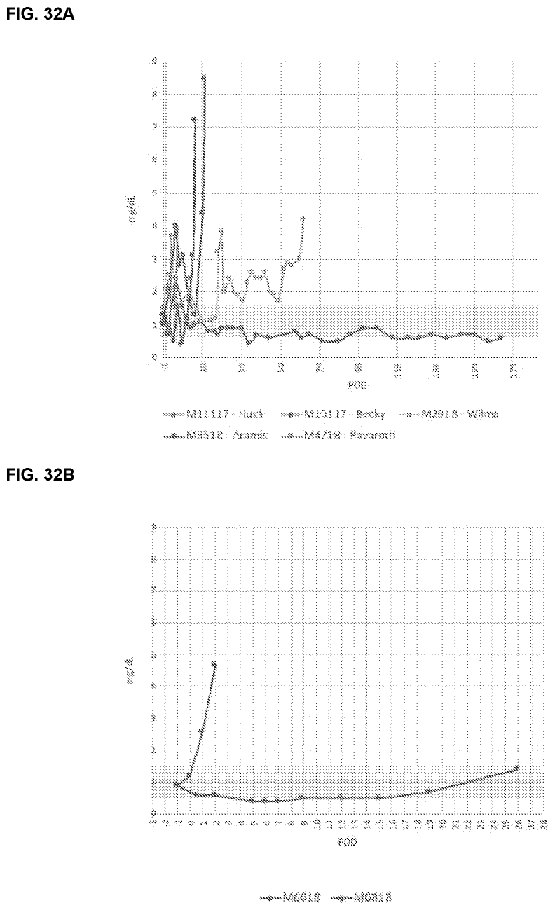

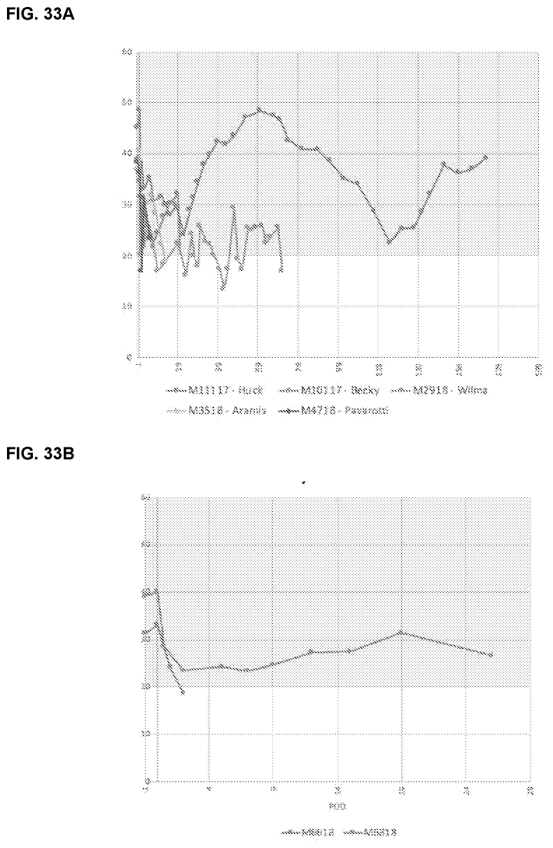

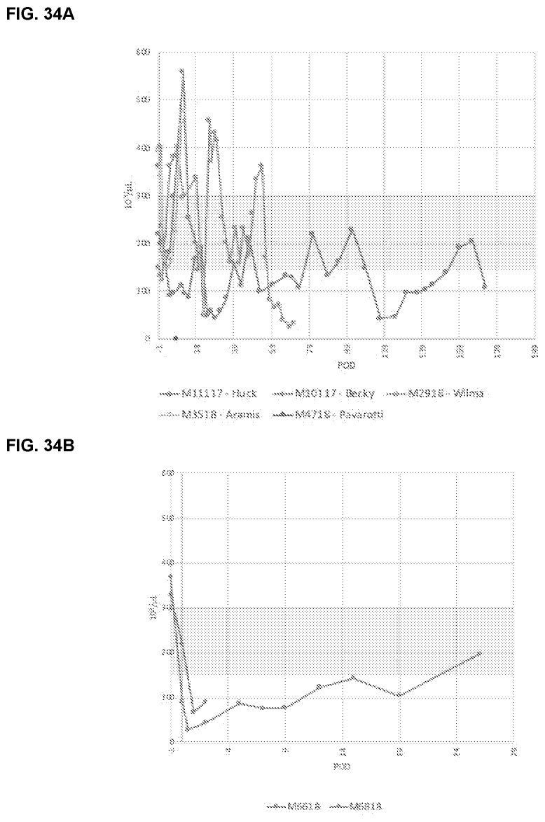

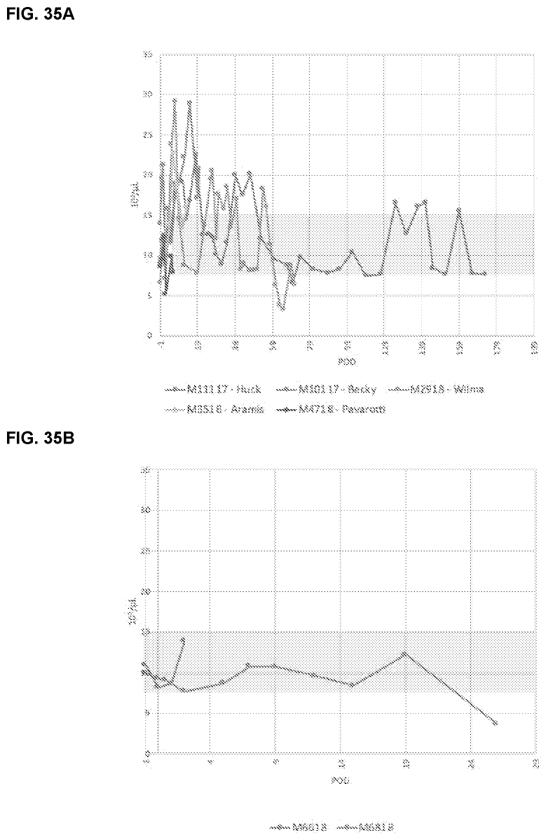

Analysis of host monkeys transplanted with kidneys isolated from Payload 9 (A) and Payload 10 (B) donor pigs demonstrated that hosts exhibit stable serum creatinine levels (FIGS. 32A and 32B). Several host monkeys also exhibited stable or recovering hematocrit levels (FIGS. 33A and 33B). Platelet counts were low in several of the host monkeys, but had recovered in others (FIGS. 34A and 34B). Fluctuations in WBC reflect the immunosuppression regimen and infection events (FIGS. 35A and 35B).

Liver Xenotransplantation. Until recently, pig-to-baboon orthotopic liver xenotransplant (OLTx) survival was limited to 9 days. Administration of human clotting factors improved survival to 25 and 29 days in two recipients of GTKO livers, but consistent survival remains elusive.

Here, four pig-to-baboon OLTx were performed. Livers were from two genetic constructs of transgenic pigs deficient in targets of xenoantibody and containing human transgenes to address complement activation and innate immune cell function (group 1: B1,132; group 2: B3,134). Immunosuppression consisted of ATG, Rituximab, corticosteroids, MMF and aCD154. All recipients received an infusion of KCentra. Unlike previous studies, splenectomy was not performed, and cobra venom factor and tacrolimus were omitted. B2 and B4 received a continuous infusion of a GpIIb/IIIa inhibitor. Graft function was assessed with daily chemistries, lactate, CBC, INR and weekly coagulation profile.

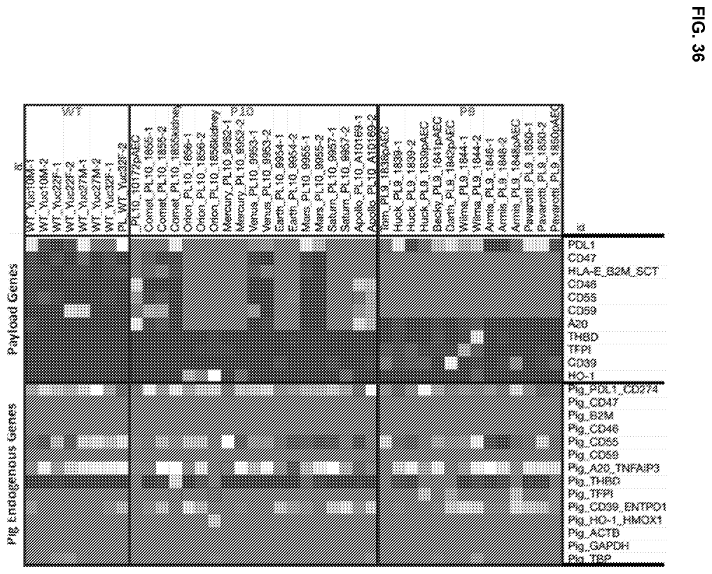

Baboons B1, B2 and B4 underwent successful OLTx with life-sustaining graft function. LFTs peaked on POD1 in all baboons and normalized between POD4-7 (FIGS. 38A-38B). Each baboon manifested thrombocytopenia, with spontaneous recovery beginning on POD8 in B2 and POD4 in B4 (FIG. 38C). Transfusions requirements (FIG. 38D) were less than historic experience. Consumption of coagulation factors occurred immediately after OLTx, with subsequent production at normal pig levels (FIG. 38E-381). B1 was euthanized on POD8 due to respiratory failure from fluid overload and abdominal compartment syndrome. Liver biopsy showed focal ischemia, no rejection and negative C4d (FIG. 38A-B). B2 recovered uneventfully and biopsy on POD8 was normal. Development of hemoptysis and increased transfusion requirement, necessitated euthanasia on POD14. Pulmonary hemorrhage was identified on necropsy. H+E staining of the liver exhibited diffuse sinusoidal neutrophil infiltrate, suggesting infectious complications versus rejection, B2 was C4dnegative and the LFTs remained normal throughout (FIG. 38C-D). B3 was hypotensive and hypoxemic intra-op after reperfusion, requiring euthanasia. Necropsy showed diffuse pulmonary hemorrhage with normal liver and patent vasculature. B4 recovered uneventfully. Only one post-op blood transfusion was required. On POD7, a rise in Tbili and LFT's prompted exploration, where a bile leak and hepatic artery thrombosis (HAT) were identified, requiring euthanasia. Biopsy showed focal subcapsular necrosis with negative C4d and no evidence of rejection, consistent with HAT (FIG. 38E-F).

Together these data of OLTx using novel, genetically modified pig organs demonstrate: reduced reperfusion injury, decreased RBC consumption, and the first antibody-mediated rejection-free survival without splenectomy or use of CVF. The absence of evident rejection suggests that this porcine strain is suitable for further OLTx studies.

Liver Xenoperfusion. Barriers to successful xenogeneic pig liver transplantation include hyperacute rejection by preformed xeno-antibody, molecular incompatibilities resulting in dysregulated complement, coagulation, and innate and adaptive immunity. Genetically modified swine may circumvent these obstacles and will require a rapid and efficient model to evaluate the effectiveness of different genetic constructs. Here, initial results are reported using ex-vivo liver xenoperfusion (EVLXP) of wild type (WT) and genetically modified swine livers perfused with human blood and plasma (hWB+P).

Briefly, livers from Pig 2.0 (EG group, n=3), WT (n=2) and GTKO.hCD55 (n=4) livers were studied. EVXLP was performed at 37° C. with fresh, heparinized hWB+P. Failure during EVXLP was defined by decreased blood flow due to elevated vascular resistance, severe metabolic derangements or gross necrosis. CBC, serum clinical chemistry, and blood gas analysis were performed. Tissue biopsies were stained with H+E and for depositions of IgG, IgM, and complement (C4d).

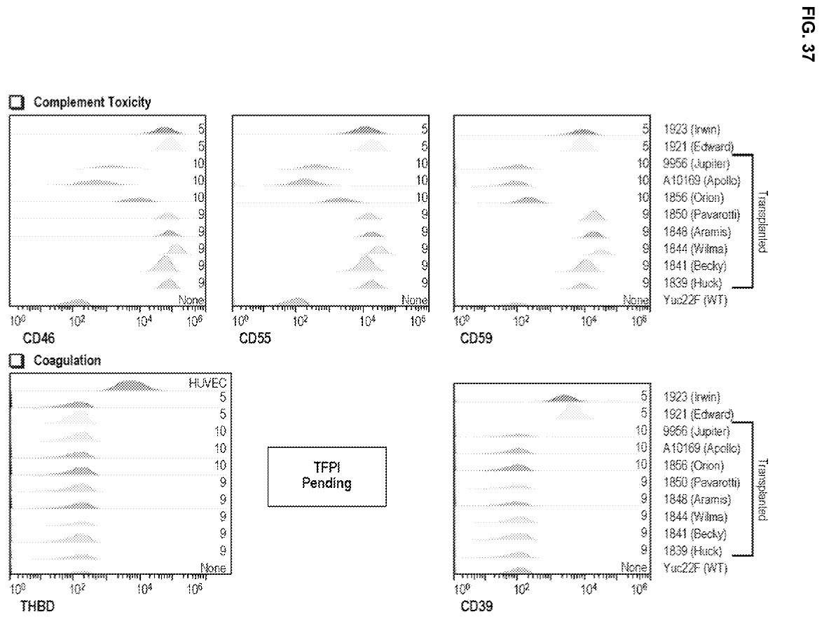

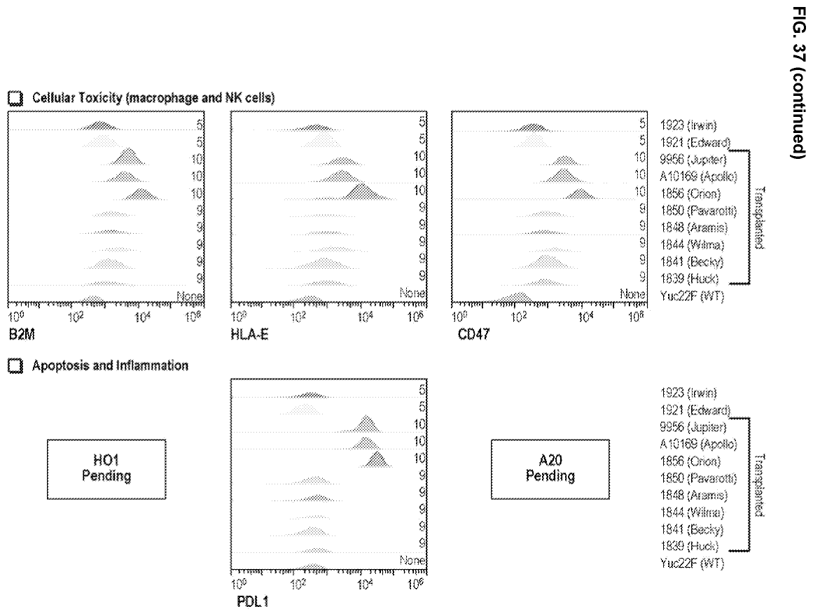

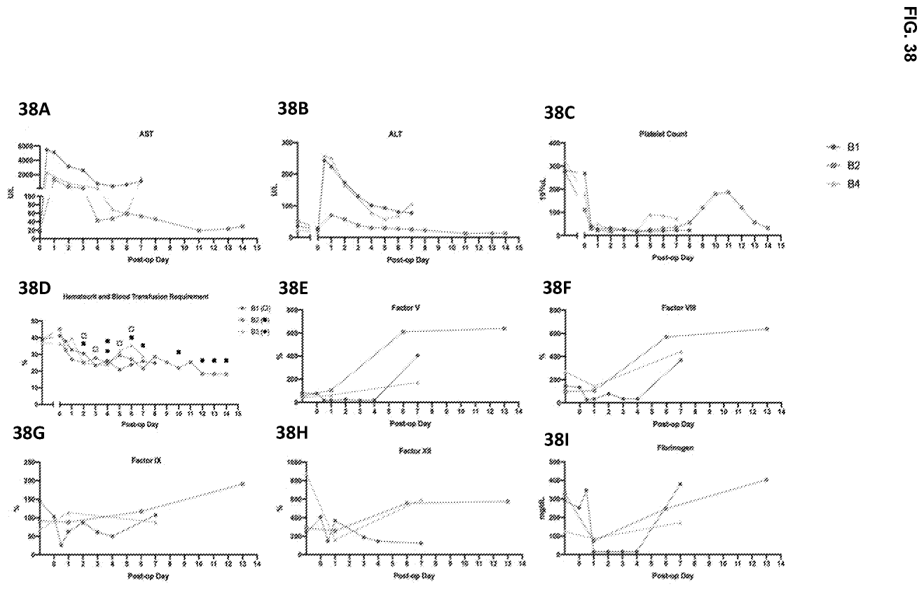

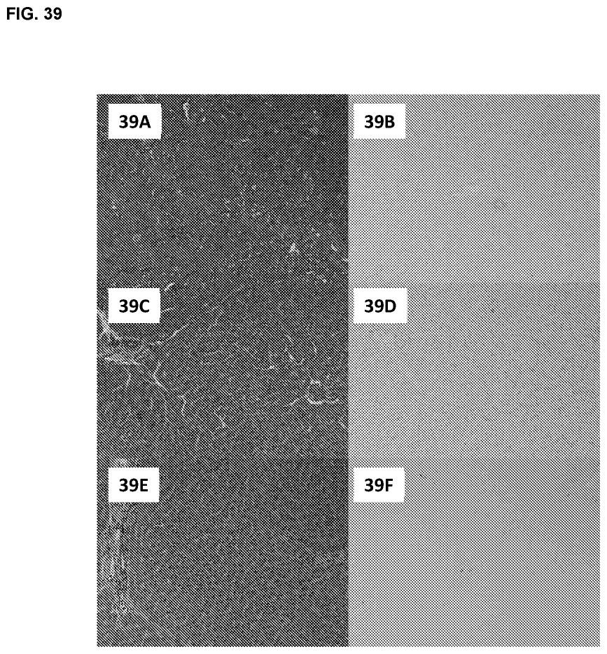

All groups manifested progressive blood flow reduction with a corresponding rise in vascular resistance. Hemodynamic deterioration occurred earlier and progressed faster in the WT and GTKO.CD55 compared to EG livers (FIGS. 39A-39B), and correlated with longer EG liver survival. Mean liver survival for WT was 5 hours (range 5-7 hours), GTKO.CD55, 4.5 hours (range 4-6 hours) and 13 hours (range 11-14 hours) in EG liver. Platelets and neutrophils decreased rapidly in all groups, with the greatest losses observed with WT, but differences did not meet statistical significance (FIGS. 39C-39D). RBC count was preserved throughout perfusion with EG and was significantly higher than WT livers and tended and to be higher than GTKO.CD55 (FIG. 39E).

EG liver tissue biopsies exhibited preserved hepatic architecture on H+E with mild diffuse portal and sinusoidal inflammation (FIG. 41A). WT livers manifested focal ischemic necrosis and vascular congestion on H+E (FIG. 41E), with strong staining for IgM and IgG (FIGS. 41F-41G) and C4d-positivity (FIG. 41H). In contrast, EG livers showed diffuse mild sinusoidal IgG and IgM deposition (FIGS. 41B-41C), with negative C4d (FIG. 41D), perhaps suggesting the reduction in pre-formed antigens and improvements in complement regulation by addition of human complement regulatory protein expression resulted in less injury.

Xenolivers from transgenic pigs deficient in xeno-specific antigens and containing humanized transgenes related to complement activation and immune cell function achieved significantly prolonged survival with less severe platelet sequestration, preserved RBC mass and diminished antibody and complement deposition compared to WT or GTKO.CD55 xenografts. This model is an efficient and informative tool to simulate pig-to-human xenotransplantation and evaluate the efficacy of specific genetic modifications.

Lung Xenoperfusion. Ex vivo lung perfusion with human blood is a standardized method to evaluate the impact of transgene combinations. Here results associated with novel transgenic pig lines, evaluated in the context of a reference cohort, are reported.

Briefly, eight pairs of lungs from pigs with combined Eight pairs of lungs from pigs with combined Gal1,3αGal, β4Gal and Neu5Gc knockouts (TKOs) and containing human transgenes addressing molecular incompatibilities in complement activation, and innate and adaptive immune cell function were perfused ex vivo with freshly collected heparinized human blood. GalTKO.hCD55 lungs served as reference group. In pairs of lungs from each pig, blood was left ‘untreated’ (n=5 Pig 2.0, and n=3 reference), or the blood was ‘treated’ with 1-BIA, a thromboxane synthase inhibitor and histamine receptor blocker (n=7 for Pig 2.0, n=4 for reference). Tissue and blood samples were collected at predefined time points and experiments were terminated electively after 8 hours of perfusion if lungs had not failed earlier.

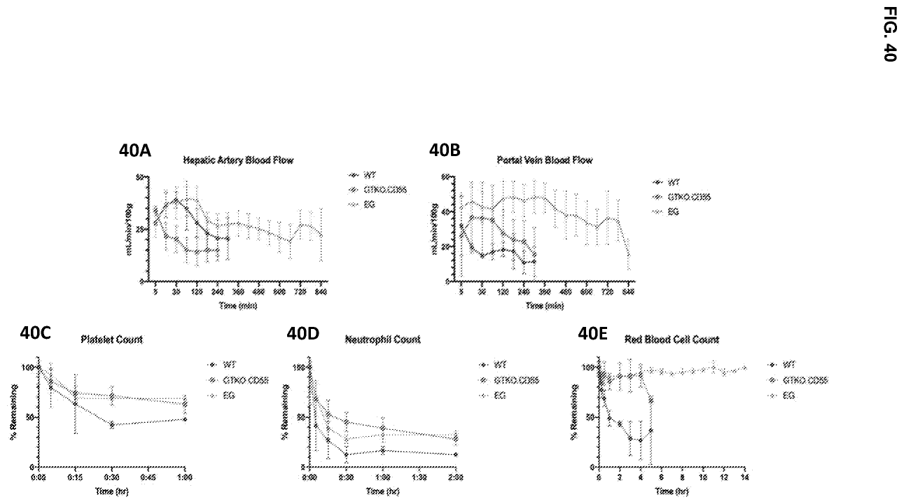

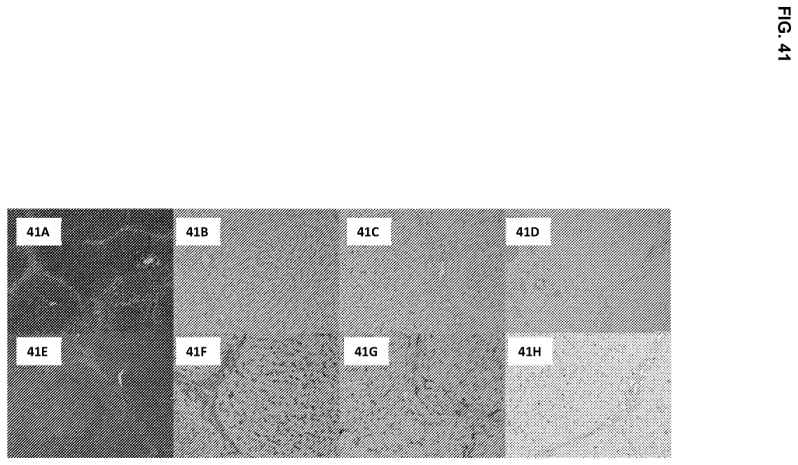

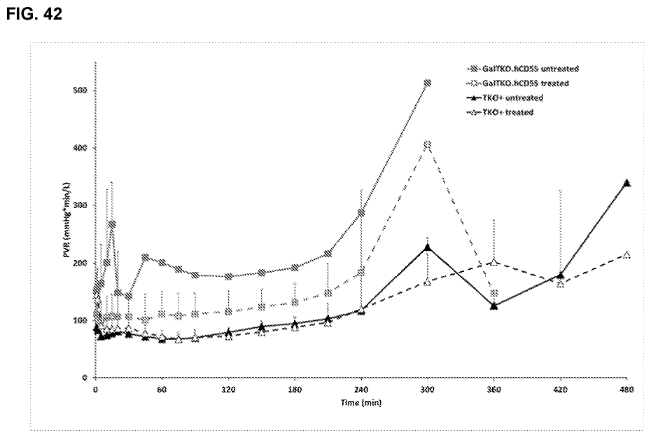

Median survival time for Pig 2.0 lungs was 450 min (range 300-480 min) vs. 30 min (range 20-300 min) for reference lungs (P=0.04) in the untreated groups and 480 min (range 360-480 min) vs. 300 min (range 145-360 min) in the treated cohorts (P=0.009). Pulmonary vascular resistance (PVR) rise was significantly attenuated and delayed in ‘untreated’ Pig 2.0 lungs, relative to GalTKO.hCD55 lungs (FIG. 42). Additional blood treatment with 1-BIA and H-blocker attenuated PVR rise within both Pig 2.0 and reference groups. Neutrophil and platelet sequestration usually occur within 5-15 min of perfusion, and were not attenuated in association with Pig 2.0 multitransgenic lungs.

These data demonstrate the novel Pig 2.0 donor genetics protect lungs from PVR rise and lung injury and were associated with significantly improved lung survival in this rigorous model. Leukocyte and white cell sequestration were not prevented, as previously described with other lung genetics. The transgene combination expressed by Pig 2.0 lungs may be helpful to accomplish successful xenotransplantation of the lung, and other organs

Transgene expression. RNAseq expression data showed complement and cellular toxicity genes are expressed in samples collected from Payload 9 and Payload 10 Pig 2.0 pigs (FIG. 36). FACS data showed complement and cellular toxicity proteins are expressed in samples collected from Payload 5, Payload 9 and Payload 10 pigs (FIG. 37). All three payloads expressed complement (CD46, CD55, and CD59) and cellular toxicity related proteins (e.g., B2M, HLA-E, CD47). In addition, Payload 5 expressed CD39, while Payload 10 expressed PDL1. Although performance in NHP drastically differs, the gene expression profiles are similar among the five pigs carrying payload 5.

The use of numerical values specified in this application, unless expressly indicated otherwise, are stated as approximations through the minimum and maximum values specified within the stated ranges, and preceded by the word “about.” The disclosure of ranges is intended as a continuous range including every value between the minimum and maximum values recited as well as any ranges that may be formed through such values. The numerical values presented in this application represent various embodiments of the present disclosure.

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown and/or described in detail to avoid unnecessarily obscuring the description of the embodiments of the present technology. Although steps of methods may be presented herein in a particular order, in alternative embodiments the steps may have another suitable order. Similarly, certain embodiments of the present technology disclosed in the context of particular embodiments may be combined or eliminated in other embodiments. Furthermore, while advantages associated with certain embodiments may have been disclosed in the context of those embodiments, other embodiments may also exhibit such advantages, and not all embodiments need necessarily exhibit such advantages or other advantages disclosed herein to fall within the scope of the present technology. Accordingly, this disclosure and associated technology may encompass other embodiments not expressly shown and/or described herein.

From the foregoing, it will be appreciated that specific embodiments of the disclosure have been described herein for purposes of illustration, but that various modifications may be made without deviating from the scope of the disclosure. Accordingly, the disclosure is not limited except as by the appended claims.

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

### ABBREVIATIONS

acute vascular rejection (AVR); activated partial thromboplastin time (APTT); adeno-associated virus integration site 1 (AAVS1); alanine aminotransferase (ALT); albumin (ALB); alpha 1,3-galactosyl-galactose (Gal or aGal); antibody-mediated rejection (AMR); anti-thymocyte globulin (ATG); asialoglycoprotein receptor 1 (ASGR1); aspartate aminotransferase (AST); β1,4 N-acetylgalactosaminyltransferase 2 (B4GalNT2); Beta-2 microglobulin (B2M); Cluster of Differentiation 39 (CD39); Cluster of Differentiation 47 (CD47); clustered regularly interspaced short palindromic repeats (CRISPR); class II transactivator dominant-negative (CIITA-DN); CMV early enhancer/chicken β actin (CAG); complement factor 3 (C3); complement factor 3 knockout (C3-KO); complete blood count (CBC); C-X-C motif chemokine receptor 3 (CXCR3); C-X-C motif chemokine receptor 12 (CXCR12); cytidine monophosphate-N-acetylneuraminic acid hydroxylase (CMAH); cytotoxic T-lymphocyte-associated immunoglobulin (CTLA-Ig); deoxyribonucleic acid (DNA); DQ Alpha (DQA); DR Alpha (DRA); droplet digital pCR (ddPCR); ecto-5′ Nucleotidase (CD73); elongation factor 1α (EF1α); endothelial cells (EC); endothelial protein C receptor (EPCR); ex-vivo liver xenoperfusion (EVLXP); Fas ligand (FasL); fibrinogen levels (FIB); fluorescence-activated cell sorting (FACS); fresh frozen plasma (FFP); green fluorescent protein (GFP); glomerular filtration rate (GFR); glucagon like peptide 1 receptor (GLP-1R); glycoprotein IIb/IIIa (GpIIb/IIIa); glycoprotein α-galactosyltransferase 1 (GGTA); GGTA knock out (GTKO); guide ribonucleic acid (gRNA); haemotoxylin and eosin (H+E); hepatic artery thrombosis (HAT); human embryonic kidney 293 (HEK293); heme oxygenase (HO-1); homology-directed repair (HDR); human blood and plasma (hWB+P); human membrane cofactor protein (hCD46); human complement decay accelerating factor (hCD55); human complement regulatory proteins (hCRPs); human leukocyte antigen (HLA); human leukocyte antigen-E (HLA-E); human MAC-inhibitor factor (hCD59); immunoglobulin G (IgG); immunoglobulin G-degrading enzyme of Streptococcus pyogenes (IdeS); immunoglobulin M (IgM); immunohistochemistry (IHC); inosine monophosphate dehydrogenase (IMDH); interleukin 12 (IL12); interleukin 35 (IL35); international normalized ratio (INR); intracellular adhesion molecule-2 (ICAM2); killer inhibitory receptors (KIR); knockin (KI); knockout (KO); Krüppel associated box (KRAB); liver functional test (LFT); long terminal repeat (LTR); major histocompatibility complex class I (MHC class I); major histocompatibility complex class II (MHC class II); major histocompatibility complex, class I, E single chain trimer (HLA-ESCT); mechanistic target of rapamycin (mTOR); messenger ribonucleic acid (mRNA); modification of diet in renal disease (MDRD); mixed lymphocyte reaction (MLR); mycophenolate mofetil (MMF); natural killer (NK); N-glycolylneuraminic acid (Neu5Gc); neurogenic differentiation 1 (NeuroD); non-human primate (NHP); non-homologous end joining (NHEJ); orthotopic liver xenotransplants (OLTx); panel reactive antibody (PRA); peripheral blood mononuclear cell (PBMC); pig kidney-15 cells (PK15); porcine endogenous retroviruses (PERV); porcine endogenous retroviruses knockout (PERV KO); programmed death-ligand 1 (PD-L1); polymerase chain reaction (PCR); porcine aortic endothelial cell line (PEC-A or pAEC); potassium (K); Prothrombin Time (PT) and International Normalized Ratio (PT-NIR); quantitative reverse transcription polymerase chain reaction (qRT-PCR); recombinase-mediated cassette exchange (RMCE); red blood cell (RBC); ribonucleic acid sequencing (RNAseq); reverse transcriptase polymerase chain reaction (RT-PCT); sgRNA (single guide RNA); small interfering ribonucleic acid (siRNA); sodium (Na); somatic cell nuclear transfer (SCNT); superoxide dismutase 3 (SOD3); swine leukocyte antigen (SLA); T-cell mediated rejection (TCMR); thrombin-antithrombin III (TAT); thrombomodulin (THBD, TBM, or TM); thrombotic microangiopathy (TMA); tissue factor pathway inhibitor (TFPI); topoisomerase (TOPO); total bilirubin (Tbili); transcription activator-like (TAL) effector and nucleases (TALEN); tumor necrosis factor α-induced protein 3 (A20); tumor necrosis factor receptor 1 immunoglobulin (TNFR1-Ig); ubiquitous chromatin opening element (UCOE); von Willebrand factor (vWF); whole genome sequencing (WGS); wild type (WT); Zinc finger nucleases (ZFN).

### REFERENCES

Ahlborg et al. N Engl J Med 349(4):327-334 (Jul. 24, 2003) (PMID: 12878739; DOI: 10.1056/NEJMoa022464)
Armstrong et al. J Gen Virol 10(2):195-198 (February 1971) (PMID: 4324256; DOI: 10.1099/0022-1317-10-2-195)
Baertschiger et al. Xenotransplantation 11(1):27-32 (January 2004) (PMID: 14962290; DOI: 10.1111/j.1399-3089.2004.00075.x)
Boron & Boulpaep, Medical Physiology: A Cellular and Molecular Approach, Elsevier/Saunders (1st ed., 2003)
Byrne et al. Xenotransplantation 21(6):543-554) (November-December 2014) (PMID: 25176027; DOI: 10.1111/xen.12124)
Cibelli et al. Science 280(5367):1256-1258 (May 22, 1998) (PMID: 9596577)
Clémenceau et al. Diabetologia 45(6):914-923 (June 2002) (PMID: 12107737; DOI: 10.1007/s00125-002-0832-7)
Cooper et al. Transplantation 77(1):1-5 (Jan. 15, 2004) (PMID: 14724427; DOI: 10.1097/01.TP.0000105116.74032.63)
Cooper et al. Int J Surg 23(Pt B):211-216 (November 2015) (PMID: 26159291; DOI: 10.1016/j.ijsu.2015.06.068)
Cowan et al. Kidney Int 85(2):265-275 (February 2014) (PMID: 24088952; DOI: 10.1038/ki.2013.381)
Davis et al. Transplantation 98(9):931-936 (Nov. 15, 2014) (PMID: 25286057; DOI: 10.1097/TP.0000000000000446)
Donati-Bourne et al. J Transplant 204:317574 (2014) (PMID: 24688785; DOI: 10.1155/2014/317574)
Dunn et al. FASEB J 29(Suppl 1):LB761 (Apr. 1, 2015)
Eggers Am J Kidney Dis 15(5):414-421 (May 1990) (PMID: 2185627)
Ekser et al. Int J Surg 23(Pt B):197-198 (November 2015) (PMID: 26318503; DOI: 10.1016/j.ijsu.2015.08.036)
FDA Guidance for Industry: Source Animal, Product, Preclinical, and Clinical Issues Concerning the Use of Xenotransplantation Products in Humans (April 2003, revised December 2016)
FDA Guidance for Industry: Gene Therapy Clinical Trials—Observing Subjects for Delayed Adverse Events (November 2006)
FDA Guidance: Preclinical Assessment of Investigational Cellular and Gene Therapy Products (November 2013)
Fiebig et al. Virology 307(2):406-413 (Mar. 15, 2003) (PMID: 12667808)
Fischer et al. Sci Rep 6:29081 (Jun. 29, 2016) (PMID: 27353424; DOI: 10.1038/srep29081)
Gourishankar et al. J Am Soc Nephrol 14(9):2387-2394 (September 2003) (PMID: 12937318)
Grams et al. Transplantation 94(7):750-756 (Oct. 15, 2012) (PMID: 22932116; DOI: 10.1097/TP.0b013e31826205b9)
Higginbotham et al. Xenotransplantation 22(3):221-230 (May-June 2015) (PMID: 25847130; DOI: 10.1111/xen.12166)
Ide et al. Proc Natl Acad Sci USA 104:5062-5066 (2007)
Iwase et al. Xenotransplantation 22(4):302-309 (July-August 2015) (PMID: 26130164; DOI: 10.1111/xen.12174)
Iwase & Kobayashi Int J Surg 23(Pt B):229-233 (November 2015) (PMID: 26305729; DOI: 10.1016/j.ijsu.2015.07.721)
Kasiske et al. Am J Kidney Dis 56(5):947-960 (November 2010) (PMID: 20801565; DOI: 10.1053/j.ajkd.2010.06.020)
Kim et al. Genome Res 24(6):1012-1019 (June 2014) (PMID: 24696461; DOI: 10.1101/gr.171322.113)
Kim et al. Am J Transplant 17 Suppl 1:174-251 (January 2017) (PMID: 28052604; DOI: 10.1111/ajt.14126)
Lai et al. Science 295(5557):1089-1092 (Feb. 8, 2002) (PMID: 11778012; DOI: 10.1126/science.1068228)
Lee et al. Anim Biotechnol 22(4):175-180 (October 2011) (PMID: 22132811; DOI: 10.1080/10495398.2011.595294)
Levey et al. Kidney Int 80(1):17-28 (July 2011) (PMID: 21150873; DOI: 10.1038/ki.2010.483)
Lilienfeld Xenotransplantation 14(2):126-134 (March 2007) (PMID: 17381687; DOI: 10.1111/j.1399-3089.2007.00378.x)
Loveland et al. Xenotransplantation 11(2):171-183 (March 2004) (PMID: 14962279; DOI: 10.1046/j.1399-3089.2003.00103.x)
Lutz et al. Xenotransplantation 20(1):27-35 (January-February 2013) (PMID: 23384142; DOI: 10.1111/xen.12019)
Marcén et al. NDT Plus 3(Suppl_2):ii2-ii8 (June 2010) (PMID: 20508857; DOI: 10.1093/ndtplus/sfq063)
Martens et al. Transplantation 101(4):e86-e92 (April 2017) (PMID: 28114170; DOI: 10.1097/TP.0000000000001646)
McGregor et al. Transplantation 93(7):686-692 (Apr. 15, 2012) (PMID: 22391577;
DOI: 10.1097/TP.0b013e3182472850)
Moalic et al. J Virol 80(22):10980-10988 (November 2006) (PMID: 16928752; DOI: 10.1128/JV1.00904-06)
Mohiuddin et al. Nat Commun 7:11138 (Apr. 5, 2016) (PMID: 27045379; DOI: 10.1038/ncomms11138)
Niu et al. Science 357(6357):1303-1307 (Sep. 22, 2017) (PMID: 28798043; DOI: 10.1126/science.aan4187)
Ojo et al. Transplantation 71(1):82-90 (Jan. 15, 2001) (PMID: 11211201)
Patience et al. Nat Med 3(3):282-286 (March 1997) (PMID: 9055854)
Patience et al. J Virol 75(6):2771-2775 (March 2001) (PMID: 11222700; DOI: 10.1128/JVI.75.6.2771-2775.2001)
Pinheiro et al. Anal Chem 84(2):1003-1011 (Jan. 17, 2012) (PMID: 22122760; DOI: 10.1021/ac202578x)
Ramsoondar et al. Xenotransplantation 16(3):164-180 (May-June 2009) (PMID: 19566656; DOI: 10.1111/j.1399-3089.2009.00525.x)
Reyes et al. J Immunol 193(11):5751-5757 (Dec. 1, 2014) (PMID: 25339675; DOI: 10.4049/jimmunol.1402059)
Robson et al. Xenotransplantation 7(3):166-176 (August 2000) (PMID: 11021661)
Schuurman Xenotransplantation 16(4):215-222 (July-August 2009) (PMID: 19799761; DOI: 10.1111/j.1399-3089.2009.00541.x)
Semaan et al. Xenotransplantation 19(2):112-121 (March-April 2012) (PMID: 22497513; DOI: 10.1111/j.1399-3089.2012.00683.x)
Semaan et al. PLoS One 10(4):e0122059 (Apr. 24, 2015) (PMID: 25909512; DOI: 10.1371/journal.pone.0122059)
Sharma et al. Br J Gen Pract 60(575):e266-e276 (June 2010) (DOI: 10.3399/bjgp10X502173)
Shen et al. Transplant Proc 43(5):1994-1997 (June 2011) (PMID: 21693314; DOI: 10.1016/j.transproceed.2011.03.037)
Starzl et al. Transplantation 2:752-756 (November 1964) (PMID: 14224657)
Starzl et al. Hepatology 17(6):1127-1152 (June 1993) (PMID: 8514264)
Tanabe et al. Am J Transplant 17(7):1778-1790 (July 2017) (PMID: 28117931; DOI: 10.1111/ajt.14210)
Tseng et al. Transplantation 81(7):1058-1062 (Apr. 15, 2006) (PMID: 16612284; DOI: 10.1097/01.tp.0000197555.16093.98)
Vagefi et al. Int J Surg 23(Pt B):291-295 (November 2015) (PMID: 26296932; DOI: 10.1016/j.ijsu.2015.07.720)
van′t Veer et al. J Biol Chem 272(12):7983-7994 (Mar. 21, 1997) (PMID: 9065469; DOI: 10.1074/jbc.272.12.7983)
Wang et al. Sci Rep 6:38854 (Dec. 16, 2016) (PMID: 27982048; DOI: 10.1038/srep38854)
Yang et al. Nucleic Acids Res 41(19):9049-9061 (October 2013) (PMID: 23907390; DOI: 10.1093/nar/gkt555)
Yang et al. Science 350(6264):1101-1104 (Nov. 27, 2015) (PMID: 26456528; DOI: 10.1126/science.aad1191)
Ye et al. Transplantation 60(1):19-22 (Jul. 15, 1995) (PMID: 7624938)

SEQUENCES

| SEQ | |
|---|---|
| ID | |
| NO | SEQUENCE |
| 1 | tcaacacaaacatatctttg |
| 2 | tgtttgtgttgatacgtcag |
| 3 | gaaactgactaggatccatg |
| 4 | ctcagtgggttaactatccg |
| 5 | tctcacctgtgaagcctgcg |
| 6 | cacagtgacttgggccacta |
| 7 | tctcacctgtgaagcctgcgcgg |
| 8 | cacagtgacttgggccactaggg |
| 9 | tcctcctgcagtcactgtgatgg |
| 10 | ggtgccccccacagaaggcccgg |
| 11 | cagccccaccacaccctacgagg |
| 12 | cttcttctgcagcaaacttctgg |
| 13 | cggctctgacaagctgtccgagg |
| 14 | cgaggccctgaaggtcttcgtgg |
| 15 | ctcccagaagcacatccgcgtgg |
| 16 | ccacgcctacatctcgctccagg |
| 17 | gaaagcggccctcggagctgcgg |
| 18 | cgccagccaggtgaagtacgcgg |
| 19 | cgaggtggcttccatcagcgagg |
| 20 | tacacgctcttccaaatctttgg |
| 21 | ccgtatagccctgctgctcatgg |
| 22 | cagccaggagccacgccggctgg |
| 23 | gaacttggcccgctacctccagg |
| 24 | gaagaaggtcaccgtgattccgg |
| 25 | gatccgcctcatcgagaagcagg |
| 26 | cccggagaacaaagcctttgtgg |
| 27 | ctacctctgcgacctcgccccgg |
| 28 | ccctacgcggcgccccctagtgg |
| 29 | actgtggcgcctgagctccccgg |
| 30 | gctcgaacccaagaagagaatgg |
| 31 | atcacagtgactgcaggaggagg |
| 32 | cggttccgcgcaggcttcacagg |
| 33 | ctgaccgggccttctgtgggggg |
| 34 | cctcctcgtagggtgtggtgggg |
| 35 | aagtcgtgcagcggcggctctgg |
| 36 | agccgtccagcaggaagaccagg |
| 37 | cagggcctcgaagtcggcctcgg |
| 38 | tgtgcttctgggagatgtgcagg |
| 39 | ggtactccaccaccgccacgcgg |
| 40 | ctggagcgagatgtaggcgtggg |
| 41 | gcagctccgagggccgctttcgg |
| 42 | gcccgcgtacttcacctggctgg |
| 43 | gtacttcaaaacctcgctgatgg |
| 44 | ggtcgaccctgccaaagatttgg |
| 45 | agggctatacgggaggcttcggg |
| 46 | cagccggcgtggctcctggctgg |
| 47 | gaggtagcgggccaagttctggg |
| 48 | cggtgaccttcttcttcttcagg |
| 49 | acgtggggtccgatgcccaccgg |
| 50 | cgatgaggcggatctgcttgagg |
| 51 | cacaaaggctttgttctccgggg |
| 52 | cttccggggcgaggtcgcagagg |
| 53 | agggggcgccgcgtaggggcggg |
| 54 | ctcaggcgccacagtgacttggg |
| 55 | gttcgagcgttgaaaccccgggg |
| 56 | atccaagaccattctcttcttgg |
| 57 | accatcacagtgactgcaggagg |
| 58 | gacaccatcacagtgactgcagg |
| 59 | ctgtgaagcctgcgcggaaccgg |
| 60 | ggggggcaccggttccgcgcagg |
| 61 | agggtgtggtggggctgaccggg |
| 62 | gaaccggtgccccccacagaagg |
| 63 | gggccttctgtggggggcaccgg |
| 64 | gctgaccgggccttctgtggggg |
| 65 | tcctcctcgtagggtgtggtggg |
| 66 | ggctgaccgggccttctgtgggg |
| 67 | gtcctcctcgtagggtgtggtgg |
| 68 | gggctgaccgggccttctgtggg |
| 69 | cgtgtcctcctcgtagggtgtgg |
| 70 | ggggctgaccgggccttctgtgg |
| 71 | tagggtgtggtggggctgaccgg |
| 72 | tctggcgtgtcctcctcgtaggg |
| 73 | cagaagaagtcgtgcagcggcgg |
| 74 | ctctggcgtgtcctcctcgtagg |
| 75 | ctgcagaagaagtcgtgcagcgg |
| 76 | ctgcagcaaacttctggacctgg |
| 77 | tctggacctggtcttcctgctgg |
| 78 | gacctggtcttcctgctggacgg |
| 79 | gctgtccgaggccgacttcgagg |
| 80 | ggtcttcgtggtgggcatgatgg |
| 81 | gcttgtcagagccgtccagcagg |
| 82 | ggccgacttcgaggccctgaagg |
| 83 | gaccttcagggcctcgaagtcgg |
| 84 | gcccaccacgaagaccttcaggg |
| 85 | ggccctgaaggtcttcgtggtgg |
| 86 | gccctgaaggtcttcgtggtggg |
| 87 | tgcccaccacgaagaccttcagg |
| 88 | cgccacgcggatgtgcttctggg |
| 89 | tgtaggcgtgggagccgtcgtgg |
| 90 | ccgagggccgctttcggtcctgg |
| 91 | ccagaagcacatccgcgtggcgg |
| 92 | gaagcacatccgcgtggcggtgg |
| 93 | gcacatccgcgtggcggtggtgg |
| 94 | gcggtggtggagtaccacgacgg |
| 95 | tctcgctccaggaccgaaagcgg |
| 96 | gctgcggcgcatcgccagccagg |
| 97 | gaagtacgcgggcagcgaggtgg |
| 98 | tcggtcctggagcgagatgtagg |
| 99 | gcgatgcgccgcagctccgaggg |
| 100 | cgctgcccgcgtacttcacctgg |
| 101 | ggcgatgcgccgcagctccgagg |
| 102 | gccagccaggtgaagtacgcggg |
| 103 | ggtgaagtacgcgggcagcgagg |
| 104 | cgctcttccaaatctttggcagg |
| 105 | gctcttccaaatctttggcaggg |
| 106 | aaatctttggcagggtcgaccgg |
| 107 | ctatacgggaggcttcgggccgg |
| 108 | ctggctggccatgagcagcaggg |
| 109 | aagttctgggccagccggcgtgg |
| 110 | cttcaggccctggaggtagcggg |
| 111 | tccgatgcccaccggaatcacgg |
| 112 | atctgcttgaggctgacgtgggg |
| 113 | gggcctgcttctcgatgaggcgg |
| 114 | gtccacaccgctgaccacaaagg |
| 115 | cagggctatacgggaggcttcgg |
| 116 | gagcagcagggctatacgggagg |
| 117 | catgagcagcagggctatacggg |
| 118 | cctgctgctcatggccagccagg |
| 119 | acgccggctggcccagaacttgg |
| 120 | ccagggcctgaagaagaagaagg |
| 121 | accgtgattccggtgggcatcgg |
| 122 | tggccagccaggagccacgccgg |
| 123 | gggccagccggcgtggctcctgg |
| 124 | gggccaagttctgggccagccgg |
| 125 | tcttcttcttcaggccctggagg |
| 126 | ggaggtagcgggccaagttctgg |
| 127 | aacttggcccgctacctccaggg |
| 128 | tcttcaggccctggaggtagcgg |
| 129 | gaaggtcaccgtgattccggtgg |
| 130 | aaggtcaccgtgattccggtggg |
| 131 | gatctgcttgaggctgacgtggg |
| 132 | ccggggcctgcttctcgatgagg |
| 133 | ggatctgcttgaggctgacgtgg |
| 134 | aacaaagcctttgtggtcagcgg |
| 135 | accacaaaggctttgttctccgg |
| 136 | agcctttgtggtcagcggtgtgg |
| 137 | ggtcagcggtgtggacgagctgg |
| 138 | cggaagtgcccgcccctacgcgg |
| 139 | cctagtggcccaagtcactgtgg |
| 140 | gggcgggcacttccggggcgagg |
| 141 | cagtgacttgggccactaggggg |
| 142 | gttgaaaccccggggagctcagg |
| 143 | tccaagaccattctcttcttggg |
| 144 | cgtaggggcgggcacttccgggg |
| 145 | gcgtaggggcgggcacttccggg |
| 146 | cgcgtaggggcgggcacttccgg |
| 147 | tagggggcgccgcgtaggggcgg |
| 148 | cactagggggcgccgcgtagggg |
| 149 | gccactagggggcgccgcgtagg |
| 150 | gctcaggcgccacagtgacttgg |
| 151 | acagtgacttgggccactagggg |
| 152 | ctgtggcgcctgagctccccggg |
| 153 | tgtggcgcctgagctccccgggg |
| 154 | ggttcgagcgttgaaaccccggg |
| 155 | gggttcgagcgttgaaaccccgg |
| 156 | acccaagaagagaatggtcttgg |
| 157 | gaagagaatggtcttggatgtgg |
| 158 | tctccagacgcaggacgttgggg |
| 159 | ggaggcccacgaagggcaagggg |
| 160 | tgcctagcatctcgtgcccctgg |
| 161 | cacccccaacgtcctgcgtctgg |
| 162 | gagtgaggagatggtggtgttgg |
| 163 | acgaagggcaaggggatattcgg |
| 164 | ggtcaccgtccatgacttcccgg |
| 165 | cctgagcaccgtcaacatcaagg |
| 166 | tcccaggggcacgagatgctagg |
| 167 | aactgcgggaggggaggacgagg |
| 168 | caggacgttgggggtgattatgg |
| 169 | aatatccccttgcccttcgtggg |
| 170 | cttggccgggaagtcatggacgg |
| 171 | gttcagcgtcgtggtctcgctgg |
| 172 | gacggtgctcaggtagttgttgg |
| 173 | ggggtggggcttcagcggtccgg |
| 174 | gcctagcatctcgtgcccctggg |
| 175 | gcgtagagctgtcatcccagggg |
| 176 | tggtgtaactgcgggaggggagg |
| 177 | ctccagacgcaggacgttggggg |
| 178 | ggcgtagagctgtcatcccaggg |
| 179 | aggcgtagagctgtcatcccagg |
| 180 | ttatggtgtaactgcgggagggg |
| 181 | attatggtgtaactgcgggaggg |
| 182 | gattatggtgtaactgcgggagg |
| 183 | ggtgattatggtgtaactgcggg |
| 184 | ctctccagacgcaggacgttggg |
| 185 | gggtgattatggtgtaactgcgg |
| 186 | actctccagacgcaggacgttgg |
| 187 | cgtcctgcgtctggagagtgagg |
| 188 | gtggtgttggaggcccacgaagg |
| 189 | gcaaggggatattcgggtttcgg |
| 190 | tgacttcccggccaagagacagg |
| 191 | tctcctcactctccagacgcagg |
| 192 | gcgtctggagagtgaggagatgg |
| 193 | tggtgttggaggcccacgaaggg |
| 194 | tctggagagtgaggagatggtgg |
| 195 | ttggaggcccacgaagggcaagg |
| 196 | tgaggagatggtggtgttggagg |
| 197 | tggaggcccacgaagggcaaggg |
| 198 | gaatatccccttgcccttcgtgg |
| 199 | cgaagggcaaggggatattcggg |
| 200 | gtctcttggccgggaagtcatgg |
| 201 | acagcacctgtctcttggccggg |
| 202 | gacagcacctgtctcttggccgg |
| 203 | gttggcgttgttcagcgtcgtgg |
| 204 | gctggacagcacctgtctcttgg |
| 205 | gagcaccgtcaacatcaaggtgg |
| 206 | cgcgcccaccttgatgttgacgg |
| 207 | agcaccgtcaacatcaaggtggg |
| 208 | aggtgggcgcgctcaacagccgg |
| 209 | aagaaggggtggggcttcagcgg |
| 210 | gagaaaataatgaatgtcaa |
| 211 | MRIFAVFIFMTYWHLLNAFTVTVPKDLYVVEYGSNMTIECKFP |
| | VEKQLDLAALIVYWEMEDKNIIQFVHGEEDLKVQHSSYRQRAR |
| | LLKDQLSLGNAALQITDVKLQDAGVYRCMISYGGADYKRITVK |
| | VNAPYNKINQRILVVDPVTSEHELTCQAEGYPKAEVIWTSSDH |
| | QVLSGKTTTTNSKREEKLFNVTSTLRINTTTNEIFYCTFRRLD |
| | PEENHTAELVIPELPLAHPPNERTHLVILGAILLCLGVALTFI |
| | FRLRKGRMMDVKKCGIQDTNSKKQSDTHLEET |

I/We claim:

1. An isolated cell, tissue, organ, or animal comprising a plurality of transgenes of at least two types selected from the group consisting of inflammatory response transgenes, immune response transgenes, immunomodulator transgenes, and combinations thereof.

2. An isolated cell, tissue, organ, or animal comprising a plurality of transgenes, wherein the plurality of transgenes comprises at least one inflammatory response transgene, at least one immune response transgene, and at least one immunomodulator transgene.

3. The isolated cell, tissue, organ, or animal of claim 1 or 2, wherein the inflammatory response transgene is selected from the group consisting of TNF α-induced protein 3 (A20), heme oxygenase (HO-1), Cluster of Differentiation 47 (CD47), and combinations thereof.

4. The isolated cell, tissue, organ, or animal of claim 1 or 2, wherein the immune response transgene is selected from the group consisting of human leukocyte antigen-E (HLA-E), beta-2 microglobulin (B2M), and combinations thereof.

5. The isolated cell, tissue, organ, or animal of any one of claim 1 or 2, wherein the immunomodulator transgene is selected from the group consisting of programmed death-ligand 1 (PD-L1), Fas ligand (FasL), and combinations thereof.

6. The isolated cell, tissue, organ, or animal of claim 1 or 2, wherein the plurality of transgenes further comprises at least one coagulation response transgene.

7. The isolated cell, tissue, organ, or animal of claim 6, wherein the coagulation response transgene is selected from the group consisting of Cluster of Differentiation 39 (CD39), thrombomodulin (THBD), tissue factor pathway inhibitor (TFPI), and combinations thereof.

8. The isolated cell, tissue, organ, or animal of claim 1 or 2, wherein the plurality of transgenes further comprises at least one complement response transgene.

9. The isolated cell, tissue, organ, or animal of claim 8, wherein the complement response transgene is selected from the group consisting of human membrane cofactor protein (hCD46), human complement decay accelerating factor (hCD55), human MAC-inhibitor factor (hCD59), and combinations thereof.

10. An isolated cell, tissue, organ, or animal comprising six or more transgenes, each independently selected from the group consisting of complement response transgenes, coagulation response transgenes, inflammatory response transgenes, immune response transgenes, and immunomodulator transgenes.

11. The isolated cell, tissue, organ, or animal of claim 10, wherein the isolated cell, tissue, organ, or animal comprises 9, 10, 11, or 12 transgenes.

12. The isolated cell, tissue, organ, or animal of claim 10, wherein the complement response transgene is selected from the group consisting of human membrane cofactor protein (hCD46), human complement decay accelerating factor (hCD55), human MAC-inhibitor factor (hCD59), and combinations thereof.

13. The isolated cell, tissue, organ, or animal of claim 10, wherein the coagulation response transgene is selected from the group consisting of Cluster of Differentiation 39 (CD39), thrombomodulin (THBD), tissue factor pathway inhibitor (TFPI), and combinations thereof.

14. The isolated cell, tissue, organ, or animal of claim 10, wherein the inflammatory response transgene is selected from the group consisting of TNF α-induced protein 3 (A20), heme oxygenase (HO-1), Cluster of Differentiation 47 (CD47), and combinations thereof.

15. The isolated cell, tissue, organ, or animal of claim 10, wherein the immune response transgene is selected from the group consisting of human leukocyte antigen-E (HLA-E), beta-2 microglobulin (B2M), and combinations thereof.

16. The isolated cell, tissue, organ, or animal of any one of claim 10, wherein the immunomodulator transgene is selected from the group consisting of programmed death-ligand 1 (PD-L1), Fas ligand (FasL), and combinations thereof.

17. The isolated cell, tissue, organ, or animal of any one of claims 10-16, wherein the six or more transgenes are selected from the group consisting of hCD46, hCD55, hCD59, HLA-E, B2M, CD47, CD39, THBD, TFPI, A20, PD-L1, and HO-1.

18. The isolated cell, tissue, organ, or animal of claim 17, wherein the cell, tissue, organ, or animal comprises hCD46, hCD55, hCD59, CD39, THBD, TFPI, A20, HO-1, CD47, HLA-E, B2M, and PD-L1 transgenes or THBD, TFPI, CD39, CD46, CD55, CD59, CD46, HO-1, A20, B2M, HLA-E SCT, and CD47 transgenes.

19. The isolated cell, tissue, organ, or animal of claim 18, comprising the vector in one of FIG. 17-20, 31, or 47-49.

20. The isolated cell, tissue, organ, or animal of any one of claims 10-19, wherein the at least six transgenes are expressed from a single locus.

21. The isolated cell, tissue, organ, or animal of any one of claims 10-20, wherein the at least six transgenes are expressed at a clinically effective level.

22. The isolated cell, tissue, organ, or animal of any one of claims 10-21, further comprising a genetically modified von Willebrand factor (vWF) gene.

23. The isolated cell, tissue, organ, or animal of claim 22, wherein the modified vWF gene is humanized.

24. The isolated cell, tissue, organ, or animal of any one of claims 10-23, further comprising a deletion, disruption, or inactivation of asialoglycoprotein receptor 1 (ASGR1).

25. The isolated cell, tissue, organ, or animal of any one of claims 1-24, further comprising a deletion, disruption, or inactivation of one or more carbohydrate antigen genes.

26. The isolated cell, tissue, organ, or animal of claim 25, wherein the one or more carbohydrate antigen genes are selected from the group consisting of glycoprotein α-galactosyltransferase 1 (GGTA), β1,4 N-acetylgalactosaminyltransferase 2 (B4GalNT2), cytidine monophosphate-N-acetylneuraminic acid hydroxylase (CMAH).

27. The isolated cell, tissue, organ, or animal of any one of claims 1-26, wherein the isolated cell, tissue, organ, or subject is a porcine cell, porcine tissue, a porcine organ, a pig or progeny thereof.

28. The isolated cell, tissue, organ, or animal of claim 27, wherein the isolated cell, tissue, organ, or animal is a PERV-free porcine cell, PERV-free porcine tissue, or a PERV-free porcine.

29. The isolated cell, tissue, organ, or animal of any one of claims 1-28, wherein the organ is a kidney or a liver.

30. A vector comprising a plurality of transgenes of at least two types selected from the group consisting of inflammatory response transgenes, immune response transgenes, immunomodulator transgenes, and combinations thereof.

31. A vector comprising a plurality of transgenes, wherein the plurality of transgenes comprises at least one inflammatory response transgene, at least one immune response transgene, and at least one immunomodulator transgene.

32. The vector of claim 30 or 31, wherein the inflammatory response transgenes are selected from the group consisting of TNF α-induced protein 3 (A20), heme oxygenase (HO-1), Cluster of Differentiation 47 (CD47), and combinations thereof.

33. The vector of any of claims 30-32, wherein expression of at least a portion of the inflammatory response transgenes is driven by a tissue-specific promoter, a ubiquitous promoter, or any combination thereof.

34. The vector of claim 33, wherein the tissue-specific promoter is an endothelial-specific promoter.

35. The vector of claim 30 or 31, wherein the immune response transgenes are selected from the group consisting of human leukocyte antigen-E (HLA-E), beta-2 microglobulin (B2M), and combinations thereof.

36. The vector of any of claim 30, 31, or 35, wherein expression of at least a portion of the immune response transgenes is driven by a ubiquitous promoter.

37. The vector of claim 30 or 31, wherein the immunomodulator transgenes are selected from the group consisting of programmed death-ligand 1 (PD-L1), Fas ligand (FasL), and combinations thereof.

38. The vector of claim 30 or 31, wherein the plurality of transgenes further comprises at least one coagulation response transgene.

39. The vector of claim 38, wherein the coagulation response transgene is selected from the group consisting of Cluster of Differentiation 39 (CD39), thrombomodulin (THBD), tissue factor pathway inhibitor (TFPI), and combinations thereof.

40. The vector of claim 38 or 39, wherein expression of at least a portion of the coagulation response transgenes is driven by a tissue-specific promoter.

41. The vector of claim 40, wherein the tissue-specific promoter is an endothelial-specific promoter.

42. The vector of claim 41, wherein the endothelial-specific promoter is a low expression endothelial-specific promoter.

43. The vector of claim 30 or 31, wherein the plurality of transgenes further comprises at least one complement response transgene.

44. The vector of claim 43, wherein the complement response transgene is selected from the group consisting of human membrane cofactor protein (hCD46), human complement decay accelerating factor (hCD55), human MAC-inhibitor factor (hCD59), and combinations thereof.

45. The vector of claim 43 or 44, wherein expression of at least a portion of the complement response transgenes is driven by a ubiquitous promoter.

46. A vector comprising six or more transgenes, each independently selected from the group consisting of complement response transgenes, coagulation response transgenes, inflammatory response transgenes, immune response transgenes, and immunomodulator transgenes.

47. The vector of claim 46, wherein the vector comprises 9, 10, 11, or 12 transgenes.

48. The vector of claim 46, wherein the complement response transgene is selected from the group consisting of human membrane cofactor protein (hCD46), human complement decay accelerating factor (hCD55), human MAC-inhibitor factor (hCD59), and combinations thereof.

49. The vector of any one of claims 46-48, wherein expression of at least a portion of the complement response transgenes is driven by a ubiquitous promoter.

50. The vector of claim 46, wherein the coagulation response transgene is selected from the group consisting of Cluster of Differentiation 39 (CD39), thrombomodulin (THBD), tissue factor pathway inhibitor (TFPI), and combinations thereof.

51. The vector of any of claims 43-50, wherein expression of at least a portion of the coagulation response transgenes is driven by a tissue-specific promoter.

52. The vector of claim 51, wherein the tissue-specific promoter is an endothelial-specific promoter.

53. The vector of claim 52, wherein the endothelial-specific promoter is a low expression endothelial-specific promoter.

54. The vector of claim 46, wherein the inflammatory response transgene is selected from the group consisting of TNF α-induced protein 3 (A20), heme oxygenase (HO-1), Cluster of Differentiation 47 (CD47), and combinations thereof.

55. The vector of any of claims 46-54, wherein expression of at least a portion of the inflammatory response transgenes is driven by a tissue-specific promoter, a ubiquitous promoter, or any combination thereof.

56. The vector of claim 55, wherein the tissue-specific promoter is an endothelial-specific promoter.

57. The vector of claim 46, wherein the immune response transgene is selected from the group consisting of human leukocyte antigen-E (HLA-E), beta-2 microglobulin (B2M), and combinations thereof.

58. The vector of any of claims 46-57, wherein expression of at least a portion of the immune response transgenes is driven by a ubiquitous promoter.

59. The vector of claim 46, wherein the immunomodulator transgene is selected from the group consisting of programmed death-ligand 1 (PD-L1), Fas ligand (FasL), and combinations thereof.

60. The vector of any one of claims 46-59, wherein the six or more transgenes are selected from the group consisting of hCD46, hCD55, hCD59, HLA-E, B2M, CD47, CD39, THBD, TFPI, A20, PD-L1, and HO-1.

61. The vector of claim 60, wherein the vector comprises hCD46, hCD55, hCD59, CD39, THBD, TFPI, A20, HO-1, CD47, HLA-E, B2M, and PD-L1 transgenes or THBD, TFPI, CD39, CD46, CD55, CD59, CD46, HO-1, A20, B2M, HLA-E SCT, and CD47 transgenes.

62. The vector of claim 61, comprising the vector in one of FIG. 17-20, 31, or 47-49.

63. The vector any one of claims 46-62, wherein the at least six transgenes are expressed from a single locus.

64. A method of generating the isolated cell, tissue, or animal of any one of claims 1 to 29.

65. The method of claim 64, comprising single copy polycistronic transgene integration through transposition, mono/bi-allelic site-specific integration through recombinase-mediated cassette exchange (RMCE), genomic replacement, endogenous gene humanization, or any combination thereof.

66. A transgenic pig liver having reduced liver damage and/or stable coagulation when exposed to non-pig blood,
wherein the reduced liver damage is assessed by determining the levels of one or more of bile production, one or more metabolic enzymes, and one or more serum electrolytes, and
wherein the stable coagulation is assessed by determining the levels of one or more of Prothrombin Time (PT) and International Normalized Ratio (PT-NIR), fibrinogen levels (FIB), and lower activated partial thromboplastin time (APTT).

67. The transgenic pig liver of claim 66, wherein the metabolic enzymes are selected from the group consisting of alanine aminotransferase (ALT), aspartate aminotransferase (AST), and albumin (ALB).

68. The transgenic pig liver of claim 66 or 67, wherein the serum electrolytes are potassium (K) and/or sodium (Na).

69. An isolated porcine cell, tissue, organ, or animal which:
(a) comprises a plurality of transgenes of at least two types selected from the group consisting of inflammatory response transgenes, immune response transgenes, immunomodulator transgenes, and any combination thereof, and
(b) is substantially free of production of xenotropic porcine endogenous retrovirus (PERV) virions.

70. An isolated porcine cell, tissue, organ, or animal which:
(a) comprises a plurality of transgenes, wherein the plurality of transgenes comprises at least one inflammatory response transgene, at least one immune response transgene, and at least one immunomodulator transgene, and
(b) is substantially free of production of xenotropic porcine endogenous retrovirus (PERV) virions.

71. The porcine isolated cell, tissue, organ, or animal of claim 69 or 70, wherein the porcine isolated cell, tissue, organ, or animal is substantially free of enzymatic activity of PERV polymerase (pol).

72. The porcine isolated cell, tissue, organ, or animal of claim 69 or 70, wherein the porcine isolated cell, tissue, organ, or animal is substantially free of expression of functional full-length PERV pol protein.

73. The porcine isolated cell, tissue, organ, or animal of claim 69 or 70, wherein coding sequences of at least about 97% of genomic PERV pol copies are disrupted.

74. The porcine isolated cell, tissue, organ, or animal of claim 69 or 70, wherein coding sequences of substantially all of genomic PERV pol copies are disrupted.

75. The porcine isolated cell, tissue, organ, or animal of claim 69 or 70, wherein coding sequences of at least about 97% of PERV pol mRNAs transcribed from genomic PERV pol copies are disrupted.

76. The porcine isolated cell, tissue, organ, or animal of any one of claims 73-75, wherein disruption comprises at least one frameshift insertion/deletion (indel) at least one nucleotide position of the PERV pol coding sequence.

77. The porcine isolated cell, tissue, organ, or animal of any one of claims 69-76, wherein the porcine isolated cell, tissue, organ, or animal expresses functional PERV gag and/or env protein.

78. The porcine isolated cell, tissue, organ, or animal of any one of claims 69-77, wherein the porcine isolated cell, tissue, organ, or animal comprise intact coding sequences of substantially all genomic copies of PERV gag and/or env genes

79. The porcine isolated cell, tissue, organ, or animal of any one of claims 69-78, wherein the porcine isolated cell, tissue, organ, or animal exhibits reduced PERV infectivity to a human cell.

80. The porcine isolated cell, tissue, organ, or animal of claim 79, wherein the porcine isolated cell, tissue, organ, or animal exhibits at least 200-fold less PERV infectivity to a human cell as compared to a wild-type porcine cell.

81. The porcine isolated cell, tissue, organ, or animal of claim 79 or 80, wherein the porcine isolated cell, tissue, organ, or animal exhibits reduced PERV infectivity to a human cell as compared to a porcine isolated porcine cell, tissue, organ, or animal lacking genomic modification targeting PERV pol genes or mRNA.

82. The porcine isolated cell, tissue, organ, or animal of any one of claims 79-81, wherein PERV infectivity is ascertained by co-culturing the porcine isolated cell, tissue, organ, or animal, or surgical explants thereof with a human cell.

83. The porcine isolated cell, tissue, organ, or animal of any one of claims 79-81 that is a porcine animal, wherein PERV infectivity is ascertained by co-culturing extracellular fluids derived from the porcine animal with a human cell.

84. The porcine isolated cell, tissue, organ, or animal of claim 82 or 83, wherein PERV infectivity is ascertained at least in part by analyzing the human cell by sequencing, PCR, or an immunoassay for presence of PERV genomic sequences or antigens following the co-culturing.

85. The porcine isolated cell, tissue, organ, or animal of any one of claims 69-84, wherein the PERV is PERV-A, PERV-B, PERV-A/C, or a recombinant variant thereof.

86. The porcine isolated cell, tissue, organ, or animal of any one of claims 69-85, wherein the inflammatory response transgene is selected from the group consisting of TNF α-induced protein 3 (A20), heme oxygenase (H0-1), Cluster of Differentiation 47 (CD47), and any combination thereof.

87. The porcine isolated cell, tissue, organ, or animal of any one of claims 69-86, wherein the immune response transgene is selected from the group consisting of human leukocyte antigen-E (HLA-E), beta-2 microglobulin (B2M), and any combination thereof.

88. The porcine isolated cell, tissue, organ, or animal of any one of claims 69-87, wherein the immunomodulator transgene is selected from the group consisting of programmed death-ligand 1 (PD-L1), Fas ligand (FasL), and any combination thereof.

89. The porcine isolated cell, tissue, organ, or animal of any one of claims 69-88, wherein the plurality of transgenes further comprises at least one coagulation response transgene.

90. The porcine isolated cell, tissue, organ, or animal of claim 89, wherein the coagulation response transgene is selected from the group consisting of Cluster of Differentiation 39 (CD39), thrombomodulin (THBD), tissue factor pathway inhibitor (TFPI), and any combination thereof.

91. The porcine isolated cell, tissue, organ, or animal of any one of claims 69-90, wherein the plurality of transgenes further comprises at least one complement response transgene.

92. The porcine isolated cell, tissue, organ, or animal of claim 91, wherein the complement response transgene is selected from the group consisting of human membrane cofactor protein (hCD46), human complement decay accelerating factor (hCD55), human MAC-inhibitor factor (hCD59), and any combination thereof.

93. The isolated porcine cell, tissue, organ, or animal of any one of claims 69-92, wherein the isolated porcine cell, tissue, organ, or animal comprises genomic integrations of the transgenes.

94. The isolated porcine cell, tissue, organ, or animal of any one of claim 93, wherein the isolated porcine cell, tissue, organ, or animal comprises germline-transmissible genomic integrations of the transgenes.

95. The isolated porcine cell, tissue, organ, or animal of any one of claims 69-94, wherein said porcine cell, tissue, organ, or animal expresses detectable levels of mRNAs transcribed from the transgenes.

96. The isolated porcine cell, tissue, organ, or animal of any one of claims 69-95, wherein said porcine cell, tissue, organ, or animal expresses detectable levels of proteins translated from the transgenes.

97. The isolated porcine cell, tissue, organ, or animal of any one of claims 69-95, wherein said porcine cell, tissue, organ, or animal expresses therapeutically effective levels of proteins translated from mRNAs transcribed from the transgenes.

98. An isolated porcine cell, tissue, organ, or animal, which:
(a) comprises six or more transgenes, each independently selected from the group consisting of complement response transgenes, coagulation response transgenes, inflammatory response transgenes, immune response transgenes, and immunomodulator transgenes, and
(b) is substantially free of production of xenotropic porcine endogenous retrovirus (PERV) virions.

99. The isolated porcine cell, tissue, organ, or animal of claim 98, wherein the isolated porcine cell, tissue, organ, or animal comprises 9, 10, 11, or 12 of the transgenes.

100. The isolated porcine cell, tissue, organ, or animal of claim 98 or 99, wherein the complement response transgene is selected from the group consisting of human membrane cofactor protein (hCD46), human complement decay accelerating factor (hCD55), human MAC-inhibitor factor (hCD59), and combinations thereof.

101. The isolated porcine cell, tissue, organ, or animal of claim 100, wherein transcription of at least a portion of the complement response transgenes is under the transcriptional control of a ubiquitous promoter.

102. The isolated porcine cell, tissue, organ, or animal of any one of claims 98-101, wherein the coagulation response transgene is selected from the group consisting of Cluster of Differentiation 39 (CD39), thrombomodulin (THBD), tissue factor pathway inhibitor (TFPI), and combinations thereof.

103. The isolated porcine cell, tissue, organ, or animal of any one of claims 98-102, wherein transcription of at least a portion of the coagulation response transgenes is under the transcriptional control of a tissue-specific promoter.

104. The isolated porcine cell, tissue, organ, or animal of claim 103, wherein the tissue-specific promoter is an endothelial-specific promoter.

105. The isolated porcine cell, tissue, organ, or animal of claim 104, wherein the endothelial-specific promoter is a low expression endothelial-specific promoter.

106. The isolated porcine cell, tissue, organ, or animal of any one of claims 98-105, wherein the inflammatory response transgene is selected from the group consisting of TNF α-induced protein 3 (A20), heme oxygenase (HO-1), Cluster of Differentiation 47 (CD47), and combinations thereof.

107. The isolated porcine cell, tissue, organ, or animal of any one of claims 98-106, wherein transcription of at least a portion of the inflammatory response transgenes is driven by a tissue-specific promoter, a ubiquitous promoter, or any combination thereof.

108. The isolated porcine cell, tissue, organ, or animal of claim 107, wherein the tissue-specific promoter is an endothelial-specific promoter.

109. The isolated porcine cell, tissue, organ, or animal of any one of claims 98-108, wherein the immune response transgene is selected from the group consisting of human leukocyte antigen-E (HLA-E), beta-2 microglobulin (B2M), and combinations thereof.

110. The isolated porcine cell, tissue, organ, or animal of any one of claims 98-109, wherein expression of at least a portion of the immune response transgenes is driven by a ubiquitous promoter.

111. The isolated porcine cell, tissue, organ, or animal of any one of claims 98-110, wherein the immunomodulator transgene is selected from the group consisting of programmed death-ligand 1 (PD-L1), Fas ligand (FasL), and combinations thereof.

112. The isolated porcine cell, tissue, organ, or animal of any one of claims 98-111, wherein the six or more transgenes are selected from the group consisting of hCD46, hCD55, hCD59, HLA-E, B2M, CD47, CD39, THBD, TFPI, A20, PD-L1, and HO-1.

113. The isolated porcine cell, tissue, organ, or animal of any one of claims 98-112, wherein the cell, tissue, organ, or animal comprises hCD46, hCD55, hCD59, CD39, THBD, TFPI, A20, HO-1, CD47, HLA-E, B2M, and PD-L1 transgenes or THBD, TFPI, CD39, CD46, CD55, CD59, CD46, HO-1, A20, B2M, HLA-E SCT, and CD47 transgenes.

114. The isolated porcine cell, tissue, organ, or animal of any one of claims 98-113, wherein the transgenes are expressed from a single locus.

115. The isolated porcine cell, tissue, organ, or animal of any one of claims 98-114, wherein the transgenes are transcribed into no more than 3 cistrons.

116. The isolated porcine cell, tissue, organ, or animal of claim 115, wherein a cistron comprises coding sequences for at least 3 distinct transgenes, wherein the at least 3 distinct transgenes are separated by coding sequences for porcine teschovirus 2A (P2A) peptide.

117. The isolated cell, tissue, organ, or animal of any one of claims 69-116, further comprising a deletion, disruption, or inactivation of one or more xenogenic carbohydrate antigen-producing genes.

118. The isolated cell, tissue, organ, or animal of claim 117, wherein the one or more xenogenic carbohydrate antigen-producing genes are selected from the group consisting of glycoprotein α-galactosyltransferase 1 (GGTA), β1,4 N-acetylgalactosaminyltransferase 2 (B4GalNT2), cytidine monophosphate-N-acetylneuraminic acid hydroxylase (CMAH).

119. The isolated cell, tissue, organ, or animal of claim 118, comprising the deletion, disruption, or inactivation in 2 copies of GGTA, 4 copies of B4GALNT2, or 2 copies of CMAH, or any combination thereof.

120. An isolated porcine cell, tissue, organ, or animal, which:
(a) comprises six or more transgenes, each independently selected from the group consisting of complement response transgenes, coagulation response transgenes, inflammatory response transgenes, immune response transgenes, and immunomodulator transgenes,
(b) is substantially free of production of xenotropic porcine endogenous retrovirus (PERV) virions, and
(c) comprises a deletion, disruption, or inactivation in 2 copies of GGTA, 4 copies of B4GALNT2, or 2 copies of CMAH, or any combination thereof.

121. The isolated porcine cell, tissue, organ, or animal of any one of claims 69-120, wherein the cell, tissue, organ, or animal exhibits reduced binding to human antibodies when exposed to human blood or fractions thereof.

122. The isolated porcine cell, tissue, organ, or animal of claim 121, wherein the cell, tissue, organ, or animal exhibits at least about 5-fold reduced binding to human antibodies when exposed to human blood or fractions thereof.

123. The isolated porcine cell, tissue, organ, or animal of claim 121, wherein the cell, tissue, organ, or animal exhibits at least about 10-fold reduced binding to human antibodies when exposed to human blood or fractions thereof.

124. The isolated porcine cell, tissue, organ, or animal of any one of claims 121-123 wherein the antibodies are IgM antibodies.

125. The isolated porcine cell, tissue, organ, or animal of any one of claims 121-123, wherein the antibodies are IgG antibodies.

126. The isolated porcine cell, tissue, organ, or animal of any one of claims 69-125, wherein the cell, tissue, organ, or animal exhibits reduced Natural Killer (NK) cell toxicity when exposed to human blood.

127. The isolated porcine cell, tissue, organ, or animal of claim 126, wherein the cell, tissue, organ, or animal exhibits at least about 20% less Natural Killer (NK) cell toxicity when exposed to human blood.

128. The isolated porcine cell, tissue, organ, or animal of any one of claims 69-127, wherein the cell, tissue, organ, or animal exhibits reduced complement toxicity when exposed to complement from human blood.

129. The isolated porcine cell, tissue, organ, or animal of claim 128, wherein the cell, tissue, organ, or animal exhibits at least about 5-fold less complement toxicity when exposed to human complement from human blood.

130. The isolated porcine cell, tissue, organ, or animal of any one of claims 69-129, wherein the cell, tissue, organ, or animal exhibits reduced TAT complex formation when exposed to human blood.

131. The isolated porcine cell, tissue, organ, or animal of claim 130, wherein the cell, tissue, organ, or animal exhibits at least about 3-fold reduced TAT complex formation when exposed to human blood.

132. The isolated porcine cell, tissue, organ, or animal of claim 130, wherein the cell, tissue, organ, or animal exhibits at least about 10-fold reduced TAT complex formation when exposed to human blood.

133. The isolated porcine cell, tissue, organ, or animal of any one of claims 69-132, which is an animal, which exhibits normal blood counts of white blood cells, platelets, monocytes, neutrophils, eosinophils, or any combination thereof.

134. The isolated porcine cell, tissue, organ, or animal of any one of claims 69-133, which is an animal, which exhibits normal liver function as assessed by serum alkaline phosphatase levels, aspartame aminoacyltransferase levels, alanine aminotransferase levels, ALT/AST level, cholesterol, total bilirubin, triglyceride, or albumin/globulin levels, or any combination thereof.

135. The isolated porcine cell, tissue, organ, or animal of any one of claims 69-134, which is an animal, which exhibits normal heart function as assessed by serum creatine kinase levels, creatine kinase-MB levels, lactate dehydrogenase levels, or any combination thereof.

136. The isolated porcine cell, tissue, organ, or animal of any one of claims 69-135, which is an animal, which exhibits normal kidney function as assessed by serum creatinine levels, urea levels, or a combination thereof.

137. The isolated porcine cell, tissue, organ, or animal of any one of claims 69-136, which is an animal, which exhibits normal coagulation function as assessed by thrombin time, prothrombin levels, or a combination thereof.

138. The isolated porcine cell, tissue, organ, or animal of any one of claims 69-137, which is an animal, which is capable of transmitting:
(a) the deletion, disruption, or inactivation of one or more xenogenic carbohydrate antigen-producing genes including α-galactosyltransferase 1 (GGTA), δ1,4 N-acetylgalactosaminyltransferase 2 (B4GalNT2), or cytidine monophosphate-N-acetylneuraminic acid hydroxylase (CMAH), or a combination thereof;
(b) the transgenes;
(c) the absence of production of xenotropic porcine endogenous retrovirus (PERV) virions; or
(d) any combination thereof;
to a progeny animal, wherein (a)-(d) are transmitted by normal mendelian inheritance.


SEQUENCE LISTING (356 sequences)

SEQ ID NO: 1 - DNA, 20 bp, Artificial Sequence
  Synthetic
tcaacacaaacatatctttg

SEQ ID NO: 2 - DNA, 20 bp, Artificial Sequence
  Synthetic
tgtttgtgttgatacgtcag

SEQ ID NO: 3 - DNA, 20 bp, Artificial Sequence
  Synthetic
gaaactgactaggatccatg

SEQ ID NO: 4 - DNA, 20 bp, Artificial Sequence
  Synthetic
ctcagtgggttaactatccg

SEQ ID NO: 5 - DNA, 20 bp, Artificial Sequence
  Synthetic
tctcacctgtgaagcctgcg

SEQ ID NO: 6 - DNA, 20 bp, Artificial Sequence
  Synthetic
cacagtgacttgggccacta

SEQ ID NO: 7 - DNA, 23 bp, Artificial Sequence
  Synthetic
tctcacctgtgaagcctgcgcgg

SEQ ID NO: 8 - DNA, 23 bp, Artificial Sequence
  Synthetic
cacagtgacttgggccactaggg

SEQ ID NO: 9 - DNA, 23 bp, Artificial Sequence
  Synthetic
tcctcctgcagtcactgtgatgg

SEQ ID NO: 10 - DNA, 23 bp, Artificial Sequence
  Synthetic
ggtgccccccacagaaggcccgg

SEQ ID NO: 11 - DNA, 23 bp, Artificial Sequence
  Synthetic
cagccccaccacaccctacgagg

SEQ ID NO: 12 - DNA, 23 bp, Artificial Sequence
  Synthetic
cttcttctgcagcaaacttctgg

SEQ ID NO: 13 - DNA, 23 bp, Artificial Sequence
  Synthetic
cggctctgacaagctgtccgagg

SEQ ID NO: 14 - DNA, 23 bp, Artificial Sequence
  Synthetic
cgaggccctgaaggtcttcgtgg

SEQ ID NO: 15 - DNA, 23 bp, Artificial Sequence
  Synthetic
ctcccagaagcacatccgcgtgg

SEQ ID NO: 16 - DNA, 23 bp, Artificial Sequence
  Synthetic
ccacgcctacatctcgctccagg

SEQ ID NO: 17 - DNA, 23 bp, Artificial Sequence
  Synthetic
gaaagcggccctcggagctgcgg

SEQ ID NO: 18 - DNA, 23 bp, Artificial Sequence
  Synthetic
cgccagccaggtgaagtacgcgg

SEQ ID NO: 19 - DNA, 23 bp, Artificial Sequence
  Synthetic
cgaggtggcttccatcagcgagg

SEQ ID NO: 20 - DNA, 23 bp, Artificial Sequence
  Synthetic
tacacgctcttccaaatctttgg

SEQ ID NO: 21 - DNA, 23 bp, Artificial Sequence
  Synthetic
ccgtatagccctgctgctcatgg

SEQ ID NO: 22 - DNA, 23 bp, Artificial Sequence
  Synthetic
cagccaggagccacgccggctgg

SEQ ID NO: 23 - DNA, 23 bp, Artificial Sequence
  Synthetic
gaacttggcccgctacctccagg

SEQ ID NO: 24 - DNA, 23 bp, Artificial Sequence
  Synthetic
gaagaaggtcaccgtgattccgg

SEQ ID NO: 25 - DNA, 23 bp, Artificial Sequence
  Synthetic
gatccgcctcatcgagaagcagg

SEQ ID NO: 26 - DNA, 23 bp, Artificial Sequence
  Synthetic
cccggagaacaaagcctttgtgg

SEQ ID NO: 27 - DNA, 23 bp, Artificial Sequence
  Synthetic
ctacctctgcgacctcgccccgg

SEQ ID NO: 28 - DNA, 23 bp, Artificial Sequence
  Synthetic
ccctacgcggcgccccctagtgg

SEQ ID NO: 29 - DNA, 23 bp, Artificial Sequence
  Synthetic
actgtggcgcctgagctccccgg

SEQ ID NO: 30 - DNA, 23 bp, Artificial Sequence
  Synthetic
gctcgaacccaagaagagaatgg

SEQ ID NO: 31 - DNA, 23 bp, Artificial Sequence
  Synthetic
atcacagtgactgcaggaggagg

SEQ ID NO: 32 - DNA, 23 bp, Artificial Sequence
  Synthetic
cggttccgcgcaggcttcacagg

SEQ ID NO: 33 - DNA, 23 bp, Artificial Sequence
  Synthetic
ctgaccgggccttctgtgggggg

SEQ ID NO: 34 - DNA, 23 bp, Artificial Sequence
  Synthetic
cctcctcgtagggtgtggtgggg

SEQ ID NO: 35 - DNA, 23 bp, Artificial Sequence
  Synthetic
aagtcgtgcagcggcggctctgg

SEQ ID NO: 36 - DNA, 23 bp, Artificial Sequence
  Synthetic
agccgtccagcaggaagaccagg

SEQ ID NO: 37 - DNA, 23 bp, Artificial Sequence
  Synthetic
cagggcctcgaagtcggcctcgg

SEQ ID NO: 38 - DNA, 23 bp, Artificial Sequence
  Synthetic
tgtgcttctgggagatgtgcagg

SEQ ID NO: 39 - DNA, 23 bp, Artificial Sequence
  Synthetic
ggtactccaccaccgccacgcgg

SEQ ID NO: 40 - DNA, 23 bp, Artificial Sequence
  Synthetic
ctggagcgagatgtaggcgtggg

SEQ ID NO: 41 - DNA, 23 bp, Artificial Sequence
  Synthetic
gcagctccgagggccgctttcgg

SEQ ID NO: 42 - DNA, 23 bp, Artificial Sequence
  Synthetic
gcccgcgtacttcacctggctgg

SEQ ID NO: 43 - DNA, 23 bp, Artificial Sequence
  Synthetic
gtacttcaaaacctcgctgatgg

SEQ ID NO: 44 - DNA, 23 bp, Artificial Sequence
  Synthetic
ggtcgaccctgccaaagatttgg

SEQ ID NO: 45 - DNA, 23 bp, Artificial Sequence
  Synthetic
agggctatacgggaggcttcggg

SEQ ID NO: 46 - DNA, 23 bp, Artificial Sequence
  Synthetic
cagccggcgtggctcctggctgg

SEQ ID NO: 47 - DNA, 23 bp, Artificial Sequence
  Synthetic
gaggtagcgggccaagttctggg

SEQ ID NO: 48 - DNA, 23 bp, Artificial Sequence
  Synthetic
cggtgaccttcttcttcttcagg

SEQ ID NO: 49 - DNA, 23 bp, Artificial Sequence
  Synthetic
acgtggggtccgatgcccaccgg

SEQ ID NO: 50 - DNA, 23 bp, Artificial Sequence
  Synthetic
cgatgaggcggatctgcttgagg

SEQ ID NO: 51 - DNA, 23 bp, Artificial Sequence
  Synthetic
cacaaaggctttgttctccgggg

SEQ ID NO: 52 - DNA, 23 bp, Artificial Sequence
  Synthetic
cttccggggcgaggtcgcagagg

SEQ ID NO: 53 - DNA, 23 bp, Artificial Sequence
  Synthetic
agggggcgccgcgtaggggcggg

SEQ ID NO: 54 - DNA, 23 bp, Artificial Sequence
  Synthetic
ctcaggcgccacagtgacttggg

SEQ ID NO: 55 - DNA, 23 bp, Artificial Sequence
  Synthetic
gttcgagcgttgaaaccccgggg

SEQ ID NO: 56 - DNA, 23 bp, Artificial Sequence
  Synthetic
atccaagaccattctcttcttgg

SEQ ID NO: 57 - DNA, 23 bp, Artificial Sequence
  Synthetic
accatcacagtgactgcaggagg

SEQ ID NO: 58 - DNA, 23 bp, Artificial Sequence
  Synthetic
gacaccatcacagtgactgcagg

SEQ ID NO: 59 - DNA, 23 bp, Artificial Sequence
  Synthetic
ctgtgaagcctgcgcggaaccgg

SEQ ID NO: 60 - DNA, 23 bp, Artificial Sequence
  Synthetic
ggggggcaccggttccgcgcagg

SEQ ID NO: 61 - DNA, 23 bp, Artificial Sequence
  Synthetic
agggtgtggtggggctgaccggg

SEQ ID NO: 62 - DNA, 23 bp, Artificial Sequence
  Synthetic
gaaccggtgccccccacagaagg

SEQ ID NO: 63 - DNA, 23 bp, Artificial Sequence
  Synthetic
gggccttctgtggggggcaccgg

SEQ ID NO: 64 - DNA, 23 bp, Artificial Sequence
  Synthetic
gctgaccgggccttctgtggggg

SEQ ID NO: 65 - DNA, 23 bp, Artificial Sequence
  Synthetic
tcctcctcgtagggtgtggtggg

SEQ ID NO: 66 - DNA, 23 bp, Artificial Sequence
  Synthetic
ggctgaccgggccttctgtgggg

SEQ ID NO: 67 - DNA, 23 bp, Artificial Sequence
  Synthetic
gtcctcctcgtagggtgtggtgg

SEQ ID NO: 68 - DNA, 23 bp, Artificial Sequence
  Synthetic
gggctgaccgggccttctgtggg

SEQ ID NO: 69 - DNA, 23 bp, Artificial Sequence
  Synthetic
cgtgtcctcctcgtagggtgtgg

SEQ ID NO: 70 - DNA, 23 bp, Artificial Sequence
  Synthetic
ggggctgaccgggccttctgtgg

SEQ ID NO: 71 - DNA, 23 bp, Artificial Sequence
  Synthetic
tagggtgtggtggggctgaccgg

SEQ ID NO: 72 - DNA, 23 bp, Artificial Sequence
  Synthetic
tctggcgtgtcctcctcgtaggg

SEQ ID NO: 73 - DNA, 23 bp, Artificial Sequence
  Synthetic
cagaagaagtcgtgcagcggcgg

SEQ ID NO: 74 - DNA, 23 bp, Artificial Sequence
  Synthetic
ctctggcgtgtcctcctcgtagg

SEQ ID NO: 75 - DNA, 23 bp, Artificial Sequence
  Synthetic
ctgcagaagaagtcgtgcagcgg

SEQ ID NO: 76 - DNA, 23 bp, Artificial Sequence
  Synthetic
ctgcagcaaacttctggacctgg

SEQ ID NO: 77 - DNA, 23 bp, Artificial Sequence
  Synthetic
tctggacctggtcttcctgctgg

SEQ ID NO: 78 - DNA, 23 bp, Artificial Sequence
  Synthetic
gacctggtcttcctgctggacgg

SEQ ID NO: 79 - DNA, 23 bp, Artificial Sequence
  Synthetic
gctgtccgaggccgacttcgagg

SEQ ID NO: 80 - DNA, 23 bp, Artificial Sequence
  Synthetic
ggtcttcgtggtgggcatgatgg

SEQ ID NO: 81 - DNA, 23 bp, Artificial Sequence
  Synthetic
gcttgtcagagccgtccagcagg

SEQ ID NO: 82 - DNA, 23 bp, Artificial Sequence
  Synthetic
ggccgacttcgaggccctgaagg

SEQ ID NO: 83 - DNA, 23 bp, Artificial Sequence
  Synthetic
gaccttcagggcctcgaagtcgg

SEQ ID NO: 84 - DNA, 23 bp, Artificial Sequence
  Synthetic
gcccaccacgaagaccttcaggg

SEQ ID NO: 85 - DNA, 23 bp, Artificial Sequence
  Synthetic
ggccctgaaggtcttcgtggtgg

SEQ ID NO: 86 - DNA, 23 bp, Artificial Sequence
  Synthetic
gccctgaaggtcttcgtggtggg

SEQ ID NO: 87 - DNA, 23 bp, Artificial Sequence
  Synthetic
tgcccaccacgaagaccttcagg

SEQ ID NO: 88 - DNA, 23 bp, Artificial Sequence
  Synthetic
cgccacgcggatgtgcttctggg

SEQ ID NO: 89 - DNA, 23 bp, Artificial Sequence
  Synthetic
tgtaggcgtgggagccgtcgtgg

SEQ ID NO: 90 - DNA, 23 bp, Artificial Sequence
  Synthetic
ccgagggccgctttcggtcctgg

SEQ ID NO: 91 - DNA, 23 bp, Artificial Sequence
  Synthetic
ccagaagcacatccgcgtggcgg

SEQ ID NO: 92 - DNA, 23 bp, Artificial Sequence
  Synthetic
gaagcacatccgcgtggcggtgg

SEQ ID NO: 93 - DNA, 23 bp, Artificial Sequence
  Synthetic
gcacatccgcgtggcggtggtgg

SEQ ID NO: 94 - DNA, 23 bp, Artificial Sequence
  Synthetic
gcggtggtggagtaccacgacgg

SEQ ID NO: 95 - DNA, 23 bp, Artificial Sequence
  Synthetic
tctcgctccaggaccgaaagcgg

SEQ ID NO: 96 - DNA, 23 bp, Artificial Sequence
  Synthetic
gctgcggcgcatcgccagccagg

SEQ ID NO: 97 - DNA, 23 bp, Artificial Sequence
  Synthetic
gaagtacgcgggcagcgaggtgg

SEQ ID NO: 98 - DNA, 23 bp, Artificial Sequence
  Synthetic
tcggtcctggagcgagatgtagg

SEQ ID NO: 99 - DNA, 23 bp, Artificial Sequence
  Synthetic
gcgatgcgccgcagctccgaggg

SEQ ID NO: 100 - DNA, 23 bp, Artificial Sequence
  Synthetic
cgctgcccgcgtacttcacctgg

SEQ ID NO: 101 - DNA, 23 bp, Artificial Sequence
  Synthetic
ggcgatgcgccgcagctccgagg

SEQ ID NO: 102 - DNA, 23 bp, Artificial Sequence
  Synthetic
gccagccaggtgaagtacgcggg

SEQ ID NO: 103 - DNA, 23 bp, Artificial Sequence
  Synthetic
ggtgaagtacgcgggcagcgagg

SEQ ID NO: 104 - DNA, 23 bp, Artificial Sequence
  Synthetic
cgctcttccaaatctttggcagg

SEQ ID NO: 105 - DNA, 23 bp, Artificial Sequence
  Synthetic
gctcttccaaatctttggcaggg

SEQ ID NO: 106 - DNA, 23 bp, Artificial Sequence
  Synthetic
aaatctttggcagggtcgaccgg

SEQ ID NO: 107 - DNA, 23 bp, Artificial Sequence
  Synthetic
ctatacgggaggcttcgggccgg

SEQ ID NO: 108 - DNA, 23 bp, Artificial Sequence
  Synthetic
ctggctggccatgagcagcaggg

SEQ ID NO: 109 - DNA, 23 bp, Artificial Sequence
  Synthetic
aagttctgggccagccggcgtgg

SEQ ID NO: 110 - DNA, 23 bp, Artificial Sequence
  Synthetic
cttcaggccctggaggtagcggg

SEQ ID NO: 111 - DNA, 23 bp, Artificial Sequence
  Synthetic
tccgatgcccaccggaatcacgg

SEQ ID NO: 112 - DNA, 23 bp, Artificial Sequence
  Synthetic
atctgcttgaggctgacgtgggg

SEQ ID NO: 113 - DNA, 23 bp, Artificial Sequence
  Synthetic
gggcctgcttctcgatgaggcgg

SEQ ID NO: 114 - DNA, 23 bp, Artificial Sequence
  Synthetic
gtccacaccgctgaccacaaagg

SEQ ID NO: 115 - DNA, 23 bp, Artificial Sequence
  Synthetic
cagggctatacgggaggcttcgg

SEQ ID NO: 116 - DNA, 23 bp, Artificial Sequence
  Synthetic
gagcagcagggctatacgggagg

SEQ ID NO: 117 - DNA, 23 bp, Artificial Sequence
  Synthetic
catgagcagcagggctatacggg

SEQ ID NO: 118 - DNA, 23 bp, Artificial Sequence
  Synthetic
cctgctgctcatggccagccagg

SEQ ID NO: 119 - DNA, 23 bp, Artificial Sequence
  Synthetic
acgccggctggcccagaacttgg

SEQ ID NO: 120 - DNA, 23 bp, Artificial Sequence
  Synthetic
ccagggcctgaagaagaagaagg

SEQ ID NO: 121 - DNA, 23 bp, Artificial Sequence
  Synthetic
accgtgattccggtgggcatcgg

SEQ ID NO: 122 - DNA, 23 bp, Artificial Sequence
  Synthetic
tggccagccaggagccacgccgg

SEQ ID NO: 123 - DNA, 23 bp, Artificial Sequence
  Synthetic
gggccagccggcgtggctcctgg

SEQ ID NO: 124 - DNA, 23 bp, Artificial Sequence
  Synthetic
gggccaagttctgggccagccgg

SEQ ID NO: 125 - DNA, 23 bp, Artificial Sequence
  Synthetic
tcttcttcttcaggccctggagg

SEQ ID NO: 126 - DNA, 23 bp, Artificial Sequence
  Synthetic
ggaggtagcgggccaagttctgg

SEQ ID NO: 127 - DNA, 23 bp, Artificial Sequence
  Synthetic
aacttggcccgctacctccaggg

SEQ ID NO: 128 - DNA, 23 bp, Artificial Sequence
  Synthetic
tcttcaggccctggaggtagcgg

SEQ ID NO: 129 - DNA, 23 bp, Artificial Sequence
  Synthetic
gaaggtcaccgtgattccggtgg

SEQ ID NO: 130 - DNA, 23 bp, Artificial Sequence
  Synthetic
aaggtcaccgtgattccggtggg

SEQ ID NO: 131 - DNA, 23 bp, Artificial Sequence
  Synthetic
gatctgcttgaggctgacgtggg

SEQ ID NO: 132 - DNA, 23 bp, Artificial Sequence
  Synthetic
ccggggcctgcttctcgatgagg

SEQ ID NO: 133 - DNA, 23 bp, Artificial Sequence
  Synthetic
ggatctgcttgaggctgacgtgg

SEQ ID NO: 134 - DNA, 23 bp, Artificial Sequence
  Synthetic
aacaaagcctttgtggtcagcgg

SEQ ID NO: 135 - DNA, 23 bp, Artificial Sequence
  Synthetic
accacaaaggctttgttctccgg

SEQ ID NO: 136 - DNA, 23 bp, Artificial Sequence
  Synthetic
agcctttgtggtcagcggtgtgg

SEQ ID NO: 137 - DNA, 23 bp, Artificial Sequence
  Synthetic
ggtcagcggtgtggacgagctgg

SEQ ID NO: 138 - DNA, 23 bp, Artificial Sequence
  Synthetic
cggaagtgcccgcccctacgcgg

SEQ ID NO: 139 - DNA, 23 bp, Artificial Sequence
  Synthetic
cctagtggcccaagtcactgtgg

SEQ ID NO: 140 - DNA, 23 bp, Artificial Sequence
  Synthetic
gggcgggcacttccggggcgagg

SEQ ID NO: 141 - DNA, 23 bp, Artificial Sequence
  Synthetic
cagtgacttgggccactaggggg

SEQ ID NO: 142 - DNA, 23 bp, Artificial Sequence
  Synthetic
gttgaaaccccggggagctcagg

SEQ ID NO: 143 - DNA, 23 bp, Artificial Sequence
  Synthetic
tccaagaccattctcttcttggg

SEQ ID NO: 144 - DNA, 23 bp, Artificial Sequence
  Synthetic
cgtaggggcgggcacttccgggg

SEQ ID NO: 145 - DNA, 23 bp, Artificial Sequence
  Synthetic
gcgtaggggcgggcacttccggg

SEQ ID NO: 146 - DNA, 23 bp, Artificial Sequence
  Synthetic
cgcgtaggggcgggcacttccgg

SEQ ID NO: 147 - DNA, 23 bp, Artificial Sequence
  Synthetic
tagggggcgccgcgtaggggcgg

SEQ ID NO: 148 - DNA, 23 bp, Artificial Sequence
  Synthetic
cactagggggcgccgcgtagggg

SEQ ID NO: 149 - DNA, 23 bp, Artificial Sequence
  Synthetic
gccactagggggcgccgcgtagg

SEQ ID NO: 150 - DNA, 23 bp, Artificial Sequence
  Synthetic
gctcaggcgccacagtgacttgg

SEQ ID NO: 151 - DNA, 23 bp, Artificial Sequence
  Synthetic
acagtgacttgggccactagggg

SEQ ID NO: 152 - DNA, 23 bp, Artificial Sequence
  Synthetic
ctgtggcgcctgagctccccggg

SEQ ID NO: 153 - DNA, 23 bp, Artificial Sequence
  Synthetic
tgtggcgcctgagctccccgggg

SEQ ID NO: 154 - DNA, 23 bp, Artificial Sequence
  Synthetic
ggttcgagcgttgaaaccccggg

SEQ ID NO: 155 - DNA, 23 bp, Artificial Sequence
  Synthetic
gggttcgagcgttgaaaccccgg

SEQ ID NO: 156 - DNA, 23 bp, Artificial Sequence
  Synthetic
acccaagaagagaatggtcttgg

SEQ ID NO: 157 - DNA, 23 bp, Artificial Sequence
  Synthetic
gaagagaatggtcttggatgtgg

SEQ ID NO: 158 - DNA, 23 bp, Artificial Sequence
  Synthetic
tctccagacgcaggacgttgggg

SEQ ID NO: 159 - DNA, 23 bp, Artificial Sequence
  Synthetic
ggaggcccacgaagggcaagggg

SEQ ID NO: 160 - DNA, 23 bp, Artificial Sequence
  Synthetic
tgcctagcatctcgtgcccctgg

SEQ ID NO: 161 - DNA, 23 bp, Artificial Sequence
  Synthetic
cacccccaacgtcctgcgtctgg

SEQ ID NO: 162 - DNA, 23 bp, Artificial Sequence
  Synthetic
gagtgaggagatggtggtgttgg

SEQ ID NO: 163 - DNA, 23 bp, Artificial Sequence
  Synthetic
acgaagggcaaggggatattcgg

SEQ ID NO: 164 - DNA, 23 bp, Artificial Sequence
  Synthetic
ggtcaccgtccatgacttcccgg

SEQ ID NO: 165 - DNA, 23 bp, Artificial Sequence
  Synthetic
cctgagcaccgtcaacatcaagg

SEQ ID NO: 166 - DNA, 23 bp, Artificial Sequence
  Synthetic
tcccaggggcacgagatgctagg

SEQ ID NO: 167 - DNA, 23 bp, Artificial Sequence
  Synthetic
aactgcgggaggggaggacgagg

SEQ ID NO: 168 - DNA, 23 bp, Artificial Sequence
  Synthetic
caggacgttgggggtgattatgg

SEQ ID NO: 169 - DNA, 23 bp, Artificial Sequence
  Synthetic
aatatccccttgcccttcgtggg

SEQ ID NO: 170 - DNA, 23 bp, Artificial Sequence
  Synthetic
cttggccgggaagtcatggacgg

SEQ ID NO: 171 - DNA, 23 bp, Artificial Sequence
  Synthetic
gttcagcgtcgtggtctcgctgg

SEQ ID NO: 172 - DNA, 23 bp, Artificial Sequence
  Synthetic
gacggtgctcaggtagttgttgg

SEQ ID NO: 173 - DNA, 23 bp, Artificial Sequence
  Synthetic
ggggtggggcttcagcggtccgg

SEQ ID NO: 174 - DNA, 23 bp, Artificial Sequence
  Synthetic
gcctagcatctcgtgcccctggg

SEQ ID NO: 175 - DNA, 23 bp, Artificial Sequence
  Synthetic
gcgtagagctgtcatcccagggg

SEQ ID NO: 176 - DNA, 23 bp, Artificial Sequence
  Synthetic
tggtgtaactgcgggaggggagg

SEQ ID NO: 177 - DNA, 23 bp, Artificial Sequence
  Synthetic
ctccagacgcaggacgttggggg

SEQ ID NO: 178 - DNA, 23 bp, Artificial Sequence
  Synthetic
ggcgtagagctgtcatcccaggg

SEQ ID NO: 179 - DNA, 23 bp, Artificial Sequence
  Synthetic
aggcgtagagctgtcatcccagg

SEQ ID NO: 180 - DNA, 23 bp, Artificial Sequence
  Synthetic
ttatggtgtaactgcgggagggg

SEQ ID NO: 181 - DNA, 23 bp, Artificial Sequence
  Synthetic
attatggtgtaactgcgggaggg

SEQ ID NO: 182 - DNA, 23 bp, Artificial Sequence
  Synthetic
gattatggtgtaactgcgggagg

SEQ ID NO: 183 - DNA, 23 bp, Artificial Sequence
  Synthetic
ggtgattatggtgtaactgcggg

SEQ ID NO: 184 - DNA, 23 bp, Artificial Sequence
  Synthetic
ctctccagacgcaggacgttggg

SEQ ID NO: 185 - DNA, 23 bp, Artificial Sequence
  Synthetic
gggtgattatggtgtaactgcgg

SEQ ID NO: 186 - DNA, 23 bp, Artificial Sequence
  Synthetic
actctccagacgcaggacgttgg

SEQ ID NO: 187 - DNA, 23 bp, Artificial Sequence
  Synthetic
cgtcctgcgtctggagagtgagg

SEQ ID NO: 188 - DNA, 23 bp, Artificial Sequence
  Synthetic
gtggtgttggaggcccacgaagg

SEQ ID NO: 189 - DNA, 23 bp, Artificial Sequence
  Synthetic
gcaaggggatattcgggtttcgg

SEQ ID NO: 190 - DNA, 23 bp, Artificial Sequence
  Synthetic
tgacttcccggccaagagacagg

SEQ ID NO: 191 - DNA, 23 bp, Artificial Sequence
  Synthetic
tctcctcactctccagacgcagg

SEQ ID NO: 192 - DNA, 23 bp, Artificial Sequence
  Synthetic
gcgtctggagagtgaggagatgg

SEQ ID NO: 193 - DNA, 23 bp, Artificial Sequence
  Synthetic
tggtgttggaggcccacgaaggg

SEQ ID NO: 194 - DNA, 23 bp, Artificial Sequence
  Synthetic
tctggagagtgaggagatggtgg

SEQ ID NO: 195 - DNA, 23 bp, Artificial Sequence
  Synthetic
ttggaggcccacgaagggcaagg

SEQ ID NO: 196 - DNA, 23 bp, Artificial Sequence
  Synthetic
tgaggagatggtggtgttggagg

SEQ ID NO: 197 - DNA, 23 bp, Artificial Sequence
  Synthetic
tggaggcccacgaagggcaaggg

SEQ ID NO: 198 - DNA, 23 bp, Artificial Sequence
  Synthetic
gaatatccccttgcccttcgtgg

SEQ ID NO: 199 - DNA, 23 bp, Artificial Sequence
  Synthetic
cgaagggcaaggggatattcggg

SEQ ID NO: 200 - DNA, 23 bp, Artificial Sequence
  Synthetic
gtctcttggccgggaagtcatgg

SEQ ID NO: 201 - DNA, 23 bp, Artificial Sequence
  Synthetic
acagcacctgtctcttggccggg

SEQ ID NO: 202 - DNA, 23 bp, Artificial Sequence
  Synthetic
gacagcacctgtctcttggccgg

SEQ ID NO: 203 - DNA, 23 bp, Artificial Sequence
  Synthetic
gttggcgttgttcagcgtcgtgg

SEQ ID NO: 204 - DNA, 23 bp, Artificial Sequence
  Synthetic
gctggacagcacctgtctcttgg

SEQ ID NO: 205 - DNA, 23 bp, Artificial Sequence
  Synthetic
gagcaccgtcaacatcaaggtgg

SEQ ID NO: 206 - DNA, 23 bp, Artificial Sequence
  Synthetic
cgcgcccaccttgatgttgacgg

SEQ ID NO: 207 - DNA, 23 bp, Artificial Sequence
  Synthetic
agcaccgtcaacatcaaggtggg

SEQ ID NO: 208 - DNA, 23 bp, Artificial Sequence
  Synthetic
aggtgggcgcgctcaacagccgg

SEQ ID NO: 209 - DNA, 23 bp, Artificial Sequence
  Synthetic
aagaaggggtggggcttcagcgg

SEQ ID NO: 210 - DNA, 20 bp, Artificial Sequence
  Synthetic
gagaaaataatgaatgtcaa

SEQ ID NO: 211 - protein, 290 aa, Artificial Sequence
  Synthetic
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 212 - DNA, 31097 bp, Artificial Sequence
  Payload 12F sequence
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccataacttcgtatagcatacattatacgaagttatagaatggaaggttgtgttgaagcgcggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaatgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgagggcacaccttttgtttatggtgtaagacaagggtctgatttcatttcattttgagggtttcaccctctcatataataaaattcttctatatacccttctatcatcacttattactgattatatgccttgatttcagaatcttgccaactagattcagaatttgactgggagagaggacaaggaccacttgagactcatattttatttccagaatctagcatctacctacctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgacagggtaataagacagtagtgaatatcaagctacaaaaagccgcctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgacccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcatatgcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttatcacatagcatacagaccaacagcaacagtagcaaccaggaaagacagggtcagaacccatcttaataaaggagcctgagatctggtgttcagaggaggtttacctctaggggtttcaactggagcagagtcctgaactttgttagaagccctttgtcttaaaccgggagccctagatggagactggtctttggtgtcatgggtcagtagttcctgtaattcttcaaacagttggatgttcagtaggaaagctgttttagcttcttcaataactctttgcctaacagcgggggtcatttccagagagttcatcctagacctgtacagttgtttgaatttggtagcagaagctatgtttgggaaggtgaagaaagccagaccttcaccagaagaaggtaggtccagagctttctgagctatttttttcagaacctgaccaccagacaggtcacccaggtatctggtgtaagcatgagcaaccagtaattctggttctgtcctaccaacttcatgaagtcttttaacatacctttgcatagcgggggtgtatgggataacttcctgccatctaggaccataccagaaagccaggtcctgttccagagcagctttcctgtgtagttcttctgggaagtaaactggagcaaaaacaggagattctttgtttctttctatttcttcttccagagcaacatagatgtggtacagagaagccataaccagtttgaaaccatctctggtaacctgacctttctggaagtttctcatgaattcagcattttcagcctgggtgtgaacttctttggtagcttctttcagtgcttcagacaggtcttgtggcatagaatctggttgtggcctttcaggcccggggttctcctcgacgtcaccggcctgtttcagtagtgagaaattggtggcgccgctgccaaccatgtctttccagaagtaagatggtttgtggaagatcagtagacctatgatagcaacggtgaacagaaccagagagaacagaaccatcaggaaaacataggtagagtgagacagtggggtagacagaggttgttcagcggggatcatgttggtcaggttcagcatgtaacccagggtccaaccagcatcagaaccctggattttaccgatgaagtggatgtgttcccaagagtcagcagtgaagtggtaaccttgcagtaataaagacaggatgtaggtaccagagaaacagtattcagacaggtatttttctttaacaccagcataagaggttttgatttcttcccagggttgagcacagaattttttcatcatttcagtaactttttcctgagaaactttttcagaggtcaggttcaggaatttcataacaaagtagaaagcagagaaagcaccaaagtcaccctgtagaggaggtaagaagataccattgaaagcacactgagagtagggacagtaagaggtgttgaacagttccaggatagactggtgacactgttggtagttaccaataccctggatttcaaactgttggaagggtaaggtcatttcaaatcttttggtacaaggggttttgtacaggtcagaaacattaacaacttttttgtaaccagggtggaaacaagggtccctaaggatttcattagaagcaacctggatgtctttagccagtttctgccacagagcctggtctttaccataacacaggaaagagtgggtgtaaacattgtagtctttaccatacagtctgaactgtagagcattgtcaggagattcaatggtctggttctgaggaacaaaggtaacctgggtagaagcaccacccaggtccagagcaccaaaggtttcctggttgttggtttcatatggaactatagagaaccacctggttttctgagagaatttacccagaaggtagttgatggtgatccaaccataagcaccttcttcctgacctgtgatgatcctagcaccctggaagtcaaaggggtagttagacagagatctttcaacaacatccagaactctgtcagccagttcttcagattccattcttagtaatctcataccagcagtagcacccaggtaaacgggggtttcctggtgttgcgaacgagggataacttccctagctctttccatacagtcagtcaggtagataccaatttcattaactttctgaacaaatttagagataccaggacctttaactctacattcttcaacctggtgaacaacacctgtgtcattttctttttcagcgggccatttgtagatgtacagagaggtgtgagaagaaccagcatccagaacaataccatatttaacattttcaggtaaagctttgttctgggtcagaccaacagccagtaaagcaataacagcaatgatagaagagaaacccaggatagccaggatgtttttagaacagaaagttttaacattagattctttggtatcctccataggcccaggattctcctccacgtccccagcctgtttcagaaggctaaagttggtcgctccgctgccaggagaggggtcaaaagcagcattggtgaaaacaccataggtacccaggtgagagtgtgggttcagtggacatttaccatcatctttctgacggaaccttttgatctgagacattctttcagcctgtctccttctgtgtcgacacctaacacagaagaagatacccagacctatgaacagaagaagcccagcaacaccacccagaacaatcagagccattggttgaacaggggtagaccaggtaggaagaactttgatgttagattccagtaaaacctgaccagagtcagacagcaggcactgccacataccagcttctgggttcagaacccaaacagctttttctcttttagaaactttagcttctttgttttccagtttcagagacagcatcagttttggagaggttggaccccaaacttcacaggtcaggtttttctgaagttgggtagctctcataacaaccaggttaacttcctggtgtagtttaccagttttagcttccagagccagggtcaggttaccagaaccagcatactgtggaagagcctgtggtagggtcaggtgaagaggtaattttttacccatctgtaattttgggtcctgggtaacccttttaacagaaacttctttgtttttcaggtcaaaggtgatccaagatttagaagaagaagccctttcagcctgccaccacagttcaccagaaccagtcagtttttcaacagtgaaagccagtgggaaagagaattcaacctgttcaccttcttttttgtaaactatagaagaagctttctggaaagccagaacaactatgtctattttgaattcaacttttttctggttctgtagaactgtacaggtccaggtaccagagtcctgaagttccagttgagaaacagacagggttttaccatttctacaaccaccagaaccacccccaccggaccctcctccccctgaacctcccagttcctgtaagtacatgtttttaacaaagatttcttcataagctattttaactctttgtttttttctttttcttttggttttgatcagaccacctttagagattctttggatgaaaccttttttacaagcccttaaacattcctgtttagaggtgaagttgttttcattaccaccacaaccagagtatttgaagggtctacatttacctataacagagttgtagtagaacctgttttcattagctctacacagacctctgtcagcaggggtcagacaccaagaaggaccatggaattcaaacagagagggaactttggtagactgaggggtcagagagttgttaacagcattcagttgggtaccatagttgtcaacctggaaaccattgggaccatcttcacagatgtttttacattcttccagggtttcaaagttgttcatgttacccagacaaccaccatatttgaatctttcacactgtttggtctggttgttgtagaagtacctggtgatgtaacctctacagataccaggatcctcttccaggaaacagaagtctggtttttcctgttgtagggtggttttgatgatcctgttagcattgtccctggtacacattttcttacattcttccagagattcaaatctgttctggttaccttcacaaccaccatagatgaattcttcacactgtctggtgaagatgttgaagaagaaccttttcatgatagctttacaaggaccatcatcagctttgaaagcacagaaagagtgcatcagtttcagtggaggaagttcagtgtctgtgatgatggtgtgttcttcatcttcttcagagtcagcattcagaggagcaggagccaggttcagaagtaaacaaacagaggcccataaagcatgtacttttttcatagtgtagataggccctggattctcctccacgtccccggcctgcttaaggagcgaaaagttggtcgcgccgctgcccagcctttgaggggtcctttcagtcctaacatgttgtaaaacaacttctttagatggagcagcacatttgtattccattttagccctagcagcaccctgttttttcctaaggtgacacagtagagccagtagagcaacaaccagacacagagaagctatagagatacctatcagtagaccagagtgaaccagaccaacagctggtggggtcagggtagaaccgggggttggagagggaggaggttcaccagaaccagagtcaccaccatcaactttaccagagtcacagtctgtaccaatgtgtctagccagagcagagtcgggaccacagatacattcaaaggtaccgggaaggttgtgacaaacaccagaacagaaaccaccattttcacattcatcaatgtctgtacagatgaaaccatcatccaggatgtaaccttctggacattcacaagaagcctgggtgttagggtcacagtcagcgggacaagctgtctggttacagaacatctgacacctgtgaggttcatgagggattggagcaaaaccttcagcacaaacacacaggtaagaggtctggttcagaggttgacactggtattcacagttagccctgaaacaggggtcaacgggttcaacacattcaccatcaaccaggtcatagttagggtaacagtgacattcaaaaccaccctgggtgttaacacagcgttgtggacatggagagggttccaggatacagtcatcaacatcttcacatctgtgttggtcagcggccagtctgtaacctgtttcacacatacaagagtaagaaccgggttggtcggggttgggaacacagaagtgttcacacaggtcattacaagactgggtagcagaagcagtacaagatctaccatcagcctgaagagcagcaccagcaggacactgacacctaggagcaccagggatagcattacaagcatgttcacaaccaccattttcaacagaacagtcccaagcaccgggagcttctctagcccagtgaccctgaacagcaccgggtggagctgtacacatcagttgtaagcccagaggagcaacagcagcagaagaaccaactggtagagcctggaagtcagcacctctagcagcaaaaggggtaccataggtgatagaaacagcagcagcagcagcaccgggttcaacagccagtggtctacaggtagcggggaagtggaattcacacaggaaaccatcagctttaacttcacactgttgttcttcccagataggttcagaaggaacagtagcttcagcagcagaaacagcaacacacagaggaccacacagaggagcaccattcaggtccagtctagcccatctagagtaagaggtgttgttgtcacctgtaacccactggaaacctctaagaggacccagtcttttagggtcaccacaacctggtggtagttgcagtccaatccacagtctcctcctaccaacaccaccatcaccattcagtaataaagagataacatcagcagcaacagagctgcgaactgtcatcaggtgacccctaagaccatcacagatctgagaagcattcaggaaggtagcgggaccggggtacagagcaaaacagtcatgttcaacacactgagaaccacctggttgtggttcagcaggagcggggaaacctaaacctgccagagccagtgcaccaagaaccagaacacctaacatggtggcattgctttgaattagcggtggttttcacaacacctaaaaaagggtttaaaagatacctttgaaccgctaagaagcccgagaattagctccgctcaaaactcaaggggacaaattccaaaaatgacttccagcgccaggctggcctgactagtctccacccaccaaatgtgaacaaactccaacgccattacatcccctccccccgccgcgactagccgtgctcaaaagcccgaggtgactattgcggccgataggaccacggggtcacaggaagcagcagccggtgagggaccaggccctcttcctttgtgtgggtgactcacccgcccgctccaccgggctgccgcgtcctccattttgagctccttgcaacagggcccgggagcggccatctttccacgcacgcaactggtgccggacgggatggcctcaccctagttagggaggcagggcaacgcggcgccgccaagccagatcgtgccgggtgctggggccacatggcctcggcacgctaaccccagcctggttgcttcgggaaaaaccccaggcctcgccccatccaggtggcgtcggacatgtgctccgaaggcgggcgggccccagccgccactcctgtccctccattcctccccaaccatgacctctccgggctccgggcgagcaagccccgcaccctccctttgttagcccctattgctgaacggcaatcgaaggcagcagggcaacaacaacaaaaaaaaaaaagaccagagtgcggccggagtagcacgcggcggcggcgcggacaccacgctaggcctcaagccggacacgaggcgaggctacggggttgccgctaggcctcgcactctgcctcccgcgccgcccgcaactcgaagcgggaatgctcgcagctaatccccgccgacgacagcggggcccggcccgctcggagcaggacctccagctcggcggcccgcggaagccacacccgcccctcacctgcgttctgacggcaaacccgttgcgaaaaagaacgtccaaggcgactgccgcacttatataccgttctcccccaccctcgggaagagggcggagccagcacacgacaccgctttcccagtttgccccgcgccagctccccggtcctcccccccacgaccagggtctatgagcttgtgtgctctgctctccccgcgctcagccctgagcgcatgctcctcgcgctgtcgtggggcggctacagggatttgggtctgtgagaatttagggacggtccctgggctctccccttgcgaaggcgatccctccttttgtatgaattactctcggctccggcgcgccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcctgggcctagggctgtgccagctgcctcgtcccgtcaccttctggcttcttctctccctccatatcttagctgttttcctcatgagaatgttccaaattcgaaatttctatttaaccattatatatttacttgtttgctattatctctgcccccagtagattgttagctccagaagagaaaggatcatgtcttttgcttatctagatatgcccatctgcctggtacaatctctggcacatgttacaggcaacaactacttgtggaattggtgaatgcatgaatagaagaatgagtgaatgaatgaatagacaaaaggcagaaatccagcctcaaagagcttacagtctggtaagaggaataaaatgtctgcaaatagccacaggacaggtcaaaggaaggaggggctatttccagctgagggcaccccatcaggaaagcaccccagacttcctacaactactagacacatctcgatgcttttcacttctctatcaatggatcgtctccctggagaataatccccaaagtgaaattacttagcacgtccagttaggtagatccttgtgtacttcttggttgttcagagatcatcaaccagtgcaaacaatccccccatcaatacacagcagtgcctgcccctctccccccgaggtcttccgaggcccttcctccgtgcctgaaccccctggacatatcatatggcaaactgaagtgtccaacgagatataggaagtgaaacacgatgtacactgaaacgtgcaatacaaatatgcagcatgaagtgcctcggttcactaacccgagctacgctgggtgcttcttttctaccactttccttaatgcctatggacacctcattctgtggctgaagttccttgtgttcaattccccccatcttcattgaacatcctgtgtagggacctcacccctgtcctgctagctttgcactgaggcaagttctgtccatgcctagtagtgccaccacctttactagatgagacttctaaagagcttggcatggaaggaaagcccgggggccttggaagccatcacttagaaactgggagagctccaggcaagccacctcatctctttgagcctcagtatcctcactcacatccctatggtcagtagagtgccaagcacatggtggacacgcagaagtattgatcatcttcctcacctcccctaggagagcccttgggaatccccatataatattctctgaagacctcaatccaatgagccaagtaattgactcggtagcaaactcatggaaaggacattagaagccaaaaggaggtagagtatgtcccagtacaataaccagcctctgatctcaaggaagaaagaacagagctgcaggtgagaagtgtgcgcctcaaatcaccaaagtgagagtggaaggagcacccaccagtcccttggaggcgatccctaagaccggtgagaatggcttcgaaaaatgtgatcgtcctcaaccccacagtcttgaacctaacaggagatcttgaagcctgaaagacaccactttaggatcaacagcagatctgtgactttccacagcaggcacacagaaccctagagttagtcgaggttggacataggaagggcttccaaacacacagagaaattcatggatcctaaattataaaggcaggttctttaaaagaaccaagatgattgtgagattacgtagagtttttttttgtttttcttgatacagagtctcgctctgttgcccaggcaggagtgcagtggcatgatctcagctcgctacaacctccacctcccgggttcaagcaattccctgccacagccacctgagtagctgggattacaggcacctaccaccatgcgcagctaatttttctatttctagtagagacgggatttcaccatcttgaccaggttggtcttgaactcctgacctcatgatccacccaccctggcctcccaaagtggtgggattacaggcatgagccactgcgcccggcctattctgagccttttgaagctgacgccagaggcgcgaagcccagcctctccccactggccatgtggaggctctccctgagtagtctctcagcctgcaggagccaaggatctggacccaagtgatgccccaacagtgggcaacttcccagtactgttaggagaatcccaagtctaatgcaaagttgattttttaccagcaatgaatgatagacatggtctccattgctcaaggctctgggaagatctagactagagaaaacgatcatcgacttctacccacacctgagggcctcagttcttccctctggtccatggctgccatggtaaccccttataaaggtgtttccccaggggtccctagagtcctctgagtcaccataattctggggtcaacagaagtggagaggtgagaggagacactcccctgccctggctggtcccacgtgttcctggcagtaacctgtctggggagcctcgccacccctgtgcccacctgcggagttatgcagtggtccccaactagggcccctgccaccctcattcctaaaggaggctggagacttcttccatggccgaaaatccacatctaagtccccagcactggctaaaaggccctgtcatgggggctccctgtcccagctgacattggaggaaggatggtgggggaggcagggaccttcttctacttggagctcagggaccctcttccattcgcacctggctcctcctctttgctggtgctggaaggagcaggggaaagtggtgactccaccctcccttggccccatatttttcggagctggtatcatcgatgacacccctcttggatcctcacacaggcctgcctggagaacagctcacagcacagtgccctcccagcaggtgatgagtctggggtgctggtccggtaatgcttcaggagtgacggcagaaaaaggagctctgtcttcccctctgaaagtagggagatggcagggccccagcattcacatcctaggccacaggggtgtgggtgttcaatgttggttgccaacaccacagccaatcacttctagaagcatttgcttgtttgcttgcttgtgtttctacagtgtcaagtgctacatgctcttaaactcagggtgggggaagatttcaccactggagagggtggtctttcctgctcttatccctgggggcatctgatgcttgtgacttccagatggtatggaaatagccccaaccctgggcaccaggagacaggaggctcagtcctggccttctccgcctcagccttgtcctgctgtgaggtttccacaaggcaccccatgccactgggccattctcacctgggaattgagaaagaacaatctgccccttatggaaagacaaaatgaggcaaagggcccctgtgttgggggcaggatccctgcactcagagggatttagaagctgaaataactgggttgggggcggtgactcacgccagtaatctaaggcgcgccaggctttgatttccttcactctggaatatactacagaatgtactaatataaatgtgcttgcctatgtctaatctagaatataattatcctgaactctacatggtattttagtctagaaagtactgtagcttctctgagttctaaatcaaaccaggaaacagcagagggcgctggtgctgtgcttttgcaagtgagatgtgtgctcatttcaagggtccacaaaattttctggcatgctgtttccatggagaggaaagtaggtgttttctttttctttttttttttcagtcaagccacctgcaaactaggccagagtgaaggaaatcaaaaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgaattaattcgccctttggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacatttatcaaccgtacatctgtttgaactggaaacattcattacagtaaccattacatttagcattaccaaagtggtcacaagcaggagctctacatctttgttttggaggatcctctggagcaggttcacccctgtgagcaccaggacccattctttggttagggtgttgacattccatacacagttcttcagacctacaagccaggatgtttttacaagaagctctagcacatttaggtctagaggtagactgggtggtcctaggaacatctcttctttgagaagatctaagttgttcttctgttcttttagcttcatggaatctttggttctgagcttcaatgaaacatttctgacagtaaccttcaaacatggtagaacccagggtaccacattctctacccagacatgggatggtgttctggaacctagaagctgtgggagaaactttaccagaagcagcagcaaagtgtttgttttctctgtattctatgaaacacagggtacagaaacctttgttttcaggggtaccaaagtaaacacaaccagctttcctacatttagaggtacctgttctgtcaccgggggtcctagcagcctgagacagacaaccagctggagcatcattaccagctctgtgacaagagtgtggagaaggagatctaaccagcctagatgggtcagatttagacctttggtgacaagagggtggtagagaggtagacagagaagaagaagcttcagcagtggtccttttgaaacaggtagaacagataccattgaaggttctggtaacatcctggaggcaagcctgacatttacctgggtccaggtgtctggtgtggtctggagcatgagaagcatgaagttgtctagcattgtgacatctttcacagaaaccattgtgttgaacattcagggtgaagggacaaccaggactgcgacatttcatagcagtggtttcagagaacaggaaaggagatggagctgttggaggagcagagtgtggaccaccagtagattcttcggggttccaagccagaggttcataagcttcacctctagaagcacccagagccataccaggtaaaccttcgggaccaggtttagagttcagttttggaagtttgttctggtttttctgccgacgttcagaacattcatgacacagtggttgggtgttaacagacatgaagaagggacagttgggggtttcacatttaacatccatcagagacagttgaggaacagatggttccatggggttctgagcatgaccttctcttctaccctgttcagagttttcctgccattttttgtattcatgttgaaccagttcaaagtagtcatcaaccaggttgatttctttaggtaagttagcttcatccagtttagcagcattgatcaggtgggtggtaccatggtcccaaccctgaacagggatttcaataaccatcaggtattctttcagtaatttttctttcatttcattttctgggtctgtcaggaagtgaactttcagatcctcaaacctacccctgtctctgttaaccagaggaacagccctgatttctggaccagagtctttcagggtaaccagtggaacaaagtggtgagagtcataacccagaactatggggtatctgtaacattcctgagctggccagtgaagaggtaagtagataccaccaactttcagaggagcaaagttagaaccagattccagtgagcgaagcattttgtcagagataactatgattggcctccgaaggatgttacacagaacaaagatgtggatttcttccagagagttgtactgtaaaccagacctagccatgggggtgtcagtagaagccattttgatcaggttgtcccattcatcattccagtttctggtgtcataacacagacctgtttcaacaaattcctgagatttcagagattccagttgccacctgaatttgaagtttctggtgtctgtttctttcagggtagagaacagagcttttcttagaaccaggtctgtgtcctgaacaccccacatgtactgagaggtagcatgcatcagacagttaccatcaccattggttttcagagcaaccagtttcctaacttccctacaccagttcagttttttctgagattccagggtagcctggatgttcctgtctatcagagctttgtggatgatttccctgaactgtggacagaactgacaggttctgaacatttccagggtgtatctgtgcatggttttgaagtggtggatgataccattggttggtttgaagatatcctcaggggtcctttctctgatttttacagcttttctcatgttagacaggtacagagcttgagggagaacttgttcagccattggtccagggttctcttccacatctccagcttgtttcagtaggctgaaatttgttgcgccactgcccgggtgtaaagaccaagcagcagccaggaaaggggtaaccagtagaagaactgttttttcagacagagaggtaccaccattttccagttgttcattgaagttacacaggtcttttttacaacagtagtaggtcagttcattttctcttaatctggtggtaacatcattgaagttacagtgttcaaatttccaacatttgttgtaaacctgcaggccagctttggtgatcagacaagcatcaaagtcagaagaacagttaacagctgttttacagtcagcagtagggttgggacagttgtaacactgaagagagtgaccagagtgacagaaaacagccagaaccagtaacagaccaaacagaacagaaccaccttggatacccataggccccgggttctcctctacgtccccggcctgcttgagcaggctgaaattcgtggctccgctgccggtcagtagccccatggtaaccagggtacccagtagaccagtcagggtgaaacaggtgtgaccagacagaagcctggtggtaccagaggtggtaccagaacctttgttaggggtggtttcatggaagtgtttggtggtcctagaaacgggggtagatctggtagcctgagcattgggggtggtggttttggtggtggttttctgagaggtgggagaaacttctgtggtgggaacattaactgtggtaggtttctgaactgtaggaggaactttagaggtcagagatttacccctacattcgggaggaggaccagaccattcaccttcatcattgttaacagtacagtagatagagtgttcaccaatcatggtgaaacctttgttacaagcataggtaacagactgcctgtaaccatagtgatcacgttcaccctggatgataccattgtctatctgaggaggagctggacagtagatttccctacattcgggtaaagggtcagaccactgaacagaagaaccagagatcagacagaaagaagaggtagaaccaaacagtttgtaaccagtgttacaagagaaagagatggtagcaccaaacaggataccaccaggaacatcaatctgaccattcctgatttcaccggggttgggacaagatttttttttacagaattcaacagcagtagaccatttcaggttctgtagacaggtcagtttgggagacagagagggttctcttctgtaaccaggtctacattcatattcaacaacagtaccaactgggaagtagttctgggtgatgtagggttgtttcagagaagcagagttcagcctggtgggaacttcacaagatctgttacagaattcttctatgtcagaccactgagaacctttcagacagataacagagtctttttcacctgggattttaacaaaagattcttcacatttgtaggtgataacagtatcctcagggaaagaggttctaccttccagagcaggttgagcattaggaacatctggaggtaaaccacagtcaccccaaacagcagggaggcacagtaaaacaagtaataaaagtcttggaagttcacctagaagtgggagtgctgctggaacagatggcctggctactgtcataggcccggggttctcctccacgtcgccagcctgcttaaggagtgagaagttggtggctccactgcccagagaggtgaatttaacttctctgtgggtttcatctgtcaggtaggtaccttttttttttctcctttgaaggtacctgtaaggaacaacacagataacagcaacaccaacaacaatagctataactataacagcaataacccaaacatccagagagtccaggataccttcttcaggtttagggtaaccagggtagttagaaacgggaggtttgtaggtaggtcttggaccagaagcagaagaagcaggagatttggtggtagaagaggtagaaacagagtgagacagagcgggtggtttggtagaagagggtggtaaaactttcagacatttgggaactggtgggtcccaggtagagttagagtcacaaacaatggtgtcagaaccatccaggtagaaacctttgtcacattcaaacataacagtagctttgtagtagaattttttaccaaaaccagagatctgtttaccattttcaacaactgggaatctacatttaacaactttacattctggagcagctctagaccaaacagagttgtcaccacagtagatggtagattcaccaatcagagagaatgggtctggaccaggagcagggtcacaagagtaggtaacagcatccaggtattcaaaaacttcaacttcagagaaggtgtgtttaccatttttgattttgggtggtggggtacacagaactttttcacagatgggaggtttaccagaccagatagcaacagaacctttcagttcacagtacaggatttcttcacctatcaggtagtaaccttcattacagatgaagtgcatctggtaaccaaattcataggtaccattagctggaacagcctgaccattcagaggatctcggatgtagggacaggtttccctgtaacaagcatcatcagaaacaggtaaccaggtgtggtttctgtcacagatggtgtgggtagccagaggtgggatgtagaagtaaccttttttacatttgtagtcaactctttcacctatttcatagtatggttttggtttaccgatcagttccatagcttcaaaggttggtggttcttcacaagcatcagagaaagagtacagtaaaagaaccatagcagccagtagtaaaccagggaatctccaagatgggaagggacactcccttctaccaggtggttccatggtggctcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctcaggcaccgaggagacaccgtggcccccgaagcagcgtgctttagaaagggaataagaattcccgcctccgcgccccactttcaccccagcggggcagcgtccgccatgtgaaagctccccatcccccacccccagtgaagggaaatggcgccgggaggctgagggtggggaagctgtttgtacgctcaggcctccgctcaagaccccgttcataaaccttaagccccactgctactgaattggtccgatttcctgcctctctcccacggaggcggctggccgacttccactgaggcgccaacggcctcgccatgcccttttcaataactcattgatttcaaacccgttacgtccatcgcggactcagtcgcttcagcccgatttcccgcagccgagcgagatgagagagatcgatctccgcggacgaacacgaaccggactcgtcctggcgctgtagtgagaactgccgctgctcgagaaacaactctgcgaggagcacctccgcacgggacccggcgctgctgctactgccgctagagccgctgccgccgcttttctagaaccttcccccccactaacgcgtcttccgctacgtcaggccgtcgcgtaaacgccctatccgccgccaatggcgggaaggctctacgccccaccttacgccaaatgcgtactcctcccacccttgcggccagagacagtacccgacgttacttccgtaaatgcgctcaatgaattgcggaaggctagagtcctgctagttactacctcttggaatagggtcccggcccctgccttggcgaaggcaggtgagaaacgtcgcgcagtttgaaattaacgccgacgggaggggcttaatccgcagcctggagatccagccccctcaacccgggaggtggtccctgcagttacgccaatgataacccccgccagaaaaatcttagtagccttccctttttgttttccgtgccccaactcggcggattgactcggccccttccggaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccggcgggcgaggaagtgggcggagtccggttttggcgccagccgctgaggctgccaagcagaaaagccaccgctgaggagactccggtcactgtcctcgccccgcctcccccttccctccccttggggaccaccgggcgccacgccgcgaacggtaagtgccgcggtcgtcggcgcctccgccctccccctagggccccaattcccagcgggcgcggcgccggcccctccccccgccgcgcgcgcgcccgctgccccgcccttcgtggccgcccggcgtgggcggtgccacccctccccccggcggccccgcgcgcagctcccggctccctcccccttcggatgtggcttgagctgtaggcgcggagggccagcgtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcgggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgccaccatgtctagatctgttgctcttgctgttcttgctctgttgtctctgtctggtctggaagctgtgatggcaccgagaaccctcatcctcgggggcggaggttcaggaggcggtgggagcggcggagggggttccatccaaagaacccctaaaatccaggtttactctaggcacccagctgaaaacggcaaatctaacttcctgaactgttatgtttctggtttccacccctctgacattgaagttgacttactgaaaaatggtgaaaggatagaaaaagttgaacactctgacctgtctttctctaaagactggtctttctacttactgtactacactgaattcacccctacagaaaaagatgaatatgcttgtagagttaaccatgttaccctgtctcaaccaaaaattgttaaatgggacagagacatgggtggcggagggtctggcgggggtggaagcgggggtggaggctccggaggtgggggcagtggtagtcattccttaaagtacttccacacctctgtttcccgaccaggtaggggtgaacctaggttcatctctgttgggtatgttgatgatacccaatttgtcagatttgataatgatgctgcttctccaagaatggttccaagggctccttggatggaacaggaagggtcagaatactgggacagagaaaccagatctgctagagacactgctcaaatattcagagttaacttgagaactttaagaggttattataatcagtctgaggcaggttcccacactcttcaatggatgcatggttgtgaattgggtccagatggtagattcctcaggggttatgaacagtttgcctatgatggaaaagattatttgacccttaatgaggacctaaggtcctggactgcagttgatacagcagcccagatttcagaacaaaagtctaatgatgcttctgaagctgaacaccaaagagcgtatttagaggatacttgtgtagaatggttacacaagtacttggaaaagggtaaggaaaccttactacacttggaacctcctaagacccatgtcacacatcatccaatctctgaccatgaagcaacactaaggtgttgggcactaggtttttaccctgctgaaattaccctcacctggcaacaagatggtgaaggtcacacccaagacactgaactagtagagacaagacctgctggggatggtacatttcaaaaatgggctgctgtggtagttcctagtggggaagaacaaaggtatacctgtcatgttcaacatgaggggttaccagaaccagttacccttaggtggaaaccagcttctcaacctactattccaattgttggtatcattgctggtcttgtcttgttgggttctgttgtttctggtgctgttgtggcagcagtgatctggaggaaaaaatcatctggtggtaagggtggttcttactctaaggctgaatggtcagattcagctcaagggagtgaatctcactctttgggcagtggcgccaccaattttagcctgttaaagcaggctggcgacgtggaggagaatcctgggcctatgtggcctctggtagcagcacttcttttaggttcagcatgttgtggttcagctcaactactgttcaacaaaaccaaatctgttgaattcaccttctgtaatgacacagttgttatcccatgttttgttaccaacatggaagctcagaacaccacagaagtttatgttaaatggaaattcaaaggtagagacatctacacctttgatggtgctctgaacaaatctacagttcccactgacttctcttctgctaaaatagaagtttctcaattactgaaaggtgatgcttctctgaaaatggacaaatctgatgctgtttctcacacaggcaactacacttgtgaagttacagaactgaccagagaaggtgaaaccatcattgaactgaaatacagagttgtttcttggttctctcctaatgaaaacatcctgattgttatcttccccatctttgctatccttctgttctggggtcagtttggtatcaaaaccctgaaatacagatctggtggtatggatgaaaaaaccattgctttactggttgctggtctggttatcactgttatagttatagttggtgctatcctgtttgttccaggtgaatactctctgaaaaatgctacaggtctgggtctgatagttacctctactggtatcctgatcttacttcactactatgttttctctacagctattggtctgacctcttttgttatagctatcctggttatccaggttattgcttacatcctggctgttgttggtctgtctctgtgtattgctgcttgtatccccatgcatggtcctcttctgatctctggtctgtctatcctggctctggctcaattactgggtctggtttacatgaaatttgttgcttctaaccaaaaaaccatccaaccacctaggaaagctgttgaagaacctctgctttcaaagaatctaaaggtatgatgaatacgaaggcagcggcgccaccaactttagcttgctgaagcaagcaggggacgtcgaagagaatcctgggcctatgaggatctttgctgtttttatcttcatgacgtactggcaccttctgaatgctttcacagttactgttccaaaagacctgtatgttgttgaatatggttctaacatgaccatagaatgtaaattcccagttgaaaaacaactggacctggctgctctgattgtttactgggaaatggaggataaaaacatcatccagtttgttcatggtgaagaggatctgaaagttcaacactcttcttacagacaaagagctaggttactgaaagaccaactgtctctgggtaatgctgctctacagatcacagatgttaaattacaggatgctggtgtttacagatgtatgatctcttatggtggtgctgactacaaaaggatcacagttaaagttaatgctccctacaacaaaatcaaccaaagaatcctggttgttgaccccgttacctctgaacatgaactgacctgtcaggctgaaggttaccctaaagctgaagttatctggacctcttctgaccaccaggttctgtctggtaaaaccaccaccaccaactctaaaagggaagaaaaactgttcaatgttacctctaccttaagaatcaacaccaccaccaatgaaatcttctactgtaccttcaggaggctggacccagaagaaaaccacactgctgaactggttatccctgaattacctctggctcaccctcccaatgaaagaacccacctggttatcctgggtgctatcctactgtgtctgggtgttgctctgaccttcatcttcaggttaaggaaaggtagaatgatggatgttaaaaaatgtggtatccaggacaccaactctaaaaaacagtctgacacccacctggaagaaacctgataaaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctcttgaggccaagtgtgactttgtggtgtggctgggttgggggcagcagagggtgaaccctgcaggagggtgaaccctgcaaaagggtggggcagtgggggccaacttgtccttacccagagtgcaggtgtgtggagatccctcctgccttgacattgagcagccttagagggtgggggaggctcaggggtcaggttcctgttcctgcttattggggagttcctggcctggcccttctatgtctccccaggtaccccagtttttctgggttcacccagagtgcagatgcttgaggaggtgggaagggactatttgggggtgtctggctcaggtgccatgcctcactggggctggttggcacctgcatttcctgggagtggggctgtctcagggtagctgggcacggtgttcccttgagtgggggtgtagtgggtgttcctagctgccacgcctttgccttcacctatgggatcgtggctgtcagccttgagggtcagcctggcccaggctcccataggcttaggagaggccgcaattcctacctgttcatccagacagagggggacctggaatcaaagtcaagttggggtagggggtccatggggccatatctggcctgcagacagctctggttagctatgggctgaggtctggattctgccttgtgactggagactgggcgccatcccgtggcctctgagggcccagtttgttcgtgacccatgctgcagagggagatacccaggctcctggacctcagggcctgcctggcactgcaggccaggttctctttttatttctgagcaggggtcttgtgttgcccaggctggagtgtaatggcacaaccatggctcactgcagcctcaactttgggctcaagcaattctcctgcctcagcctcccaagtagctgaaactacacaggcatgccccagcacgcccagctagttgcaggtcagattctcattgttctacagggccctgagttaaatactctcgcaagaagaaaccaagcctggcctgccttccctctgctagaagctgtgattgacaccagcagagaatgaaggaagaaaagggggtgaggatcccttgcagggatgccgagtttgcaggtggcttgactgaaaaaaaaaagaaaaagaaaacacctactttcctctccatggaaacagcatgccagaaaattttgtggacccttgaaatgagcacacatctcacttgcaaaagcacagcaccagcgccctctgctgtttcctggtttgatttagaactcagagaagctacagtactttctagactaaaataccatgtagagttcaggataattatattctagattagacataggcaagcacatttatattagtacattctgtagtatattccagagtgaaggaaatcaaagtttatgactcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacattttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgcctcacacgtacgcgtacgatgcataacttcgtataatgtgtactatacgaagttatataacttcgtataggatactttatacgaagttatgtttcagcggccgcgattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 213 - DNA, 26999 bp, Artificial Sequence
  Payload 12G sequence
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccataacttcgtatagcatacattatacgaagttatagaatggaaggttgtgttgaagcgcggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaatgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgagggcacaccggcgcgccggagccgagagtaattcatacaaaaggagggatcgccttcgcaaggggagagcccagggaccgtccctaaattctcacagacccaaatccctgtagccgccccacgacagcgcgaggagcatgcgctcagggctgagcgcggggagagcagagcacacaagctcatagaccctggtcgtgggggggaggaccggggagctggcgcggggcaaactgggaaagcggtgtcgtgtgctggctccgccctcttcccgagggtgggggagaacggtatataagtgcggcagtcgccttggacgttctttttcgcaacgggtttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtctttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaatgccaccatgttaggtgttctggttcttggtgcactggctctggcaggtttaggtttccccgctcctgctgaaccacaaccaggtggttctcagtgtgttgaacatgactgttttgctctgtaccccggtcccgctaccttcctgaatgcttctcagatctgtgatggtcttaggggtcacctgatgacagttcgcagctctgttgctgctgatgttatctctttattactgaatggtgatggtggtgttggtaggaggagactgtggattggactgcaactaccaccaggttgtggtgaccctaaaagactgggtcctcttagaggtttccagtgggttacaggtgacaacaacacctcttactctagatgggctagactggacctgaatggtgctcctctgtgtggtcctctgtgtgttgctgtttctgctgctgaagctactgttccttctgaacctatctgggaagaacaacagtgtgaagttaaagctgatggtttcctgtgtgaattccacttccccgctacctgtagaccactggctgttgaacccggtgctgctgctgctgctgtttctatcacctatggtaccccttttgctgctagaggtgctgacttccaggctctaccagttggttcttctgctgctgttgctcctctgggcttacaactgatgtgtacagctccacccggtgctgttcagggtcactgggctagagaagctcccggtgcttgggactgttctgttgaaaatggtggttgtgaacatgcttgtaatgctatccctggtgctcctaggtgtcagtgtcctgctggtgctgctcttcaggctgatggtagatcttgtactgcttctgctacccagtcttgtaatgacctgtgtgaacacttctgtgttcccaaccccgaccaacccggttcttactcttgtatgtgtgaaacaggttacagactggccgctgaccaacacagatgtgaagatgttgatgactgtatcctggaaccctctccatgtccacaacgctgtgttaacacccagggtggttttgaatgtcactgttaccctaactatgacctggttgatggtgaatgtgttgaacccgttgacccctgtttcagggctaactgtgaataccagtgtcaacctctgaaccagacctcttacctgtgtgtttgtgctgaaggttttgctccaatccctcatgaacctcacaggtgtcagatgttctgtaaccagacagcttgtcccgctgactgtgaccctaacacccaggcttcttgtgaatgtccagaaggttacatcctggatgatggtttcatctgtacagacattgatgaatgtgaaaatggtggtttctgttctggtgtttgtcacaaccttcccggtacctttgaatgtatctgtggtcccgactctgctctggctagacacattggtacagactgtgactctggtaaagttgatggtggtgactctggttctggtgaacctcctccctctccaacccccggttctaccctgaccccaccagctgttggtctggttcactctggtctactgataggtatctctatagcttctctgtgtctggttgttgctctactggctctactgtgtcaccttaggaaaaaacagggtgctgctagggctaaaatggaatacaaatgtgctgctccatctaaagaagttgttttacaacatgttaggactgaaaggacccctcaaaggctgggcagcggcgcgaccaacttttcgctccttaagcaggccggggacgtggaggagaatccagggcctatctacactatgaaaaaagtacatgctttatgggcctctgtttgtttacttctgaacctggctcctgctcctctgaatgctgactctgaagaagatgaagaacacaccatcatcacagacactgaacttcctccactgaaactgatgcactctttctgtgctttcaaagctgatgatggtccttgtaaagctatcatgaaaaggttcttcttcaacatcttcaccagacagtgtgaagaattcatctatggtggttgtgaaggtaaccagaacagatttgaatctctggaagaatgtaagaaaatgtgtaccagggacaatgctaacaggatcatcaaaaccaccctacaacaggaaaaaccagacttctgtttcctggaagaggatcctggtatctgtagaggttacatcaccaggtacttctacaacaaccagaccaaacagtgtgaaagattcaaatatggtggttgtctgggtaacatgaacaactttgaaaccctggaagaatgtaaaaacatctgtgaagatggtcccaatggtttccaggttgacaactatggtacccaactgaatgctgttaacaactctctgacccctcagtctaccaaagttccctctctgtttgaattccatggtccttcttggtgtctgacccctgctgacagaggtctgtgtagagctaatgaaaacaggttctactacaactctgttataggtaaatgtagacccttcaaatactctggttgtggtggtaatgaaaacaacttcacctctaaacaggaatgtttaagggcttgtaaaaaaggtttcatccaaagaatctctaaaggtggtctgatcaaaaccaaaagaaaaagaaaaaaacaaagagttaaaatagcttatgaagaaatctttgttaaaaacatgtacttacaggaactgggaggttcagggggaggagggtccggtgggggtggttctggtggttgtagaaatggtaaaaccctgtctgtttctcaactggaacttcaggactctggtacctggacctgtacagttctacagaaccagaaaaaagttgaattcaaaatagacatagttgttctggctttccagaaagcttcttctatagtttacaaaaaagaaggtgaacaggttgaattctctttcccactggctttcactgttgaaaaactgactggttctggtgaactgtggtggcaggctgaaagggcttcttcttctaaatcttggatcacctttgacctgaaaaacaaagaagtttctgttaaaagggttacccaggacccaaaattacagatgggtaaaaaattacctcttcacctgaccctaccacaggctcttccacagtatgctggttctggtaacctgaccctggctctggaagctaaaactggtaaactacaccaggaagttaacctggttgttatgagagctacccaacttcagaaaaacctgacctgtgaagtttggggtccaacctctccaaaactgatgctgtctctgaaactggaaaacaaagaagctaaagtttctaaaagagaaaaagctgtttgggttctgaacccagaagctggtatgtggcagtgcctgctgtctgactctggtcaggttttactggaatctaacatcaaagttcttcctacctggtctacccctgttcaaccaatggctctgattgttctgggtggtgttgctgggcttcttctgttcataggtctgggtatcttcttctgtgttaggtgtcgacacagaaggagacaggctgaaagaatgtctcagatcaaaaggttccgtcagaaagatgatggtaaatgtccactgaacccacactctcacctgggtacctatggtgttttcaccaatgctgcttttgacccctctcctggcagcggagcgaccaactttagccttctgaaacaggctggggacgtggaggagaatcctgggcctatggaggataccaaagaatctaatgttaaaactttctgttctaaaaacatcctggctatcctgggtttctcttctatcattgctgttattgctttactggctgttggtctgacccagaacaaagctttacctgaaaatgttaaatatggtattgttctggatgctggttcttctcacacctctctgtacatctacaaatggcccgctgaaaaagaaaatgacacaggtgttgttcaccaggttgaagaatgtagagttaaaggtcctggtatctctaaatttgttcagaaagttaatgaaattggtatctacctgactgactgtatggaaagagctagggaagttatccctcgttcgcaacaccaggaaacccccgtttacctgggtgctactgctggtatgagattactaagaatggaatctgaagaactggctgacagagttctggatgttgttgaaagatctctgtctaactacccctttgacttccagggtgctaggatcatcacaggtcaggaagaaggtgcttatggttggatcaccatcaactaccttctgggtaaattctctcagaaaaccaggtggttctctatagttccatatgaaaccaacaaccaggaaacctttggtgctctggacctgggtggtgcttctacccaggttacctttgttcctcagaaccagaccattgaatctcctgacaatgctctacagttcagactgtatggtaaagactacaatgtttacacccactctttcctgtgttatggtaaagaccaggctctgtggcagaaactggctaaagacatccaggttgcttctaatgaaatccttagggacccttgtttccaccctggttacaaaaaagttgttaatgtttctgacctgtacaaaaccccttgtaccaaaagatttgaaatgaccttacccttccaacagtttgaaatccagggtattggtaactaccaacagtgtcaccagtctatcctggaactgttcaacacctcttactgtccctactctcagtgtgctttcaatggtatcttcttacctcctctacagggtgactttggtgctttctctgctttctactttgttatgaaattcctgaacctgacctctgaaaaagtttctcaggaaaaagttactgaaatgatgaaaaaattctgtgctcaaccctgggaagaaatcaaaacctcttatgctggtgttaaagaaaaatacctgtctgaatactgtttctctggtacctacatcctgtctttattactgcaaggttaccacttcactgctgactcttgggaacacatccacttcatcggtaaaatccagggttctgatgctggttggaccctgggttacatgctgaacctgaccaacatgatccccgctgaacaacctctgtctaccccactgtctcactctacctatgttttcctgatggttctgttctctctggttctgttcaccgttgctatcataggtctactgatcttccacaaaccatcttacttctggaaagacatggttggcagcggcgccaccaatttctcactactgaaacaggccggtgacgtcgaggagaaccccgggcctgaaaggccacaaccagattctatgccacaagacctgtctgaagcactgaaagaagctaccaaagaagttcacacccaggctgaaaatgctgaattcatgagaaacttccagaaaggtcaggttaccagagatggtttcaaactggttatggcttctctgtaccacatctatgttgctctggaagaagaaatagaaagaaacaaagaatctcctgtttttgctccagtttacttcccagaagaactacacaggaaagctgctctggaacaggacctggctttctggtatggtcctagatggcaggaagttatcccatacacccccgctatgcaaaggtatgttaaaagacttcatgaagttggtaggacagaaccagaattactggttgctcatgcttacaccagatacctgggtgacctgtctggtggtcaggttctgaaaaaaatagctcagaaagctctggacctaccttcttctggtgaaggtctggctttcttcaccttcccaaacatagcttctgctaccaaattcaaacaactgtacaggtctaggatgaactctctggaaatgacccccgctgttaggcaaagagttattgaagaagctaaaacagctttcctactgaacatccaactgtttgaagaattacaggaactactgacccatgacaccaaagaccagtctccatctagggctcccggtttaagacaaagggcttctaacaaagttcaggactctgctccagttgaaacccctagaggtaaacctcctctgaacaccagatctcaggctcctttattaagatgggttctgaccctgtctttcctggttgctactgttgctgttggtctgtatgctatgtgataagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcatatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacaggggtcatggtttgactgtcctgtgagcccttcttccctgcctcccccactcacagtgacccggaatctgcagtgctagtctcccggaactatcactctttcacagtctgctttggaaggactgggcttagtatgaaaagttaggactgagaagaatttgaaaggcggctttttgtagcttgatattcactactgtcttattaccctgtcataggcccaccccaaatggaagtcccattcttcctcaggatgtttaagattagcattcaggaagagatcagaggtctgctggctcccttatcatgtcccttatggtgcttctggctctgcagttattagcatagtgttaccatcaaccaccttaacttcatttttcttattcaatacctaggtaggtagatgctagattctggaaataaaatatgagtctcaagtggtccttgtcctctctcccagtcaaattctgaatctagttggcaagattctgaaatcaaggcatataatcagtaataagtgatgatagaagggtatatagaagaattttattatatgagagggtgaaaccctcaaaatgaaatgaaatcagacccttgtcttacaccataaacaaaaaggctttgatttccttcactctggaatatactacagaatgtactaatataaatgtgcttgcctatgtctaatctagaatataattatcctgaactctacatggtattttagtctagaaagtactgtagcttctctgagttctaaatcaaaccaggaaacagcagagggcgctggtgctgtgcttttgcaagtgagatgtgtgctcatttcaagggtccacaaaattttctggcatgctgtttccatggagaggaaagtaggtgttttctttttctttttttttttcagtcaagccacctgcaaactaggccagagtgaaggaaatcaaaaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgaattaattcgccctttggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacatttatcaaccgtacatctgtttgaactggaaacattcattacagtaaccattacatttagcattaccaaagtggtcacaagcaggagctctacatctttgttttggaggatcctctggagcaggttcacccctgtgagcaccaggacccattctttggttagggtgttgacattccatacacagttcttcagacctacaagccaggatgtttttacaagaagctctagcacatttaggtctagaggtagactgggtggtcctaggaacatctcttctttgagaagatctaagttgttcttctgttcttttagcttcatggaatctttggttctgagcttcaatgaaacatttctgacagtaaccttcaaacatggtagaacccagggtaccacattctctacccagacatgggatggtgttctggaacctagaagctgtgggagaaactttaccagaagcagcagcaaagtgtttgttttctctgtattctatgaaacacagggtacagaaacctttgttttcaggggtaccaaagtaaacacaaccagctttcctacatttagaggtacctgttctgtcaccgggggtcctagcagcctgagacagacaaccagctggagcatcattaccagctctgtgacaagagtgtggagaaggagatctaaccagcctagatgggtcagatttagacctttggtgacaagagggtggtagagaggtagacagagaagaagaagcttcagcagtggtccttttgaaacaggtagaacagataccattgaaggttctggtaacatcctggaggcaagcctgacatttacctgggtccaggtgtctggtgtggtctggagcatgagaagcatgaagttgtctagcattgtgacatctttcacagaaaccattgtgttgaacattcagggtgaagggacaaccaggactgcgacatttcatagcagtggtttcagagaacaggaaaggagatggagctgttggaggagcagagtgtggaccaccagtagattcttcggggttccaagccagaggttcataagcttcacctctagaagcacccagagccataccaggtaaaccttcgggaccaggtttagagttcagttttggaagtttgttctggtttttctgccgacgttcagaacattcatgacacagtggttgggtgttaacagacatgaagaagggacagttgggggtttcacatttaacatccatcagagacagttgaggaacagatggttccatggggttctgagcatgaccttctcttctaccctgttcagagttttcctgccattttttgtattcatgttgaaccagttcaaagtagtcatcaaccaggttgatttctttaggtaagttagcttcatccagtttagcagcattgatcaggtgggtggtaccatggtcccaaccctgaacagggatttcaataaccatcaggtattctttcagtaatttttctttcatttcattttctgggtctgtcaggaagtgaactttcagatcctcaaacctacccctgtctctgttaaccagaggaacagccctgatttctggaccagagtctttcagggtaaccagtggaacaaagtggtgagagtcataacccagaactatggggtatctgtaacattcctgagctggccagtgaagaggtaagtagataccaccaactttcagaggagcaaagttagaaccagattccagtgagcgaagcattttgtcagagataactatgattggcctccgaaggatgttacacagaacaaagatgtggatttcttccagagagttgtactgtaaaccagacctagccatgggggtgtcagtagaagccattttgatcaggttgtcccattcatcattccagtttctggtgtcataacacagacctgtttcaacaaattcctgagatttcagagattccagttgccacctgaatttgaagtttctggtgtctgtttctttcagggtagagaacagagcttttcttagaaccaggtctgtgtcctgaacaccccacatgtactgagaggtagcatgcatcagacagttaccatcaccattggttttcagagcaaccagtttcctaacttccctacaccagttcagttttttctgagattccagggtagcctggatgttcctgtctatcagagctttgtggatgatttccctgaactgtggacagaactgacaggttctgaacatttccagggtgtatctgtgcatggttttgaagtggtggatgataccattggttggtttgaagatatcctcaggggtcctttctctgatttttacagcttttctcatgttagacaggtacagagcttgagggagaacttgttcagccattggtccagggttctcttccacatctccagcttgtttcagtaggctgaaatttgttgcgccactgcccgggtgtaaagaccaagcagcagccaggaaaggggtaaccagtagaagaactgttttttcagacagagaggtaccaccattttccagttgttcattgaagttacacaggtcttttttacaacagtagtaggtcagttcattttctcttaatctggtggtaacatcattgaagttacagtgttcaaatttccaacatttgttgtaaacctgcaggccagctttggtgatcagacaagcatcaaagtcagaagaacagttaacagctgttttacagtcagcagtagggttgggacagttgtaacactgaagagagtgaccagagtgacagaaaacagccagaaccagtaacagaccaaacagaacagaaccaccttggatacccataggccccgggttctcctctacgtccccggcctgcttgagcaggctgaaattcgtggctccgctgccggtcagtagccccatggtaaccagggtacccagtagaccagtcagggtgaaacaggtgtgaccagacagaagcctggtggtaccagaggtggtaccagaacctttgttaggggtggtttcatggaagtgtttggtggtcctagaaacgggggtagatctggtagcctgagcattgggggtggtggttttggtggtggttttctgagaggtgggagaaacttctgtggtgggaacattaactgtggtaggtttctgaactgtaggaggaactttagaggtcagagatttacccctacattcgggaggaggaccagaccattcaccttcatcattgttaacagtacagtagatagagtgttcaccaatcatggtgaaacctttgttacaagcataggtaacagactgcctgtaaccatagtgatcacgttcaccctggatgataccattgtctatctgaggaggagctggacagtagatttccctacattcgggtaaagggtcagaccactgaacagaagaaccagagatcagacagaaagaagaggtagaaccaaacagtttgtaaccagtgttacaagagaaagagatggtagcaccaaacaggataccaccaggaacatcaatctgaccattcctgatttcaccggggttgggacaagatttttttttacagaattcaacagcagtagaccatttcaggttctgtagacaggtcagtttgggagacagagagggttctcttctgtaaccaggtctacattcatattcaacaacagtaccaactgggaagtagttctgggtgatgtagggttgtttcagagaagcagagttcagcctggtgggaacttcacaagatctgttacagaattcttctatgtcagaccactgagaacctttcagacagataacagagtctttttcacctgggattttaacaaaagattcttcacatttgtaggtgataacagtatcctcagggaaagaggttctaccttccagagcaggttgagcattaggaacatctggaggtaaaccacagtcaccccaaacagcagggaggcacagtaaaacaagtaataaaagtcttggaagttcacctagaagtgggagtgctgctggaacagatggcctggctactgtcataggcccggggttctcctccacgtcgccagcctgcttaaggagtgagaagttggtggctccactgcccagagaggtgaatttaacttctctgtgggtttcatctgtcaggtaggtaccttttttttttctcctttgaaggtacctgtaaggaacaacacagataacagcaacaccaacaacaatagctataactataacagcaataacccaaacatccagagagtccaggataccttcttcaggtttagggtaaccagggtagttagaaacgggaggtttgtaggtaggtcttggaccagaagcagaagaagcaggagatttggtggtagaagaggtagaaacagagtgagacagagcgggtggtttggtagaagagggtggtaaaactttcagacatttgggaactggtgggtcccaggtagagttagagtcacaaacaatggtgtcagaaccatccaggtagaaacctttgtcacattcaaacataacagtagctttgtagtagaattttttaccaaaaccagagatctgtttaccattttcaacaactgggaatctacatttaacaactttacattctggagcagctctagaccaaacagagttgtcaccacagtagatggtagattcaccaatcagagagaatgggtctggaccaggagcagggtcacaagagtaggtaacagcatccaggtattcaaaaacttcaacttcagagaaggtgtgtttaccatttttgattttgggtggtggggtacacagaactttttcacagatgggaggtttaccagaccagatagcaacagaacctttcagttcacagtacaggatttcttcacctatcaggtagtaaccttcattacagatgaagtgcatctggtaaccaaattcataggtaccattagctggaacagcctgaccattcagaggatctcggatgtagggacaggtttccctgtaacaagcatcatcagaaacaggtaaccaggtgtggtttctgtcacagatggtgtgggtagccagaggtgggatgtagaagtaaccttttttacatttgtagtcaactctttcacctatttcatagtatggttttggtttaccgatcagttccatagcttcaaaggttggtggttcttcacaagcatcagagaaagagtacagtaaaagaaccatagcagccagtagtaaaccagggaatctccaagatgggaagggacactcccttctaccaggtggttccatggtggctcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctcaggcaccgaggagacaccgtggcccccgaagcagcgtgctttagaaagggaataagaattcccgcctccgcgccccactttcaccccagcggggcagcgtccgccatgtgaaagctccccatcccccacccccagtgaagggaaatggcgccgggaggctgagggtggggaagctgtttgtacgctcaggcctccgctcaagaccccgttcataaaccttaagccccactgctactgaattggtccgatttcctgcctctctcccacggaggcggctggccgacttccactgaggcgccaacggcctcgccatgcccttttcaataactcattgatttcaaacccgttacgtccatcgcggactcagtcgcttcagcccgatttcccgcagccgagcgagatgagagagatcgatctccgcggacgaacacgaaccggactcgtcctggcgctgtagtgagaactgccgctgctcgagaaacaactctgcgaggagcacctccgcacgggacccggcgctgctgctactgccgctagagccgctgccgccgcttttctagaaccttcccccccactaacgcgtcttccgctacgtcaggccgtcgcgtaaacgccctatccgccgccaatggcgggaaggctctacgccccaccttacgccaaatgcgtactcctcccacccttgcggccagagacagtacccgacgttacttccgtaaatgcgctcaatgaattgcggaaggctagagtcctgctagttactacctcttggaatagggtcccggcccctgccttggcgaaggcaggtgagaaacgtcgcgcagtttgaaattaacgccgacgggaggggcttaatccgcagcctggagatccagccccctcaacccgggaggtggtccctgcagttacgccaatgataacccccgccagaaaaatcttagtagccttccctttttgttttccgtgccccaactcggcggattgactcggccccttccggaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccggcgggcgaggaagtgggcggagtccggttttggcgccagccgctgaggctgccaagcagaaaagccaccgctgaggagactccggtcactgtcctcgccccgcctcccccttccctccccttggggaccaccgggcgccacgccgcgaacggtaagtgccgcggtcgtcggcgcctccgccctccccctagggccccaattcccagcgggcgcggcgccggcccctccccccgccgcgcgcgcgcccgctgccccgcccttcgtggccgcccggcgtgggcggtgccacccctccccccggcggccccgcgcgcagctcccggctccctcccccttcggatgtggcttgagctgtaggcgcggagggccagcgtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcggggggggggggggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgccaccatgtctagatctgttgctcttgctgttcttgctctgttgtctctgtctggtctggaagctgtgatggcaccgagaaccctcatcctcgggggcggaggttcaggaggcggtgggagcggcggagggggttccatccaaagaacccctaaaatccaggtttactctaggcacccagctgaaaacggcaaatctaacttcctgaactgttatgtttctggtttccacccctctgacattgaagttgacttactgaaaaatggtgaaaggatagaaaaagttgaacactctgacctgtctttctctaaagactggtctttctacttactgtactacactgaattcacccctacagaaaaagatgaatatgcttgtagagttaaccatgttaccctgtctcaaccaaaaattgttaaatgggacagagacatgggtggcggagggtctggcgggggtggaagcgggggtggaggctccggaggtgggggcagtggtagtcattccttaaagtacttccacacctctgtttcccgaccaggtaggggtgaacctaggttcatctctgttgggtatgttgatgatacccaatttgtcagatttgataatgatgctgcttctccaagaatggttccaagggctccttggatggaacaggaagggtcagaatactgggacagagaaaccagatctgctagagacactgctcaaatattcagagttaacttgagaactttaagaggttattataatcagtctgaggcaggttcccacactcttcaatggatgcatggttgtgaattgggtccagatggtagattcctcaggggttatgaacagtttgcctatgatggaaaagattatttgacccttaatgaggacctaaggtcctggactgcagttgatacagcagcccagatttcagaacaaaagtctaatgatgcttctgaagctgaacaccaaagagcgtatttagaggatacttgtgtagaatggttacacaagtacttggaaaagggtaaggaaaccttactacacttggaacctcctaagacccatgtcacacatcatccaatctctgaccatgaagcaacactaaggtgttgggcactaggtttttaccctgctgaaattaccctcacctggcaacaagatggtgaaggtcacacccaagacactgaactagtagagacaagacctgctggggatggtacatttcaaaaatgggctgctgtggtagttcctagtggggaagaacaaaggtatacctgtcatgttcaacatgaggggttaccagaaccagttacccttaggtggaaaccagcttctcaacctactattccaattgttggtatcattgctggtcttgtcttgttgggttctgttgtttctggtgctgttgtggcagcagtgatctggaggaaaaaatcatctggtggtaagggtggttcttactctaaggctgaatggtcagattcagctcaagggagtgaatctcactctttgggcagtggcgccaccaattttagcctgttaaagcaggctggcgacgtggaggagaatcctgggcctatgtggcctctggtagcagcacttcttttaggttcagcatgttgtggttcagctcaactactgttcaacaaaaccaaatctgttgaattcaccttctgtaatgacacagttgttatcccatgttttgttaccaacatggaagctcagaacaccacagaagtttatgttaaatggaaattcaaaggtagagacatctacacctttgatggtgctctgaacaaatctacagttcccactgacttctcttctgctaaaatagaagtttctcaattactgaaaggtgatgcttctctgaaaatggacaaatctgatgctgtttctcacacaggcaactacacttgtgaagttacagaactgaccagagaaggtgaaaccatcattgaactgaaatacagagttgtttcttggttctctcctaatgaaaacatcctgattgttatcttccccatctttgctatccttctgttctggggtcagtttggtatcaaaaccctgaaatacagatctggtggtatggatgaaaaaaccattgctttactggttgctggtctggttatcactgttatagttatagttggtgctatcctgtttgttccaggtgaatactctctgaaaaatgctacaggtctgggtctgatagttacctctactggtatcctgatcttacttcactactatgttttctctacagctattggtctgacctcttttgttatagctatcctggttatccaggttattgcttacatcctggctgttgttggtctgtctctgtgtattgctgcttgtatccccatgcatggtcctcttctgatctctggtctgtctatcctggctctggctcaattactgggtctggtttacatgaaatttgttgcttctaaccaaaaaaccatccaaccacctaggaaagctgttgaagaacctctgaatgctttcaaagaatctaaaggtatgatgaatgacgaaggcagcggcgccaccaactttagcttgctgaagcaagcaggggacgtcgaagagaatcctgggcctatgaggatctttgctgtttttatcttcatgacgtactggcaccttctgaatgctttcacagttactgttccaaaagacctgtatgttgttgaatatggttctaacatgaccatagaatgtaaattcccagttgaaaaacaactggacctggctgctctgattgtttactgggaaatggaggataaaaacatcatccagtttgttcatggtgaagaggatctgaaagttcaacactcttcttacagacaaagagctaggttactgaaagaccaactgtctctgggtaatgctgctctacagatcacagatgttaaattacaggatgctggtgtttacagatgtatgatctcttatggtggtgctgactacaaaaggatcacagttaaagttaatgctccctacaacaaaatcaaccaaagaatcctggttgttgaccccgttacctctgaacatgaactgacctgtcaggctgaaggttaccctaaagctgaagttatctggacctcttctgaccaccaggttctgtctggtaaaaccaccaccaccaactctaaaagggaagaaaaactgttcaatgttacctctaccttaagaatcaacaccaccaccaatgaaatcttctactgtaccttcaggaggctggacccagaagaaaaccacactgctgaactggttatccctgaattacctctggctcaccctcccaatgaaagaacccacctggttatcctgggtgctatcctactgtgtctgggtgttgctctgaccttcatcttcaggttaaggaaaggtagaatgatggatgttaaaaaatgtggtatccaggacaccaactctaaaaaacagtctgacacccacctggaagaaacctgataaaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctcttgaggccaagtgtgactttgtggtgtggctgggttgggggcagcagagggtgaaccctgcaggagggtgaaccctgcaaaagggtggggcagtgggggccaacttgtccttacccagagtgcaggtgtgtggagatccctcctgccttgacattgagcagccttagagggtgggggaggctcaggggtcaggttcctgttcctgcttattggggagttcctggcctggcccttctatgtctccccaggtaccccagtttttctgggttcacccagagtgcagatgcttgaggaggtgggaagggactatttgggggtgtctggctcaggtgccatgcctcactggggctggttggcacctgcatttcctgggagtggggctgtctcagggtagctgggcacggtgttcccttgagtgggggtgtagtgggtgttcctagctgccacgcctttgccttcacctatgggatcgtggctgtcagccttgagggtcagcctggcccaggctcccataggcttaggagaggccgcaattcctacctgttcatccagacagagggggacctggaatcaaagtcaagttggggtagggggtccatggggccatatctggcctgcagacagctctggttagctatgggctgaggtctggattctgccttgtgactggagactgggcgccatcccgtggcctctgagggcccagtttgttcgtgacccatgctgcagagggagatacccaggctcctggacctcagggcctgcctggcactgcaggccaggttctctttttatttctgagcaggggtcttgtgttgcccaggctggagtgtaatggcacaaccatggctcactgcagcctcaactttgggctcaagcaattctcctgcctcagcctcccaagtagctgaaactacacaggcatgccccagcacgcccagctagttgcaggtcagattctcattgttctacagggccctgagttaaatactctcgcaagaagaaaccaagcctggcctgccttccctctgctagaagctgtgattgacaccagcagagaatgaaggaagaaaagggggtgaggatcccttgcagggatgccgagtttgcaggtggcttgactgaaaaaaaaaagaaaaagaaaacacctactttcctctccatggaaacagcatgccagaaaattttgtggacccttgaaatgagcacacatctcacttgcaaaagcacagcaccagcgccctctgctgtttcctggtttgatttagaactcagagaagctacagtactttctagactaaaataccatgtagagttcaggataattatattctagattagacataggcaagcacatttatattagtacattctgtagtatattccagagtgaaggaaatcaaagtttatgactcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacattttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgcctcacacgtacgcgtacgatgcataacttcgtataatgtgtactatacgaagttatataacttcgtataggatactttatacgaagttatgtttcagcggccgcgattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 214 - DNA, 18447 bp, Artificial Sequence
  Payload 13A sequence
ttttgtttatggtgtaagacaagggtctgatttcatttcattttgagggtttcaccctctcatataataaaattcttctatatacccttctatcatcacttattactgattatatgccttgatttcagaatcttgccaactagattcagaatttgactgggagagaggacaaggaccacttgagactcatattttatttccagaatctagcatctacctacctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgacagggtaataagacagtagtgaatatcaagctacaaaaagccgcctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgacccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcatatgcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttactataccatatctttccagaaatatgaaggcttgtgaaagataagcaagcctatgatggccactgtgaaaaggaccagggagaatagaaccatgaggaagacataggtggagtgggagagaggtgtggacaatggttgctcagctgggatcatgttggtcaggttcagcatgtagcccaaagtccagccggcgtcgctgccctggatcttgccaatgaaatggatgtgctcccaggaatcagctgtgaaatgatagccttgcagaaggagggagagaatgtaggtaccagaaaagcagtattcactcaggtacttctcctttactccagcgtaagatgtttttatctcctcccaaggctgagcacagaactttttcatcatctcagtcaccttttcctgagagactttctctgatgtcaagtttaaaaacttcatcacaaagtaaaaagctgaaaatgccccaaaatccccctggagtggtggcaagaaaatcccattgaaggcacactgggagtaagggcagtaactggtgttgaagagctccaggatgctttgatggcattgttgatagtttccaataccctggatttcaaactgctggaatggaagagtcatctcaaatctcttggtgcagggggtcttgtaaaggtcacttacgttcactaccttcttatatccaggatgaaagcatgggtccctgagaatttcattacttgcaacctgaatgtccttggccagtttctgccagagtgcctgatccttcccatagcacaagaagctatgtgtgtagacattgtagtccttgccatagaggcgaaattgcagagcattatctggggactcgatagtctggttttggggtacaaaagtgacttgtgtagaggctcccccaaggtccaaagctccaaaggtttcctgattattggtttcatatgggactatgctgaaccaccttgttttctgactgaatttgcccagcagatagttgatagtaatccagccataggcaccttcctcttggccagtaatgatcctggcaccctggaagtcaaaggggtagttgctgaggctcctctccaccacatccagaaccctgtctgccaactcttcactttccatcctgagcaaccgcatgcctgccgtggctcccaggtaaacgggtgtctcttggtgctgggaccttggaatcacttccctagctctttccatgcaatcagtcaggtaaatgcctatttcatttactttctgaacaaattttgagattccaggacctttaaccctgcattcttctacttgatgcaccacgcctgtgtcattctccttttctgctggccacttatagatgtataaacttgtgtgagaagaacccgcatccagcacaatcccatacttaacgttttctggcaatgctttgttctgggtcaaccccacagcaagcaaagctatcacagctatgatagaggagaagccaaggatggctaggatattcttggagcaaaatgtcttcacgttagactcctttgtatcttccataggtccagggttctcctccacgtctccagcctgcttcagcaggctgaagttagtagctccgcttccaggactcgggtcaaatgcagcgtttgtaaaaactccatatgttcctaggtggctgtgaggattcaaggggcatttcccatcatctttttgtctgaaacgcttgatctgagacatccgctctgcttggcgccttcggtgccggcacctgacacagaagaagatgcctagcccaatgaaaagcaggaggccggcgacgccccccagcacaatcagggccattggctgcaccggggtggaccatgtgggcagaaccttgatgttggattccagcaggacctgtcccgagtcactcagcagacactgccacatccccgcctcagggttcagcacccacaccgccttctcccgcttcgagacctttgcctccttgttctccagtttcaaactcagcatcagcttaggggaggtgggtccccacacctcacaggtcaaatttttctggagctgagtggctctcatcaccaccaggttcacttcctgatgcaactttcctgttttcgcttcaagggccagggtgaggtttccagagccagcatactgaggcaaggcctggggcagggtgaggtggagcgggagcttcttgcccatctggagcttagggtcctgggtaacccgttttacagacacttccttgttcttcaggtcaaaggtgatccaagacttggaggaggaagccctctccgcctgccaccacagctcgccactgcccgtcagcttttcaactgtaaaggcgagtgggaaggagaactccacctgttccccctctttcttatagactatgctggaggccttctggaaagctagcaccacgatgtctattttgaactccaccttcttctggttctgcaagacagtgcatgtccaggtgccactatcctggagctccagctgagacacggagagggtcttcccagatccaccaccaccgcttccgcctccgccacttcctccgccgccagatcctccgcctcccatatttttaacaaaaatttcttcatatgctattttcactctctgcttctttctttttcttttggttttaattaggcctccttttgatattctttggatgaaaccttttttacatgccctcagacattcttgtttggaagtaaaattgttttcatttcccccacatccactgtacttaaatgggcggcatttcccaatgactgaattgtagtagaatctgttctcattggcacgacacaatcctctgtctgctggagtgagacaccatgagggaccgtgaaattcaaaaaggctgggaaccttggttgattgcggagtcagggagttattcacagcattgagctgggttccataattatccacctggaaaccattcggaccatcttcacaaatgttcttgcattcttccagtgtctcaaaattgttcatattgcccaggcatccaccatacttgaaacgttcacactgttttgtctgattgttataaaaatacctggtaatataacctcgacatattccaggatcttcttccaaaaagcagaaatctggcttttcttgttgcaatgttgtctttataatcctgtttgcattatctcttgtacacatttttttgcactcttccagactttcaaatcgattctgatttccttcacatcccccatatataaattcttcgcactgtcgagtgaaaatattgaagaaaaatcttttcatgattgctttacatgggccatcatccgccttgaatgcacaaaatgaatgcataagtttcagtggtggcaactccgtatctgtgataattgtgtgttcttcatcttcctcagaatcagcattaagaggggcaggggcaagattaagcagcaggcatacagaagcccaaagtgcatgtactttcttcattgtgtaaatcataggtccagggttctcctccacgtctccagcctgcttcagcaggctgaagttagtagctccgcttccgagtctctgcggcgtccgctcggtccgcacgtgctgcagcactacctccttggaaggggccgcgcacttgtactccatcttggccctggcggcgccctgcttcttgcgcaggtggcagaggagcgccaaaagcgccaccaccaggcacaggctcgcgatggagatgcctatgagcaagcccgaatgcacgagccccacggccggaggagtcaaggtggagccgggcgtcgggctgggcgggggctcgccagagccgctgtcgccaccgtccaccttgccggagtcacagtcggtgccaatgtggcgggcaagggccgagtcgggcccgcagatgcactcgaaggtaccggggaggttgtggcacaccccggagcagaagccgccgttttcgcactcgtcgatgtccgtgcagatgaaaccgtcgtccaggatgtagccttcagggcactcacagctagcctgggtgttggggtcgcagtcggctggacaggcagtctggttgcaaaacatctggcacctgtgcggctcgtggggaatgggcgcgaagccctcggcgcagacgcagaggtagctagtttggttcaggggctggcactggtactcgcagttggctctgaagcacgggtccacgggctccacacactcgccgtccaccaggtcgtagttagggtagcagtggcactcgaagccaccctgtgtgttgacacagcgctgcggacacggactgggctccagtatgcagtcatccacgtcctcgcaccggtgttggtcggccgccagccggtagccggtctcgcacatgcacgagtaggagcccggctggtcggggttgggaacgcagaagtgctcgcagaggtcgttgcaggactgcgtcgcggatgcggtgcaggagcgcccgtctgcctgcagggcggcgccggctgggcactggcagcggggagccccagggatcgcattgcacgcgtgctcgcagccgccgttctccacgctgcagtcccaagcgcccggcgcctccctggcccagtgcccctggaccgctccgggcggcgcggtgcacattagctgtaagccgaggggagccaccgcggcggagctgcccaccggcagcgcctggaagtccgctccgcgggccgcgaacggggtgccgtaggtgatcgagacggcggcagccgcggcgccgggctccacagccagtggcctgcaggtggctgggaagtggaactcgcagaggaagccatcggccttcacttcgcactgctgctcctcccagatcggctcgctgggcacagtggcctcagcagcggagacagcgacgcacaacgggccgcagaggggagccccattgaggtcgagccgtgcccacctgctatagctggtgttgttgtctcccgtaacccactggaagccgcgcaggggcccgaggcgcttggggtcgccgcagccgggtggcagctgcaggccgatccagaggcgccggcggccaacgccgccgtcgccgttcagtagcaaggaaatgacatcggcagccaccgaggagcgcactgtcattaggtggccccgcagtccgtcgcagatctgactggcattgaggaaggtcgcggggcccgggtagagcgcgaagcagtcgtgctcgacgcactggctgccacccggctgcggctctgcgggtgcggggaaccccaggccggccagggccagcgcgccaaggaccaggaccccaagcatgttacccaggcgttgctttgaattagcggtggttttcacaacacctaaaaaagggtttaaaagatacctttgaaccgctaagaagcccgagaattagctccgctcaaaactcaaggggacaaattccaaaaatgacttccagcgccaggctggcctgactagtctccacccaccaaatgtgaacaaactccaacgccattacatcccctccccccgccgcgactagccgtgctcaaaagcccgaggtgactattgcggccgataggaccacggggtcacaggaagcagcagccggtgagggaccaggccctcttcctttgtgtgggtgactcacccgcccgctccaccgggctgccgcgtcctccattttgagctccttgcaacagggcccgggagcggccatctttccacgcacgcaactggtgccggacgggatggcctcaccctagttagggaggcagggcaacgcggcgccgccaagccagatcgtgccgggtgctggggccacatggcctcggcacgctaaccccagcctggttgcttcgggaaaaaccccaggcctcgccccatccaggtggcgtcggacatgtgctccgaaggcgggcgggccccagccgccactcctgtccctccattcctccccaaccatgacctctccgggctccgggcgagcaagccccgcaccctccctttgttagcccctattgctgaacggcaatcgaaggcagcagggcaacaacaacaacaaaaaaaaaaagaccagagtgcggccggagtagcacgcggcggcggcgcggacaccacgctaggcctcaagccggacacgaggcgaggctacggggttgccgctaggcctcgcactctgcctcccgcgccgcccgcaactcgaagcgggaatgctcgcagctaatccccgccgacgacagcggggcccggcccgctcggagcaggacctccagctcggcggcccgcggaagccacacccgcccctcacctgcgttctgacggcaaacccgttgcgaaaaagaacgtccaaggcgactgccgcacttatataccgttctcccccaccctcgggaagagggcggagccagcacacgacaccgctttcccagtttgccccgcgccagctccccggtcctcccccccatgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaacccggcgcgccatgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgtcttctatctgggcacacgtgtttcacgttgacaggtttgcttgggacgctagtaaccatgggcttgctgactggaagcggagctactaacttcagcctgctgaagcaggctggagacgtggaggagaaccctggacctatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatcccggaagcggagctactaacttcagcctgctgaagcaggctggagacgtggaggagaaccctggacctatggagcctcccggccgccgcgagtgtccctttccttcctggcgctttcctgggttgcttctggcggccatggtgttgctgctgtactccttctccgatgcctgtgaggagccaccaacatttgaagctatggagctcattggtaaaccaaaaccctactatgagattggtgaacgagtagattataagtgtaaaaaaggatacttctatatacctcctcttgccacccatactatttgtgatcggaatcatacatggctacctgtctcagatgacgcctgttatagagaaacatgtccatatatacgggatcctttaaatggccaagcagtccctgcaaatgggacttacgagtttggttatcagatgcactttatttgtaatgagggttattacttaattggtgaagaaattctatattgtgaacttaaaggatcagtagcaatttggagcggtaagcccccaatatgtgaaaaggttttgtgtacaccacctccaaaaataaaaaatggaaaacacacctttagtgaagtagaagtatttgagtatcttgatgcagtaacttatagttgtgatcctgcacctggaccagatccattttcacttattggagagagcacgatttattgtggtgacaattcagtgtggagtcgtgctgctccagagtgtaaagtggtcaaatgtcgatttccagtagtcgaaaatggaaaacagatatcaggatttggaaaaaaattttactacaaagcaacagttatgtttgaatgcgataagggtttttacctcgatggcagcgacacaattgtctgtgacagtaacagtacttgggatcccccagttccaaagtgtcttaaagtgctgcctccatctagtacaaaacctccagctttgagtcattcagtgtcgacttcttccactacaaaatctccagcgtccagtgcctcaggtcctaggcctacttacaagcctccagtctcaaattatccaggatatcctaaacctgaggaaggaatacttgacagtttggatgtttgggtcattgctgtgattgttattgccatagttgttggagttgcagtaatttgtgttgtcccgtacagatatcttcaaaggaggaagaagaaaggcacatacctaactgatgagacccacagagaagtaaaatttacttctctctgataacccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgattatatggccacaaccatggctgaacaagtccttcctcaggctttgtatttgagcaatatgcggaaagctgtgaagatacgggagagaactccagaagacatttttaaacctactaatgggatcattcatcattttaaaaccatgcaccgatacacactggaaatgttcagaacttgccagttttgtcctcagtttcgggagatcatccacaaagccctcatcgacagaaacatccaggccaccctggaaagccagaagaaactcaactggtgtcgagaagtccggaagcttgtggcgctgaaaacgaacggtgacggcaattgcctcatgcatgccacttctcagtacatgtggggcgttcaggacacagacttggtactgaggaaggcgctgttcagcacgctcaaggaaacagacacacgcaactttaaattccgctggcaactggagtctctcaaatctcaggaatttgttgaaacggggctttgctatgatactcggaactggaatgatgaatgggacaatcttatcaaaatggcttccacagacacacccatggcccgaagtggacttcagtacaactcactggaagaaatacacatatttgtcctttgcaacatcctcagaaggccaatcattgtcatttcagacaaaatgctaagaagtttggaatcaggttccaatttcgcccctttgaaagtgggtggaatttacttgcctctccactggcctgcccaggaatgctacagataccccattgttctcggctatgacagccatcattttgtacccttggtgaccctgaaggacagtgggcctgaaatccgagctgttccacttgttaacagagaccggggaagatttgaagacttaaaagttcactttttgacagatcctgaaaatgagatgaaggagaagctcttaaaagagtacttaatggtgatagaaatccccgtccaaggctgggaccatggcacaactcatctcatcaatgccgcaaagttggatgaagctaacttaccaaaagaaatcaatctggtagatgattactttgaacttgttcagcatgagtacaagaaatggcaggaaaacagcgagcaggggaggagagaggggcacgcccagaatcccatggaaccttccgtgccccagctttctctcatggatgtaaaatgtgaaacgcccaactgccccttcttcatgtctgtgaacacccagcctttatgccatgagtgctcagagaggcggcaaaagaatcaaaacaaactcccaaagctgaactccaagccgggccctgaggggctccctggcatggcgctcggggcctctcggggagaagcctatgagcccttggcgtggaaccctgaggagtccactggggggcctcattcggccccaccgacagcacccagcccttttctgttcagtgagaccactgccatgaagtgcaggagccccggctgccccttcacactgaatgtgcagcacaacggattttgtgaacgttgccacaacgcccggcaacttcacgccagccacgccccagaccacacaaggcacttggatcccgggaagtgccaagcctgcctccaggatgttaccaggacatttaatgggatctgcagtacttgcttcaaaaggactacagcagaggcctcctccagcctcagcaccagcctccctccttcctgtcaccagcgttccaagtcagatccctcgcggctcgtccggagcccctccccgcattcttgccacagagctggaaacgacgcccctgctggctgcctgtctcaagctgcacggactcctggggacaggacggggacgagcaagtgcagaaaagccggctgcgtgtattttgggactccagaaaacaagggcttttgcacactgtgtttcatcgagtacagagaaaacaaacattttgctgctgcctcagggaaagtcagtcccacagcgtccaggttccagaacaccattccgtgcctggggagggaatgcggcacccttggaagcaccatgtttgaaggatactgccagaagtgtttcattgaagctcagaatcagagatttcatgaggccaaaaggacagaagagcaactgagatcgagccagcgcagagatgtgcctcgaaccacacaaagcacctcaaggcccaagtgcgcccgggcctcctgcaagaacatcctggcctgccgcagcgaggagctctgcatggagtgtcagcatcccaaccagaggatgggccctggggcccaccggggtgagcctgcccccgaagacccccccaagcagcgttgccgggcccccgcctgtgatcattttggcaatgccaagtgcaacggctactgcaacgaatgctttcagttcaagcagatgtatggcggaagcggagctactaacttcagcctgctgaagcaggctggagacgtggaggagaaccctggacctatgtctcgctccgtggccttagctgtgctcgcgctactctctctttctggcctcgaggctgttatggctccgcggactttaattttaggttgcggcggttccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgggtggtggcggttctggtggtggcggtagtggcggcggaggaagcggtggtggcggttccggatctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctgctacaatcagagcgaggccgggtctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggctctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactataaggctgagtggagcgacagtgcccaggggtctgagtctcacagcttgggaagcggagctactaacttcagcctgctgaagcaggctggagacgtggaggagaaccctggacctatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattcgttttgtgcagaaattttaaggtgactacacgcttagcccaactacccttgggaaatgtgtgtgtgttagtcactcaatcgtgtccagctctttgtgaccccatggactgtagcctgccaggctcctctgtccatgggattctccaggcaagaatactggagtgggttgccattccccaggggattttcccaacccaaggatcaaacccgagtttcctgcattgcaggcagattctttactctctgagccatcagggaagccctgtgggaaatgggaaccatgcaaaaacggctttgggaccaataagaccagaatatttgggatctgaactggcgcaagaaatgtggaagagagattctaaatgcatgtgtacatactaagttgcttcagtcatgtccgactatttgcaaccccatggaccacagcctgccaggctcctctgtccatgggattctccagtcaagaatactggagtgagtttccatttcctcttccaggggatcttccagacccagggatcaaaccagtgtctcttatgtctcctgtgttgacaggcaggttctttaccactagtgccacctgagacccaattctaaacgaggggttgcaaattcagtgccaacaggtgccaggtataagtggcaagggctgca

SEQ ID NO: 215 - DNA, 86 bp, Artificial Sequence
  Synthetic
caccataatcacccccaacgtcctgcgtctggagagtgaggagatggtggtgttggaggcccacgaagggcaaggggatattcggg

SEQ ID NO: 216 - DNA, 35 bp, Artificial Sequence
  Synthetic
caccataatcacccccaagcaaggggatattcggg

SEQ ID NO: 217 - DNA, 35 bp, Artificial Sequence; identical to SEQ ID NO: 216
  Synthetic

SEQ ID NO: 218 - DNA, 35 bp, Artificial Sequence; identical to SEQ ID NO: 216
  Synthetic

SEQ ID NO: 219 - DNA, 35 bp, Artificial Sequence; identical to SEQ ID NO: 216
  Synthetic

SEQ ID NO: 220 - DNA, 34 bp, Artificial Sequence
  Synthetic
caccataatcacccccaacaaggggatattcggg

SEQ ID NO: 221 - DNA, 34 bp, Artificial Sequence; identical to SEQ ID NO: 220
  Synthetic

SEQ ID NO: 222 - DNA, 34 bp, Artificial Sequence; identical to SEQ ID NO: 220
  Synthetic

SEQ ID NO: 223 - DNA, 34 bp, Artificial Sequence; identical to SEQ ID NO: 220
  Synthetic

SEQ ID NO: 224 - DNA, 34 bp, Artificial Sequence; identical to SEQ ID NO: 220
  Synthetic

SEQ ID NO: 225 - DNA, 34 bp, Artificial Sequence; identical to SEQ ID NO: 220
  Synthetic

SEQ ID NO: 226 - DNA, 34 bp, Artificial Sequence; identical to SEQ ID NO: 220
  Synthetic

SEQ ID NO: 227 - DNA, 35 bp, Artificial Sequence; identical to SEQ ID NO: 216
  Synthetic

SEQ ID NO: 228 - DNA, 34 bp, Artificial Sequence; identical to SEQ ID NO: 220
  Synthetic

SEQ ID NO: 229 - DNA, 34 bp, Artificial Sequence
  Synthetic
caccataatctcccccaacaaggggatattcggg

SEQ ID NO: 230 - DNA, 35 bp, Artificial Sequence; identical to SEQ ID NO: 216
  Synthetic

SEQ ID NO: 231 - DNA, 35 bp, Artificial Sequence; identical to SEQ ID NO: 216
  Synthetic

SEQ ID NO: 232 - DNA, 35 bp, Artificial Sequence; identical to SEQ ID NO: 216
  Synthetic

SEQ ID NO: 233 - DNA, 35 bp, Artificial Sequence; identical to SEQ ID NO: 216
  Synthetic

SEQ ID NO: 234 - DNA, 35 bp, Artificial Sequence; identical to SEQ ID NO: 216
  Synthetic

SEQ ID NO: 235 - DNA, 34 bp, Artificial Sequence; identical to SEQ ID NO: 220
  Synthetic

SEQ ID NO: 236 - DNA, 35 bp, Artificial Sequence; identical to SEQ ID NO: 216
  Synthetic

SEQ ID NO: 237 - DNA, 35 bp, Artificial Sequence; identical to SEQ ID NO: 216
  Synthetic

SEQ ID NO: 238 - DNA, 35 bp, Artificial Sequence; identical to SEQ ID NO: 216
  Synthetic

SEQ ID NO: 239 - DNA, 34 bp, Artificial Sequence; identical to SEQ ID NO: 220
  Synthetic

SEQ ID NO: 240 - DNA, 34 bp, Artificial Sequence; identical to SEQ ID NO: 220
  Synthetic

SEQ ID NO: 241 - DNA, 34 bp, Artificial Sequence; identical to SEQ ID NO: 220
  Synthetic

SEQ ID NO: 242 - DNA, 34 bp, Artificial Sequence; identical to SEQ ID NO: 220
  Synthetic

SEQ ID NO: 243 - DNA, 35 bp, Artificial Sequence; identical to SEQ ID NO: 216
  Synthetic

SEQ ID NO: 244 - DNA, 34 bp, Artificial Sequence; identical to SEQ ID NO: 220
  Synthetic

SEQ ID NO: 245 - DNA, 35 bp, Artificial Sequence; identical to SEQ ID NO: 216
  Synthetic

SEQ ID NO: 246 - DNA, 34 bp, Artificial Sequence; identical to SEQ ID NO: 220
  Synthetic

SEQ ID NO: 247 - DNA, 34 bp, Artificial Sequence; identical to SEQ ID NO: 220
  Synthetic

SEQ ID NO: 248 - DNA, 35 bp, Artificial Sequence; identical to SEQ ID NO: 216
  Synthetic

SEQ ID NO: 249 - DNA, 34 bp, Artificial Sequence; identical to SEQ ID NO: 220
  Synthetic

SEQ ID NO: 250 - DNA, 35 bp, Artificial Sequence
  Synthetic
caccataatcacccccaagcaaggggatattcggt

SEQ ID NO: 251 - DNA, 34 bp, Artificial Sequence; identical to SEQ ID NO: 220
  Synthetic

SEQ ID NO: 252 - DNA, 161 bp, Artificial Sequence
  Synthetic
atgaggttcttttctctccctttgttgtccaccttcatgctgaccccgacctagccgaccatgttgcctcctatggcttaaatgtctaccagtcttacggtcccagcggctattatacccatgaatttgatggcgacgaggaattctatgtggacctggag

SEQ ID NO: 253 - DNA, 162 bp, Artificial Sequence
  Synthetic
atgcggttcttttctctccctttgttgtccaccttcatgctgaccccgacctagccgaccatgttgcctcctaatggcttaaatgtctaccagtcttacggtcccagcggctattatacccatgaatttgatggcgacgaggaattctatgtggacctggag

SEQ ID NO: 254 - DNA, 162 bp, Artificial Sequence
  Synthetic
atgaggttcttttctctccctttgttgtccaccttcatgctgaccccgacctagccgaccatgttgcctcctaatggcttaaatgtctaccagtcttacggccccagcggctattatacccatgaatttgatggcgacgaggaattctatgtggacctggag

SEQ ID NO: 255 - DNA, 162 bp, Artificial Sequence
  Synthetic
atgaggttcttttctctccctttgttgtccaccttcatgctgaccccgacctagccgaccatgttgcctcctaatggcttaaatgtctaccagtcttacggtcccagcggctattatacccatgaatttgatggcgacgaggaattctatgtggacctggag

SEQ ID NO: 256 - DNA, 162 bp, Artificial Sequence; identical to SEQ ID NO: 255
  Synthetic

SEQ ID NO: 257 - DNA, 162 bp, Artificial Sequence; identical to SEQ ID NO: 255
  Synthetic

SEQ ID NO: 258 - DNA, 162 bp, Artificial Sequence
  Synthetic
atgaggttcttttctctccctttgttgtccaccttcatgctgaccccgacctagccgaccatgttgcctcctaatggcttaaatgtctaccagtcttacggtcccagcggctattatacccatgaatttggtggcgacgaggaattctatgtggacctggag

SEQ ID NO: 259 - DNA, 162 bp, Artificial Sequence; identical to SEQ ID NO: 255
  Synthetic

SEQ ID NO: 260 - DNA, 162 bp, Artificial Sequence; identical to SEQ ID NO: 255
  Synthetic

SEQ ID NO: 261 - DNA, 162 bp, Artificial Sequence; identical to SEQ ID NO: 255
  Synthetic

SEQ ID NO: 262 - DNA, 162 bp, Artificial Sequence
  Synthetic
atgaggttcttttctctacctttgttgtccaccctcatgctgactacgacctagccgaccatgttgcctcttaatggctgaaatgtctaccagtcttacggtcccagtggctattatacccatgaatttgatgtcgacgaggaattctatgtggacctggag

SEQ ID NO: 263 - DNA, 162 bp, Artificial Sequence; identical to SEQ ID NO: 255
  Synthetic

SEQ ID NO: 264 - DNA, 162 bp, Artificial Sequence; identical to SEQ ID NO: 255
  Synthetic

SEQ ID NO: 265 - DNA, 162 bp, Artificial Sequence
  Synthetic
atgaggttcttttctctccctttgttgtccaccttcatgctgaccccgacctagccgaccatgttgcctcctaatggcttaaatgtctaccagtcttacggtcccagcggctagtatacccatgaatttgacggcgacgaggaattctatgtagacctggag

SEQ ID NO: 266 - DNA, 162 bp, Artificial Sequence
  Synthetic
atgaggttcttttctctccctttgttgtccaccttcatgctgaccccgacctagccgaccatgttgcctcctaatggcttaaatgtctaccagtcttacggtcccagcggctattatacccatgaatttgatggcgacgaggaattctatgtagacctggag

SEQ ID NO: 267 - DNA, 162 bp, Artificial Sequence
  Synthetic
atgaggttcttttctctccctttgttgtccaccttcatgctgaccccgacctagccgaccatgttgcctcctaatggcttaaatgtctactagtcttacggtcccagcggctattatacccatgaatttgatggcgacgaggaattctatgtggacctggag

SEQ ID NO: 268 - DNA, 162 bp, Artificial Sequence; identical to SEQ ID NO: 267
  Synthetic

SEQ ID NO: 269 - DNA, 162 bp, Artificial Sequence; identical to SEQ ID NO: 255
  Synthetic

SEQ ID NO: 270 - DNA, 162 bp, Artificial Sequence
  Synthetic
atgaggttcttttctctccctttgttgtcaaccttcatgctgaccccgacctagccgaccatgttgcctcctaatggcttaaatgtctaccagtcttacggtcccagcggctattacacccatgaatttgatggcgacgaggaattctatgtggacctggag

SEQ ID NO: 271 - DNA, 162 bp, Artificial Sequence; identical to SEQ ID NO: 255
  Synthetic

SEQ ID NO: 272 - DNA, 162 bp, Artificial Sequence; identical to SEQ ID NO: 255
  Synthetic

SEQ ID NO: 273 - DNA, 162 bp, Artificial Sequence; identical to SEQ ID NO: 255
  Synthetic

SEQ ID NO: 274 - DNA, 162 bp, Artificial Sequence; identical to SEQ ID NO: 255
  Synthetic

SEQ ID NO: 275 - DNA, 162 bp, Artificial Sequence; identical to SEQ ID NO: 255
  Synthetic

SEQ ID NO: 276 - DNA, 162 bp, Artificial Sequence; identical to SEQ ID NO: 255
  Synthetic

SEQ ID NO: 277 - DNA, 162 bp, Artificial Sequence
  Synthetic
atgaggttcttttctctccctttgttgtccaccttcatgctgactccgacctagccgaccatgttgcctcctaatggcttaaatgtctaccagtcttacggtcccagcggctattatacccatgaatttgatggcgacgaggaattctatgtggacctggag

SEQ ID NO: 278 - DNA, 162 bp, Artificial Sequence
  Synthetic
atgaggttcttttctctccctttgttgtccaccttcatgctgaccccgacctagccgaccacgttgcctcctaatggcttaaatgtctaccagtcttacggtcccagcggctattatacccatgaatttgatggcgacgaggaattctatgtggacctggag

SEQ ID NO: 279 - DNA, 162 bp, Artificial Sequence
  Synthetic
atgaggttcttttctctccctttgttgtccaccttcatgctgactacgacctagccgaccatgttgcctcctaatggcttaaatgtctaccagtcttacggtcccagcggctattatacccatgaattggatggcgacgaggaattctatgtggaactggag

SEQ ID NO: 280 - DNA, 162 bp, Artificial Sequence
  Synthetic
atgaggttcttttctctccctttgttgtccaccttcatgctgaccccgacctagccgaccatgttgcctcctaatgtcttaaatgtctaccagtcttacggtcccagcggctattatacccatgaatttgatggcgacgagggattctatgtggacctggag

SEQ ID NO: 281 - DNA, 162 bp, Artificial Sequence; identical to SEQ ID NO: 280
  Synthetic

SEQ ID NO: 282 - DNA, 162 bp, Artificial Sequence; identical to SEQ ID NO: 255
  Synthetic

SEQ ID NO: 283 - DNA, 162 bp, Artificial Sequence; identical to SEQ ID NO: 255
  Synthetic

SEQ ID NO: 284 - DNA, 162 bp, Artificial Sequence; identical to SEQ ID NO: 255
  Synthetic

SEQ ID NO: 285 - DNA, 162 bp, Artificial Sequence; identical to SEQ ID NO: 255
  Synthetic

SEQ ID NO: 286 - DNA, 162 bp, Artificial Sequence; identical to SEQ ID NO: 255
  Synthetic

SEQ ID NO: 287 - DNA, 162 bp, Artificial Sequence; identical to SEQ ID NO: 255
  Synthetic

SEQ ID NO: 288 - DNA, 162 bp, Artificial Sequence; identical to SEQ ID NO: 255
  Synthetic

SEQ ID NO: 289 - DNA, 162 bp, Artificial Sequence; identical to SEQ ID NO: 255
  Synthetic

SEQ ID NO: 290 - DNA, 130 bp, Artificial Sequence
  Synthetic
ccatagctctaccgaccctcatcgaggcatctaaggagaaaatgaccatacttggggtcccagtcctaggatttgtcatcaccatcttgaaccttcagaaatcatgggctatcgtaggtaagttctgaga

SEQ ID NO: 291 - DNA, 131 bp, Artificial Sequence
  Synthetic
ccatagctctaccgaccctcatcgaggcatctaaggagaaaatgaccataccttggggtcccagtcctaggatttgtcatcaccatcttgaaccttcagaaatcatgggctatcgtaggtaagttctgaga

SEQ ID NO: 292 - DNA, 131 bp, Artificial Sequence; identical to SEQ ID NO: 291
  Synthetic

SEQ ID NO: 293 - DNA, 131 bp, Artificial Sequence; identical to SEQ ID NO: 291
  Synthetic

SEQ ID NO: 294 - DNA, 131 bp, Artificial Sequence
  Synthetic
ccatagctctaccgaccctcatcgaggcatctaaggagaaagtgaccataccttggggtcccagtcctaggatttgtcatcaccatcttgaaccttcagaaatcatgggctatcgtaggtaagttctgaga

SEQ ID NO: 295 - DNA, 131 bp, Artificial Sequence; identical to SEQ ID NO: 291
  Synthetic

SEQ ID NO: 296 - DNA, 131 bp, Artificial Sequence; identical to SEQ ID NO: 291
  Synthetic

SEQ ID NO: 297 - DNA, 131 bp, Artificial Sequence; identical to SEQ ID NO: 291
  Synthetic

SEQ ID NO: 298 - DNA, 131 bp, Artificial Sequence; identical to SEQ ID NO: 291
  Synthetic

SEQ ID NO: 299 - DNA, 131 bp, Artificial Sequence; identical to SEQ ID NO: 291
  Synthetic

SEQ ID NO: 300 - DNA, 131 bp, Artificial Sequence; identical to SEQ ID NO: 291
  Synthetic

SEQ ID NO: 301 - DNA, 131 bp, Artificial Sequence; identical to SEQ ID NO: 291
  Synthetic

SEQ ID NO: 302 - DNA, 131 bp, Artificial Sequence
  Synthetic
ccatagctctaccgaccctcatcgaggcatctaaggaggaaatgaccataccttggggtcccagtcctaggatttgtcatcaccatcttgaaccttcagaaatcatgggctatcgtaggtaagttctgaga

SEQ ID NO: 303 - DNA, 131 bp, Artificial Sequence; identical to SEQ ID NO: 291
  Synthetic

SEQ ID NO: 304 - DNA, 131 bp, Artificial Sequence
  Synthetic
ccatagctctaccgaccctcatcgaggcatctaaggagaaaatgaccataccttgggatcccagtcctaggatttgtcatcaccatcttgaaccttcagaaatcatgggctatcgtaggtaagttctgaga

SEQ ID NO: 305 - DNA, 131 bp, Artificial Sequence
  Synthetic
ccatagctctaccgaccctcatcgaggcatctaaggagaaaatgaccataccttggggtcccagtcctaggatttgtcatcgccatcttgaaccttcagaaatcatgggctatcgtaggtaagttctgaga

SEQ ID NO: 306 - DNA, 131 bp, Artificial Sequence; identical to SEQ ID NO: 291
  Synthetic

SEQ ID NO: 307 - DNA, 131 bp, Artificial Sequence; identical to SEQ ID NO: 291
  Synthetic

SEQ ID NO: 308 - DNA, 131 bp, Artificial Sequence; identical to SEQ ID NO: 291
  Synthetic

SEQ ID NO: 309 - DNA, 131 bp, Artificial Sequence; identical to SEQ ID NO: 291
  Synthetic

SEQ ID NO: 310 - DNA, 131 bp, Artificial Sequence; identical to SEQ ID NO: 291
  Synthetic

SEQ ID NO: 311 - DNA, 131 bp, Artificial Sequence; identical to SEQ ID NO: 291
  Synthetic

SEQ ID NO: 312 - DNA, 131 bp, Artificial Sequence; identical to SEQ ID NO: 291
  Synthetic

SEQ ID NO: 313 - DNA, 131 bp, Artificial Sequence
  Synthetic
ccatagctctaccgaccctcatcgaggcatctaaggagaaaatgaccataccttggggtcccagtcctaggattcgtcatcaccatcttgaaccttcagaaatcatgggctatcgtaggtaagttctgaga

SEQ ID NO: 314 - DNA, 131 bp, Artificial Sequence; identical to SEQ ID NO: 291
  Synthetic

SEQ ID NO: 315 - DNA, 131 bp, Artificial Sequence
  Synthetic
ccatagctctaccgaccctcatcgaggcatctaaggagaaaatgaccataccttggggtcccagccctaggatttgtcatcaccatcttgaaccttcagaaatcatgggctatcgtaggtaagttctgaga

SEQ ID NO: 316 - DNA, 131 bp, Artificial Sequence; identical to SEQ ID NO: 291
  Synthetic

SEQ ID NO: 317 - DNA, 131 bp, Artificial Sequence; identical to SEQ ID NO: 291
  Synthetic

SEQ ID NO: 318 - DNA, 131 bp, Artificial Sequence
  Synthetic
ccatagctctaccgaccctcatcgaggcatctaaggagaagatgaccataccttggggtcccagtcctaggatttgtcatcaccatcttgaaccttcagaaatcatgggctatcgtaggtaagttccgaga

SEQ ID NO: 319 - DNA, 131 bp, Artificial Sequence; identical to SEQ ID NO: 291
  Synthetic

SEQ ID NO: 320 - DNA, 131 bp, Artificial Sequence
  Synthetic
ccatagctctaccgaccctcatcgaggcatctaaggagaaaatgaccataccttggggtcccagtcctaggatttgtcatcaccatcttgaaccttcagaaatcatgggctattgtaggtaagttctgaga

SEQ ID NO: 321 - DNA, 131 bp, Artificial Sequence
  Synthetic
ccatagctctaccgaccctcatcgaggcatctaaggagaaaatgaccatacctgggggtcccagtcctaggatttgtcatcaccatcttgaaccttcagaaatcatgggctattgtaggtaagttctgaga

SEQ ID NO: 322 - DNA, 131 bp, Artificial Sequence; identical to SEQ ID NO: 291
  Synthetic

SEQ ID NO: 323 - DNA, 131 bp, Artificial Sequence; identical to SEQ ID NO: 291
  Synthetic

SEQ ID NO: 324 - DNA, 131 bp, Artificial Sequence; identical to SEQ ID NO: 291
  Synthetic

SEQ ID NO: 325 - DNA, 131 bp, Artificial Sequence; identical to SEQ ID NO: 291
  Synthetic

SEQ ID NO: 326 - DNA, 131 bp, Artificial Sequence; identical to SEQ ID NO: 291
  Synthetic

SEQ ID NO: 327 - DNA, 131 bp, Artificial Sequence; identical to SEQ ID NO: 291
  Synthetic

SEQ ID NO: 328 - protein, 480 aa, Homo sapiens
ECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPTLPPHMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLP

SEQ ID NO: 329 - protein, 475 aa, Sus scrofa
QCEWRYNSCAPACPVTCQHPEPLACPVSCVEGCHAHCPPGKILDELLQTCVSPEDCPVCEAAGRRLAPGKKIILNPRDPAHCQICHCDGVNLTCEACAEPVPPTEGPVSPTTPYEEDTPEPPLHDFFCSKLLDLVFLLDGSDKLSEADFEALKVFVVGMMEHLHISQKHIRVAVVEYHDGSHAYISLQDRKRPSELRRIASQVKYAGSEVASISEVLKYTLFQIFGRVDRPEASRIALLLMASQEPRRLAQNLARYLQGLKKKKVTVIPVGIGPHVSLKQIRLIEKQAPENKAFVVSGVDELEQRKNEIISYLCDLAPEVPAPTRRPLVAQVTVAPELPGVSTLEPKKRMVLDVVFVLEGSDKVGEANFNRSTEFVEEVIRRMDVGRDSVHVTVLQYSYVVAVEHSFREAQSKGEVLQRVREIRFQGGNRTNTGLALQYLSEHSFSASQGDREEAPNLVYMVTGNPASDEIKRMP

SEQ ID NO: 330 - DNA, 579 bp, Unknown
  biallelic HDR clone
tgggaggtcgggttcagtgggctgggaagggcccagaggctctccctgcaccgttctggtctttcctctcctgcagtcactgtgatggtgtcaatctcacctgtgaagcatgccaagagccgggaggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcaccctgctcctgatggccagccaggagccccaacgga

SEQ ID NO: 331 - protein, 193 aa, Unknown
  biallelic HDR clone
WEVGFSGLGRAQRLSLHRSGLSSSCSHCDGVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQR

SEQ ID NO: 332 - DNA, 463 bp, Unknown
  biallelic HDR clone
ggcctccaccagcgaggtcttgaaatacacactgttccaaatcttcagcaagatcgaccgccctgaagcctcccgcatcaccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgcctccggacatggcccaagtcactgtggcgcctgagctccccggggttcgacgctcgaacccaagaagaaaatggtcttggatgtggtgtttgtgctggaagggtccgacaaggtcg

SEQ ID NO: 333 - protein, 154 aa, Unknown
  biallelic HDR clone
ASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVAPELPGVSTLEPKKKMVLDVVFVLEGSDKV

SEQ ID NO: 334 - DNA, 20 bp, Artificial Sequence
  Synthetic
gcaacacaaacatatctttg

SEQ ID NO: 335 - DNA, 104 bp, Artificial Sequence
  Synthetic
ctattatttctagtatggaaaatatccactgaagtatcaacacaaacatatctccggggttgccatgagctatggtgtgtcacagatgcagctcaggtcccatg

SEQ ID NO: 336 - DNA, 104 bp, Artificial Sequence
  Synthetic
ctattatttctagtatggaaaatatccactgacgtatcaacacaaacatatctccggggttgccatgagctatggtgtgtcacagatgcagctcaggtcccatg

SEQ ID NO: 337 - DNA, 104 bp, Artificial Sequence; identical to SEQ ID NO: 335
  Synthetic

SEQ ID NO: 338 - DNA, 104 bp, Artificial Sequence; identical to SEQ ID NO: 335
  Synthetic

SEQ ID NO: 339 - DNA, 104 bp, Artificial Sequence; identical to SEQ ID NO: 335
  Synthetic

SEQ ID NO: 340 - DNA, 104 bp, Artificial Sequence; identical to SEQ ID NO: 335
  Synthetic

SEQ ID NO: 341 - DNA, 106 bp, Artificial Sequence
  Synthetic
ctattacttctagtatggaaaatatccactgaagtatcaacacaaacatatctatccggggttgccatgagctatggtgtgtcacagatgcagctcaggtcccatg

SEQ ID NO: 342 - DNA, 104 bp, Artificial Sequence; identical to SEQ ID NO: 335
  Synthetic

SEQ ID NO: 343 - DNA, 121 bp, Artificial Sequence
  Synthetic
ctattatttctagtatggaaaatatccactgaagtatcaacacaaacatatctctcagtgggttaactatccggggttgccatgagctatggtgtgtcacagatgcagctcaggtcccatg

SEQ ID NO: 344 - DNA, 47 bp, Artificial Sequence
  Synthetic
tctagctgcatcaggatcatatcgtcgggtcttttttccggctcagt

SEQ ID NO: 345 - DNA, 47 bp, Artificial Sequence
  Synthetic
ccattgctacaggcatcgtggtgtcacgctcgtcgtttggtatggct

SEQ ID NO: 346 - DNA, 54 bp, Artificial Sequence
  Synthetic
cttttcttttcccaggagaaaataatgaatgtcaaaggaagagtggttctgtca

SEQ ID NO: 347 - DNA, 44 bp, Artificial Sequence
  Synthetic
cttttcttttcccaggagaaaataatggaagagtggttctgtca

SEQ ID NO: 348 - DNA, 54 bp, Artificial Sequence; identical to SEQ ID NO: 346
  Synthetic

SEQ ID NO: 349 - DNA, 429 bp, Artificial Sequence
  Synthetic
  misc_feature (15)..(365): n is a, c, g, or t
aggaagctgtgtcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntctgctaaaacatttgccttttcatagcatcgaacaaacgacgcagatcctgttgtgcctctca

SEQ ID NO: 350 - DNA, 38 bp, Artificial Sequence
  Synthetic
aggaagctgtgtcaacgcagatcctgttgtgcctctca

SEQ ID NO: 351 - DNA, 429 bp, Artificial Sequence; identical to SEQ ID NO: 349
  Synthetic
  misc_feature (15)..(365): n is a, c, g, or t

SEQ ID NO: 352 - DNA, 54 bp, Artificial Sequence
  Synthetic
cagcctgtctcctcaggttcactgcggggaggtcacgcgggtcgtaggcatcct

SEQ ID NO: 353 - DNA, 41 bp, Artificial Sequence
  Synthetic
cagcctgtctcctcaggttcactgcgggtcgtaggcatcct

SEQ ID NO: 354 - DNA, 40 bp, Artificial Sequence
  Synthetic
cagcctgtctcctcaggttcactgcggtcgtaggcatcct

SEQ ID NO: 355 - DNA, 41 bp, Artificial Sequence; identical to SEQ ID NO: 353
  Synthetic

SEQ ID NO: 356 - DNA, 40 bp, Artificial Sequence; identical to SEQ ID NO: 354
  Synthetic